(19) United States
(12) Patent Application Publication
(10) Pub. No.: US 2006/0257396 A1
(43) Pub. Date: Nov. 16, 2006

(54) Abeta antibodies for use in improving cognition

(76) Inventor: Jack Steven Jacobsen, Ramsey, NJ (US)

Correspondence Address: LAHIVE & COCKFIELD, 28 STATE STREET, BOSTON, MA, 02109, US

(21) Appl. No.: 11/305,899

(22) Filed: Dec. 15, 2005

Related U.S. Application Data
(60) Provisional application No. 60/636,810, filed on Dec. 15, 2004.
(60) Provisional application No. 60/637,138, filed on Dec. 16, 2004.
(60) Provisional application No. 60/735,687, filed on Nov. 10, 2005.

(51) Int. Cl. A61K 39/395

(52) U.S. Cl. 424/141.1; 424/145.1

(57) ABSTRACT

The invention provides improved agents and methods for treatment of diseases associated with beta amyloid (Aβ). Preferred agents include antibodies, e.g., humanized antibodies specific for Aβ.

### RELATED APPLICATIONS

This application claims the benefit of priority to U.S. provisional patent applications bearing Ser. No. 60/636,810 (filed Dec. 15, 2004), Ser. No. 60/637,138 (filed Dec. 16, 2004), and Ser. No. 60/735,687 (filed Nov. 10, 2005), all entitled “Aβ Antibodies for Use in Improving Cognition.” The entire contents of each of the above-referenced provisional patent applications are incorporated herein by reference.

### BACKGROUND OF THE INVENTION

Memory is a key cognitive function involving the storage and/or retrieval by the brain of information received from past experiences. Learning, also referred to as conditioning, is the process by which new information is acquired and stored by the nervous system to form a memory. In patients with dementia, the cognitive pathways for learning and/or memory are impaired, such that the patient fails to learn or effectively form new memories or recall old ones. The number of individuals exhibiting dementia is rising rapidly, and the rate of rise is expected to increase as the general population continues to age and life expectancy continues to lengthen. Patients with dementia require increasingly costly and intensive caregiving as their symptoms worsen. As such, medical interventions that delay institutionalization would help reduce the demands on healthcare systems, in addition to alleviating the sufferings of the subject with the dementia.

The development of profound dementia is characteristic of several amyloidogenic disorders noted for the accumulation of amyloid protein deposits in the brain tissue of affected subjects, including Down's syndrome, cerebral amyloid angiopathy, vascular dementias, and Alzheimer's disease (AD). AD is a progressive disease resulting in senile dementia. Broadly speaking, the disease falls into two categories: late onset, which occurs in old age (65+years) and early onset, which develops well before the senile period, i.e., between 35 and 60 years.

Neurodegeneration is associated with amyloidogenic disorders and other dementia disorders such that the cognitive symptoms progressively worsen with age. The diagnosis of an amyloidogenic disorder can usually only be confirmed by the distinctive cellular pathology that is evident on post-mortem examination of the brain. The histopathology consists of at least one of three principal features including the presence of neurofibrillary tangles (NT), the diffuse loss of synapses and neurons in central nervous system tissues, and the presence of amyloid plaques (also called senile plaques). See generally Selkoe, TINS 16:403 (1993); Hardy et al., WO 92/13069; Selkoe, J. Neuropathol. Exp. Neurol. 53:438 (1994); Duff et al., Nature 373:476 (1995); Games et al., Nature 373:523 (1995).

The principal constituent of the plaques is a peptide termed Aβ or β-amyloid peptide. Aβ peptide is an approximately 4-kDa internal fragment of 39-43 amino acids of a larger transmembrane glycoprotein named protein termed amyloid precursor protein (APP). As a result of proteolytic processing of APP by different secretase enzymes, Aβ is primarily found in both a short form, 40 amino acids in length, and a long form, ranging from 42-43 amino acids in length. Part of the hydrophobic transmembrane domain of APP is found at the carboxy end of Aβ, and may account for the ability of Aβ to aggregate into plaques, particularly in the case of the long form. Accumulation of amyloid plaques in the brain eventually leads to neuronal cell death. The physical symptoms associated with this type of neural deterioration characterize AD.

Several mutations within the APP protein have been correlated with the presence of AD. See, e.g., Goate et al., Nature 349:704 (1991) (valine⁷¹⁷ to isoleucine); Chartier Harlan et al., Nature 353:844 (1991) (valine⁷¹⁷ to glycine); Murrell et al., Science 254:97 (1991) (valine⁷¹⁷ to phenylalanine); Mullan et al., Nature Genet. 1:345 (1992) (a double mutation changing lysine⁵⁹⁵-methionine⁵⁹⁶ to asparagine⁵⁹⁵-leucine⁵⁹⁶). Such mutations are thought to cause AD by increased or altered processing of APP to Aβ, particularly processing of APP to increased amounts of the long form of Aβ (i.e., Aβ1-42 and Aβ1-43). Mutations in other genes, such as the presenilin genes, PS1 and PS2, are thought indirectly to affect processing of APP to generate increased amounts of long form Aβ (see Hardy, TINS 20: 154 (1997)).

Mouse models have been used successfully to determine the significance of amyloid plaques in AD (Games et al., supra, Johnson-Wood et al., Proc. Natl. Acad. Sci. USA 94:1550 (1997)). In particular, when PDAPP transgenic mice, (which express a mutant form of human APP and develop AD pathology at a young age), are injected with the long form of Aβ, they display both a decrease in the progression of AD Pathology and an increase in antibody titers to the Aβ peptide (Schenk et al., Nature 400, 173 (1999)). The above findings implicate Aβ, particularly in its long form, as a causative element in AD.

Aβ peptide can exist in solution and can be detected in the central nervous system (CNS) (e.g., in cerebral spinal fluid (CSF)) and plasma. Under certain conditions, soluble Aβ is transformed into fibrillary, toxic, β-sheet forms found in neuritic plaques and cerebral blood vessels of patients with AD. Several treatments have been developed which attempt to prevent the formation of Aβ peptide, for example, the use of chemical inhibitors to prevent the cleavage of APP. Immunotherapeutic treatments have also been investigated as a means to reduce the density and size of existing plaques. These strategies include passive immunization with various anti-Aβ antibodies that induce clearance of amyloid deposits, as well as active immunization with soluble forms of Aβ peptide to promote a humoral response that includes generation of anti-Aβ antibodies and cellular clearance of the deposits. Both active and passive immunization have been tested as in mouse models of AD. In PDAPP mice, immunization with Aβ was shown to prevent the development of plaque formation, neuritic dystrophy and astrogliosis. Treatment of older animals also markedly reduced the extent and progression of these AD-like neuropathologies. Schenk et al., supra. Aβimmunization was also shown to reduce plaques and behavioral impairment in the TgCRND8 murine model of AD. Janus et al. (2000) Nature 408:979-982. Aβ immunization also improved cognitive performance and reduced amyloid burden in Tg 2576 APP/PS1 mutant mice. Morgan et al. (2000) Nature 408:982-985. Passive immunization of PDAPP transgenic mice has also been investigated. It was found, for example, that peripherally administered antibodies enter the central nervous system (CNS) and induced plaque clearance in vivo. Bard et al. (2000) Nat. Med. 6:916-919. The antibodies were further shown to induce Fc receptor-mediated phagocytosis in an ex vivo assay. Antibodies specific for the N-terminus of Aβ42 have been demonstrated to be particularly effective in reducing plaque both ex vivo and in vivo. See U.S. Pat. No. 6,761,888 and Bard et al. (2003) Proc. Natl. Acad. Sci. USA 100:2023-2028. Antibodies specific for the mid-region of Aβ42 also showed efficacy. U.S. Pat. No. 6,761,888

Two mechanisms are proposed for effective plaque clearance by immunotherapeutics, i.e., central degradation and peripheral degradation. The central degradation mechanism relies on antibodies being able to cross the blood-brain barrier, bind to plaques, and induce clearance of pre-existing plaques. Clearance has been shown to be promoted through an Fc-receptor-mediated phagocytosis (Bard, et al. (2000) Nat. Med. 6:916-19). The peripheral degradation mechanism of Aβ clearance relies on a disruption of the dynamic equilibrium of Aβ between brain, CSF, and plasma by anti-Aβ antibodies, leading to transport of Aβ from one compartment to another. Centrally derived Aβ is transported into the CSF and the plasma where it is degraded. Recent studies have concluded that soluble and unbound Aβ are involved in the memory impairment associated with AD, even without reduction in amyloid deposition in the brain. Further studies are needed to determine the action and/or interplay of these pathways for Aβ clearance (Dodel, et al., The Lancet, 2003, 2:215)

While the majority of treatments to date have been aimed at reducing amyloid plaque buildup, it has been recently noted that certain cognitive impairments (e.g. hippocampal-dependent conditioning defects) associated with amyloidogenic disorders begin to appear before amyloid deposits and gross neuropathology are evident (Dineley et al., J. Biol. Chem., 2002, 227: 22768). Furthermore, while the pathogenic role of amyloid peptide aggregated into plaques has been known for many years, the severity of dementia or cognitive deficits is only somewhat correlated with the density of plaques whereas a significant correlation exists with the levels of soluble Aβ. (see, e.g., McLean et al., Ann Neurol, 46:860-866 (1999). Some studies have shown or suggested that soluble Aβ oligomers are implicated in synaptotoxicity and memory impairment in APP transgenic mice due to mechanisms including increased oxidative stress and induction of programmed cell death. (See, e.g., Lambert, et al., (1998), PNAS, 95: 6448-53; Naslund et al., (2000), JAMA, 283: 1571; Mucke et al., J Neurosci, 20:4050-4058 (2000); Morgan et al., Nature, 408:982-985 (2000); Dodart et al., Nat Neurosci, 5:452-457 (2002); Selkoe et al., (2002), Science, 298: 789-91; Walsh et al., Nature, 416:535-539 (2002)). These results indicate that neurodegeneration may begin prior to, and is not solely the result of, amyloid deposition. Accordingly, there exists the need for new therapies and reagents for the treatment of AD, in particular, therapies and reagents capable of effecting a therapeutic benefit via intervention with various mechanisms of Aβ-induced neurotoxicity.

### SUMMARY OF THE INVENTION

The present invention features immunological reagents, in particular, therapeutic antibody reagents for the prevention and treatment of Aβ-related diseases or disorders, in particular, for therapeutically effecting improvement in cognition (e.g., rapid improvement in cognition) in patients having or at risk for an Aβ-related disease or disorder. The invention is based, at least in part, on the identification and characterization of several monoclonal antibodies that specifically bind to epitopes within Aβ peptide. The invention features selection of Aβ antibodies having particular activities, in particular, the ability to preferentially bind to soluble, oligomeric Aβ and/or the ability to rapidly improve cognition as determined in an appropriate animal of Aβ-related cognitive deficit.

Structural and functional analysis of these antibodies leads to the design of various humanized antibodies for prophylactic and/or therapeutic use. In particular, the invention features humanization of the variable regions of this antibody and, accordingly, provides for humanized immunoglobulin or antibody chains, intact humanized immunoglobulins or antibodies, and functional immunoglobulin or antibody fragments, in particular, antigen binding fragments, of the featured antibody.

The immunoglobulins described herein are particularly suited for use in therapeutic methods aimed at preventing or treating Aβ-related diseases or disorders or symptoms or indications related thereto. In certain embodiments, the invention features therapeutic and/or prophylactic methods effective in preventing or ameliorating the dementia and/or cognitive deficits that are observed in patients having or at risk for Aβ-related disease or disorders. In particular, the invention provides improved therapeutic and/or prophylactic methods comprising administration of therapeutic agents that interfere at early stages in the pathogenesis of the diseases or disorders and prevent irreversible neural damage and/or dementia. Featured aspects of the present invention provide methods for rapidly improving cognition in a subject that involve administration of an immunological reagent of the invention, or pharmaceutical composition comprising said immunological reagent. Preferred immunological reagents include, but are not limited to antibodies, humanized antibodies, chimeric antibodies, single-chain antibodies, bispecific antibodies, antibody fragments, antibody chains, antibody or antibody claim variants thereof (e.g. Fc antibody variants), or combinations thereof.

In other aspects of the invention, methods for effecting prolonged improvement of cognition in a subject are featured that involve administration of an immunological reagent or pharmaceutical composition comprising said reagent.

In exemplary embodiments, the methods of the invention involve the administration of an immunological reagent, which is effective at binding Aβ, in particular, Aβ oligomers and/or rapidly improving cognition in a subject having or at risk for an Aβ-related disease or disorder.

In some embodiments, an immunoglobulin of the invention comprises one or more alterations in the hinge region, for example, at EU positions 234, 235, 236 and/or 237. In a particular embodiment, an immunoglobulin according to the invention is a humanized 12A11 antibody including amino acid alterations at positions 234 and 237 of the hinge region (i.e., L234A and G237A).

In further embodiments, immunoglobulins of the invention comprise pegylated antibody fragments, e.g., Fabs and Fab's. In yet other embodiments, immunoglobulins of the invention comprise an aglycosylated constant region. In an exemplary embodiment, an immunoglobulin includes an amino acid substitution of an asparagine at position 297 to an alanine, thereby preventing glycosylation of the immunoglobulin.

In some embodiments, a humanized immunoglobulin of the invention comprises complementarity determining regions of a 6C6, 2B1, 1C2 or 9G8 antibody produced by the cell line having ATCC Accession Number ______, ______, ______, or ______, respectively. In some embodiments, a humanized immunoglobulin is a humanized version of a monoclonal antibody 6C6, 2B1, 1C2 or 9G8 produced by a cell line having the ATCC Accession Number ______, ______, ______, or ______, respectively.

Also featured herein are methods of increasing expression of immunoglobulins by deleting one or more introns in a gene which encodes the heavy chain of the immunoglobulin.

Additionally, this invention relates to methods of treatment, as described herein, using one or more immunoglobulins of the invention.

### BRIEF DESCRIPTION OF THE DRAWINGS

FIG. 1 depicts an alignment of the amino acid sequences of the light chain of mouse 3D6 (SEQ ID NO:2), humanized 3D6 version 1 (SEQ ID NO: 6), Kabat ID 109230 (SEQ ID NO: 60) and germline A19 (SEQ ID NO: 61) antibodies. CDR regions are indicated by arrows. Bold italics indicate rare murine residues. Bold indicates packing (VH+VL) residues. Solid fill indicates canonical/CDR interacting residues. Asterisks indicate residues selected for backmutation in humanized 3D6, version 1.

FIG. 2 depicts an alignment of the amino acid sequences of the heavy chain of mouse 3D6 (SEQ ID NO: 4), humanized 3D6 version 1 (SEQ ID NO:8), Kabat ID 045919 (SEQ ID NO: 62) and germline VH3-23 (SEQ ID NO: 63) antibodies. Annotation is the same as for FIG. 1.

FIG. 3 depicts an alignment of the murine 10D5 VL (SEQ ID NO:14) and 3D6 VL (SEQ ID NO:2) amino acid sequences. Bold indicates residues that match 10D5 exactly. CDRs are boxed. Numbering is according to Kabat.

FIG. 4 depicts an alignment of the murine 10D5 VH (SEQ ID NO:16) and 3D6 VH (SEQ ID NO:4) amino acid sequences. Annotation is the same as for FIG. 3.

FIG. 5A-B depicts an alignment of the amino acid sequences of the light chain of mouse 12B4 (mature peptide, SEQ ID NO:18), humanized 12B4 version 1 (mature peptide, SEQ ID NO:22), Kabat ID 005036 (mature peptide, SEQ ID NO:64) and germline A19 (X63397, mature peptide, SEQ ID NO:61) antibodies. CDR regions are stippled and overlined. The single backmutation of a human→mouse residue is indicated by the asterisk. The importance of the shaded residues is shown in the legend. Numbering is according to Kabat.

FIG. 6A-B depicts an alignment of the amino acid sequences of the heavy chain of mouse 12B4 (mature peptide, SEQ ID NO:20), humanized 12B4 (version 1) (mature peptide, SEQ ID NO:24), Kabat ID 000333 (mature peptide, SEQ ID NO:65), and germline VH4-39 and VH4-61 antibodies (mature peptides, SEQ ID NOs: 66 and 67, respectively). Annotation is the same as for FIG. 5.

FIG. 7 depicts a Western blot of immunoprecipitates of peroxynitrite treated oligomeric Aβ₁₋₄₂ preparation precipitated with various Aβ antibodies (3D6, 6C6, 12A11, 12B4, 3A3, 266, 9G8, 15C11, and 6H9) and imaged with 3D6. The approximate positions of Aβ₁₋₄₂ monomer, dimer, trimer and tetramer bands are indicated on the left-hand side of the figure. Indicated below each Aβ antibody is the Aβ epitope recognized by the antibody and contextual fear conditioning (CFC) assay results for the antibody, a “+” notation indicates an observation of increased cognition upon treatment with the antibody, a “−” notation indicates an observation of no change in cognition upon treatment with the antibody, and a “+/−” notation indicates an observation of a trend of increased cognition upon treatment with the antibody but the observed trend was not statistically significant enough to be indicated as an observation of increased cognition.

FIG. 8 depicts a Western blot of immunoprecipitates of peroxynitrite treated oligomeric Aβ₁₄₂ preparation precipitated with various Aβ antibodies (3D6, 6C6, 12A11, 12B4, 10D5, 3A3, 266 and 6H9) and imaged with 3D6. Annotation is the same as for FIG. 7.

FIG. 9A depicts the results of a CFC assay in which rapid improvement in cognition is observed following the administration of single doses of murine 12A11 (1, 10, and 30 mg/kg) to Tg2576 mice. FIG. 9B depicts the results of a CFC assay in which rapid improvement in cognition is observed following the administration of single, low doses of murine 12A11 (0.3 and 1 mg/kg) to Tg2576 mice.

FIG. 10 depicts the effect of thee N-terminal anti-Aβ antibodies (3D6, 12A11, and 266), on contextual-dependent memory in wild-type and Tg2576 mice as determined by a contextual fear conditioning (CFC) assay.

FIG. 11A depicts the results of a study in which the duration of improved cognition following the administration of a single dose of murine 12A11 (1 mg/kg) is assessed at 1, 10, and 17 days post-administration in a contextual fear conditioning (CFC) assay.

FIG. 11B depicts the results of a study in which the duration of improved cognition following the administration of a single dose of murine 266 (3 mg/kg) is assessed at 1, 5, 10, and 17 days post-administration in a contextual fear conditioning (CFC) assay.

FIG. 12 depicts the effect of anti-Aβ antibodies (12A11 and 266), on contextual-dependent memory in wild-type and a doubly transgenic AD mouse model as determined by a contextual fear conditioning (CFC) assay.

FIG. 13 depicts an alignment of the amino acid sequences of the light chain of murine (or chimeric) 12A11 (SEQ ID NO:28), humanized 12A11 version 1 (mature peptide, SEQ ID NO:32), GenBank BAC01733 (SEQ ID NO: 68) and germline A19 (X63397, SEQ ID NO: 61) antibodies. CDR regions are boxed. Packing residues are underlined. Numbering is according to Kabat.

FIG. 14 depicts an alignment of the amino acid sequences of the heavy chain of murine (or chimeric) 12A11 (SEQ ID NO:30), humanized 12A11 (version 1) (mature peptide, SEQ ID NO:34), GenBank AAA69734 (SEQ ID NO:69), and germline GenBank 567123 antibodies (SEQ ID NO:70). Packing residues are underlined, canonical residues are in solid fill and vernier residues are in dotted fill. Numbering is according to Kabat.

FIGS. 15A-B depict an alignment of the amino acid sequences of the heavy chains of humanized 12A11 v1 (SEQ ID NO:34), v2 (SEQ ID NO:35), v2.1 (SEQ ID NO:36), v3 (SEQ ID NO:37), v3.1 (SEQ ID NO:39), v4.1 (SEQ ID NO:40), v4.2 (SEQ ID NO:41), v4.3 (SEQ ID NO:42), v4.4 (SEQ ID NO:43), v5.1 (SEQ ID NO:44), v5.2 (SEQ ID NO:45), v5.3 (SEQ ID NO:46), v5.4 (SEQ ID NO:47), v5.5 (SEQ ID NO:48), v5.6 (SEQ ID NO:49), v6.1 (SEQ ID NO:50), v6.2 (SEQ ID NO:51), v6.3 (SEQ ID NO:52), v6.4 (SEQ ID NO:53), v7 (SEQ ID NO:54) and v8 (SEQ ID NO:55). FIG. 15C sets forth the backmutations made in humanized 12A11 v1 to v8.

FIG. 16 depicts the results of a CFC assay in which rapid improvement in cognition is observed following the administration of single doses of the humanized 12A11 antibody v3.1 h12A11 (1, 10, and 30 mg/kg) to Tg2576 mice.

FIG. 17 is an alignment of the heavy chain variable domains of 15C11, 9G8, 266 and 6H9 anti-Aβ antibodies. Kabat numbering of the amino acids for 15C11 is shown above the sequence. The leader sequence is shown in lower case and the CDRs are bolded.

FIG. 18 is an alignment of the light chain variable domains of 15C11, 9G8 and 266 anti-Aβ antibodies. Kabat numbering of the amino acids for 15C11 is shown above the sequence. The leader sequence is shown in lower case and the CDRs are bolded.

### DETAILED DESCRIPTION OF THE INVENTION

Prior to describing the invention, it may be helpful to an understanding thereof to set forth definitions of certain terms to be used hereinafter.

I) Definitions

The term “Aβ-related disease or disorder” as used herein refers to a disease or disorder associated with, or characterized by, the development or presence of an Aβ peptide. In one embodiment, the Aβ-related disease or disorder is associated with or characterized by the presence of soluble Aβ. In another embodiment, the Aβ-related disease or disorder is associated with or characterized by the presence of insoluble Aβ. In another embodiment, the Aβ-related disease or disorder is associated with or characterized by the presence of a neuroactive Aβ species (NAβ). In another embodiment, the Aβ-related disease or disorder is also an amyloidogenic disorder. In another embodiment, the Aβ-related disease or disorder is characterized by an Aβ-related cognitive deficit or disorder, for example, an Aβ-related dementia disorder. Exemplary Aβ-related diseases or disorders include Alzheimer's disease (AD), Down's syndrome, cerebral amyloid angiopathy, certain vascular dementias, and mild cognitive impairment (MCI).

The terms “β-amyloid protein”, “β-amyloid peptide”, “β-amyloid”, “Aβ” and “Aβ peptide” are used interchangeably herein. Aβ peptide (e.g., Aβ39, Aβ40, Aβ41, Aβ42 and Aβ43) is a ˜4-kDa internal fragment of 39-43 amino acids of the larger transmembrane glycoprotein termed Amyloid Precursor Protein (APP). Multiple isoforms of APP exist, for example APP⁶⁹⁵, APP⁷⁵′, and APP⁷⁷⁰. Amino acids within APP are assigned numbers according to the sequence of the APP⁷⁷⁰ isoform (see e.g., GenBank Accession No. P05067). Examples of specific isotypes of APP which are currently known to exist in humans are the 695 amino acid polypeptide described by Kang et. al. (1987) Nature 325:733-736 which is designated as the “normal” APP; the 751 amino acid polypeptide described by Ponte et al. (1988) Nature 331:525-527 (1988) and Tanzi et al. (1988) Nature 331:528-530; and the 770-amino acid polypeptide described by Kitaguchi et. al. (1988) Nature 331:530-532. As a result of proteolytic processing of APP by different secretase enzymes in vivo or in situ, Aβ is found in both a “short form”, 40 amino acids in length, and a “long form”, ranging from 4243 amino acids in length. The short form, Aβ₄₀, consists of residues 672-711 of APP. The long form, e.g., Aβ₄₂ or Aβ₄₃, consists of residues 672-713 or 672-714, respectively. Part of the hydrophobic domain of APP is found at the carboxy end of Aβ, and may account for the ability of Aβ to aggregate, particularly in the case of the long form. Aβ peptide can be found in, or purified from, the body fluids of humans and other mammals, e.g. cerebrospinal fluid, including both normal individuals and individuals suffering from amyloidogenic disorders.

The terms “β-amyloid protein”, “β-amyloid peptide”, “β-amyloid”, “Aβ” and “Aβ peptide” include peptides resulting from secretase cleavage of APP and synthetic peptides having the same or essentially the same sequence as the cleavage products. Aβ peptides of the invention can be derived from a variety of sources, for example, tissues, cell lines, or body fluids (e.g. sera or cerebrospinal fluid). For example, an Aβ can be derived from APP-expressing cells such as Chinese hamster ovary (CHO) cells stably transfected with APP_{717V→F}, as described, for example, in Walsh et al., (2002), Nature, 416, pp 535-539. An Aβ preparation can be derived from tissue sources using methods previously described (see, e.g., Johnson-Wood et al., (1997), Proc. Natl. Acad. Sci. USA 94:1550). Alternatively, Aβ peptides can be synthesized using methods which are well known to those in the art. See, for example, Fields et al., Synthetic Peptides: A User's Guide, ed. Grant, W.H. Freeman & Co., New York, N.Y., 1992, p 77). Hence, peptides can be synthesized using the automated Merrifield techniques of solid phase synthesis with the α-amino group protected by either t-Boc or F-moc chemistry using side chain protected amino acids on, for example, an Applied Biosystems Peptide Synthesizer Model 430A or 431. Longer peptide antigens can be synthesized using well known recombinant DNA techniques. For example, a polynucleotide encoding the peptide or fusion peptide can be synthesized or molecularly cloned and inserted in a suitable expression vector for the transfection and heterologous expression by a suitable host cell. Aβ peptide also refers to related Aβsequences that results from mutations in the Aβ region of the normal gene.

The term “soluble Aβ” or “dissociated Aβ” refers to non-aggregating or disaggregated Aβ polypeptide, including monomeric soluble as well as oligomeric soluble Aβ polypeptide (e.g., soluble Aβ dimers, trimers, and the like). Soluble Aβ can be found in vivo in biological fluids such as cerebrospinal fluid and/or serum. Soluble Aβ can also be prepared in vitro, e.g., by solubilizing Aβ peptide in appropriate solvents and/or solutions. For example, soluble Aβ can be prepared by dissolving lyophilized peptide in alcohol, e.g., HFIP followed by dilution into cold aqueous solution. Alternatively, soluble Aβ can be prepared by dissolving lyophilized peptide in neat DMSO with sonication. The resulting solution can be centrifuged (e.g., at 14,000×g, 4° C., 10 minutes) to remove any insoluble particulates.

The term “insoluble Aβ” or “aggregated Aβ” refers to aggregated Aβpolypeptide, for example, Aβ held together by noncovalent bonds and which can occur in the fibrillary, toxic, β-sheet form of Aβ peptide that is found in neuritic plaques and cerebral blood vessels of patients with AD. Aβ (e.g., Aβ42) is believed to aggregate, at least in part, due to the presence of hydrophobic residues at the C-terminus of the peptide (part of the transmembrane domain of APP).

As used herein, the phrase “neuroactive Aβ species” refers to an Aβspecies (e.g., an Aβ peptide or form of Aβ peptide) that effects at least one activity or physical characteristic of a neuronal cell. Neuroactive Aβ species effect, for example, the function, biological activity, viability, morphology and/or architecture of a neuronal cell. The effect on neuronal cells can be cellular, for example, effecting the long-term-potentiation (LPT) of a neuronal cell or viability of a neuronal cell (neurotoxicity). Alternatively, the effect can be on an in vivo neuronal system, for example, effecting a behavioral outcome in an appropriate animal test (e.g., a cognitive test). The term “neutralize” as used herein means to make neutral, counteract or make ineffective an activity or effect.

As used herein, the term “neurodegenerative disease” refers broadly to disorders or diseases associated with or characterized by degeneration of neurons and/or nervous tissues, e.g. an amyloidogenic disease.

The term “amyloidogenic disease” or “amyloidogenic disorder” includes any disease associated with (or caused by) the formation or deposition of insoluble amyloid fibrils. Exemplary amyloidogenic diseases include, but are not limited to systemic amyloidosis, Alzheimer's disease (AD), cerebral amyloid angiopathy (CAA), mature onset diabetes, Parkinson's disease, Huntington's disease, fronto-temporal dementia, and the prion-related transmissible spongiform encephalopathies (kuru and Creutzfeldt-Jacob disease in humans and scrapie and BSE in sheep and cattle, respectively). Different amyloidogenic diseases are defined or characterized by the nature of the polypeptide component of the fibrils deposited. For example, in subjects or patients having Alzheimer's disease, β-amyloid protein (e.g., wild-type, variant, or truncated β-amyloid protein) is the principal polypeptide component of the amyloid deposit. Accordingly, Alzheimer's disease is an example of a “disease characterized by deposits of Aβ” or a “disease associated with deposits of Aβ”, e.g., in the brain of a subject or patient. Other diseases characterized by deposits of Aβ can include uncharacterized diseases where amyloidogenic deposits are found in one or more regions of the brain associated with learning and/or memory, e.g., the hippocampus, amygdala, subiculum, cingulated cortex, prefrontal cortex, perirhinal cortex, sensory cortex, and medial temporal lobe.

The term “cognition” refers to cognitive mental processes performed by a subject including, but not limited to, learning or memory (e.g., short-term or long term learning or memory), knowledge, awareness, attention and concentration, judgment, visual recognition, abstract thinking, executive functions, language, visual-spatial (i.e., visuo-spatial orientation) skills, visual recognition, balance/agility and sensorimotor activity. Exemplary cognitive processes include learning and memory.

The terms “cognitive disorder”, “cognitive deficit”, or “cognitive impairment” are used interchangeably herein and refer to a deficiency or impairment in one or more cognitive mental processes of a subject. Cognitive deficits may have a number of origins: a functional mechanism (anxiety, depression), physiological aging (age-associated memory impairment), brain injury, psychiatric disorders (e.g. schizophrenia), drugs, infections, toxicants, or anatomical lesions. Exemplary cognitive deficits include deficiency or impairment in learning or memory (e.g., in short-term or long term learning and/or memory loss of intellectual abilities, judgment, language, motor skills, and/or abstract thinking).

As used herein, the term “Aβ-related cognitive disorder” (or “deficit” or “impairment”) refers to a cognitive disorder associated with, or characterized by, the development or presence of an Aβ peptide. In one embodiment, the Aβ-related disease or disorder is associated with or characterized by the presence of soluble Aβ. In another embodiment, the Aβ-related disease or disorder is associated with or characterized by the presence of insoluble Aβ. In another embodiment, the Aβ-related disease or disorder is associated with or characterized by the presence of a neuroactive Aβ species (NAβ).

The term “dementia disorder”, as used herein, refers to a disorder characterized by dementia (i.e., general deterioration or progressive decline of cognitive abilities or dementia-like symptoms). Dementia disorders are often associated with, or caused by, one or more aberrant processes in the brain or central nervous system (e.g. neurodegeneration). Dementia disorders commonly progress from mild through severe stages and interfere with the ability of a subject to function independently in everyday life. Dementia may be classified as cortical or subcortical depending on the area of the brain affected. Dementia disorders do not include disorders characterized by a loss of consciousness (as in delirium) or depression, or other functional mental disorders (pseudodementia). Dementia disorders include the irreversible dementias such Alzheimer's disease, vascular dementia, Lewy body dementia, Jakob-Creutzfeldt disease, Pick's disease, progressive supranuclear palsy, Frontal lobe dementia, idiopathic basal ganglia calcification, Huntington disease, multiple sclerosis, and Parkinson's disease, as well as reversible dementias due to trauma (posttraumatic encephalopathy), intracranial tumors (primary or metastatic), subdural hematomas, metabolic and endocrinologic conditions (hypo- and hyperthyroidism, Wilson's disease, uremic encephalopathy, dialysis dementia, anoxic and post-anoxic dementia, and chronic electrolyte disturbances), deficiency states (Vitamin B12 deficiency and pellagra (vitamin B6)), infections (AIDS, syphilitic meningoencephalitis, limbic encephalitis, progressive multifocal leukoencephalopathy, fungal infections, tuberculosis), and chronic exposure to alcohol, aluminum, heavy metals (arsenic, lead, mercury, manganese), or prescription drugs (anticholinergics, sedatives, barbiturates, etc.).

As used herein, the term “Aβ-related dementia disorder” refers to a dementia disorder associated with, or characterized by, the development or presence of an Aβ peptide.

As used herein, the phrase “improvement in cognition” refers to an enhancement or increase in a cognitive skill or function. Likewise, the phrase “improving cognition” refers to the enhancing or increasing of a cognitive skill or function. An improvement in cognition is relative, for example, to cognition in the subject before a treatment according to the instant invention. Preferably, the improvement in cognition trends towards that of a normal subject or towards a standard or expected level.

The term “rapid”, as used, for example, in the phrase “rapid improvement in cognition” (or “rapidly improving cognition”) means taking a relatively or comparatively short time or occurring within a comparatively short time interval; i.e., that an effect (e.g., improvement) is accomplished, observed or achieved comparatively quickly, in terms of clinical relevance.

An exemplary “rapid improvement in cognition” is accomplished, observed or achieved within one day (i.e., within 24 hours). A “rapid improvement in cognition” may be accomplished, observed or achieved in less than one day (i.e., less than 24 hours), for example, within 23, 22, 21, 20, 29, 18, 17, 16, 15, 14, 13, 12, 11, 10, 9, 8, 7, 6, 5, 4, 3, 2 or 1 hour(s). A “rapid improvement in cognition” may alternatively be accomplished, observed or achieved in more than one day but preferably within one month, for example, within 31, 30, 29, 28, 27, 26, 25, 24, 23, 22, 21, 20, 19, 18, 17, 16, 15, 14, 13, 12, 11, 10, 9, 8, 7, 6, 5, 4, 3 or 2 days. Exemplary time intervals for accomplishing, observing or achieving a rapid improvement in cognition are within weeks, e.g., within three weeks, within two weeks or within one week or within, for example, 120 hours, 96 hours, 72 hours, 48 hours, 24 hours, 18 hours, 12 hours and/or 6 hours.

The term “prolonged”, as used, for example, in the phrase “prolonged improvement in cognition” means occurring over a comparatively or relatively longer time interval than a suitable control; i.e., that a desired effect (e.g., improvement) occurs or is observed to be sustained without interruption for an extended or protracted time period, in terms of clinical relevance.

An exemplary “prolonged improvement in cognition” is accomplished, observed or achieved for at least one week. A “prolonged improvement in cognition” may be accomplished, observed or achieved for more than one day (i.e., more than 24 hours), for example, for more than 36 hours, 48 hours (i.e., 2 days), 72 hours (i.e., 3 days), 96 hours (i.e., 4 days) 108 hours (i.e., 5 days) or 132 hours (i.e., 6 days). A “prolonged improvement in cognition” may alternatively be accomplished, observed or achieved for more than one week, e.g., for 8, 9, 10, 11, 12, 13, or 14 days (i.e., two weeks), three weeks, four weeks, five weeks, six weeks, or more. Exemplary time intervals over which a prolonged improvement in cognition is accomplished, observed or achieved include 5, 6, 7, 8, 9, 10, 11, 12, 13, 14, 15, 16, 17, 18, 19 or 20 days.

The term “modulation” as used herein refers to both upregulation, i.e. stimulation, and downregulation, i.e. suppression, of a response.

The term “treatment” as used herein, is defined as the application or administration of a therapeutic reagent to a patient, or application or administration of a therapeutic reagent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the disease, the patient's general physiology. e.g., the patient's body mass, age, gender, the route of administration, and other factors well known to physicians and/or pharmacologists. Effective doses may be expressed, for example, as the total mass of antibody (e.g., in grams, milligrams or micrograms) or as a ratio of mass of antibody to body mass (e.g., as grams per kilogram (g/kg), milligrams per kilogram (mg/kg), or micrograms per kilogram (μg/kg). An effective dose of antibody used in the present methods will range, for example, between 1 μg/kg and 500 mg/kg. An exemplary range for effective doses of antibodies used in the methods of the present invention is between 0.1 mg/kg and 100 mg/kg. Exemplary effective doses include, but are not limited to, 10 μg/kg, 30 μg/kg, 100 μg/kg, 300 μg/kg, 1 mg/kg, 30 mg/kg and 100 mg/kg.

As used herein, the term “administering” refers to the act of introducing a pharmaceutical agent into a subject's body. An exemplary route of administration in the parenteral route, e.g., subcutaneous, intravenous or intraperitoneal administration.

The terms “patient” includes human and other mammalian subjects that receive either prophylactic or therapeutic treatment with one or more agents (e.g., immunotherapeutic agents) of the invention. Exemplary patients receive either prophylactic or therapeutic treatment with the immunotherapeutic agents of the invention.

The term “animal model” or “model animal”, as used herein, includes a member of a mammalian species such as rodents, non-human primates, sheep, dogs, and cows that exhibit features or characteristics of a certain system of disease or disorder, e.g., a human system, disease or disorder. Exemplary non-human animals selected from the rodent family include rabbits, guinea pigs, rats and mice, most preferably mice. An “animal model” of, or “model animal” having, a dementia disorder exhibits, for example, prominent cognitive deficits associated with a dementia-related disorder (e.g., AD). Preferably the model animal exhibits a progressive worsening of the cognitive deficit with increasing age, such that the disease progression in the model animal parallels the disease progression in a subject suffering from the dementia disorder.

The term “immunological reagent” refers to an agent that comprises or consists of one or more immunoglobulins, antibodies, antibody fragments or antibody chains, as defined herein, or combinations thereof. The term “immunological reagent” also includes nucleic acids encoding immunoglobulins, antibodies, antibody fragments, or antibody chains. Such nucleic acids can be DNA or RNA. A nucleic acid encoding an immunoglobulin is typically linked to regulatory elements, such as a promoter and enhancer, that allow expression of the nucleic acid in an appropriate cell or tissue.

When referencing (e.g., preceded by) the name of a particular monoclonal Aβ antibody, the term “immunological reagent” refers to an immunological reagent having one or more characteristics of the referenced monoclonal antibody. Characteristics of the Aβ monoclonal antibodies can include, for example, specific binding to Aβ peptide, preferential binding for soluble oligomeric AD, the ability to reduce plaque burden associated with amyloidogenic disorders in a subject and improve cognition in an animal model of AD. In certain aspects, immunological reagents have conserved structural features with the referenced monoclonal antibody, for example, conserved antigen binding domains or regions (e.g., one or more CDRs of the referenced monoclonal antibody). Exemplary immunological reagents include monoclonal antibodies, humanized versions of said antibodies, chimeric versions of said antibodies, single-chains of said antibodies, bispecific versions of said antibodies, fragments, variants of said antibodies (e.g. affinity matured antibody variants and Fc antibody variants), or combinations thereof. When referencing (e.g., preceeded by) the name of a particular monoclonal Aβ antibody, the term “immunological reagent” also includes any antibody (e.g. humanized antibody, chimeric antibody, single-chain antibody, bispecific antibody), antibody fragment, or antibody chain comprising at least one domain, region, or fragment derived from the referenced antibody, a fragment of said antibody, or chain of said antibody.

The term “immunotherapeutic reagent” refers to an immunological reagent suitable for therapeutic use.

The term “immunoglobulin” or “antibody” (used interchangeably herein) refers to a protein having a basic four-polypeptide chain structure consisting of two heavy and two light chains, said chains being stabilized, for example, by interchain disulfide bonds, which has the ability to specifically bind antigen. It is intended that the term “antibody” encompass any Ig class or any Ig subclass (e.g. the IgG1, IgG2, IgG3, and IgG4 subclasses of IgG) obtained from any source (e.g., in exemplary embodiments, humans and non-human primates, and in additional embodiments, rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD and IgE. The term “Ig subclass” refers to the two subclasses of IgM (H and L), three subclasses of IgA (IgA1, IgA2, and secretory IgA), and four subclasses of IgG (IgG₁, IgG₂, IgG₃, and IgG₄) that have been identified in humans and higher mammals.

The term “IgG subclass” refers to the four subclasses of immunoglobulin class IgG-IgG₁, IgG₂, IgG₃, and IgG₄ that have been identified in humans and higher mammals by the γ heavy chains of the immunoglobulins, γ₁-γ₄, respectively.

The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen.

The term “domain” refers to a globular region of a heavy or light chain polypeptide comprising peptide loops (e.g., comprising 3 to 4 peptide loops) stabilized, for example, by α-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

As used herein, the term “antigen binding site” refers to a site that specifically binds (immunoreacts with) an antigen (e.g., a cell surface or soluble antigen). Antibodies of the invention preferably comprise at least two antigen binding sites. An antigen binding site commonly includes immunoglobulin heavy chain and light chain CDRs and the binding site formed by these CDRs determines the specificity of the antibody. An “antigen binding region” or “antigen binding domain” is a region or domain (e.g., an antibody region or domain that includes an antibody binding site as defined supra.

Immunoglobulins or antibodies can exist in monomeric or polymeric form, for example, IgM antibodies which exist in pentameric form and/or IgA antibodies which exist in monomeric, dimeric or multimeric form. The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc and/or Fv fragments. The term “antigen-binding fragment” refers to a polypeptide fragment of an immunoglobulin or antibody that binds antigen or competes with intact antibody (i.e., with the intact antibody from which they were derived) for antigen binding (i.e., specific binding). Binding fragments are produced by recombinant DNA techniques, or by enzymatic or chemical cleavage of intact immunoglobulins. Binding fragments include Fab, Fab′, F(ab′)₂, Fabc, Fv, single chains, and single chain antibodies. Other than “bispecific” or “bifunctional” immunoglobulins or antibodies, an immunoglobulin or antibody is understood to have each of its antigen-binding sites identical. A “bispecific” or “bifunctional antibody” is an artificial hybrid antibody having two different heavy/light chain pairs and two different antigen-binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody. See, Queen et al., Proc. Natl. Acad. Sci. USA 86:10029-10033 (1989), U.S. Pat. No. 5,530,101, U.S. Pat. No. 5,585,089, U.S. Pat. No. 5,693,761, U.S. Pat. No. 5,693,762, Selick et al., WO 90/07861, and Winter, U.S. Pat. No. 5,225,539 (incorporated by reference in their entirety for all purposes).

A “humanized immunoglobulin” or “humanized antibody” of this invention can be made using any of the methods described herein or those that are well known in the art.

The phrase “substantially from a human immunoglobulin or antibody” or “substantially human” means that, when aligned to a human immunoglobulin or antibody amino sequence for comparison purposes, the region shares at least 80-90%, 90-95%, or 95-99% identity (i.e., local sequence identity) with the human framework or constant region sequence, allowing, for example, for conservative substitutions, consensus sequence substitutions, germline substitutions, backmutations, and the like. The introduction of conservative substitutions, consensus sequence substitutions, germline substitutions, backmutations, and the like, is often referred to as “optimization” of a humanized antibody or chain. The phrase “substantially from a non-human immunoglobulin or antibody” or “substantially non-human” means having an immunoglobulin or antibody sequence at least 80-95%, preferably at least 90-95%, more preferably, 96%, 97%, 98%, or 99% identical to that of a non-human organism, e.g., a non-human mammal.

Accordingly, all regions or residues of a humanized immunoglobulin or antibody, or of a humanized immunoglobulin or antibody chain, except possibly the CDRs, are substantially identical to the corresponding regions or residues of one or more native human immunoglobulin sequences. The term “corresponding region” or “corresponding residue” refers to a region or residue on a second amino acid or nucleotide sequence which occupies the same (i.e., equivalent) position as a region or residue on a first amino acid or nucleotide sequence, when the first and second sequences are optimally aligned for comparison purposes.

The assignment of amino acids to each domain is in accordance with the definitions of Kabat, Sequences of Proteins of Immunological Interest (National Institutes of Health, Bethesda, Md., 1987 and 1991). An alternative structural definition has been proposed by Chothia et al., J. Mol. Biol. 196:901 (1987); Nature 342:878 (1989); and J. Mol. Biol. 186:651 (1989) (hereinafter collectively referred to as “Chothia et al.” and incorporated by reference in their entirety for all purposes).

The term “significant identity” means that two polypeptide sequences, when optimally aligned, such as by the programs GAP or BESTFIT using default gap weights, share at least 50-60% sequence identity, preferably at least 60-70% sequence identity, more preferably at least 70-80% sequence identity, more preferably at least 80-90% identity, even more preferably at least 90-95% identity, and even more preferably at least 95% sequence identity or more (e.g., 99% sequence identity or more). The term “substantial identity” means that two polypeptide sequences, when optimally aligned, such as by the programs GAP or BESTFIT using default gap weights, share at least 80-90% sequence identity, preferably at least 90-95% sequence identity, and more preferably at least 95% sequence identity or more (e.g., 99% sequence identity or more). For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

Preferably, residue positions which are not identical differ by conservative amino acid substitutions. For purposes of classifying amino acids substitutions as conservative or nonconservative, amino acids are grouped as follows: Group I (hydrophobic sidechains): leu, met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Preferably, humanized immunoglobulins or antibodies bind antigen with an affinity that is within a factor of three, four, or five of that of the corresponding non-humanized antibody. For example, if the nonhumanized antibody has a binding affinity of 10⁹ M⁻¹, humanized antibodies will have a binding affinity of at least 3×10⁹ M⁻¹, 4×10⁹ M⁻¹ or 5×10⁹ M⁻¹. When describing the binding properties of an immunoglobulin or antibody chain, the chain can be described based on its ability to “direct antigen (e.g., Aβ) binding”. A chain is said to “direct antigen binding” when it confers upon an intact immunoglobulin or antibody (or antigen binding fragment thereof) a specific binding property or binding affinity. A mutation (e.g., a backmutation) is said to substantially affect the ability of a heavy or light chain to direct antigen binding if it affects (e.g., decreases) the binding affinity of an intact immunoglobulin or antibody (or antigen binding fragment thereof) comprising said chain by at least an order of magnitude compared to that of the antibody (or antigen binding fragment thereof) comprising an equivalent chain lacking said mutation. A mutation “does not substantially affect (e.g., decrease) the ability of a chain to direct antigen binding” if it affects (e.g., decreases) the binding affinity of an intact immunoglobulin or antibody (or antigen binding fragment thereof) comprising said chain by only a factor of two, three, or four of that of the antibody (or antigen binding fragment thereof) comprising an equivalent chain lacking said mutation.

The term “chimeric immunoglobulin” or antibody refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species. The terms “humanized immunoglobulin” or “humanized antibody” are not intended to encompass chimeric immunoglobulins or antibodies, as defined infra. Although humanized immunoglobulins or antibodies are chimeric in their construction (i.e., comprise regions from more than one species of protein), they include additional features (i.e., variable regions comprising donor CDR residues and acceptor framework residues) not found in chimeric immunoglobulins or antibodies, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

“Specific binding” of an antibody means that the antibody exhibits appreciable affinity for a particular antigen or epitope and, generally, does not exhibit significant crossreactivity. In exemplary embodiments, the antibody exhibits no crossreactivity (e.g., does not crossreact with non-Aβ peptides or with remote epitopes on Aβ). “Appreciable” or preferred binding includes binding with an affinity of at least 10⁶, 10⁷, 10⁸, 10⁹ M⁻¹, or 10¹⁰ M⁻¹. Affinities greater than 10⁷ M⁻¹, preferably greater than 10⁸ M⁻¹ are more preferred. Values intermediate of those set forth herein are also intended to be within the scope of the present invention and a preferred binding affinity can be indicated as a range of affinities, for example, 10⁶ to 10¹⁰ M⁻¹, preferably 10⁷ to 10¹⁰ M⁻¹, more preferably 10⁸ to 10¹⁰ M⁻¹. An antibody that “does not exhibit significant crossreactivity” is one that will not appreciably bind to an undesirable entity (e.g., an undesirable proteinaceous entity). For example, an antibody that specifically binds to Aβ will appreciably bind Aβ but will not significantly react with non-Aβ proteins or peptides (e.g., non-Aβ proteins or peptides included in plaques). An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The BIACORE™ method relies on the phenomenon of surface plasmon resonance (SPR), which occurs when surface plasmon waves are excited at a metal/liquid interface. Light is directed at, and reflected from, the side of the surface not in contact with sample, and SPR causes a reduction in the reflected light intensity at a specific combination of angle and wavelength. Bimolecular binding events cause changes in the refractive index at the surface layer, which are detected as changes in the SPR signal.

The dissociation constant, KD, and the association constant, KA, are quantitative measures of affinity. At equilibrium, free antigen (Ag) and free antibody (Ab) are in equilibrium with antigen-antibody complex (Ag−Ab), and the rate constants, ka and kd, quantitate the rates of the individual reactions:

$$\begin{matrix}ka \\ Ag + Ab ⇄ Ag - Ab \\ kd\end{matrix}$$

At equilibrium, ka [Ab][Ag]=kd [Ag−Ab]. The dissociation constant, KD, is given by: KD=kd/ka=[Ag][Ab]/[Ag−Ab]. KD has units of concentration, most typically M, mM, μM, nM, pM, etc. When comparing antibody affinities expressed as KD, having greater affinity for Aβ is indicated by a lower value. The association constant, KA, is given by: KA=KA/KD=[Ag−Ab]/[Ag][Ab]. KA has units of inverse concentration, most typically M⁻¹, mM⁻¹, μM⁻¹, nM⁻¹, pM⁻¹, etc. As used herein, the term “avidity” refers to the strength of the antigen-antibody bond after formation of reversible complexes.

The term “Fc immunoglobulin variant” or “Fc antibody variant” includes immunoglobulins or antibodies (e.g., humanized immunoglobulins, chimeric immunoglobulins, single chain antibodies, antibody fragments, etc.) having an altered Fc region. Fc regions can be altered, for example, such that the immunoglobulin has an altered effector function. An amino acid alteration includes an amino acid substitution, addition, deletion and/or modification of one or more amino acids of an immunoglobulin, for example, in the Fc region of the immunoglobulin. In some embodiments, immunoglobulins of the invention include one or more mutations in the Fc region. In some embodiments, the Fc region includes one or more amino acid alterations in the hinge region, for example, at EU numbering positions 234, 235, 236 and/or 237. Antibodies including hinge mutations at one or more of the amino acid positions 234, 235, 236 and/or 237, can be made, as described in, for example, U.S. Pat. No. 5,624,821, and U.S. Pat. No. 5,648,260, incorporated by reference herein.

The term “effector function” refers to an activity that resides in the Fc region of an antibody (e.g., an IgG antibody) and includes, for example, the ability of the antibody to bind effector molecules such as complement and/or Fc receptors, which can control several immune functions of the antibody such as effector cell activity, lysis, complement-mediated activity, antibody clearance, and antibody half-life.

The term “effector molecule” refers to a molecule that is capable of binding to the Fc region of an antibody (e.g., an IgG antibody) including, but not limited to, a complement protein or a Fc receptor.

The term “effector cell” refers to a cell capable of binding to the Fc portion of an antibody (e.g., an IgG antibody) typically via an Fc receptor expressed on the surface of the effector cell including, but not limited to, lymphocytes, e.g., antigen presenting cells and T cells.

The term “Fc region” refers to a C-terminal region of an IgG antibody, in particular, the C-terminal region of the heavy chain(s) of said IgG antibody. Although the boundaries of the Fc region of an IgG heavy chain can vary slightly, a Fc region is typically defined as spanning from about amino acid residue Cys226 to the carboxyl-terminus of a human IgG heavy chain(s).

The term “aglycosylated” antibody refers to an antibody lacking one or more carbohydrates by virtue of a chemical or enzymatic process, mutation of one or more glycosylation sites, expression in bacteria, etc. An aglycosylated antibody may be a deglycosylated antibody, that is an antibody for which the Fc carbohydrate has been removed, for example, chemically or enzymatically. Alternatively, the aglycosylated antibody may be a nonglycosylated or unglycosylated antibody, that is an antibody that was expressed without Fc carbohydrate, for example by mutation of one or more residues that encode the glycosylation pattern or by expression in an organism that does not attach carbohydrates to proteins, for example bacteria.

“Kabat numbering” unless otherwise stated, is as taught in Kabat et al. (Sequences of Proteins of Immunological Interest, 5th Ed. Public Health Service, National Institutes of Health, Bethesda, Md. (1991)), expressly incorporated herein by reference. “EU numbering” unless otherwise stated, is also taught in Kabat et al. (Sequences of Proteins of Immunological Interest, 5th Ed. Public Health Service, National Institutes of Health, Bethesda, Md. (1991)) and, for example, refers to the numbering of the residues in heavy chain antibody sequences using the EU index as described therein. This numbering system is based on the sequence of the Eu antibody described in Edelman et al., 63(1):78-85 (1969).

The term “Fc receptor” or “FcR” refers to a receptor that binds to the Fc region of an antibody. Typical Fc receptors which bind to an Fc region of an antibody (e.g., an IgG antibody) include, but are not limited to, receptors of the FcγRI, FcγRII, and FcγRIII subclasses, including allelic variants and alternatively spliced forms of these receptors. Other Fc receptors include the neonatal Fc receptors (FcRn) which regulate antibody half-life. Fc receptors are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995).

An “antigen” is an entity (e.g., a proteinaceous entity or peptide) to which an immunoglobulin or antibody (or antigen-binding fragment thereof) specifically binds.

The term “epitope” or “antigenic determinant” refers to a site on an antigen to which an immunoglobulin or antibody (or antigen binding fragment thereof) specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, G. E. Morris, Ed. (1996).

Antibodies that recognize the same epitope can be identified in a simple immunoassay showing the ability of one antibody to block the binding of another antibody to a target antigen, i.e., a competitive binding assay. Competitive binding is determined in an assay in which the immunoglobulin under test inhibits specific binding of a reference antibody to a common antigen, such as Aβ. Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al, Scand. J. Immunol. 32:77 (1990)). Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more.

An epitope is also recognized by immunologic cells, for example, B cells and/or T cells. Cellular recognition of an epitope can be determined by in vitro assays that measure antigen-dependent proliferation, as determined by ³H-thymidine incorporation, by cytokine secretion, by antibody secretion, or by antigen-dependent killing (cytotoxic T lymphocyte assay).

Exemplary epitopes or antigenic determinants to which an antibody of the invention binds can be found within the human amyloid precursor protein (APP), but are preferably found within the Aβ peptide of APP. Exemplary epitopes or antigenic determinants within Aβ, as described herein, are located within the N-terminus, central region, or C-terminus of Aβ.

An “N-terminal epitope”, is an epitope or antigenic determinant comprising residues located within the N-terminus of Aβ peptide. Exemplary N-terminal epitopes include residues within amino acids 1-10 or 1-12 of Aβ, preferably from residues 1-3, 1-4, 1-5, 1-6, 1-7, 2-6, 3-6, or 3-7 of Aβ42. Other exemplary N-terminal epitopes start at residues 1-3 and end at residues 7-11 of Aβ. Additional exemplary N-terminal epitopes include residues 2-4, 5, 6, 7 or 8 of Aβ, residues 3-5, 6, 7, 8 or 9 of Aβ, or residues 4-7, 8, 9 or 10 of Aβ42.

“Central epitopes” are epitopes or antigenic determinants comprising residues located within the central or mid-portion of the Aβ peptide. Exemplary central epitopes include residues within amino acids 13-28, preferably 16-21, 16-22, 16-23, 16-24, 18-21, 19-21, 19-22, 19-23, or 19-24 of Aβ.

“C-terminal epitopes” are epitopes or antigenic determinants comprising residues located within the C-terminus of the Aβ peptide (e.g., within about amino acids 30-40 or 30-42 of Aβ)). Additional exemplary epitopes or antigenic determinants include residues 33-40 or 33-42 of Aβ. Such epitopes can be referred to as “C-terminal epitopes”.

When an antibody is said to bind to an epitope within specified residues, such as Aβ 3-7, what is meant is that the antibody specifically binds to a polypeptide containing the specified residues (i.e., Aβ 3-7 in this an example). Such an antibody does not necessarily contact every residue within Aβ 3-7. Nor does every single amino acid substitution or deletion within Aβ 3-7 necessarily significantly affect binding affinity.

The terms “Aβ antibody” and “anti-Aβ” are used interchangeably herein to refer to an antibody that binds to one or more epitopes or antigenic determinants within Aβ protein. Exemplary Aβ antibodies include N-terminal Aβ antibodies, central Aβ antibodies, and C-terminal Aβ antibodies. As used herein, the term “N-terminal Aβantibody” shall refer to an Aβ antibody that recognizes at least one N-terminal epitope or antigenic determinant. As used herein, the term “central Aβ antibody” shall refer to an Aβ antibody that recognizes at least one central epitope or antigenic determinant. As used herein, the term “C-terminal Aβ antibody” shall refer to an Aβ antibody that recognizes at least one C-terminal epitope or antigenic determinant.

The term “immunological” or “immune” response is the development of a humoral (antibody mediated) and/or a cellular (mediated by antigen-specific T cells or their secretion products) response directed against an antigen in a subject. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, natural killer (“NK”) cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays (see Burke, REF; Tigges, REF). The relative contributions of humoral and cellular responses to the protective or therapeutic effect of an immunogen can be distinguished by separately isolating antibodies and T-cells from an immunized animal or individual and measuring protective or therapeutic effect in a second subject.

As used herein, the term “immunotherapy” refers to a treatment, for example, a therapeutic or prophylactic treatment, of a disease or disorder intended to and/or producing an immune response (e.g., an active or passive immune response).

An “immunogenic agent” or “immunogen” is capable of inducing an immunological response against itself on administration to a patient, optionally in conjunction with an adjuvant. An “immunogenic composition” is one that comprises an immunogenic agent.

The term “adjuvant” refers to a compound that when administered in conjunction with an antigen augments the immune response to the antigen, but when administered alone does not generate an immune response to the antigen. Adjuvants can augment an immune response by several mechanisms including lymphocyte recruitment, stimulation of B and/or T cells, and stimulation of macrophages.

As used herein, the term “kit” is used in reference to a combination of reagents and other materials which facilitate sample analysis. In some embodiments, the immunoassay kit of the present invention includes a suitable antigen, binding agent comprising a detectable moiety, and detection reagents. A system for amplifying the signal produced by detectable moieties may or may not also be included in the kit. Furthermore, in other embodiments, the kit includes, but is not limited to, components such as apparatus for sample collection, sample tubes, holders, trays, racks, dishes, plates, instructions to the kit user, solutions or other chemical reagents, and samples to be used for standardization, normalization, and/or control samples.

Various methodologies of the instant invention include a step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing a methodology of the invention, as described herein. In another embodiment, a “suitable control” or “appropriate control” is a value, level, feature, characteristic, property, etc. determined in a subject, e.g., a control or normal subject exhibiting, for example, normal traits. In yet another embodiment, a “suitable control” or “appropriate control” is a predefined value, level, feature, characteristic, property, etc.

II. Immunological and Therapeutic Reagents

Immunological and therapeutic reagents of the invention comprise or consist of immunogens or antibodies, or functional or antigen binding fragments thereof, as defined herein. The basic antibody structural unit is known to comprise a tetramer of subunits. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Light chains are classified as either kappa or lambda and are about 230 residues in length. Heavy chains are classified as gamma (γ), mu (μ), alpha (α), delta (δ), or epsilon (ε), are about 450-600 residues in length, and define the antibody's isotype as IgG, IgM, IgA, IgD and IgE, respectively. Both heavy and light chains are folded into domains. The term “domain” refers to a globular region of a protein, for example, an immunoglobulin or antibody. Immunoglobulin or antibody domains include, for example, three or four peptide loops stabilized by β-pleated sheet and an interchain disulfide bond. Intact light chains have, for example, two domains (V_{L} and C_{L}) and intact heavy chains have, for example, four or five domains (V_{H}, C_{H}1, C_{H}2, and C_{H}3).

Within light and heavy chains, the variable and constant regions are joined by a “J” region of about 12 or more amino acids, with the heavy chain also including a “D” region of about 10 more amino acids. (See generally, Fundamental Immunology (Paul, W., ed., 2nd ed. Raven Press, N.Y. (1989), Ch. 7, incorporated by reference in its entirety for all purposes).

The variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. Naturally-occurring chains or recombinantly produced chains can be expressed with a leader sequence which is removed during cellular processing to produce a mature chain. Mature chains can also be recombinantly produced having a non-naturally occurring leader sequence, for example, to enhance secretion or alter the processing of a particular chain of interest.

The CDRs of the two mature chains of each pair are aligned by the framework regions, enabling binding to a specific epitope. From N-terminal to C-terminal, both light and heavy chains comprise the domains FR1, CDR1, FR2, CDR2, FR3, CDR3 and FR4. “FR4” also is referred to in the art as the D/J region of the variable heavy chain and the J region of the variable light chain. The assignment of amino acids to each domain is in accordance with the definitions of Kabat.

A. Aβ Antibodies

Therapeutic agents of the invention include antibodies that specifically bind to Aβ. Preferred antibodies are monoclonal antibodies. In some embodiments, antibodies of the invention for use in passive immunotherapy bind soluble Aβ peptide including monomeric soluble and/or oligomeric soluble Aβ polypeptide (e.g., soluble Aβ dimers, trimers, and the like). In other embodiments, the antibody is capable of capturing soluble Aβ, including monomeric soluble as well as oligomeric soluble Aβpolypeptide (e.g., soluble Aβ dimers, trimers, and the like), and preventing accumulation of Aβ and/or promoting removal of Aβ from the CNS. In a particularly preferred embodiment, the antibodies of the invention are capable of binding to both soluble and insoluble Aβ peptides or fragments thereof, and are capable of preventing the formation of additional amyloid plaque while also decreasing the size and density of existing amyloid plaques. In other exemplary embodiments, antibodies demonstrating efficacy in an appropriate animal model for Aβ-related cognitive deficit are selected as reagents for use in the therapeutic methods of the invention.

In addition to the above activities, some antibodies selected for use in the methodologies of the invention bind to aggregated Aβ. Some bind to soluble Aβ. Some bind to both aggregated and soluble forms. Some antibodies bind Aβ in plaques. Some antibodies can cross the blood-brain barrier. Some antibodies can reduce amyloid burden in a subject. Some antibodies can reduce neuritic dystrophy in a subject. Some antibodies can maintain synaptic architecture (e.g., synaptophysin). Some antibodies can neutralize one or more neuroactive forms of Aβ.

In certain embodiments of the invention, the antibody is capable of binding aggregated or insoluble Aβ deposited in plaques to decrease the size or density of amyloid plaques. Where plaque-clearing is desired, antibodies can be selected which have an intact constant region or at least sufficient of the constant region to interact with an Fc receptor. Exemplary antibodies are those efficacious at stimulating Fc-mediated phagocytosis of Aβ in plaques. Human isotype IgG1 can be used in humanized antibodies of the invention because of it having highest affinity of human isotypes for the FcRI receptor on phagocytic cells (e.g., on brain resident macrophages or microglial cells). Human IgG1 is the equivalent of murine IgG2a, the latter thus suitable for testing in vivo efficacy in animal (e.g., mouse) models of Alzheimer's. Bispecific Fab fragments can also be used, in which one arm of the antibody has specificity for Aβ, and the other for an Fc receptor. Preferred antibodies bind to Aβ with a binding affinity greater than (or equal to) about 10⁶, 10⁷, 10⁸, 10⁹, or 10¹⁰ M⁻¹ (including affinities intermediate of these values).

Antibodies of the present invention also include those antibodies which are capable of binding and/or clearing soluble AD in the CNS or brain of a subject. Exemplary antibodies also include those antibodies which are capable of capturing soluble Aβ, e.g., in the bloodstream of a subject. Preferred antibodies are capable of rapidly improving cognition in a subject, e.g., via clearance and/or capture of soluble Aβ.

Monoclonal antibodies bind to a specific epitope within Aβ that can be a conformational or nonconformational epitope. Prophylactic and therapeutic efficacy of antibodies can be tested using the transgenic animal model procedures described in the Examples. Exemplary monoclonal antibodies bind to an epitope within residues 1-10 of Aβ (with the first N terminal residue of natural Aβ designated 1), for example, to an epitope within residues 3-7 of Aβ. Other exemplary monoclonal antibodies bind to an epitope within residues 13-28 of Aβ, for example, to an epitope within residues 16-24 of Aβ. In some methods, multiple monoclonal antibodies having binding specificities to different epitopes are used, for example, an antibody specific for an epitope within residues 3-7 of Aβ can be co-administered with an antibody specific for an epitope outside of residues 3-7 of Aβ (e.g., an antibody specific for an epitope within residues 16-24 of Aβ). Such antibodies can be administered sequentially or simultaneously. Antibodies to amyloid components other than Aβ can also be used (e.g., administered or co-administered).

Epitope specificity of an antibody can be determined, for example, by forming a phage display library in which different members display different subsequences of Aβ. The phage display library is then selected for members specifically binding to an antibody under test. A family of sequences is isolated. Typically, such a family contains a common core sequence, and varying lengths of flanking sequences in different members. The shortest core sequence showing specific binding to the antibody defines the epitope bound by the antibody. Antibodies can also be tested for epitope specificity in a competition assay with an antibody whose epitope specificity has already been determined.

Antibodies that specifically bind to a preferred segment of Aβ without binding to other regions of Aβ have a number of advantages relative to monoclonal antibodies binding to other regions or polyclonal sera to intact Aβ. First, for equal mass dosages, dosages of antibodies that specifically bind to preferred segments contain a higher molar dosage of antibodies effective in clearing amyloid plaques. Second, antibodies specifically binding to preferred segments can induce a clearing response against amyloid deposits without inducing a clearing response against intact APP polypeptide, thereby reducing the potential side effects.

1. Production of Nonhuman Antibodies

The present invention features non-human antibodies, for example, antibodies having specificity for the preferred Aβ epitopes of the invention. Such antibodies can be used in formulating various therapeutic compositions of the invention or, preferably, provide complementarity determining regions for the production of humanized or chimeric antibodies (described in detail below). The production of non-human monoclonal antibodies, e.g., murine, guinea pig, primate, rabbit or rat, can be accomplished by, for example, immunizing the animal with Aβ. A longer polypeptide comprising Aβ or an immunogenic fragment of Aβ or anti-idiotypic antibodies to an antibody to Aβ can also be used. See Harlow & Lane, supra, incorporated by reference for all purposes). Such an immunogen can be obtained from a natural source, by peptide synthesis or by recombinant expression. Optionally, the immunogen can be administered fused or otherwise complexed with a carrier protein, as described below. Optionally, the immunogen can be administered with an adjuvant. The term “adjuvant” refers to a compound that when administered in conjunction with an antigen augments the immune response to the antigen, but when administered alone does not generate an immune response to the antigen. Adjuvants can augment an immune response by several mechanisms including lymphocyte recruitment, stimulation of B and/or T cells, and stimulation of macrophages. Several types of adjuvant can be used as described below. Complete Freund's adjuvant followed by incomplete adjuvant is preferred for immunization of laboratory animals.

Rabbits or guinea pigs are typically used for making polyclonal antibodies. Exemplary preparation of polyclonal antibodies, e.g., for passive protection, can be performed as follows. 125 non-transgenic mice are immunized with 100 μg Aβ1-42, plus CFA/IFA adjuvant, and euthanized at 4-5 months. Blood is collected from immunized mice. IgG is separated from other blood components. Antibody specific for the immunogen may be partially purified by affinity chromatography. An average of about 0.5-1 mg of immunogen-specific antibody is obtained per mouse, giving a total of 60-120 mg.

Mice are typically used for making monoclonal antibodies. Monoclonals can be prepared against a fragment by injecting the fragment or longer form of Aβ into a mouse, preparing hybridomas and screening the hybridomas for an antibody that specifically binds to Aβ. Optionally, antibodies are screened for binding to a specific region or desired fragment of Aβ without binding to other nonoverlapping fragments of Aβ. The latter screening can be accomplished by determining binding of an antibody to a collection of deletion mutants of an Aβ peptide and determining which deletion mutants bind to the antibody. Binding can be assessed, for example, by Western blot or ELISA. The smallest fragment to show specific binding to the antibody defines the epitope of the antibody. Alternatively, epitope specificity can be determined by a competition assay is which a test and reference antibody compete for binding to Aβ. If the test and reference antibodies compete, then they bind to the same epitope or epitopes sufficiently proximal such that binding of one antibody interferes with binding of the other. The preferred isotype for such antibodies is mouse isotype IgG2a or equivalent isotype in other species. Mouse isotype IgG2a is the equivalent of human isotype IgG1 (e.g., human IgG1).

2. Chimeric and Humanized Antibodies

The present invention also features chimeric and/or humanized antibodies (i.e., chimeric and/or humanized immunoglobulins) specific for beta amyloid peptide. Chimeric and/or humanized antibodies have the same or similar binding specificity and affinity as a mouse or other nonhuman antibody that provides the starting material for construction of a chimeric or humanized antibody.

a. Production of Chimeric Antibodies

The term “chimeric antibody” refers to an antibody whose light and heavy chain genes have been constructed, typically by genetic engineering, from immunoglobulin gene segments belonging to different species. For example, the variable (V) segments of the genes from a mouse monoclonal antibody may be joined to human constant (C) segments, such as IgG1 and IgG4. Human isotypes IgG1 and IgG4 are exemplary. A typical chimeric antibody is thus a hybrid protein consisting of the V or antigen-binding domain from a mouse antibody and the C or effector domain from a human antibody.

b. Production of Humanized Antibodies

The term “humanized antibody” refers to an antibody comprising at least one chain comprising variable region framework residues substantially from a human antibody chain (referred to as the acceptor immunoglobulin or antibody) and at least one complementarity determining region substantially from a mouse antibody, (referred to as the donor immunoglobulin or antibody). See, Queen et al., Proc. Natl. Acad. Sci. USA 86:10029-10033 (1989), U.S. Pat. No. 5,530,101, U.S. Pat. No. 5,585,089, U.S. Pat. No. 5,693,761, U.S. Pat. No. 5,693,762, Selick et al., WO 90/07861, and Winter, U.S. Pat. No. 5,225,539 (incorporated by reference in their entirety for all purposes). The constant region(s), if present, are also substantially or entirely from a human immunoglobulin.

The substitution of mouse CDRs into a human variable domain framework is most likely to result in retention of their correct spatial orientation if the human variable domain framework adopts the same or similar conformation to the mouse variable framework from which the CDRs originated. This is achieved by obtaining the human variable domains from human antibodies whose framework sequences exhibit a high degree of sequence identity with the murine variable framework domains from which the CDRs were derived. The heavy and light chain variable framework regions can be derived from the same or different human antibody sequences. The human antibody sequences can be the sequences of naturally occurring human antibodies or can be consensus sequences of several human antibodies. See Kettleborough et al., Protein Engineering 4:773 (1991); Kolbinger et al., Protein Engineering 6:971 (1993) and Carter et al., WO 92/22653.

Having identified the complementarity determining regions of the murine donor immunoglobulin and appropriate human acceptor immunoglobulins, the next step is to determine which, if any, residues from these components should be substituted to optimize the properties of the resulting humanized antibody. In general, substitution of human amino acid residues with murine should be minimized, because introduction of murine residues increases the risk of the antibody eliciting a human-anti-mouse-antibody (HAMA) response in humans. Art-recognized methods of determining immune response can be performed to monitor a HAMA response in a particular patient or during clinical trials. Patients administered humanized antibodies can be given an immunogenicity assessment at the beginning and throughout the administration of said therapy. The HAMA response is measured, for example, by detecting antibodies to the humanized therapeutic reagent, in serum samples from the patient using a method known to one in the art, including surface plasmon resonance technology (BIACORE) and/or solid-phase ELISA analysis.

Certain amino acids from the human variable region framework residues are selected for substitution based on their possible influence on CDR conformation and/or binding to antigen. The unnatural juxtaposition of murine CDR regions with human variable framework region can result in unnatural conformational restraints, which, unless corrected by substitution of certain amino acid residues, lead to loss of binding affinity.

The selection of amino acid residues for substitution is determined, in part, by computer modeling. Computer hardware and software are described herein for producing three-dimensional images of immunoglobulin molecules. In general, molecular models are produced starting from solved structures for immunoglobulin chains or domains thereof. The chains to be modeled are compared for amino acid sequence similarity with chains or domains of solved three-dimensional structures, and the chains or domains showing the greatest sequence similarity is/are selected as starting points for construction of the molecular model. Chains or domains sharing at least 50% sequence identity are selected for modeling, and preferably those sharing at least 60%, 70%, 80%, 90% sequence identity or more are selected for modeling. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

The selection of amino acid residues for substitution can also be determined, in part, by examination of the characteristics of the amino acids at particular locations, or empirical observation of the effects of substitution or mutagenesis of particular amino acids. For example, when an amino acid differs between a murine variable region framework residue and a selected human variable region framework residue, the human framework amino acid should usually be substituted by the equivalent framework amino acid from the mouse antibody when it is reasonably expected that the amino acid:

(1) noncovalently binds antigen directly,

(2) is adjacent to a CDR region,

(3) otherwise interacts with a CDR region (e.g., is within about 3-6 Å of a CDR region as determined by computer modeling), or

(4) participates in the VL-VH interface.

Residues which “noncovalently bind antigen directly” include amino acids in positions in framework regions which have a good probability of directly interacting with amino acids on the antigen according to established chemical forces, for example, by hydrogen bonding, Van der Waals forces, hydrophobic interactions, and the like.

CDR and framework regions are as defined by Kabat et al. or Chothia et al., supra. When framework residues, as defined by Kabat et al., supra, constitute structural loop residues as defined by Chothia et al., supra, the amino acids present in the mouse antibody may be selected for substitution into the humanized antibody. Residues which are “adjacent to a CDR region” include amino acid residues in positions immediately adjacent to one or more of the CDRs in the primary sequence of the humanized immunoglobulin chain, for example, in positions immediately adjacent to a CDR as defined by Kabat, or a CDR as defined by Chothia (See e.g., Chothia and Lesk JMB 196:901 (1987)). These amino acids are particularly likely to interact with the amino acids in the CDRs and, if chosen from the acceptor, to distort the donor CDRs and reduce affinity. Moreover, the adjacent amino acids may interact directly with the antigen (Amit et al., Science, 233:747 (1986), which is incorporated herein by reference) and selecting these amino acids from the donor may be desirable to keep all the antigen contacts that provide affinity in the original antibody.

Residues that “otherwise interact with a CDR region” include those that are determined by secondary structural analysis to be in a spatial orientation sufficient to affect a CDR region. In one embodiment, residues that “otherwise interact with a CDR region” are identified by analyzing a three-dimensional model of the donor immunoglobulin (e.g., a computer-generated model). A three-dimensional model, typically of the original donor antibody, shows that certain amino acids outside of the CDRs are close to the CDRs and have a good probability of interacting with amino acids in the CDRs by hydrogen bonding, Van der Waals forces, hydrophobic interactions, etc. At those amino acid positions, the donor immunoglobulin amino acid rather than the acceptor immunoglobulin amino acid may be selected. Amino acids according to this criterion will generally have a side chain atom within about 3 angstrom units (Å) of some atom in the CDRs and must contain an atom that could interact with the CDR atoms according to established chemical forces, such as those listed above.

In the case of atoms that may form a hydrogen bond, the 3 Å is measured between their nuclei, but for atoms that do not form a bond, the 3 Å is measured between their Van der Waals surfaces. Hence, in the latter case, the nuclei must be within about 6 Å (3 Å plus the sum of the Van der Waals radii) for the atoms to be considered capable of interacting. In many cases the nuclei will be from 4 or 5 to 6 Å apart. In determining whether an amino acid can interact with the CDRs, it is preferred not to consider the last 8 amino acids of heavy chain CDR 2 as part of the CDRs, because from the viewpoint of structure, these 8 amino acids behave more as part of the framework.

Amino acids that are capable of interacting with amino acids in the CDRs, may be identified in yet another way. The solvent accessible surface area of each framework amino acid is calculated in two ways: (1) in the intact antibody, and (2) in a hypothetical molecule consisting of the antibody with its CDRs removed. A significant difference between these numbers of about 10 square angstroms or more shows that access of the framework amino acid to solvent is at least partly blocked by the CDRs, and therefore that the amino acid is making contact with the CDRs. Solvent accessible surface area of an amino acid may be calculated based on a three-dimensional model of an antibody, using algorithms known in the art (e.g., Connolly, J. Appl. Cryst. 16:548 (1983) and Lee and Richards, J. Mol. Biol. 55:379 (1971), both of which are incorporated herein by reference). Framework amino acids may also occasionally interact with the CDRs indirectly, by affecting the conformation of another framework amino acid that in turn contacts the CDRs.

The amino acids at several positions in the framework are known to be important for determining CDR conformation (e.g., capable of interacting with the CDRs) in many antibodies (Chothia and Lesk, supra, Chothia et al., supra and Tramontano et al., J. Mol. Biol. 215:175 (1990), all of which are incorporated herein by reference). These authors identified conserved framework residues important for CDR conformation by analysis of the structures of several known antibodies. The antibodies analyzed fell into a limited number of structural or “canonical” classes based on the conformation of the CDRs. Conserved framework residues within members of a canonical class are referred to as “canonical” residues. Canonical residues include residues 2, 25, 29, 30, 33, 48, 64, 71, 90, 94 and 95 of the light chain and residues 24, 26, 29, 34, 54, 55, 71 and 94 of the heavy chain. Additional residues (e.g., CDR structure-determining residues) can be identified according to the methodology of Martin and Thorton (1996) J. Mol. Biol. 263:800. Notably, the amino acids at positions 2, 48, 64 and 71 of the light chain and 26-30, 71 and 94 of the heavy chain (numbering according to Kabat) are known to be capable of interacting with the CDRs in many antibodies. The amino acids at positions 35 in the light chain and 93 and 103 in the heavy chain are also likely to interact with the CDRs. Additional residues which may effect conformation of the CDRs can be identified according to the methodology of Foote and Winter (1992) J. Mol. Biol. 224:487. Such residues are termed “vernier” residues and are those residues in the framework region closely underlying (i.e., forming a “platform” under) the CDRs. At all these numbered positions, choice of the donor amino acid rather than the acceptor amino acid (when they differ) to be in the humanized immunoglobulin is preferred. On the other hand, certain residues capable of interacting with the CDR region, such as the first 5 amino acids of the light chain, may sometimes be chosen from the acceptor immunoglobulin without loss of affinity in the humanized immunoglobulin.

Residues which “participate in the VL-VH interface” or “packing residues” include those residues at the interface between VL and VH as defined, for example, by Novotny and Haber, Proc. Natl. Acad. Sci. USA, 82:4592-66 (1985) or Chothia et al, supra. Generally, rare packing residues should be retained in the humanized antibody if they differ from those in the human frameworks.

In general, one or more of the amino acids fulfilling the above criteria can be substituted. In some embodiments, all or most of the amino acids fulfilling the above criteria are substituted. Occasionally, there is some ambiguity about whether a particular amino acid meets the above criteria, and alternative variant immunoglobulins are produced, one of which has that particular substitution, the other of which does not. Alternative variant immunoglobulins so produced can be tested in any of the assays described herein for the desired activity, and the preferred immunoglobulin selected.

Usually the CDR regions in humanized antibodies are substantially identical, and more usually, identical to the corresponding CDR regions of the donor antibody. However, in certain embodiments, it may be desirable to modify one or more CDR regions to modify the antigen binding specificity of the antibody and/or reduce the immunogenicity of the antibody. Typically, one or more residues of a CDR are altered to modify binding to achieve a more favored on-rate of binding, a more favored off-rate of binding, or both, such that an idealized binding constant is achieved. Using this strategy, an antibody having ultra high binding affinity of, for example, 10¹⁰ M⁻¹ or more, can be achieved. Briefly, the donor CDR sequence is referred to as a base sequence from which one or more residues are then altered. Affinity maturation techniques, as described herein, can be used to alter the CDR region(s) followed by screening of the resultant binding molecules for the desired change in binding. The method may also be used to alter the donor CDR, typically a mouse CDR, to be less immunogenic such that a potential human anti-mouse antibody (HAMA) response is minimized or avoided. Accordingly, as CDR(s) are altered, changes in binding affinity as well as immunogenicity can be monitored and scored such that an antibody optimized for the best combined binding and low immunogenicity are achieved (see, e.g., U.S. Pat. No. 6,656,467 and U.S. Pat. Pub. US20020164326A1).

In another approach, the CDR regions of the antibody are analyzed to determine the contributions of each individual CDR to antibody binding and/or immunogenicity by systemically replacing each of the donor CDRs with a human counterpart. The resultant panel of humanized antibodies is then scored for antigen affinity and potential immunogenicity of each CDR. In this way, the two clinically important properties of a candidate binding molecule, i.e., antigen binding and low immunogenicity, are determined. If patient sera against a corresponding murine or CDR-grafted (humanized) form of the antibody is available, then the entire panel of antibodies representing the systematic human CDR exchanges can be screened to determine the patients anti-idiotypic response against each donor CDR (for technical details, see, e.g., Iwashi et al., Mol. Immunol. 36:1079-91 (1999). Such an approach allows for identifying essential donor CDR regions from non-essential donor CDRs. Nonessential donor CDR regions may then be exchanged with a human counterpart CDR. Where an essential CDR region cannot be exchanged without unacceptable loss of function, identification of the specificity-determining residues (SDRs) of the CDR is performed by, for example, site-directed mutagenesis. In this way, the CDR can then be reengineered to retain only the SDRs and be human and/or minimally immunogenic at the remaining amino acid positions throughout the CDR. Such an approach, where only a portion of the donor CDR is grafted, is also referred to as abbreviated CDR-grafting (for technical details on the foregoing techniques, see, e.g., Tamura et al., J. of Immunology 164(3):1432-41. (2000); Gonzales et al., Mol. Immunol 40:337-349 (2003); Kashmiri et al., Crit. Rev. Oncol. Hematol. 38:3-16 (2001); and De Pascalis et al, J. of Immunology 169(6):3076-84. (2002).

Moreover, it is sometimes possible to make one or more conservative amino acid substitutions of CDR residues without appreciably affecting the binding affinity of the resulting humanized immunoglobulin. By conservative substitutions are intended combinations such as gly, ala; val, ile, leu; asp, glu; asn, gln; ser, thr; lys, arg; and phe, tyr.

Additional candidates for substitution are acceptor human framework amino acids that are “rare” for a human immunoglobulin at that position. These amino acids can be substituted with amino acids from the equivalent position of the mouse donor antibody or from the equivalent positions of more typical human immunoglobulins. For example, substitution may be desirable when the amino acid in a human framework region of the acceptor immunoglobulin is rare for that position and the corresponding amino acid in the donor immunoglobulin is common for that position in human immunoglobulin sequences; or when the amino acid in the acceptor immunoglobulin is rare for that position and the corresponding amino acid in the donor immunoglobulin is also rare, relative to other human sequences. Whether a residue is rare for acceptor human framework sequences should also be considered when selecting residues for backmutation based on contribution to CDR conformation. For example, if backmutation results in substitution of a residue that is rare for acceptor human framework sequences, a humanized antibody may be tested with and without for activity. If the backmutation is not necessary for activity, it may be eliminated to reduce immunogenicity concerns. For example, backmutation at the following residues may introduce a residue that is rare in acceptor human framework sequences; v1=V2(2.0%), L3 (0.4%), T7 (1.8%), Q18 (0.2%), L83 (1.2%), 185 (2.9%), A100 (0.3%) and L106 (1.1%); and vh=T3 (2.0%), K5 (1.8%), 111 (0.2%), S23 (1.5%), F24 (1.5%), S41 (2.3%), K71 (2.4%), R75 (1.4%), 182 (1.4%), D83 (2.2%) and L109 (0.8%). These criteria help ensure that an atypical amino acid in the human framework does not disrupt the antibody structure. Moreover, by replacing a rare human acceptor amino acid with an amino acid from the donor antibody that happens to be typical for human antibodies, the humanized antibody may be made less immunogenic.

The term “rare”, as used herein, indicates an amino acid occurring at that position in less than about 20%, preferably less than about 10%, more preferably less than about 5%, even more preferably less than about 3%, even more preferably less than about 2% and even more preferably less than about 1% of sequences in a representative sample of sequences, and the term “common”, as used herein, indicates an amino acid occurring in more than about 25% but usually more than about 50% of sequences in a representative sample. For example, when deciding whether an amino acid in a human acceptor sequence is “rare” or “common”, it will often be preferable to consider only human variable region sequences and when deciding whether a mouse amino acid is “rare” or “common”, only mouse variable region sequences. Moreover, all human light and heavy chain variable region sequences are respectively grouped into “subgroups” of sequences that are especially homologous to each other and have the same amino acids at certain critical positions (Kabat et al., supra). When deciding whether an amino acid in a human acceptor sequence is “rare” or “common” among human sequences, it will often be preferable to consider only those human sequences in the same subgroup as the acceptor sequence.

Additional candidates for substitution are acceptor human framework amino acids that would be identified as part of a CDR region under the alternative definition proposed by Chothia et al., supra. Additional candidates for substitution are acceptor human framework amino acids that would be identified as part of a CDR region under the AbM and/or contact definitions.

Additional candidates for substitution are acceptor framework residues that correspond to a rare donor framework residue. Rare donor framework residues are those that are rare (as defined herein) for murine antibodies at that position. For murine antibodies, the subgroup can be determined according to Kabat and residue positions identified which differ from the consensus. These donor specific differences may point to somatic mutations in the murine sequence which enhance activity. Rare residues that are predicted to affect binding (e.g., packing canonical and/or vernier residues) are retained, whereas residues predicted to be unimportant for binding can be substituted.

Additional candidates for substitution are non-germline residues occurring in an acceptor framework region. For example, when an acceptor antibody chain (i.e., a human antibody chain sharing significant sequence identity with the donor antibody chain) is aligned to a germline antibody chain (likewise sharing significant sequence identity with the donor chain), residues not matching between acceptor chain framework and the germline chain framework can be substituted with corresponding residues from the germline sequence.

In exemplary embodiments, a humanized antibody of the present invention (see, for example, subsections c-g infra) contains (i) a light chain comprising a variable domain comprising murine VL CDRs and a human acceptor framework, the framework having at least one, two, three, four, five, six, seven, eight, nine or more residues backmutated (i.e., substituted with the corresponding murine residue), wherein the backmutation(s) are at a canonical, packing and/or vernier residues and (ii) a heavy chain comprising murine VH CDRs and a human acceptor framework, the framework having at least one, two, three, four, five, six, seven, eight, nine or more residues backmutated, wherein the backmutation(s) are at a canonical, packing and/or vernier residues. In certain embodiments, backmutations are only at packing and/or canonical residues or are primarily at canonical and/or packing residues (e.g., only 1 or 2 vernier residues of the vernier residues differing between the donor and acceptor sequence are backmutated).

In other embodiments, humanized antibodies include the fewest number of backmutations possible while retaining a binding affinity comparable to that of the donor antibody (or a chimeric version thereof). To arrive at such versions, various combinations of backmutations can be eliminated and the resulting antibodies tested for efficacy (e.g., binding affinity). For example, backmutations (e.g., 1, 2, 3, or 4 backmutations) at vernier residues can be eliminated or backmutations at combinations of vernier and packing, vernier and canonical or packing and canonical residues can be eliminated.

Other than the specific amino acid substitutions discussed above, the framework regions of humanized immunoglobulins are usually substantially identical, and more usually, identical to the framework regions of the human antibodies from which they were derived. Of course, many of the amino acids in the framework region make little or no direct contribution to the specificity or affinity of an antibody. Thus, many individual conservative substitutions of framework residues can be tolerated without appreciable change of the specificity or affinity of the resulting humanized immunoglobulin. Thus, in one embodiment the variable framework region of the humanized immunoglobulin shares at least 85% sequence identity to a human variable framework region sequence or consensus of such sequences. In another embodiment, the variable framework region of the humanized immunoglobulin shares at least 90%, preferably 95%, more preferably 96%, 97%, 98% or 99% sequence identity to a human variable framework region sequence or consensus of such sequences. In general, however, such substitutions are undesirable.

In exemplary embodiments, the humanized antibodies of the invention exhibit a specific binding affinity for antigen of at least 10⁷, 10⁸, 10⁹ or 10¹⁰ M⁻¹. In other embodiments, the antibodies of the invention can have binding affinities of at least 10¹⁰, 10¹¹ or 10¹² M⁻¹. Usually the upper limit of binding affinity of the humanized antibodies for antigen is within a factor of three, four or five of that of the donor immunoglobulin. Often the lower limit of binding affinity is also within a factor of three, four or five of that of donor immunoglobulin. Alternatively, the binding affinity can be compared to that of a humanized antibody having no substitutions (e.g., an antibody having donor CDRs and acceptor FRs, but no FR substitutions). In such instances, the binding of the optimized antibody (with substitutions) is preferably at least two- to three-fold greater, or three- to four-fold greater, than that of the unsubstituted antibody. For making comparisons, activity of the various antibodies can be determined, for example, by BIACORE (i.e., surface plasmon resonance using unlabelled reagents) or competitive binding assays.

In one embodiment, humanized antibodies of this invention disclosure include a variable region framework sequence selected from human antibody genes (e.g., germline antibody gene segments) which include one or more canonical CDR structure types that are identical or similar to the canonical CDR structure types for the corresponding non-human antibody (e.g., murine) which is humanized. See, U.S. Pat. No. 6,881,557 and Tan et al., Journal of Immunol 169:1119-1125 (2002) (incorporated by reference in their entirety for all purposes).

Also featured are humanized antibodies comprising a framework region having a consensus amino acid sequence, for example, as described in U.S. Pat. No. 6,300,064, incorporated by reference herein in its entirety for all purposes. The following table lists various consensus sequences that can be used as framework regions in the humanized antibodies described herein. Therefore, any one of the consensus sequences shown below can be used as in combination with one or more CDRs described herein, thereby resulting in a humanized immunoglobulin or humanized antibody of this invention.

| | |
|---|---|
| Consensus Sequences | |
| for light chain | Amino Acid Sequence |

| | |
|---|---|
| framework regions | (SEQ ID NO) |

| Kappa chain | DIQMTQSPSSLSASVGDRVTITCRASQGISS |
|---|---|
| | YLAWYQQKPGKAPKLLIYAASSLQSGVPSRF |
| | SGSGSGTDFTLTISSLQPEDFATYYCQQHYT |
| | TPPTFGQGTKVEIKRT |
| | (SEQ ID NO:73) |
| Kappa chain | DIVMTQSPLSLPVTPGEPASISCRSSQSLLH |
| | SNGYNYLDWYLQKPGQSPQLLIYLGSNRASG |
| | VPDRFSGSGSGTDFTLKISRVEAEDVGVYYC |
| | QQHYTTPPTFGQGTKVEIKRT |
| | (SEQ ID NO:74) |
| Kappa chain | DIVLTQSPATLSLSPGERATLSCRASQSVSS |
| | SYLAWYQQKPGQAPRLLIYGASSRATGVPAR |
| | FSGSGSGTDFTLTISSLEPEDFAVYYCQQHY |
| | TTPPTFGQGTKVEIKRT |
| | (SEQ ID NO:75) |
| Kappa chain | DIVMTQSPDSLAVSLGERATINCRSSQSVLY |
| | SSNNKNYLAWYQQKPGQPPKLLIYWASTRES |
| | GVPDRFSGSGSGTDFTLTISSLQAEDVAVYY |
| | CQQHYTTPPTFGQGTKVEIKRT |
| | (SEQ ID NO:76) |
| Lambda chain | QSVLTQPPSVSGAPGQRVTISCSGSSSNIGS |
| | NYVSWYQQLPGTAPKLLIYDNNQRPSGVPDR |
| | FSGSKSGTSASLAITGLQSEDEADYYCQQHY |
| | TTPPVFGGGTKLTVLG |
| | (SEQ ID NO:77) |
| Lambda chain | QSALTQPASVSGSPGQSITISCTGTSSDVGG |
| | YNYVSWYQQHPGKAPKLMIYDVSNRPSGVSN |
| | RFSGSKSGNTASLTISGLQAEDEADYYCQQH |
| | YTTPPVFGGGTKLTVLG |
| | (SEQ ID NO:78) |
| Lambda chain | SYELTQPPSVSVAPGQTARISCSGDALGDKY |
| | ASWYQQKPGQAPVLVIYDDSDRPSGIPERFS |
| | GSNSGNTATLTISGTQAEDEADYYCQQHYTT |
| | PPVFGGGTKLTVLG |
| | (SEQ ID NO:79) |
| Consensus Sequences | |
| for heavy chain | Amino Acid Sequence |

| | |
|---|---|
| framework regions | (SEQ ID NO) |

| | |
|---|---|
| Heavy chain | QVQLVQSGAEVKKPGSSVKVSCKASGGTFSS |
| | YAISWVRQAPGQGLEWMGGIIPIFGTANYAQ |
| | KFQGRVTITADESTSTAYMELSSLRSEDTAV |
| | YYCARWGGDGFYAMDYWGQGTLVTVSS |
| | (SEQ ID NO:80) |
| Heavy chain | QVQLVQSGAEVKKPGASVKVSCKASGYTFTS |
| | YYMHWVRQAPGQGLEWMGWINPNSGGTNYAQ |
| | KFQGRVTMTRDTSISTAYMELSSLRSEDTAV |
| | YYCARWGGDGFYAMDYWGQGTLVTVSS |
| | (SEQ ID NO:81) |
| Heavy chain | QVQLKESGPALVKPTQTLTLTCTFSGFSLST |
| | SGVGVGWIRQPPGKALEWLALIDWDDDKYYS |
| | TSLKTRLTISKDTSKNQVVLTMTNMDPVDTA |
| | TYYCARWGGDGFYAMDYWGQGTLVTVSS |
| | (SEQ ID NO:82) |
| Heavy chain | EVQLVESGGGLVQPGGSLRLSCAASGFTFSS |
| | YAMSWVRQAPGKGLEWVSAISGSGGSTYYAD |
| | SVKGRFTISRDNSKNTLYLQMNSLRAEDTAV |
| | YYCARWGGDGFYAMDYWGQGTLVTVSS |
| | (SEQ ID NO:83) |
| Heavy chain | QVQLQESGPGLVKPSETLSLTCTVSGGSISS |
| | YYWSWIRQPPGKGLEWIGYIYYSGSTNYNPS |
| | LKSRVTISVDTSKNQFSLKLSSVTAADTAVY |
| | YCARWGGDGFYAMDYWGQGTLVTVSS |
| | (SEQ ID NO:84) |
| Heavy chain | EVQLVQSGAEVKKPGESLKISCKGSGYSFTS |
| | YWIGWVRQMPGKGLEWMGIIYPGDSDTRYSP |
| | SFQGQVTISADKSISTAYLQWSSLKASDTAM |
| | YYCARWGGDGFYAMDYWGQGTLVTVSS |
| | (SEQ ID NO:85) |
| Heavy chain | QVQLQQSGPGLVKPSQTLSLTCAISGDSVSS |
| | NSAAWNWIRQSPGRGLEWLGRTYYRSKWYND |
| | YAVSVKSRITINPDTSKNQFSLQLNSVTPED |
| | TAVYYCARWGGDGFYAMDYWGQGTLVTVSS |
| | (SEQ ID NO:86) |

Yet another strategy to produce the humanized antibodies of this invention is to select the closest human germline sequence as the framework which receives the CDRs from a murine antibody to be humanized. See, Mercken et al., US 2005/0129695 (incorporated by reference in their entirety for all purposes). Germline sequences originate from un-rearranged immunoglobulin genes and therefore do not present somatic hypermtuation that is potentially immunogenic. This approach is based on the search for the closest human germline sequence. In particular, variable domains from germline sequences that exhibit a high degree of sequence identity with the murine VL and VH framework regions can be identified using the V-Base and/or IMGT databases (publicly accessible through the Medical Research Council Center for Protein Engineering internet server and the European Bioinformatics Institute internet server, respectively). The murine CDRs are then grafted on to the chosen human germline variable region acceptor sequences.

Additional exemplary humanization techniques that can be used for humanizing the immunoglobulins of the invention are described in, for example, Presta et al., J. Immunol., 151: 2623-2632 (1993); Carter et al., Proc. Natl. Acad. Sci. USA., 89: 4285-4289 (1992); Couto et al., Cancer Res., 55: 5973s-77s (1995); O'Conner et al., Protein Eng., 11: 321-328 (1998); and Antibody Engineering-Methods and Protocols by Lo, Vol. 248 (2004).

Additionally, framework residues can be analyzed using any of the techniques as described above to determine which, if any, residues should be substituted to optimize the properties of the resulting humanized antibody. For example, computer modeling can be used to identify residues which have a good probability of directly or indirectly influencing antigen binding.

c. Production of Humanized 3D6 Antibodies

In exemplary aspects of the present invention, humanized 3D6 antibodies are featured for use in the therapeutic and/or diagnostic methodologies described herein. 3D6 is specific for the N-terminus of Aβ and has been shown to mediate phagocytosis (e.g., induce phagocytosis) of amyloid plaque (see Examples I-II). 3D6 has also been shown to preferentially bind soluble, oligomeric Aβ and is effective for rapid improvement in cognition in mammalian subjects (see Examples XII and XIII).

Suitable human acceptor antibody sequences for use in the humanization of murine 3D6 are identified by computer comparisons of the amino acid sequences of the mouse variable regions with the sequences of known human antibodies. The comparison is performed separately for heavy and light chains but the principles are similar for each. In particular, variable domains from human antibodies whose framework sequences exhibit a high degree of sequence identity with the murine VL and VH framework regions are identified by query of the Kabat Database using NCBI BLAST (publicly accessible through the National Institutes of Health NCBI internet server) with the respective murine framework sequences. In one embodiment, acceptor sequences sharing greater than 50% sequence identity with murine donor sequences are selected. Preferably, acceptor antibody sequences sharing greater than 60%, 70%, 80%, 90% or more sequence identity are selected.

The cloning and sequencing of cDNA encoding the murine 3D6 antibody heavy and light chain variable regions is described in Example III. A computer comparison of murine 3D6 with human variable sequences revealed that the 3D6 light chain shows the greatest sequence identity to human light chains of subtype kappa II, and that the 3D6 heavy chain shows greatest sequence identity to human heavy chains of subtype III, as defined by Kabat et al., supra. Thus, light and heavy human framework regions are preferably derived from human antibodies of these subtypes, or from consensus sequences of such subtypes. The preferred light chain human variable regions showing greatest sequence identity to the corresponding region from 3D6 are from antibodies having Kabat ID Numbers 019230, 005131, 005058, 005057, 005059, U21040 and U41645, with 019230 being more preferred. The preferred heavy chain human variable regions showing greatest sequence identity to the corresponding region from 3D6 are from antibodies having Kabat ID Numbers 045919, 000459, 000553, 000386 and M23691, with 045919 being more preferred.

Residues are next selected for substitution, as follows. When an amino acid differs between a 3D6 variable framework region and an equivalent human variable framework region, the human framework amino acid should usually be substituted by the equivalent mouse amino acid if it is reasonably expected that the amino acid:

(1) noncovalently binds antigen directly,

(2) is adjacent to a CDR region, is part of a CDR region under the alternative definition proposed by Chothia et al., supra, or otherwise interacts with a CDR region (e.g., is within about 3A of a CDR region) (e.g., amino acids at positions L2, H49 and H94 of 3D6), or

(3) participates in the VL-VH interface (e.g., amino acids at positions L36, L46 and H93 of 3D6).

Computer modeling of the 3D6 antibody heavy and light chain variable regions, and humanization of the 3D6 antibody is described in Example VI. Briefly, a three-dimensional model was generated based on the closest solved murine antibody structures for the heavy and light chains. For this purpose, an antibody designated 1CR9 (Protein Data Bank (PDB) ID: 1CR9, Kanyo et al., J. Mol. Biol. 293:855 (1999)) was chosen as a template for modeling the 3D6 light chain, and an antibody designated 1OPG (PDB ID: 1OPG, Kodandapani et al., J. Biol. Chem. 270:2268 (1995)) was chosen as the template for modeling the heavy chain. The model was further refined by a series of energy minimization steps to relieve unfavorable atomic contacts and optimize electrostatic and van der Waals interactions. The solved structure of 1qkz (PDB ID: 1QKZ, Derrick et al., J. Mol. Biol. 293:81 (1999)) was chosen as a template for modeling CDR3 of the heavy chain as 3D6 and 1OPG did not exhibit significant sequence homology in this region when aligned for comparison purposes.

Three-dimensional structural information for the antibodies described herein is publicly available, for example, from the Research Collaboratory for Structural Bioinformatics' Protein Data Bank (PDB). The PDB is freely accessible via the World Wide Web internet and is described by Berman et al. (2000) Nucleic Acids Research, 28:235. Computer modeling allows for the identification of CDR-interacting residues. The computer model of the structure of 3D6 can in turn serve as a starting point for predicting the three-dimensional structure of an antibody containing the 3D6 complementarity determining regions substituted in human framework structures. Additional models can be constructed representing the structure as further amino acid substitutions are introduced.

In general, substitution of one, most or all of the amino acids fulfilling the above criteria is desirable. Accordingly, the humanized antibodies of the present invention will usually contain a substitution of a human light chain framework residue with a corresponding 3D6 residue in at least 1, 2 or 3, and more usually 4, of the following positions: L1, L2, L36 and L46. The humanized antibodies also usually contain a substitution of a human heavy chain framework residue with a corresponding 3D6 residue in at least 1, 2, and sometimes 3, of the following positions: H49, H93 and H94. Humanized antibodies can also contain a substitution of a heavy chain framework residue with a corresponding germline residue in at least 1, 2, and sometimes 3, of the following positions: H74, H77 and H89.

Occasionally, however, there is some ambiguity about whether a particular amino acid meets the above criteria, and alternative variant immunoglobulins are produced, one of which has that particular substitution, the other of which does not. In instances where substitution with a murine residue would introduce a residue that is rare in human immunoglobulins at a particular position, it may be desirable to test the antibody for activity with or without the particular substitution. If activity (e.g., binding affinity and/or binding specificity) is about the same with or without the substitution, the antibody without substitution may be preferred, as it would be expected to elicit less of a HAHA response, as described herein.

Other candidates for substitution are acceptor human framework amino acids that are rare for a human immunoglobulin at that position. These amino acids can be substituted with amino acids from the equivalent position of more typical human immunoglobulins. Alternatively, amino acids from equivalent positions in the mouse 3D6 can be introduced into the human framework regions when such amino acids are typical of human immunoglobulin at the equivalent positions.

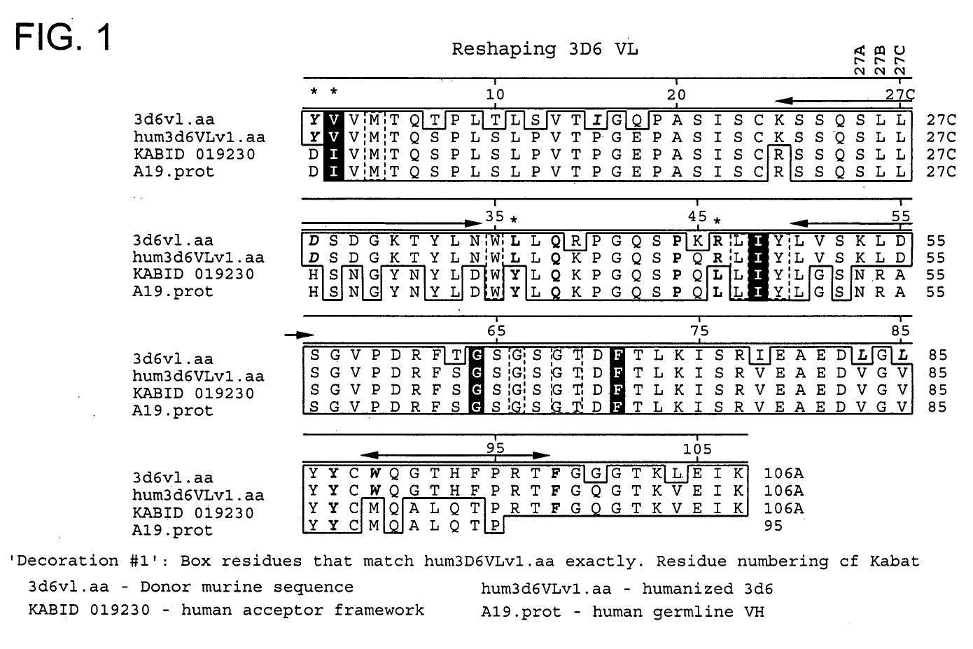

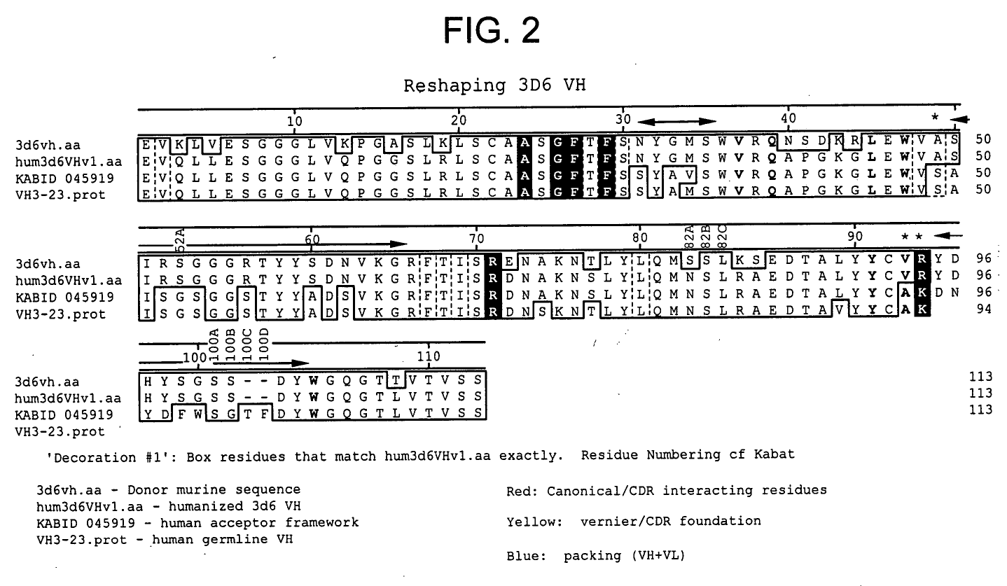

In additional embodiments, when the human light chain framework acceptor immunoglobulin is Kabat ID Number 019230, the light chain contains substitutions in at least 1, 2, 3, 4, 5, 6, 7, 8, 9, 10, 11, 12 or more usually 13, of the following positions: L7, L10, L12, L15, L17, L39, L45, L63, L78, L83, L85, L100 or L104. In additional embodiments when the human heavy chain framework acceptor immunoglobulin is Kabat ID Number 045919, the heavy chain contains substitutions in at least 1, 2, 3, 4, 5, 6, 7, 8, 9, 10, 11, 12, 13, 14, or more usually 15, of the following positions: H3, H5, H13, H16, H19, H40, H41, H42, H44, H72, H77, H82A, H83, H84, or H108. These positions are substituted with the amino acid from the equivalent position of a human immunoglobulin having a more typical amino acid residue. Examples of appropriate amino acids to substitute are shown in FIGS. 1 and 2.

Other candidates for substitution are non-germline residues occurring in a framework region. A computer comparison of 3D6 with known germline sequences revealed that heavy chains showing the greatest degree of sequence identity include germline variable region sequences VH3-48, VH3-23, VH3-7, VH3-21 and VH3-11, with VH3-23 being more preferred. Alignment of Kabat ID 045919 with VH3-23 reveals that residues H74, H77 and/or H89 may be selected for substitution with corresponding germline residues (e.g., residues H74, H77 and/or H89 when comparing Kabat ID 045919 and VH3-23). Likewise, germline sequences having the greatest degree of identity to the 3D6 light chain include A1, A17, A18, A2 and A19, with A19 being most preferred. Residues not matching between a selected light chain acceptor framework and one of these germline sequences could be selected for substitution with the corresponding germline residue.

Table 1 summarizes the sequence analysis of the 3D6 VH and VL regions. Additional mouse and human structures that can be used for computer modeling of the 3D6 antibody and additional human antibodies are set forth as well as germline sequences that can be used in selecting amino acid substitutions. Rare mouse residues are also set forth in Table 1. Rare mouse residues are identified by comparing the donor VL and/or VH sequences with the sequences of other members of the subgroup to which the donor VL and/or VH sequences belong (according to Kabat) and identifying the residue positions which differ from the consensus. These donor specific differences may point to somatic mutations which enhance activity. Rare residues close to the binding site may possibly contact the antigen, making it desirable to retain the mouse residue. However, if the rare mouse residue is not important for binding, use of the corresponding acceptor residue is preferred as the mouse residue may create immunogenic neoepitopes in the humanized antibody. In the situation where a rare residue in the donor sequence is actually a common residues in the corresponding acceptor sequence, the preferred residue is clearly the acceptor residue.

TABLE 1
Summary of 3D6 V-region sequence

| Chain | Heavy | Light |
|---|---|---|
| Mouse subgroup | IIID (002688) | II (005840-005844, 005851-005853, |
| (Kabat seq ID#) | | 005857, 005863) |
| Mouse homologs | 002727/163.1′CL | 005840/1210.7 |
| (Kabat/Genbank) | 002711/H35-C6′CL | 005843/42.4b.12.2′CL |
| | 002733/8-1-12-5-3-1(A2-1)′CL | 005842/BXW-14′CL |
| | 002715/ASWA2′CL | 005841/42.7B3.2′CL |
| | 020669/#14′CL | 005851/36-60CRI- |
| Rare amino acids (% | N40 (0.233%) | Y1(.035%) |
| frequency of | D42 (0.699%) | I15 (3.3%) |
| occurrence in class) | | D27 (0.867%)-CDR1 |
| | | I78 (0.677%) |
| | | L85 (0.625%) |
| | | W89 (0.815%)-CDR3 |
| | | K106A (0.295%) |
| Human Subgroup | III (000488-000491, 000503, 000624) | II (005046) |
| Chothia canonical | H1: class 1 [2fbj] | L1: class 4 [1rmf] |
| CDR groupings [pdb | H2: class 3 [1igc] | L2: class 1 [1lmk] |
| example] | | L3: class 1 [1tet] |
| Closest solved mouse | PDB ID: 1OPG Kodandapani et al., | PDB ID: 1CR9; Kanyo et al., supra; |
| structures | supra; (72% 2 Å) | (94%, 2 Å) |
| | | PDB ID: 1NLD; Davies et al., Acta |
| | | Crystallogr. D. Biol. Crystallog. 53: 186 |
| | | (1997); (98%, 2.8 Å) |
| Closest solved human | 1VH (68%, nmr) | 1LVE (57%, LEN) |
| structures | 443560 (65%, IgG, λ myeloma, 1.8 Å) | 1B6DA (54%, B-J dimer, 2.8 Å); |
| | KOL/2FB4H (60%, myeloma, 3 Å) | 1VGEL (54%, autoAb) |
| Germline query (Hu) | VH3-48 (4512283/BAA75032.1) | A1(x63402) |
| results (top 4) | VH3-23 (4512287/BAA75046.1) | A17 (x63403) |
| | VH3-7 (4512300/BAA75056.1) | A18 (X63396) |
| | VH3-21 (4512287/BAA75047.1) | A2 (m31952) |
| | VH3-11 (4152300/BAA75053.1) | A19 (x63397) |

*heavy chain and light chain from the same antibody (O-81, Hirabayashi et al. NAR 20: 2601).

Kabat ID sequences referenced herein are publicly available, for example, from the Northwestern University Biomedical Engineering Department's Kabat Database of Sequences of Proteins of Immunological Interest. Three-dimensional structural information for antibodies described herein is publicly available, for example, from the Research Collaboratory for Structural Bioinformatics' Protein Data Bank (PDB). The PDB is freely accessible via the World Wide Web internet and is described by Berman et al. (2000) Nucleic Acids Research, p235-242. Germline gene sequences referenced herein are publicly available, for example, from the National Center for Biotechnology Information (NCBI) database of sequences in collections of Igh, Ig kappa and Ig lambda germline V genes (as a division of the National Library of Medicine (NLM) at the National Institutes of Health (NIH)). Homology searching of the NCBI “Ig Germline Genes” database is provided by IgG BLAST™.

In a preferred embodiment, a humanized antibody of the present invention contains (i) a light chain comprising a variable domain comprising murine 3D6 VL CDRs and a human acceptor framework, the framework having at least one, preferably two, three or four residues selected from the group consisting of L1, L2, L36, and L46 substituted with the corresponding 3D6 residue and (ii) a heavy chain comprising 3D6 VH CDRs and a human acceptor framework, the framework having at least one, preferably two or three residues selected from the group consisting of H49, H93 and H94 substituted with the corresponding 3D6 residue, and, optionally, at least one, preferably two or three residues selected from the group consisting of H74, H77 and H89 is substituted with a corresponding human germline residue.

In a more preferred embodiment, a humanized antibody of the present invention contains (i) a light chain comprising a variable domain comprising murine 3D6 VL CDRs and a human acceptor framework, the framework having residue 1 substituted with a tyr (Y), residue 2 substituted with a val (V), residue 36 substituted with a leu (L) and/or residue 46 substituted with an arg (R), and (ii) a heavy chain comprising 3D6 VH CDRs and a human acceptor framework, the framework having residue 49 substituted with an ala (A), residue 93 substituted with a val (V) and/or residue 94 substituted with an arg (R), and, optionally, having residue 74 substituted with a ser (S), residue 77 substituted with a thr (T) and/or residue 89 substituted with a val (V).

In a particularly preferred embodiment, a humanized antibody of the present invention has structural features, as described herein, and further has at least one (preferably two, three, four or all) of the following activities: (1) binds aggregated Aβ1-42 (e.g., as determined by ELISA); (2) binds Aβ in plaques (e.g., staining of AD and/or PDAPP plaques); (3) binds Aβ with two- to three-fold higher binding affinity as compared to chimeric 3D6 (e.g., 3D6 having murine variable region sequences and human constant region sequences); (4) mediates phagocytosis of Aβ (e.g., in an ex vivo phagocytosis assay, as described herein); and (5) crosses the blood-brain barrier (e.g., demonstrates short-term brain localization, for example, in a PDAPP animal model, as described herein).

In another embodiment, a humanized antibody of the present invention has structural features, as described herein, and further binds Aβ in a manner or with an affinity sufficient to elicit at least one of the following in vivo effects: (1) reduce Aβplaque burden; (2) prevent plaque formation; (3) reduce levels of soluble Aβ (e.g., soluble oligomeric Aβ); (4) reduce the neuritic pathology associated with an amyloidogenic disorder; (5) lessens or ameliorate at least one physiological symptom associated with an amyloidogenic disorder; and/or (6) improves cognitive function (e.g., rapid improvement).

In another embodiment, a humanized antibody of the present invention has structural features, as described herein, and specifically binds to an epitope comprising residues 1-5 or 3-7 of Aβ.

d. Production of Humanized 12B4 Antibodies

In further exemplary examples of the present invention, humanized 12B4 antibodies are featured for use in the therapeutic and/or diagnostic methodologies described herein. 12B4 is specific for the N-terminus of Aβ and has been shown to mediate phagocytosis (e.g., induce phagocytosis) of amyloid plaque. 12B4 has also been shown to appreciably capture soluble Aβ. The cloning and sequencing of cDNA encoding the 12B4 antibody heavy and light chain variable regions is described in Example V.

Identification of suitable human acceptor antibody sequences for humanization of 12B4 is the same as described in subsection c, supra.

A computer comparison of 12B4 revealed that the 12B4 light chain shows the greatest sequence identity to human light chains of subtype kappa II, and that the 12B4 heavy chain shows greatest sequence identity to human heavy chains of subtype II, as defined by Kabat et al., supra. Thus, light and heavy human framework regions are preferably derived from human antibodies of these subtypes, or from consensus sequences of such subtypes. The preferred light chain human variable regions showing greatest sequence identity to the corresponding region from 12B4 are from an antibody having Kabat ID Number 005036. The preferred heavy chain human variable regions showing greatest sequence identity to the corresponding region from 12B4 are from an antibody having Kabat ID Number 000333, an antibody having Genbank Accession No. AAB35009, and an antibody having Genbank Accession No. AAD53816, with the antibody having Kabat ID Number 000333 being more preferred.

Residues are next selected for substitution, as follows. When an amino acid differs between a 12B4 variable framework region and an equivalent human variable framework region, the human framework amino acid should usually be substituted by the equivalent mouse amino acid if it is reasonably expected that the amino acid:

(1) noncovalently binds antigen directly,

(2) is adjacent to a CDR region, is part of a CDR region under the alternative definition proposed by Chothia et al., supra, or otherwise interacts with a CDR region (e.g., is within about 3A of a CDR region), or

(3) participates in the VL-VH interface.

Computer modeling of the 12B4 antibody heavy and light chain variable regions, and humanization of the 12B4 antibody is described in Example V. Briefly, a three-dimensional model is generated based on the closest solved murine antibody structures for the heavy and light chains. The model is further refined by a series of energy minimization steps to relieve unfavorable atomic contacts and optimize electrostatic and van der Waals interactions.

Computer modeling allows for the identification of CDR-interacting residues. The computer model of the structure of 12B4 can in turn serve as a starting point for predicting the three-dimensional structure of an antibody containing the 12B4 complementarity determining regions substituted in human framework structures. Additional models can be constructed representing the structure as further amino acid substitutions are introduced.

In general, substitution of one, most or all of the amino acids fulfilling the above criteria is desirable. Accordingly, the humanized antibodies of the present invention will usually contain a substitution of a human light chain framework residue with a corresponding 12B4 residue in at least 1, 2, 3 or more of the chosen positions. The humanized antibodies also usually contain a substitution of a human heavy chain framework residue with a corresponding 12B4 residue in at least 1, 2, 3 or more of the chosen positions.

Occasionally ambiguities about whether a particular amino acid meets the above criteria can be addressed as described in subsection c, supra.

Other candidates for substitution are acceptor human framework amino acids that are rare for a human immunoglobulin at that position. These amino acids can be substituted with amino acids from the equivalent position of more typical human immunoglobulins. Alternatively, amino acids from equivalent positions in the mouse 12B4 can be introduced into the human framework regions when such amino acids are typical of human immunoglobulin at the equivalent positions.

Other candidates for substitution are non-germline residues occurring in a framework region. By performing a computer comparison of 12B4 with known germline sequences, germline sequences with the greatest degree of sequence identity to the heavy or light chain can be identified. Alignment of the framework region and the germline sequence will reveal which residues may be selected for substitution with corresponding germline residues. Residues not matching between a selected light chain acceptor framework and one of these germline sequences could be selected for substitution with the corresponding germline residue.

Table 2 summarizes the sequence analysis of the 12B4 VH and VL regions. Additional mouse and human structures that can be used for computer modeling of the 12B4 antibody and additional human antibodies are set forth as well as germline sequences that can be used in selecting amino acid substitutions. Rare mouse residues are also set forth in Table 2. Rare mouse residues are identified by comparing the donor VL and/or VH sequences with the sequences of other members of the subgroup to which the donor VL and/or VH sequences belong (according to Kabat) and identifying the residue positions which differ from the consensus. These donor specific differences may point to somatic mutations which enhance activity. Rare residues close to the binding site may possibly contact the antigen, making it desirable to retain the mouse residue. However, if the rare mouse residue is not important for binding, use of the corresponding acceptor residue is preferred as the mouse residue may create immunogenic neoepitopes in the humanized antibody. In the situation where a rare residue in the donor sequence is actually a common residue in the corresponding acceptor sequence, the preferred residue is clearly the acceptor residue.

TABLE 2
Summary of 12B4 V region sequence

| Chain | VL | VH |
|---|---|---|
| Mouse Subgroup | II | Ib |
| Human Subgroup | II | II |
| Rare amino acids (% frequency) | K107 (0.542%) | T3, I11, L12, F24, |
| | | S41, N75, D83, A85 |
| Chothia canonical class | L1: ˜class 4[1rmf] | H1: class 3 [1ggi] |
| | L2: class 1[1lmk] | H2: ˜class 1 |
| | L3: class 1[1tet] | |
| Closest mouse MAb solved | 2PCP (2.2 Å) | 1ETZ (2.6 Å) |
| structure | | |
| Homology with modeling template | 94% | 80% |
| Human Framework seq | KABID 005036 | 1-KABID 000333 |
| | | 2-AAB35009/1F7 |
| | | 3-AAD53816 |
| Germline ref for Hu Fr | A3/x12690 & | 1: VH4-39/AB019439/BAA75036.1 |
| | A19/X63397 | 2: VH2-5/AB019440/ |
| | | BAA75057.1 |

In a preferred embodiment, a humanized antibody of the present invention contains (i) a light chain comprising a variable domain comprising murine 12B4 VL CDRs and a human acceptor framework, the framework having at least one, residue substituted with the corresponding 12B4 residue and (ii) a heavy chain comprising 12B4 VH CDRs and a human acceptor framework, the framework having at least one, two, three, four, five, six, seven, eight, or nine residues substituted with the corresponding 12B4 residue, and, optionally, at least one, two or three residues substituted with a corresponding human germline residue.

In another preferred embodiment, a humanized antibody of the present invention has structural features, as described herein, and further has at least one (preferably two, three, four or all) of the following activities: (1) binds soluble Aβ; (2) binds aggregated Aβ1-42 (e.g., as determined by ELISA); (3) binds Aβ in plaques (e.g., staining of AD and/or PDAPP plaques); (4) binds Aβ with two- to three-fold higher binding affinity as compared to chimeric 12B4 (e.g., 12B4 having murine variable region sequences and human constant region sequences); (5) mediates phagocytosis of Aβ (e.g., in an ex vivo phagocytosis assay, as described herein); and (6) crosses the blood-brain barrier (e.g., demonstrates short-term brain localization, for example, in a PDAPP animal model, as described herein).

In another preferred embodiment, a humanized antibody of the present invention has structural features, as described herein, and further binds Aβ in a manner or with an affinity sufficient to elicit at least one of the following and further in vivo effects: (1) reduce Aβ plaque burden; (2) prevent plaque formation; (3) reduce levels of soluble Aβ; (4) reduce the neuritic pathology associated with an amyloidogenic disorder; (5) lessen or ameliorate at least one physiological symptom associated with an amyloidogenic disorder; and/or (6) improve cognitive function.

In another preferred embodiment, a humanized antibody of the present invention has structural features, as described herein, and specifically binds to an epitope comprising residues 3-7 of Aβ.

In another preferred embodiment, a humanized antibody of the present invention has structural features, as described herein, binds to an N-terminal epitope within Aβ (e.g., binds to an epitope within amino acids 3-7 of Aβ), and is capable of reducing (1) Aβ peptide levels; (2) Aβ plaque burden; and (3) the neuritic burden or neuritic dystrophy associated with an amyloidogenic disorder.

e. Production of Humanized 12A11 Antibodies

In further exemplary aspects of the present invention, 12A11 humanized antibodies are featured for use in the therapeutic and/or diagnostic methodologies described herein. 12A11 is specific for the N-terminus of Aβ and has been shown to (1) have a high avidity for aggregated Aβ1-42, (2) have the ability to capture soluble Aβ, and (3) mediate phagocytosis (e.g., induce phagocytosis) of amyloid plaque (see Examples IX and XI). The in vivo efficacy of the 12A11 antibody is described in Example X. 12A11 has also been shown to preferentially bind soluble, oligomeric Aβand is effective for rapid improvement of cognition in mammalian subjects (see Examples XII, XIII, and XIV). The cloning and sequencing of cDNA encoding the 12A11 antibody heavy and light chain variable regions is described in Example XV.

Identification of suitable human acceptor antibody sequences is the same as described in subsection c, supra. A computer comparison of 12A11 revealed that the 12A11 light chain (mouse subgroup II) shows the greatest sequence identity to human light chains of subtype kappa II, and that the 12A11 heavy chain (mouse subgroup Ib) shows greatest sequence identity to human heavy chains of subtype II, as defined by Kabat et al., supra. Light and heavy human framework regions can be derived from human antibodies of these subtypes, or from consensus sequences of such subtypes. In a first humanization effort, light chain variable framework regions were derived from human subgroup II antibodies. Based on previous experiments designed to achieve high levels of expression of humanized antibodies having heavy chain variable framework regions derived from human subgroup II antibodies, it had been discovered that expression levels of such antibodies were sometimes low. Accordingly, based on the reasoning described in Saldanha et al. (1999) Mol Immunol. 36:709-19, framework regions from human subgroup III antibodies were chosen rather than human subgroup II.

A human subgroup II antibody K64(AIMS4) (accession no. BAC01733) was identified from the NCBI non-redundant database having significant sequence identity within the light chain variable regions of 12A11. A human subgroup III antibody M72 (accession no. AAA69734) was identified from the NCBI non-redundant database having significant sequence identity within the heavy chain variable regions of 12A11 (see also Schroeder and Wang (1990) Proc. Natl. Acad. Sci. U.S.A. 872: 6146-6150.

Alternative light chain acceptor sequences include, for example, PDB Accession No. 1KFA (gi24158782), PDB Accession No. 1KFA (gi24158784), EMBL Accession No. CAE75574.1 (gi38522587), EMBL Accession No. CAE75575.1 (gi38522590), EMBL Accession No. CAE84952.1 (gi39838891), DJB Accession No. BAC01734.1 (gi21669419), DJB Accession No. BAC01730.1 (gi21669411), PIR Accession No. S40312 (gi481978), EMBL Accession No. CAA51090.1 (gi3980118), GenBank Accession No. AAH63599.1 (gi39794308), PIR Accession No. S22902 (gi106540), PIR Accession No. S42611 (gi631215), EMBL Accession No. CAA38072.1 (gi433890), GenBank Accession No. AAD00856.1 (gi4100384), EMBL Accession No. CAA39072.1 (gi34000), PIR Accession No. S23230 (gi284256), DBJ Accession No. BAC01599.1 (gi21669149), DBJ Accession No. BAC01729.1 (gi21669409), DBJ Accession No. BAC01562.1 (gi21669075), EMBL Accession No. CAA85590.1 (gi587338), GenBank Accession No. AAQ99243.1 (gi37694665), GenBank Accession No. AAK94811.1 (gi18025604), EMBL Accession No. CAB51297.1 (gi5578794), DBJ Accession No. BAC01740.1 (gi21669431), and DBJ Accession No. BAC01733.1 (gi21669417). Alternative heavy chain acceptor sequences include, for example, GenBank Accession No. AAB35009.1 (gi1041885), DBJ Accession No. BAC01904.1 (gi21669789), GenBank Accession No. AAD53816.1 (gi5834100), GenBank Accession No. AAS86081.1 (gi46254223), DBJ Accession No. BAC01462.1 (gi21668870), GenBank Accession No. AAC18191.1 (gi3170773), DBJ Accession No. BAC02266.1 (gi21670513), GenBank Accession No. AAD56254.1 (gi5921589), GenBank Accession No. AAD53807.1 (gi5834082), DBJ Accession No. BAC02260.1 (gi21670501), GenBank Accession No. AAC18166.1 (gi3170723), EMBL Accession No. CAA49495.1 (gi33085), PIR Accession No. S31513 (gi345903), GenBank Accession No. AAS86079.1 (gi46254219), DBJ Accession No. BAC01917.1 (gi21669815), DBJ Accession No. BAC01912.1 (gi21669805), GenBank Accession No. AAC18283.1 (gi3170961), DBJ Accession No. BAC01903 (gi21669787), DBJ Accession NO. BAC01887.1 (gi21669755), DBJ Accession No. BAC02259.1 (gi21370499), DBJ Accession No. BAC01913.1 (gi21669807), DBJ Accession No. BAC01910.1 (gi21669801), DJB Accession No. BAC02267.1 (gi21670515), GenBank Accession No. AAC18306.1 (gi31171011), GenBank Accession No. AAD53817.1 (gi5834102), PIR Accession No. E36005 (gi10423), EMBL CAB37129.1 (gi4456494) and GenBank AAA68892.1 (gi186190).

In exemplary embodiments, humanized antibodies of the invention include 12A11 CDRs and FRs from an acceptor sequence listed supra. Residues within the framework regions important for CDR conformation and/or activity as described herein are selected for backmutation (if differing between donor and acceptor sequences).

Residues are next selected for substitution, as follows. When an amino acid differs between a 12A11 variable framework region and an equivalent human variable framework region, the human framework amino acid should usually be substituted by the equivalent mouse amino acid if it is reasonably expected that the amino acid:

(1) noncovalently binds antigen directly,

(2) is adjacent to a CDR region, is part of a CDR region under the alternative definition proposed by Chothia et al., supra, or otherwise interacts with a CDR region (e.g., is within about 3A of a CDR region), or

(3) participates in the VL-VH interface.

Structural analysis of the 12A11 antibody heavy and light chain variable regions, and humanization of the 12A11 antibody is described in Example V. Briefly, three-dimensional models for the solved murine antibody structures 1KTR for the light chain and 1JRH and 1ETZ for the heavy chain were studied. Alternative three-dimensional models which can be studied for identification of residues, important for CDR confirmation (e.g., vernier residues), include PDB Accession No. 2JEL (gi3212688), PDB Accession No. 1TET (gi494639), PDB Accession No. IJP5 (gi16975307), PDB Accession No. 1CBV (gi493917), PDB Accession No. 2PCP (gi4388943), PDB Accession No. 1I9I (gi2050118), PDB Accession No. 1CLZ (gi1827926), PDB Accession No. 1FL6 (gi 17942615) and PDB Accession No. 1KEL (gi1942968) for the light chain and PDB 1GGI (gi442938), PDB Accession No. 1GGB (gi442934), PDB Accession No. 1N5Y (gi28373913), PDB Accession No. 2HMI (gi3891821), PDB Accession No. 1FDL (gi229915), PDB Accession No. 1KIP (gi1942788), PDB Accession No. 1KIQ (gi 1942791) and PDB Accession No. 1VFA (gi576325) for the heavy chain.

Study of solved three-dimensional structures allows for the identification of CDR-interacting residues within 12A11. Alternatively, three-dimensional models for the 12A11 VH and VL chains can be generated using computer modeling software. Briefly, a three-dimensional model is generated based on the closest solved murine antibody structures for the heavy and light chains. For this purpose, 1KTR can be used as a template for modeling the 12A11 light chain, and 1ETZ and 1JRH used as templates for modeling the heavy chain. The model can be further refined by a series of energy minimization steps to relieve unfavorable atomic contacts and optimize electrostatic and van der Waals interactions. Additional three-dimensional analysis and/or modeling can be performed using 2JEL (2.5 Å) and/or 1TET (2.3 Å) for the light chain and 1GGI (2.8 Å) for the heavy chain (or other antibodies set forth supra) based on the similarity between these solved murine structures and the respective 12A11 chains.

The computer model of the structure of 12A11 can further serve as a starting point for predicting the three-dimensional structure of an antibody containing the 12A11 complementarity determining regions substituted in human framework structures. Additional models can be constructed representing the structure as further amino acid substitutions are introduced.

In general, substitution of one, most or all of the amino acids fulfilling the above criteria is desirable. Accordingly, the humanized antibodies of the present invention will usually contain a substitution of a human light chain framework residue with a corresponding 12A11 residue in at least 1, 2, 3 or more of the chosen positions. The humanized antibodies also usually contain a substitution of a human heavy chain framework residue with a corresponding 12A11 residue in at least 1, 2, 3 or more of the chosen positions.

Ambiguities about whether a particular amino acid meets the above criteria can be addressed as described in subsection c, supra.

Other candidates for substitution are acceptor human framework amino acids that are rare for a human immunoglobulin at that position. These amino acids can be substituted with amino acids from the equivalent position of more typical human immunoglobulins. Alternatively, amino acids from equivalent positions in the mouse 12A11 can be introduced into the human framework regions when such amino acids are typical of human immunoglobulin at the equivalent positions.

Other candidates for substitution are non-germline residues occurring in a framework region. By performing a computer comparison of 12A11 with known germline sequences, germline sequences with the greatest degree of sequence identity to the heavy or light chain can be identified. Alignment of the framework region and the germline sequence will reveal which residues may be selected for substitution with corresponding germline residues. Residues not matching between a selected light chain acceptor framework and one of these germline sequences could be selected for substitution with the corresponding germline residue.

Rare mouse residues are identified by comparing the donor VL and/or VH sequences with the sequences of other members of the subgroup to which the donor VL and/or VH sequences belong (according to Kabat) and identifying the residue positions which differ from the consensus. These donor specific differences may point to somatic mutations which enhance activity. Rare residues close to the binding site may possibly contact the antigen, making it desirable to retain the mouse residue. However, if the rare mouse residue is not important for binding, use of the corresponding acceptor residue is preferred as the mouse residue may create immunogenic neoepitopes in the humanized antibody. In the situation where a rare residue in the donor sequence is actually a common residue in the corresponding acceptor sequence, the preferred residue is clearly the acceptor residue.

Table 3 summarizes the sequence analysis of the 12A11 VH and VL regions.

TABLE 3
Summary of 12A11 V region sequence

| Chain | VL | VH |
|---|---|---|
| Mouse Subgroup | II | Ib |
| Human Subgroup | II | II |
| Rare amino acids in mouse vk (% | I85 (3.6%) | I11 (1.7%) |
| frequency) | | T3 (1.0%), L12 (1.7%), S41 |
| | | (2.8%), D83 (1.8%), A85 |
| | | (1.8%) |
| Chothia canonical class | L1: class 4[16f] | H1: class 3 [7] |
| | L2: class 1[7] | H2: class 1[16] |
| | L3: class 1[9] | H3¹ |
| Closest mouse MAb solved | 1KTR² | 1ETZ³ (2.6 Å) and |
| structure | | 1JRH⁴ |
| Homology with Modeling template | 94% | 83% and 86% |
| Human Framework seq | K64 (BAC01733) | M72 (AAA69734) |
| | (87% FR, 67% overall) | (61% FR, 45% overall) |
| Donor notes | Hu k LC subgroup II | HU HC subgroup III |
| | CDRs from same canonical | CDRs from same canonical |
| | Structural group as 12A11 | structural group as 12A11 |
| Backmutation Notes | none | A24F, F29L: H1 |
| | | R71K: Canonical, H2 |
| | | V371: Packing |
| | | T28S, V48L, F67L, N73T, |
| | | L78V: Vernier |
| Germline ref for Hu Fr | A19 VL Vk2-28 | AAA69731.1 (GI: 567123) |
| | mRNA: X63397.1 (GI: 33774) | |

¹No canonical class but might form a kinked base according to the rules of Shirai et al. (1999) FEBS Lett. 4 55: 188-197.
²Kaufmann et al. (2002) J Mol Biol. 318: 135-147.
³Guddat et al. (2000) J Mol Biol. 302: 853-872.
⁴Sogabe et al. (1997) J Mol Biol. 273: 882-897.

Germline sequences are set forth that can be used in selecting amino acid substitutions.

In an exemplary embodiment, a humanized antibody of the present invention contains (i) a light chain comprising a variable domain comprising murine 12A11 VL CDRs and a human acceptor framework, the framework having zero, one, two, three, four, five, six, seven, eight, nine or more residues substituted with the corresponding 12A11 residue and (ii) a heavy chain comprising 12A11 VH CDRs and a human acceptor framework, the framework having at least one, two, three, four, five, six, seven, eight, nine or more residues substituted with the corresponding 12A11 residue, and, optionally, at least one, preferably two or three residues substituted with a corresponding human germline residue.

In another embodiment, a humanized antibody of the present invention has structural features, as described herein, and further has at least one (preferably two, three, four or all) of the following activities: (1) binds soluble Aβ; (2) binds aggregated Aβ1-42 (e.g., as determined by ELISA); (3) captures soluble Aβ; (4) binds Aβ in plaques (e.g., staining of AD and/or PDAPP plaques); (5) binds Aβ with an affinity no less than two to three fold lower than chimeric 12A11 (e.g., 12A11 having murine variable region sequences and human constant region sequences); (6) mediates phagocytosis of Aβ (e.g., in an ex vivo phagocytosis assay, as described herein); and (7) crosses the blood-brain barrier (e.g., demonstrates short-term brain localization, for example, in a PDAPP animal model, as described herein).

In another embodiment, a humanized antibody of the present invention has structural features, as described herein, such that it binds Aβ in a manner or with an affinity sufficient to elicit at least one of the following in vivo effects: (1) reduce Aβ plaque burden; (2) prevent plaque formation; (3) reduce levels of soluble Aβ (e.g., soluble oligomeric Aβ); (4) reduce the neuritic pathology associated with an amyloidogenic disorder; (5) lessen or ameliorate at least one physiological symptom associated with an amyloidogenic disorder; and/or (6) improve cognitive function (e.g., rapid improvement).

In another embodiment, a humanized antibody of the present invention has structural features, as described herein, and specifically binds to an epitope comprising residues 3-7 of Aβ.

In yet another embodiment, a humanized antibody of the present invention has structural features, as described herein, such that it binds to an N-terminal epitope within Aβ (e.g., binds to an epitope within amino acids 3-7 of Aβ), and is capable of reducing (1) Aβ peptide levels; (2) Aβ plaque burden; and (3) the neuritic burden or neuritic dystrophy associated with an amyloidogenic disorder.

In one embodiment, a humanized antibody of the invention includes the 12A11v.1 VH region linked to an IgG1 constant region, as shown below in SEQ ID NO:88).

QVQLVESGGGVVQPGRSLRLSCAFSGFSLSTSGMSVGWIRQAPGKGLEWL
AHIWWDDDKYYNPSLKSRLTISKDTSKNTVYLQMNSLRAEDTAVYYCARR
TTTADYFAYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
(K)

In another embodiment, a humanized antibody of the invention includes the 12A11v.1 VH region linked to an IgG4 constant region, as shown below in SEQ ID NO:89.

QVQLVESGGGVVQPGRSLRLSCAFSGFSLSTSGMSVGWIRQAPGKGLEWL
AHIWWDDDKYYNPSLKSRLTISKDTSKNTVYLQMNSLRAEDTAVYYCARR
TTTADYFAYWGQGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLG(K)

In yet another embodiment, a humanized antibody of the invention includes a 12A11v3.1 VH region linked to an IgG1 or an IgG4 constant region, as shown below in SEQ ID NOs:90 and 91, respectively.

QVQLVESGGGVVQPGRSLRLSCAFSGFTLSTSGMSVGWIRQAPGKGLEWL
AHIWWDDDKYYNPSLKSRFTISKDNSKNTLYLQMNSLRAEDTAVYYCARR
TTTADYFAYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
(K)
QVQLVESGGGVVQPGRSLRLSCAFSGFTLSTSGMSVGWIRQAPGKGLEWL
AHIWWDDDKYYNPSLKSRFTISKDNSKNTLYLQMNSLRAEDTAVYYCARR
TTTADYFAYWGQGTTVTVSSQVQLVESGGGVVQPGRSLRLSCAFSGFSLS
TSGMSVGWIRQAPGKGLEWLAHIWWDDDKYYNPSLKSRLTISKDTSKNTV
YLQMNSLRAEDTAVYYCARRTTTADYFAYWGQGTTVTVSSASTKGPSVFP
LAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCP
APEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHE
ALHNHYTQKSLSLSLG(K)

In some embodiments, the terminal lysine, as shown in parenthesis, is optionally expressed.

f. Production of Humanized 10D5 and 15C11 Antibodies

In further exemplary examples of the present invention, humanized 10D5 and 15C11 antibodies are featured for use in the therapeutic and/or diagnostic methodologies described herein. 10D5 and 15C11 are specific for the N-terminus and central region of Aβ, respectively

The cloning and sequencing of cDNA encoding the antibody heavy and light chain variable regions of 10D5 and 15C11 are described in Examples IV and XVI, respectively.

Identification of suitable human acceptor antibody sequences for humanization of murine 10D5 and 15C11 can be performed as described in subsection c, supra. Briefly, sequence analysis can be performed to identify the human light chains to which the murine light chain exhibits the greatest sequence identity. Light and heavy human framework regions are preferably derived from human antibodies of these subtypes, or from consensus sequences of such subtypes.

Residues are next selected for substitution, as follows. When an amino acid differs between a 10D5 and 15C11 variable framework region and an equivalent human variable framework region, the human framework amino acid should usually be substituted by the equivalent mouse amino acid if it is reasonably expected that the amino acid:

(1) noncovalently binds antigen directly,

(2) is adjacent to a CDR region, is part of a CDR region under the alternative definition proposed by Chothia et al., supra, or otherwise interacts with a CDR region (e.g., is within about 3A of a CDR region), or

(3) participates in the VL-VH interface.

Computer modeling of the 10D5 and 15C11 antibody heavy and light chain variable regions, and humanization of the 110D5 and 15C11 can be performed in the same manner as in subsection c, supra. Briefly, a three-dimensional model is generated based on the closest solved murine antibody structures for the heavy and light chains. The model is further refined by a series of energy minimization steps to relieve unfavorable atomic contacts and optimize electrostatic and van der Waals interactions.

Computer modeling allows for the identification of CDR-interacting residues. The computer model of the structure of 10D5 or 15C11 can in turn serve as a starting point for predicting the three-dimensional structure of an antibody containing the 10D5 or 15C11 complementarity determining regions substituted in human framework structures. Additional models can be constructed representing the structure as further amino acid substitutions are introduced.

In general, substitution of one, most or all of the amino acids fulfilling the above criteria is desirable. Accordingly, the humanized antibodies of the present invention will often contain a substitution of a human light chain framework residue with a corresponding 10D5 or 15C11 residue in at least 1, 2, 3 or more of the chosen positions. The humanized antibodies also often contain a substitution of a human heavy chain framework residue with a corresponding 10D5 or 15C11 residue in at least 1, 2, 3 or more of the chosen positions.

Occasionally ambiguities about whether a particular amino acid meets the above criteria can be addressed as described in subsection c, supra.

Other candidates for substitution are acceptor human framework amino acids that are rare for a human immunoglobulin at that position. These amino acids can be substituted with amino acids from the equivalent position of more typical human immunoglobulins. Alternatively, amino acids from equivalent positions in the mouse 10D5 or 15C11 can be introduced into the human framework regions when such amino acids are typical of human immunoglobulin at the equivalent positions.

Other candidates for substitution are non-germline residues occurring in a framework region. By performing a computer comparison of 10D5 or 15C11 with known germline sequences, germline sequences with the greatest degree of sequence identity to the heavy or light chain can be identified. Alignment of the framework region and the germline sequence will reveal which residues may be selected for substitution with corresponding germline residues. Residues not matching between a selected light chain acceptor framework and one of these germline sequences could be selected for substitution with the corresponding germline residue.

Mouse and human structures that can be used for computer modeling of 10D5 or 15C11 antibody, as well as germline sequences that can be used in selecting amino acid substitutions, can be obtained use methods described in subsection c, supra. Methods for identifying the mouse and human subgroup and closely related homologs of murine 10D5 or 15C11 antibody are also described supra. Rare mouse residues may be identified by comparing the donor VL and/or VH sequences with the sequences of other members of the subgroup to which the donor VL and/or VH sequences belong (according to Kabat) and identifying the residue positions which differ from the consensus. These donor specific differences may point to somatic mutations which enhance activity. Rare residues close to the binding site may possibly contact the antigen, making it desirable to retain the mouse residue. However, if the rare mouse residue is not important for binding, use of the corresponding acceptor residue is preferred as the mouse residue may create immunogenic neoepitopes in the humanized antibody. In the situation where a rare residue in the donor sequence is actually a common residue in the corresponding acceptor sequence, the preferred residue is clearly the acceptor residue.

In preferred embodiments, a humanized antibody of the present invention contains (i) a light chain comprising a variable domain comprising murine 10D5 or 15C11 VL CDRs and a human acceptor framework, the framework having at least one, residue substituted with the corresponding 10D5 or 15C11 residue and (ii) a heavy chain comprising 10D5 or 15C11 VH CDRs and a human acceptor framework, the framework having at least one, preferably two, three, four, five, six, seven, eight, or nine residues substituted with the corresponding 10D5 or 15C11 residue, and, optionally, at least one, preferably two or three residues substituted with a corresponding human germline residue.

In other preferred embodiments, a 10D5 or 15C11 humanized antibody of the present invention has structural features, as described herein, and further has at least one (preferably two, three, four or all) of the following activities: (1) binds soluble Aβ; (2) binds aggregated Aβ1-42 (e.g., as determined by ELISA); (3) binds Aβ in plaques (e.g., staining of AD and/or PDAPP plaques); (4) binds Aβ with two- to three-fold higher binding affinity as compared to chimeric 10D5 or 15C11 (e.g., 10D5 or 15C11 having murine variable region sequences and human constant region sequences); (5) mediates phagocytosis of Aβ (e.g., in an ex vivo phagocytosis assay, as described herein); and (6) crosses the blood-brain barrier (e.g., demonstrates short-term brain localization, for example, in a PDAPP animal model, as described herein).

In another preferred embodiment, a 10D5 or 15C11 humanized antibody of the present invention has structural features, as described herein, and further binds Aβ in a manner or with an affinity sufficient to elicit at least one of the following in vivo effects: (I) reduce Aβ plaque burden; (2) prevent plaque formation; (3) reduce levels of soluble Aβ; (4) reduce the neuritic pathology associated with an amyloidogenic disorder; (5) lessen or ameliorate at least one physiological symptom associated with an amyloidogenic disorder; and/or (6) improve cognitive function.

In another preferred embodiment, a 10D5 humanized antibody of the present invention has structural features, as described herein, and specifically binds to an epitope comprising residues 3-6 of Aβ. In another preferred embodiment, a 15C11 humanized antibody of the present invention has structural features, as described herein, and specifically binds to an epitope comprising residues 19-22 of Aβ.

In another preferred embodiment, a 10D5 humanized antibody of the present invention has structural features, as described herein, and further binds to an N-terminal epitope within Aβ (e.g., binds to an epitope within amino acids 3-6 of Aβ), and is capable of reducing (1) Aβ peptide levels; (2) Aβ plaque burden; and (3) the neuritic burden or neuritic dystrophy associated with an amyloidogenic disorder.

In another preferred embodiment, a 15C11 humanized antibody of the present invention has structural features, as described herein, and further binds to a central epitope within Aβ (e.g., binds to an epitope within amino acids 19-22 of Aβ), and is capable of reducing (1) Aβ peptide levels; (2) Aβ plaque burden; and (3) the neuritic burden or neuritic dystrophy associated with an amyloidogenic disorder.

g. Exemplary Humanized Antibodies

The present invention features immunological reagents and improved methods for treating Aβ-related diseases or disorders, in particular, for effecting rapid improvement in cognition in patients having or at risk for an Aβ-related disease or disorder. In particular, the reagents and methods are useful in treating patients having or at risk for AD or other amyloidogenic diseases. The invention is based, at least in part, on the identification and characterization of various monoclonal immunoglobulins, having distinctive biological activities, as determined in particular in vitro and/or in vivo activity assays. In exemplary embodiments, antibodies which preferentially bind (or have an increased affinity for) soluble, oligomeric Aβ as compared to monomeric Aβ are selected as reagents for use in the therapeutic methods of the invention. In other exemplary embodiments, antibodies demonstrating efficacy in an appropriate animal model for Aβ-related cognitive deficit are selected as reagents for use in the therapeutic methods of the invention. Antibodies may further have at least one of the following activities: effective at binding beta amyloid protein (Aβ) (e.g., binding soluble and/or aggregated Aβ), mediating phagocytosis (e.g., of aggregated Aβ), reducing plaque burden, reducing neuritic dystrophy and/or improving cognition (e.g., in a subject).

In preferred aspects, the invention features compositions including immunological reagents, in particular Aβ antibodies. In certain embodiments, the compositions include an antibody in an amount effective to rapidly improve cognition in a subject, wherein the antibody is specific for an epitope within Aβ and preferentially binds to soluble oligomeric Aβ as compared to monomeric Aβ. In other embodiments, the compositions include an antibody in an amount effective to rapidly improve cognition in a subject, wherein the antibody is specific for an epitope within Aβ and preferentially binds to soluble oligomeric Aβ as compared to monomeric Aβ, provided that the antibody is not a 266 antibody. In other embodiments, the compositions include an antibody in an amount effective to rapidly improve cognition in a subject, wherein the antibody is specific for an epitope within Aβ and preferentially binds to soluble oligomeric Aβ as compared to monomeric Aβ, provided that the antibody is not a 266 antibody or a 3D6 antibody. In other embodiments, the compositions include an antibody in an amount effective to rapidly improve cognition in a subject, wherein the antibody is specific for an epitope within Aβ and preferentially binds to soluble oligomeric Aβ as compared to monomeric Aβ, wherein the antibody is capable of effecting the improvement in cognition in less that six hours or less than three hours or less than 1 hour (i.e., effects the improvement in cognition in an extremely rapid manner). In other embodiments, the compositions include an antibody in an amount effective to rapidly improve cognition in a subject, wherein the antibody is specific for an epitope within residues 1-5,2-7, 3-6 or 3-7 of Aβ or within 16-23, 16-24, 19-22 or 19-23 of Aβ, and preferentially binds to soluble oligomeric Aβ as compared to monomeric Aβ. In certain embodiments, the antibody binds to the same epitope as the 3D6, 6C6, 2H3, 10D5, 12A11, 2B1, 1C2 or 15C11 antibodies described herein or any other antibody described herein that is capable of effecting rapid improvement in cognition in a subject. Other antibodies of interest are described, for example, in U.S. patent application Ser. No. 10/789,273, and International Patent Application No. WO01/62801A2.

In certain embodiments, the compositions include an Aβ antibody in an amount effective to rapidly improve cognition in a subject, wherein the antibody is specific for an epitope within residues 1-10 of Aβ and preferentially binds to soluble oligomeric Aβ as compared to monomeric Aβ. In other embodiments, the compositions include an Aβ antibody in an amount effective to rapidly improve cognition in a subject, wherein the antibody is specific for an epitope within residues 1-10 of Aβ and effects a rapid improvement in cognition in an animal model of an Aβ-related disorder as determined in a Contextual Fear Conditioning (CFC) assay. In additional embodiments, the compositions include an Aβ antibody in an amount effective to rapidly improve cognition in a subject, wherein the antibody is specific for an epitope within residues 1-10 of Aβ, preferentially binds to soluble oligomeric Aβ as compared to monomeric Aβ and effects a rapid improvement in cognition in an animal model of an Aβ-related disorder as determined in a Contextual Fear Conditioning (CFC) assay. In certain embodiments, the Aβ antibody binds to an epitope within residues 3-7 of Aβ. In other embodiments, the immunological reagent is selected from the group consisting of a 3D6 immunological reagent, a 6C6 immunological reagent, a 10D5 immunological reagent, and a 12A11 immunological reagent. In other embodiments, the Aβ antibody is selected from the group consisting of a 3D6 antibody, a 6C6 antibody, a 10D5 antibody, and a 12A11 antibody. In other embodiments, the Aβ antibody is not a 3D6 antibody.

In one embodiment, the antibody may be a 3D6 antibody or variant thereof, or a 10D5 antibody or variant thereof, both of which are described in U.S. Patent Publication No. 20030165496A1, U.S. Patent Publication No. 20040087777A1, International Patent Publication No. WO02/46237A3. Description of 3D6 and 10D5 can also be found, for example, in International Patent Publication No. WO02/088306A2 and International Patent Publication No. WO02/088307A2. In yet another embodiment, the antibody may be a 12A11 antibody or a variant thereof, as described in U.S. patent application Ser. No. 10/858,855 and International Patent Application No. PCT/US04/17514.

In certain embodiments, the compositions include an Aβ antibody in an amount effective to rapidly improve cognition in a subject, wherein the antibody is specific for an epitope within residues 13-28 of Aβ and preferentially binds to soluble oligomeric Aβ as compared to monomeric Aβ. In other embodiments, the compositions include an Aβ antibody in an amount effective to rapidly improve cognition in a subject, wherein the antibody is specific for an epitope within residues 13-28 of Aβ and effects a rapid improvement in cognition in an animal model of an Aβ-related disorder as determined in a Contextual Fear Conditioning (CFC) assay. In additional embodiments, the compositions include an Aβ antibody in an amount effective to rapidly improve cognition in a subject, wherein the antibody is specific for an epitope within residues 13-28 of Aβ, preferentially binds to soluble oligomeric Aβ as compared to monomeric Aβand effects a rapid improvement in cognition in an animal model of an Aβ-related disorder as determined in a Contextual Fear Conditioning (CFC) assay. In certain embodiments, the Aβ antibody binds to an epitope within residues 16-24 of Aβ. In other embodiments, the immunological reagent is selected from the group consisting of a 2B1 immunological reagent, a 1C2 immunological reagent, a 15C11 immunological reagent, and a 9G8 immunological reagent. In other embodiments, the Aβ antibody is selected from the group consisting of a 2B1 antibody, a 1C2 antibody, a 15C11 antibody and a 9G8 antibody. In other embodiments, the Aβ antibody is not a 266 antibody. In certain embodiments, the antibody may be a 15C11 or 9G8 antibody or variants thereof, as described in a U.S. Patent Application corresponding to Attorney Docket No. ELN-055-1, filed on even date herewith, and entitled “Humanized Antibodies that Recognize Beta Amyloid Peptide.”

In yet other embodiments, the Aβ antibody neutralizes one or more neuroactive Aβ species. In other embodiments, the Aβ antibody clears plaques. In certain embodiments, the compositions of the invention are formulated for single dose administration. In other embodiments, the compositions of the invention are formulated for multiple dose administration.

The invention also features compositions including combinations of any of the foregoing immunological reagents, as well as methods for effecting rapid improvement in cognition in a subject by administering an effective amount of a combination of such immunological reagents. In certain embodiments, a combination of antibodies are administered (e.g., a composition including a combination of antibodies or compositions including each antibody formulated for separate administration), wherein each antibody is specific for an epitope within Aβ, and preferentially binds to soluble oligomeric Aβ as compared to monomeric Aβ. In other embodiments, each antibody is capable of effecting improvement in cognition in a subject in less than six hours, less than three hours, or less than 1 hour (i.e., effects the improvement in cognition in an extremely rapid manner). Other embodiments feature administration of effective amounts of an antibody that is specific for an N-terminal epitope of Aβ and an antibody that is specific for a central epitope of Aβ, wherein each antibody is capable of effecting rapid improvement in cognition in a subject. Yet other embodiments feature administration of effective amounts of an antibody specific for an epitope within residues 1-10, 1-5,2-7, 3-6 or 3-7 of Aβ and an antibody specific for an epitope within 16-23, 16-24, 19-22 or 19-23 of Aβ. Other embodiments of the invention feature combinations of immunological reagents selected from the group consisting of a 3D6 immunological reagent, a 6C6 immunological reagent, a 10D5 immunological reagent, a 12A11 immunological reagent, a 2B1 immunological reagent, a 1C2 immunological reagent, a 15C11 immunological reagent, a 9G8 immunological reagent and a 266 immunological reagent. Yet other embodiments feature combinations of antibodies, wherein each antibody binds to the same epitope as the 3D6, 6C6, 2H3, 10D5, 12A11, 2B1, 1C2, 15C11 or 266 antibody. In certain embodiments, a combination of antibodies are administered in which each antibody is selected from the group consisting of a 3D6 antibody, a 6C6 antibody, a 10D5 antibody, a 12A11 antibody, a 2B1 antibody, a 1C2 antibody, a 15C11 antibody, a 9G8 antibody and a 266 antibody.

The invention is further based on the determination and structural characterization of the primary and secondary structure of the variable light and heavy chains of the selected immunoglobulins and the identification of residues important for activity and immunogenicity.

Immunoglobulins are featured which include a variable light and/or variable heavy chain of the monoclonal immunoglobulins described herein. Preferred immunoglobulins, e.g., therapeutic immunoglobulins, are featured which include a humanized variable light and/or humanized variable heavy chain. Preferred variable light and/or variable heavy chains include a complementarity determining region (CDR) from a select immunoglobulin (e.g., donor immunoglobulin) and variable framework regions from or substantially from a human acceptor immunoglobulin. The phrase “substantially from a human acceptor immunoglobulin” means that the majority or key framework residues are from the human acceptor sequence, allowing however, for substitution of residues at certain positions with residues selected to improve activity of the humanized immunoglobulin (e.g., alter activity such that it more closely mimics the activity of the donor immunoglobulin) or selected to decrease the immunogenicity of the humanized immunoglobulin.

In one embodiment, the invention features a humanized immunoglobulin light or heavy chain that includes murine monoclonal antibody variable region complementarity determining regions (CDRs) (i.e., includes one, two or three CDRs from the light chain variable region sequence set forth as SEQ ID NO:2, 14, 18, 28 or 57 and/or includes one, two or three CDRs from the heavy chain variable region sequence set forth as SEQ ID NO:4, 16, 20, 30 or 59, respectively), and includes a variable framework region from a human acceptor immunoglobulin light or heavy chain sequence, optionally having at least one residue of the framework residue backmutated to a corresponding murine residue, wherein said backmutation does not substantially affect the ability of the chain to direct Aβ binding.

In one embodiment, the invention features a humanized immunoglobulin light or heavy chain that includes murine monoclonal antibody variable region complementarity determining regions (CDRs) (i.e., includes one, two or three CDRs from the light chain variable region sequence set forth as SEQ ID NO:2, 14, 18, 28 or 57 and/or includes one, two or three CDRs from the heavy chain variable region sequence set forth as SEQ ID NO:4, 16, 20, 30 or 59, respectively), and includes a variable framework region substantially from a human acceptor immunoglobulin light or heavy chain sequence, optionally having at least one residue of the framework residue backmutated to a corresponding murine residue, wherein said backmutation does not substantially affect the ability of the chain to direct Aβ binding.

In another embodiment, the invention features a humanized immunoglobulin light or heavy chain that includes murine monoclonal antibody variable region complementarity determining regions (CDRs) (e.g., includes one, two or three CDRs from the light chain variable region sequence set forth as SEQ ID NO:2, 14, 18, 28 or 57 and/or includes one, two or three CDRs from the heavy chain variable region sequence set forth as SEQ ID NO:4, 16, 20, 30 or 59, respectively), and includes a variable framework region substantially from a human acceptor immunoglobulin light or heavy chain sequence, optionally having at least one framework residue substituted with the corresponding amino acid residue from the murine light or heavy chain variable region sequence, where the framework residue is selected from the group consisting of (a) a residue that non-covalently binds antigen directly; (b) a residue adjacent to a CDR; (c) a CDR-interacting residue (e.g., identified by modeling the light or heavy chain on the solved structure of a homologous known immunoglobulin chain); and (d) a residue participating in the VL-VH interface.

In another embodiment, the invention features a humanized immunoglobulin light or heavy chain that includes murine monoclonal antibody variable region CDRs and variable framework regions from a human acceptor immunoglobulin light or heavy chain sequence, optionally having at least one framework residue substituted with the corresponding amino acid residue from the murine light or heavy chain variable region sequence, where the framework residue is a residue capable of affecting light chain variable region conformation or function as identified by analysis of a three-dimensional model of the variable region, for example a residue capable of interacting with antigen, a residue proximal to the antigen binding site, a residue capable of interacting with a CDR, a residue adjacent to a CDR, a residue within 6 Å of a CDR residue, a canonical residue, a vernier zone residue, an interchain packing residue, a rare residue, or a glycoslyation site residue on the surface of the structural model.

In another embodiment, the invention features, in addition to the substitutions described above, a substitution of at least one rare human framework residue. For example, a rare residue can be substituted with an amino acid residue which is common for human variable chain sequences at that position. Alternatively, a rare residue can be substituted with a corresponding amino acid residue from a homologous germline variable chain sequence.

In other embodiments, the methods of the invention feature polypeptides comprising the complementarity determining regions (CDRs) of a monoclonal antibody featured herein, including polynucleotide reagents, vectors and host cells suitable encoding said polypeptides.

In another embodiment, the invention features pharmaceutical compositions that include a humanized immunoglobulin as described herein and a pharmaceutical carrier. Also featured are isolated nucleic acid molecules, vectors and host cells for producing the immunoglobulins or immunoglobulin fragments or chains described herein, as well as methods for producing said immunoglobulins, immunoglobulin fragments or immunoglobulin chains.

The present invention further features a method for identifying residues amenable to substitution when producing a humanized immunoglobulins of the invention. For example, a method for identifying variable framework region residues amenable to substitution involves modeling the three-dimensional structure of a selected murine monoclonal antibody variable region on a solved homologous immunoglobulin structure and analyzing said model for residues capable of affecting immunoglobulin variable region conformation or function, such that residues amenable to substitution are identified. The invention further features use of the variable region sequence set forth as SEQ ID NO:2, 14, 18, 28 or 37 or SEQ ID NO:4, 16, 20, 30 or 59, or any portion thereof, in producing a three-dimensional image of a immunoglobulin, immunoglobulin chain, or domain thereof.

The activities of the antibodies described above can be determined utilizing any one of a variety of assays described herein or in the art (e.g., binding assays, phagocytosis assays, etc.). Activities can be assayed either in vivo (e.g., using labeled assay components and/or imaging techniques) or in vitro (e.g., using samples or specimens derived from a subject). Activities can be assayed either directly or indirectly. In certain preferred embodiments, neurological endpoints (e.g., amyloid burden, neuritic burden, etc) are assayed. Such endpoints can be assayed in living subjects (e.g., in animal models of Alzheimer's disease or in human subjects, for example, undergoing immunotherapy) using non-invasive detection methodologies. Alternatively, such endpoints can be assayed in subjects post mortem. Assaying such endpoints in animal models and/or in human subjects post mortem is useful in assessing the effectiveness of various agents (e.g., humanized antibodies) to be utilized in similar immunotherapeutic applications. In other preferred embodiments, behavioral or neurological parameters can be assessed as indicators of the above neuropathological activities or endpoints.

3. Production of Variable Regions

Having conceptually selected the CDR and framework components of humanized immunoglobulins, a variety of methods are available for producing such immunoglobulins. In general, one or more of the murine complementarity determining regions (CDR) of the heavy and/or light chain of the antibody can be humanized, for example, placed in the context of one or more human framework regions, using primer-based polymerase chain reaction (PCR). Briefly, primers are designed which are capable of annealing to target murine CDR region(s) which also contain sequence which overlaps and can anneal with a human framework region. Accordingly, under appropriate conditions, the primers can amplify a murine CDR from a murine antibody template nucleic acid and add to the amplified template a portion of a human framework sequence. Similarly, primers can be designed which are capable of annealing to a target human framework region(s) where a PCR reaction using these primers results in an amplified human framework region(s). When each amplification product is then denatured, combined, and annealed to the other product, the murine CDR region, having overlapping human framework sequence with the amplified human framework sequence, can be genetically linked. Accordingly, in one or more such reactions, one or more murine CDR regions can be genetically linked to intervening human framework regions.

In some embodiments, the primers may also comprise desirable restriction enzyme recognition sequences to facilitate the genetic engineering of the resultant PCR amplified sequences into a larger genetic segment, for example, a variable light or heavy chain segment, heavy chain, or vector. In addition, the primers used to amplify either the murine CDR regions or human framework regions may have desirable mismatches such that a different codon is introduced into the murine CDR or human framework region. Typical mismatches introduce alterations in the human framework regions that preserve or improve the structural orientation of the murine CDR and thus its binding affinity, as described herein.

It should be understood that the foregoing approach can be used to introduce one, two, or all three murine CDR regions into the context of intervening human framework regions. Methods for amplifying and linking different sequences using primer-based PCR are described in, for example, Sambrook, Fritsch and Maniatis, Molecular Cloning: Cold Spring Harbor Laboratory Press (1989); DNA Cloning, Vols. 1 and 2, (D. N. Glover, Ed. 1985); PCR Handbook Current Protocols in Nucleic Acid Chemistry, Beaucage, Ed. John Wiley & Sons (1999) (Editor); Current Protocols in Molecular Biology, eds. Ausubel et al., John Wiley & Sons (1992).

Because of the degeneracy of the code, a variety of nucleic acid sequences will encode each immunoglobulin amino acid sequence. The desired nucleic acid sequences can be produced by de novo solid-phase DNA synthesis or by PCR mutagenesis of an earlier prepared variant of the desired polynucleotide. Oligonucleotide-mediated mutagenesis is a preferred method for preparing substitution, deletion and insertion variants of target polypeptide DNA. See Adelman et al., DNA 2:183 (1983). Briefly, the target polypeptide DNA is altered by hybridizing an oligonucleotide encoding the desired mutation to a single-stranded DNA template. After hybridization, a DNA polymerase is used to synthesize an entire second complementary strand of the template that incorporates the oligonucleotide primer, and encodes the selected alteration in the target polypeptide DNA.

4. Selection of Constant Regions

The variable segments of antibodies produced as described supra (e.g., the heavy and light chain variable regions of chimeric or humanized antibodies) are typically linked to at least a portion of an immunoglobulin constant region (Fc region), typically that of a human immunoglobulin. Human constant region DNA sequences can be isolated in accordance with well known procedures from a variety of human cells, but preferably immortalized B cells (see Kabat et al., supra, and Liu et al., WO87/02671) (each of which is incorporated by reference in its entirety for all purposes). Ordinarily, the antibody will contain both light chain and heavy chain constant regions. The heavy chain constant region usually includes CH1, hinge, CH2, CH3, and CH4 regions. The antibodies described herein include antibodies having all types of constant regions, including IgM, IgG, IgD, IgA and IgE, and any isotype, including IgG1, IgG2, IgG3 and IgG4. When it is desired that the antibody (e.g., humanized antibody) exhibit cytotoxic activity, the constant domain is usually a complement fixing constant domain and the class is typically IgG1. Human isotypes IgG1 and IgG4 are exemplary. Light chain constant regions can be lambda or kappa. The humanized antibody may comprise sequences from more than one class or isotype. Antibodies can be expressed as tetramers containing two light and two heavy chains, as separate heavy chains, light chains, as Fab, Fab′ F(ab′)2, and Fv, or as single chain antibodies in which heavy and light chain variable domains are linked through a spacer.

In some embodiments, humanized antibodies described herein are modified to enhance their antigen dependent cellular cytotoxicity (ADCC) activity using techniques, such as, for example, those described in U.S. Pat. No. 6,946,292, the entire contents of which are incorporated by reference herein. ADCC activity of antibodies is generally thought to require the binding of the Fc region of an antibody to an antibody receptor existing on the surface of an effector cell, such as, for example, a killer cell, a natural killer cell and an activated macrophage. By altering fucosylation (e.g., reducing or eliminating) of the carbohydrate structure of a humanized antibody (i.e., in the Fc region), the ADCC activity of the antibody can be enhanced in vitro by, for example, 10-fold, or 20-fold, or 30-fold, or 40-fold, or 50-fold, or 100-fold, relative to an unmodified humanized antibody. Because of increased ADCC activity, such modified antibodies can be used at lower dosages than their unmodified counterparts and generally have fewer or reduced side effects in patients.

In some embodiments, aglycosyl versions of humanized antibodies are featured, wherein such antibodies include an aglycosylated constant region. Oligosaccharide at Asn-297 is a characteristic feature of normal human IgG antibodies (See, Kabat et al., 1987, Sequence of Proteins of Immunological Interest, U.S. Department of Health Human Services Publication). Each of the two heavy chains in IgG molecules have a single branched chain carbohydrate group which is linked to the amide group of the asparagine residue, for example, at position 297. Substitution of, for example, asparagine with alanine prevents the glycosylation of the antibody, as described in, for example, U.S. Pat. No. 6,706,265, incorporated by reference herein. In a particular embodiment, the amino acid residue Asn at position 297 is mutated to alanine.

5. Expression of Recombinant Antibodies

Chimeric and humanized antibodies are typically produced by recombinant expression. Nucleic acids encoding light and heavy chain variable regions, optionally linked to constant regions, are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences. Preferably, the expression control sequences are eukaryotic promoter systems in vectors capable of transforming or transfecting eukaryotic host cells (e.g., COS or CHO cells). Once the vector has been incorporated into the appropriate host, the host is maintained under conditions suitable for high level expression of the nucleotide sequences, and the collection and purification of the crossreacting antibodies.

These expression vectors are typically replicable in the host organisms either as episomes or as an integral part of the host chromosomal DNA. Commonly, expression vectors contain selection markers (e.g., ampicillin-resistance, hygromycin-resistance, tetracycline resistance, kanamycin resistance or neomycin resistance) to permit detection of those cells transformed with the desired DNA sequences (see, e.g., Itakura et al., U.S. Pat. No. 4,704,362).

E. coli is one prokaryotic host particularly useful for cloning the polynucleotides (e.g., DNA sequences) of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Other microbes, such as yeast, are also useful for expression. Saccharomyces is a preferred yeast host, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

In addition to microorganisms, mammalian tissue cell culture may also be used to express and produce the polypeptides of the present invention (e.g., polynucleotides encoding immunoglobulins or fragments thereof). See Winnacker, From Genes to Clones, VCH Publishers, N.Y., N.Y. (1987). Eukaryotic cells are actually preferred, because a number of suitable host cell lines capable of secreting heterologous proteins (e.g., intact immunoglobulins) have been developed in the art, and include CHO cell lines, various Cos cell lines, HeLa cells, preferably, myeloma cell lines, or transformed B-cells or hybridomas. Preferably, the cells are nonhuman. Expression vectors for these cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like. See Co et al., J. Immunol. 148:1149 (1992).

Alternatively, antibody-coding sequences can be incorporated in transgenes for introduction into the genome of a transgenic animal and subsequent expression in the milk of the transgenic animal (see, e.g., Deboer et al., U.S. Pat. No. 5,741,957, Rosen, U.S. Pat. No. 5,304,489, and Meade et al., U.S. Pat. No. 5,849,992). Suitable transgenes include coding sequences for light and/or heavy chains in operable linkage with a promoter and enhancer from a mammary gland specific gene, such as casein or beta lactoglobulin.

Alternatively, antibodies (e.g., humanized antibodies) of the invention can be produced in transgenic plants (e.g., tobacco, maize, soybean and alfalfa). Improved ‘plantibody’ vectors (Hendy et al. (1999) J. Immunol. Methods 231:137-146) and purification strategies coupled with an increase in transformable crop species render such methods a practical and efficient means of producing recombinant immunoglobulins not only for human and animal therapy, but for industrial applications as well (e.g., catalytic antibodies). Moreover, plant produced antibodies have been shown to be safe and effective and avoid the use of animal-derived materials and therefore the risk of contamination with a transmissible spongiform encephalopathy (TSE) agent. Further, the differences in glycosylation patterns of plant and mammalian cell-produced antibodies have little or no effect on antigen binding or specificity. In addition, no evidence of toxicity or HAMA has been observed in patients receiving topical oral application of a plant-derived secretory dimeric IgA antibody (see Larrick et al. (1998) Res. Immunol. 149:603-608).

Various methods may be used to express recombinant antibodies in transgenic plants. For example, antibody heavy and light chains can be independently cloned into expression vectors (e.g., Agrobacterium tumefaciens vectors), followed by the transformation of plant tissue in vitro with the recombinant bacterium or direct transformation using, e.g., particles coated with the vector which are then physically introduced into the plant tissue using, e.g., ballistics. Subsequently, whole plants expressing individual chains are reconstituted followed by their sexual cross, ultimately resulting in the production of a fully assembled and functional antibody. Similar protocols have been used to express functional antibodies in tobacco plants (see Hiatt et al. (1989) Nature 342:76-87). In various embodiments, signal sequences may be utilized to promote the expression, binding and folding of unassembled antibody chains by directing the chains to the appropriate plant environment (e.g., the aqueous environment of the apoplasm or other specific plant tissues including tubers, fruit or seed) (see Fiedler et al. (1995) Bio/Technology 13:1090-1093). Plant bioreactors can also be used to increase antibody yield and to significantly reduce costs.

The vectors containing the polynucleotide sequences of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. (See generally Sambrook et al., Molecular Cloning: A Laboratory Manual (Cold Spring Harbor Press, 2nd ed., 1989) (incorporated by reference in its entirety for all purposes). Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

When heavy and light chains are cloned on separate expression vectors, the vectors are co-transfected to obtain expression and assembly of intact immunoglobulins. Once expressed, the whole antibodies, their dimers, individual light and heavy chains, or other immunoglobulin forms of the present invention can be purified according to standard procedures of the art, including ammonium sulfate precipitation, affinity columns, column chromatography, HPLC purification, gel electrophoresis and the like (see generally Scopes, Protein Purification (Springer-Verlag, N.Y., (1982)). Substantially pure immunoglobulins of at least about 90 to 95% homogeneity are preferred, and 98 to 99% or more homogeneity most preferred, for pharmaceutical uses.

6. Antibody Fragments

Also contemplated within the scope of the instant invention are antibody fragments. In one embodiment, fragments of non-human, and/or chimeric antibodies are provided. In another embodiment, fragments of humanized antibodies are provided. Typically, these fragments exhibit specific binding to antigen with an affinity of at least 10⁷, and more typically 10⁸ or 10⁹ M⁻¹. Humanized antibody fragments include separate heavy chains, light chains, Fab, Fab′, F(ab′)2, Fabc, and Fv. Fragments are produced by recombinant DNA techniques, or by enzymatic or chemical separation of intact immunoglobulins.

In some embodiments, the generally short half-life of antibody fragments (e.g., Fabs or Fab's) is extended by pegylation. This is generally achieved by fusion to polyethylene glycol (PEG), as described by, for example, Leong, et al. Cytokine 16, 106-119 (2001). Pegylation has the added advantage of eliminating Fc receptor mediated function, where desired, and/or reducing immunogenicity. In exemplary embodiments, 2-20 kDa PEG molecules are covalently attached, for example, to an antibody heavy chain hinge region via a K-linker-C (See, e.g., Choy et al., Rheumatol. 41:1133-1137 (2002)).

7. Epitope Mapping

Epitope mapping can be performed to determine which antigenic determinant or epitope of Aβ is recognized by the antibody. In one embodiment, epitope mapping is performed according to Replacement NET (rNET) analysis. The rNET epitope map assay provides information about the contribution of individual residues within the epitope to the overall binding activity of the antibody. rNET analysis uses synthesized systematic single substituted peptide analogs. Binding of an antibody being tested is determined against native peptide (native antigen) and against 19 alternative “single substituted” peptides, each peptide being substituted at a first position with one of 19 non-native amino acids for that position. A profile is generated reflecting the effect of substitution at that position with the various non-native residues. Profiles are likewise generated at successive positions along the antigenic peptide. The combined profile, or epitope map, (reflecting substitution at each position with all 19 non-native residues) can then be compared to a map similarly generated for a second antibody. Substantially similar or identical maps indicate that antibodies being compared have the same or similar epitope specificity.

8. Testing Antibodies for Therapeutic Efficacy (e.g., Plague Clearing Activity) in Animal Models

Groups of 7-9 month old PDAPP mice each are injected with 0.5 mg in PBS of polyclonal anti-Aβ or specific anti-Aβ monoclonal, humanized, or chimeric antibodies. All antibody preparations are purified to have low endotoxin levels. Monoclonals can be prepared against a fragment by injecting the fragment or longer form of Aβ into a mouse, preparing hybridomas and screening the hybridomas for an antibody that specifically binds to a desired fragment of Aβ without binding to other nonoverlapping fragments of Aβ. Humanized and/or chimeric antibodies are prepared as described herein.

Mice are injected intraperitoneally as needed over a 4 month period to maintain a circulating antibody concentration measured by ELISA titer of greater than 1/1000 defined by ELISA to Aβ42 or other immunogen. Titers are monitored and mice are euthanized at the end of 6 months of injections. Histochemistry, Aβ levels and toxicology are performed post mortem. Ten mice are used per group.

9. Testing Antibodies for Binding to Soluble Oligomeric Aβ

The invention also provides methods of testing the ability of an antibody to bind to soluble, oligomeric Aβ in a biochemical assay. The biochemical assay is based, at least in part, on a comparison of the binding of an antibody to one or more forms of soluble, oligomeric Aβ (e.g., Aβ dimers, Aβ trimers, Aβ tetramers, Aβpentamers, and the like) as compared to the binding of the antibody to monomeric Aβ. This comparison can be used to determine a relative binding of the antibody to soluble, oligomeric Aβ as compared to monomeric Aβ. In various embodiments, this relative binding is compared to a corresponding relative binding of a control reagent to one or more soluble oligomeric Aβ species versus monomeric Aβ. In other aspects, the affinity of an antibody for one or more oligomeric Aβ species is compared to the antibody's affinity for monomeric Aβ in the Aβ preparation. It has been discovered that a strong correlation exists between an Aβ antibody's ability to preferentially bind soluble, oligomeric Aβ species and the ability of the antibody to rapidly improve cognition as assessed by a CFC assay in an appropriate model animal, as described in detail infra. An antibody's ability to improve cognition in the CFC assay is further believed to be a strong indicator or predictor of the antibody's ultimate human therapeutic efficacy (in particular, efficacy in rapidly improving cognition in a patient). Accordingly, a comparison of Aβ antibody binding preferences and/or affinities leads to the identification of certain antibodies as candidates for use in the therapeutic methods of the invention, in particular, for use in method for effecting rapid improvement in cognition in a patient.

Candidate antibodies exhibit a preferential or greater binding to one or more soluble oligomeric Aβ species as compared to monomeric Aβ. Antibodies that preferentially bind to, for example, Aβ dimers, trimers and tetramers as compared to monomeric Aβ are preferred candidates for use in methods for effecting rapid improvement in cognition in a patient. For example, candidate antibodies exhibiting a two-fold, three-fold, four-fold, five-fold, ten-fold, twenty-fold or more greater binding to soluble oligomeric Aβ species as compared to monomeric Aβ are selected for use in the therapeutic methods.

The binding of an antibody to one or more soluble, oligomeric Aβ species or to monomeric Aβ can be determined qualitatively, quantitatively, or combination of both. In general, any technique capable of distinguishing oligomeric Aβ species from monomeric Aβ in an Aβ preparation comprising the species can be used. In exemplary embodiments, one or more of immunoprecipitation, electrophoretic separation, and chromatographic separation (e.g., liquid chromatography), can be used to distinguish oligomeric Aβ species from monomeric Aβ in an Aβ preparation comprising the species.

In preferred embodiments, the binding of the antibody to one or more soluble, oligomeric Aβ species or to monomeric Aβ is determined by immunoprecipitating the Aβ species from the preparation. The immunoprecipitate is then subjected to an electrophoretic separation (e.g., SDS-PAGE) to distinguish oligomeric species from monomeric Aβ in the precipitate. The amount of oligomeric Aβ species and monomeric Aβ present in the electrophoretic bands can be visualized, for example, by immunoblotting of the electrophoretic gel or by direct quantitation of the respective species in the bands of the electrophoretic gel. The amount of precipitate for an Aβ species can be determined, for example, from the intensity of the corresponding electrophoretic bands, immunoblot bands, or a combination of both. The intensity determination can be qualitative, quantitative, or a combination of both.

Assessment of band intensity can be performed, for example, using appropriate film exposures which can be scanned and the density of bands determined with software, for example, AlphaEase™ software (AlphaInnotech™). Assessment of band intensity can be performed, for example, using any of a number of labels incorporated into the antibody, an imaging reagent (e.g., an antibody used in an immunoblot), or both. Suitable labels include, but are not limited to, fluorescent labels, radioactive labels, paramagnetic labels, or combinations thereof.

In various embodiments, the amount of one or more oligomeric Aβ species and/or monomeric Aβ which bind to an antibody can be assessed using mass spectrometry, for example, on the Aβ preparation itself a suitable time after it has been contacted with the antibody, or on monomeric Aβ and/or one or more soluble, oligomeric Aβ species bound to the antibody which have been extracted from the Aβ preparation.

In certain aspects, the affinity of an antibody for one or more oligomeric Aβ species is compared to the antibody's affinity for monomeric Aβ to identify the antibody as a candidate for use in the therapeutic methods of the invention, in particular, for use in method for effecting rapid improvement in cognition in a patient. The affinity of the test antibody (e.g., an Aβ antibody) for oligomeric Aβ as compared to monomeric Aβ can be compared to the binding affinities of a control reagent. Labels can be used to assess the affinity of an antibody for monomeric Aβ, oligomeric Aβ, or both. In various embodiments, a primary reagent with affinity for Aβ is unlabelled and a secondary labeling agent is used to bind to the primary reagent. Suitable labels include, but are not limited to, fluorescent labels, paramagnetic labels, radioactive labels, and combinations thereof.

In certain aspects, the methods of the invention feature the administration of an anti-Aβ antibody that is capable of rapidly improving cognition in a subject wherein the anti-Aβ antibody has been identified in using an assay which is suitably predictive of immunotherapeutic efficacy in the subject. In exemplary embodiments, the assay is a biochemical assay that is based, at least in part, on a comparison of the binding of one or more Aβ oligomers in an Aβ preparation to a test immunotherapeutic agent to the binding of Aβ monomers in the Aβ preparation to the test immunotherapeutic agent. The one or more Aβ oligomers can include, for example, one or more of Aβ dimers, Aβtrimers, Aβ tetramers, and Aβ pentamers. In various embodiments, the test immunotherapeutic agent is identified when the binding of one or more Aβ oligomers in the Aβ preparation to the test immunotherapeutic agent is greater than the binding of Aβ monomers in the Aβ preparation to the test immunotherapeutic agent. The amount of Aβ monomers and one or more Aβ oligomer species in an Aβ preparation which bind to a test immunological reagent can be assessed using biochemical methods, for example using immunoprecipitation to precipitate from the Aβ preparation the Aβ monomers and one or more Aβ oligomer species bound to the test immunological reagent followed by an electrophoretic separation of the immunoprecipitates. Such biochemical assays are discussed further herein and in U.S. provisional patent application Ser. Nos. 60/636,687, filed Dec. 15, 2004, and 60/736,045, filed Nov. 10, 2005, both entitled “AN IMMUNOPRECIPITATION-BASED ASSAY FOR PREDICTING IN VIVO EFFICACY OF BETA-AMYLOID ANTIBODIES,” the entire contents of each are incorporated by reference herein.

10. Screening Antibodies for Clearing Activity

The invention also provides methods of screening an antibody for activity in clearing an amyloid deposit or any other antigen, or associated biological entity, for which clearing activity is desired. To screen for activity against an amyloid deposit, a tissue sample from a brain of a patient with Alzheimer's disease or an animal model having characteristic Alzheimer's pathology is contacted with phagocytic cells bearing an Fc receptor, such as microglial cells, and the antibody under test in a medium in vitro. The phagocytic cells can be a primary culture or a cell line, and can be of murine (e.g., BV-2 or C8-B4 cells) or human origin (e.g., THP-1 cells). In some methods, the components are combined on a microscope slide to facilitate microscopic monitoring. In some methods, multiple reactions are performed in parallel in the wells of a microtiter dish. In such a format, a separate miniature microscope slide can be mounted in the separate wells, or a nonmicroscopic detection format, such as ELISA detection of Aβ can be used. Preferably, a series of measurements is made of the amount of amyloid deposit in the in vitro reaction mixture, starting from a baseline value before the reaction has proceeded, and one or more test values during the reaction. The antigen can be detected by staining, for example, with a fluorescently labeled antibody to Aβ or other component of amyloid plaques. The antibody used for staining may or may not be the same as the antibody being tested for clearing activity. A reduction relative to baseline during the reaction of the amyloid deposits indicates that the antibody under test has clearing activity. Such antibodies are likely to be useful in preventing or treating Alzheimer's and other amyloidogenic diseases. Particularly useful antibodies for preventing or treating Alzheimer's and other amyloidogenic diseases include those capable of clearing both compact and diffuse amyloid plaques, for example, the 12A11 antibody of the instant invention, or chimeric or humanized versions thereof.

Analogous methods can be used to screen antibodies for activity in clearing other types of biological entities. The assay can be used to detect clearing activity against virtually any kind of biological entity. Typically, the biological entity has some role in human or animal disease. The biological entity can be provided as a tissue sample or in isolated form. If provided as a tissue sample, the tissue sample is preferably unfixed to allow ready access to components of the tissue sample and to avoid perturbing the conformation of the components incidental to fixing. Examples of tissue samples that can be tested in this assay include cancerous tissue, precancerous tissue, tissue containing benign growths such as warts or moles, tissue infected with pathogenic microorganisms, tissue infiltrated with inflammatory cells, tissue bearing pathological matrices between cells (e.g., fibrinous pericarditis), tissue bearing aberrant antigens, and scar tissue. Examples of isolated biological entities that can be used include Aβ, viral antigens or viruses, proteoglycans, antigens of other pathogenic microorganisms, tumor antigens, and adhesion molecules. Such antigens can be obtained from natural sources, recombinant expression or chemical synthesis, among other means. The tissue sample or isolated biological entity is contacted with phagocytic cells bearing Fc receptors, such as monocytes or microglial cells, and an antibody to be tested in a medium. The antibody can be directed to the biological entity under test or to an antigen associated with the entity. In the latter situation, the object is to test whether the biological entity is phagocytosed with the antigen. Usually, although not necessarily, the antibody and biological entity (sometimes with an associated antigen), are contacted with each other before adding the phagocytic cells. The concentration of the biological entity and/or the associated antigen remaining in the medium, if present, is then monitored. A reduction in the amount or concentration of antigen or the associated biological entity in the medium indicates the antibody has a clearing response against the antigen and/or associated biological entity in conjunction with the phagocytic cells.

11. Testing Antibodies for a Rapid or Prolonged Improvement in Cognition in a CFC Assay

In various aspects, an antibody of the invention can be tested for the ability to improve cognition in an appropriate animal model. For example, the ability of an antibody to improve cognition in an animal model for AD, as assessed via a contextual fear conditioning (CFC) assay, can be used to select the antibody as a candidate for use in the therapeutic methods of the invention, in particular, in methods for effecting rapid improvement in cognition in a patient.

Contextual fear conditioning is a common form of learning that is exceptionally reliable and rapidly acquired in most animals, for example, mammals. Test animals learn to fear a previously neutral stimulus and/or enviornment because of its association with an aversive experience. (see, e.g., Fanselow, Anim. Learn. Behav. 18:264-270 (1990); Wehner et al., Nature Genet. 17:331-334. (1997); Caldarone et al., Nature Genet. 17:335-337 (1997)).

Contextual fear conditioning is especially useful for determining cognitive function or dysfunction, e.g., as a result of disease or a disorder, such as a neurodegenerative disease or disorder, an Aβ-related disease or disorder, an amyloidogenic disease or disorder, the presence of an unfavorable genetic alteration affecting cognitive function (e.g., genetic mutation, gene disruption, or undesired genotype), and/or the efficacy of an agent, e.g., an immunological reagent, on cognitive ability. Accordingly, the CFC assay provides a method for independently testing and/or validating the therapeutic effect of agents for preventing or treating a cognitive disease or disorder, and in particular, a disease or disorder affecting one or more regions of the brains, e.g., the hippocampus, subiculum, cingulated cortex, prefrontal cortex, perirhinal cortex, sensory cortex, and medial temporal lobe.

Typically, the CFC assay is performed using standard animal chambers and the employment of conditioning training comprising a mild shock (e.g., 0.35 mA foot shock) paired with an auditory (e.g., a period of 85 db white noise), olfactory (e.g., almond or lemon extract), touch (e.g., floor cage texture), and/or visual cue (light flash). Alternatively, conditioning training comprises administration of the shock absent a paired cue (i.e., shock associated with context). The response to the aversive experience (shock) is typically one of freezing (absence of movement except for respiration) but may also include eye blink, or change in the nictitating membrane reflex, depending on the test animal selected. The aversive response is usually characterized on the first day of training to determine a baseline for unconditioned fear with aversive response results on subsequent test days (e.g., freezing in the same context but in the absence of the aversive stimulus and/or freezing in presence of the cue but in the absence of the aversive experience) being characterized as contextually conditioned fear. For improved reliability, test animals are typically tested separately by independent technicians and scored over time. Additional experimental design details can be found in the art, for example, in Crawley, J N, What's Wrong with my Mouse; Behavioral Phenotyping of Transgenic and Knockout Mice, Wiley-Liss, NY (2000).

Exemplary test animals (e.g., model animals) include mammals (e.g. rodents or non-human primates) that exhibit prominent symptoms or pathology that is characteristic of an amyloidogenic disorder such as Alzheimer's. Model animals may be created by selective inbreeding for a desired or they may genetically engineered using transgenic techniques that are well-known in the art, such that a targeted genetic alteration (e.g. a genetic mutation, gene disruption) in a gene that is associated with the dementia disorder, leading to aberrant expression or function of the targeted gene. For example, several transgenic mouse strains are available that overexpress APP and develop amyloid plaque pathology and/or develop cognitive deficits that are characteristic of Alzheimer's disease (see for example, Games et al., supra, Johnson-Wood et al., Proc. Natl. Acad. Sci. USA 94:1550 (1997); Masliah E and Rockenstein E. (2000) J Neural Transm Suppl.; 59: 175-83).

Alternatively, the model animal can be created using chemical compounds (e.g. neurotoxins, anesthetics) or surgical techniques (e.g. stereotactic ablation, axotomization, transection, aspiration) that ablate or otherwise interfere with the normal function of an anatomical brain region (e.g. hippocampus, amygdala, perirhinal cortex, medial septal nucleus, locus coeruleus, mammalary bodies) or specific neurons (e.g. serotonergic, cholinergic, or dopaminergic neurons) that are associated with characteristic symptoms or pathology of the amyloidogenic disorder. In certain preferred embodiments, the animal model exhibits a prominent cognitive deficit associated with learning or memory in addition to the neurodegenerative pathology that associated with a amyloidogenic disorder. More preferably, the cognitive deficit progressively worsens with increasing age, such that the disease progression in the model animal parallels the disease progression in a subject suffering from the amyloidogenic disorder.

Contextual fear conditioning and other in vivo assays to test the functionality of the antibodies described herein may be performed using wild-type mice or mice having a certain genetic alteration leading to impaired memory or mouse models of neurodegenerative disease, e.g., Alzheimer's disease, including mouse models which display elevated levels of soluble Aβ in the cerebrospinal fluid (CSF) or plasma. For example, animal models for Alzheimer's disease include transgenic mice that overexpress the “Swedish” mutation of human amyloid precursor protein (hAPPswe; Tg2576) which show age-dependent memory deficits and plaques (Hsiao et al. (1996) Science 274:99-102). The in vivo functionality of the antibodies described herein can also be tested using PDAPP transgenic mice, which express a mutant form of human APP (APP^{V71F}) and develop Alzheimer's disease at a young age (Bard, et al. (2000) Nature Medicine 6:916-919; Masliah E, et al. (1996) J Neurosci. 15;16(18):5795-811). Other mouse models for Alzheimer's disease include the PS-1 mutant mouse, described in Duff, et al. (1996) Nature 383, 710-713 and the PSAPP mouse, a doubly transgenic mouse (PSAPP) overexpressing mutant APP (e.g. Swedish-type mutant APP (hAPPswe)) and PS1 transgenes, described in Holcomb, et al. (1998) Nature Medicine 4:97-110. PSAPP mouse models exhibit age-related development of amyloid plaques that are similar to those observed in AD (Kumar-Singh et al., Am J Pathol. (2005), 167(2):527-43). Deposition of Aβ in the frontal cortex and hippocampus of PSAPP mice as early as 3 months of age progresses to cover substantial portions of these areas of the brain at 12 months (Takachi et al., Am J Pathol. (2000), 157(1):331-9; McGowan et al., Neurobiol Dis. (1999), 6(4):231-44). PSAPP mice can be evaluated in a CFC when they are greater than 10 months of age, for example, when they are approximately 20 months of age. In particular, 20 month old PSAPP mice have particularly prominent contextual memory deficit and dense accumulation of plaque, and are accordingly exemplary model animals for the methods of the invention. Other genetically altered transgenic models of Alzheimer's disease are described in Masliah E and Rockenstein E. (2000) J Neural Transm Suppl. 59:175-83.

In certain embodiments, model animals are evaluated using the methods of the invention at an age when they display symptoms of the disease (e.g. memory deficits), but lack disease pathology (e.g. plaque formation). In exemplary embodiments, model animals are evaluated using the methods of the invention when they are approximately 10 weeks of age or older, more preferably when they are approximately 20 weeks of age or older. In particular, approximately 20 week old transgenic AD mice have particularly prominent contextual memory deficits, and are accordingly preferred model animals for the methods of the invention.

In other embodiments, model animals are evaluated using the methods of the invention at an age when they display both the symptoms of the disease (e.g. memory deficits) and the disease pathology. In exemplary embodiments, model animals are evaluated when they are approximately 10 months of age or older, or when they are greater that 15 months of age or older. In a preferred embodiment, approximately 18-20 month old transgenic AD mice are evaluated using the methods of the invention. 18-20 month old transgenic AD mice are relatively aged animals and are likely to have dense plaque formations in their brains, as well prominent memory deficits.

In various aspects, the methods of the invention comprise the administration of an anti-Aβ antibody that is capable of rapidly improving cognition in a subject wherein the anti-Aβ antibody has been identified in using an assay which is suitably predictive of immunotherapeutic efficacy in the subject. In exemplary embodiments, the assay is a model animal assay that is based, at least in part, on comparing cognition, as determined from a contextual fear conditioning study, of an animal after administration of a test immunological reagent to the animal, as compared to a suitable control. The CFC assay evaluates changes in cognition of an animal (typically a mouse or rat) upon treatment with a potential therapeutic compound. In certain embodiments, the change in cognition evaluated is an improvement in memory impairment status or a reversal of memory deficit. Accordingly, the CFC assay provides a direct method for determining the therapeutic effect of agents for preventing or treating cognitive disease, and in particular, a disease or disorder affecting one or more regions of the brains, e.g., the hippocampus, subiculum, cingulated cortex, prefrontal cortex, perirhinal cortex, sensory cortex, and medial temporal lobe. Such CFC assays are discussed further herein and in U.S. provisional patent application Ser. Nos. 60/636,842 filed on Dec. 15, 2004, 60/637,253, filed on Dec. 16, 2004, and 60/736,119, filed on Nov. 10, 2005, the entire content of each of which is incorporated by reference herein.

12. Chimeric/Humanized Antibodies Having Altered Effector Function

For the above-described antibodies of the invention comprising a constant region (Fc region), it may also be desirable to alter the effector function of the molecule. Generally, the effector function of an antibody resides in the constant or Fc region of the molecule which can mediate binding to various effector molecules, e.g., complement proteins or Fc receptors. The binding of complement to the Fc region is important, for example, in the opsonization and lysis of cell pathogens and the activation of inflammatory responses. The binding of antibody to Fc receptors, for example, on the surface of effector cells can trigger a number of important and diverse biological responses including, for example, engulfment and destruction of antibody-coated pathogens or particles, clearance of immune complexes, lysis of antibody-coated target cells by killer cells (i.e., antibody-dependent cell-mediated cytotoxicity, or ADCC), release of inflammatory mediators, placental transfer of antibodies, and control of immunoglobulin production.

Accordingly, depending on a particular therapeutic or diagnostic application, the above-mentioned immune functions, or only selected immune functions, may be desirable. By altering the Fc region of the antibody, various aspects of the effector function of the molecule, including enhancing or suppressing various reactions of the immune system, with beneficial effects in diagnosis and therapy, are achieved.

The antibodies of the invention can be produced which react only with certain types of Fc receptors, for example, the antibodies of the invention can be modified to bind to only certain Fc receptors, or if desired, lack Fc receptor binding entirely, by deletion or alteration of the Fc receptor binding site located in the Fc region of the antibody. Other desirable alterations of the Fc region of an antibody of the invention are cataloged below. Typically the EU numbering system (ie. the EU index of Kabat et al., supra) is used to indicate which amino acid residue(s) of the Fc region (e.g., of an IgG antibody) are altered (e.g., by amino acid substitution) in order to achieve a desired change in effector function. The numbering system is also employed to compare antibodies across species such that a desired effector function observed in, for example, a mouse antibody, can then be systematically engineered into a human, humanized, or chimeric antibody of the invention.

For example, it has been observed that antibodies (e.g., IgG antibodies) can be grouped into those found to exhibit tight, intermediate, or weak binding to an Fc receptor (e.g., an Fc receptor on human monocytes (FcγRI)). By comparison of the amino-acid sequences in these different affinity groups, a monocyte-binding site in the hinge-link region (Leu234-Ser239 according to EU numbering system) has been identified. Moreover, the human FcγRI receptor binds human IgG1 and mouse IgG2a as a monomer, but the binding of mouse IgG2b is 100-fold weaker. A comparison of the sequence of these proteins in the hinge-link region shows that the sequence from EU numbering positions 234 to 238, i.e., Leu-Leu-Gly-Gly-Pro (SEQ ID NO:71) in the strong binders becomes Leu-Glu-Gly-Gly-Pro (SEQ ID NO:72) in mouse gamma 2b, i.e., weak binders. Accordingly, a corresponding change in a human antibody hinge sequence can be made if reduced FcγI receptor binding is desired. It is understood that other alterations can be made to achieve the same or similar results. For example, the affinity of FcγRI binding can be altered by replacing the specified residue with a residue having an inappropriate functional group on its sidechain, or by introducing a charged functional group (e.g., Glu or Asp) or for example an aromatic non-polar residue (e.g., Phe, Tyr, or Trp).

These changes can be equally applied to the murine, human, and rat systems given the sequence homology between the different immunoglobulins. It has been shown that for human IgG3, which binds to the human FcγRI receptor, changing Leu at EU position 235 to Glu destroys the interaction of the mutant for the receptor. The binding site for this receptor can thus be switched on or switched off by making the appropriate mutation.

Mutations on adjacent or close sites in the hinge link region (e.g., replacing residues at EU positions 234, 236 or 237 by Ala) indicate that alterations in residues 234, 235, 236, and 237 at least affect affinity for the FcγRI receptor. Accordingly, the antibodies of the invention can also have an altered Fc region with altered binding affinity for FcγRI as compared with the unmodified antibody. Such an antibody conveniently has a modification at EU amino acid positions 234, 235, 236, or 237. In some embodiments, an antibody of the invention is a humanized antibody including amino acid alterations at one or more EU positions 234, 235, 236 and 237. In a particular embodiment of the invention, a humanized antibody includes amino acid alterations at EU positions 234 and 237 of the hinge link region derived from IgG1 (i.e., L234A and G237A).

Affinity for other Fc receptors can be altered by a similar approach, for controlling the immune response in different ways.

As a further example, the lytic properties of IgG antibodies following binding of the Cl component of complement can be altered.

The first component of the complement system, Cl, comprises three proteins known as Clq, Clr and Cls which bind tightly together. It has been shown that Clq is responsible for binding of the three protein complex to an antibody.

Accordingly, the Clq binding activity of an antibody can be altered by providing an antibody with an altered CH 2 domain in which at least one of the amino acid residues at EU amino acid positions 318, 320, and 322 of the heavy chain has been changed to a residue having a different side chain. Other suitable alterations for altering, e.g., reducing or abolishing specific Clq-binding to an antibody include changing any one of residues at EU positions 318 (Glu), 320 (Lys) and 322 (Lys), to Ala.

Moreover, by making mutations at these residues, it has been shown that Clq binding is retained as long as residue 318 has a hydrogen-bonding side chain and residues 320 and 322 both have a positively charged side chain.

Clq binding activity can be abolished by replacing any one of the three specified residues with a residue having an inappropriate functionality on its side chain. It is not necessary to replace the ionic residues only with Ala to abolish Clq binding. It is also possible to use other alkyl-substituted non-ionic residues, such as Gly, Ile, Leu, or Val, or such aromatic non-polar residues as Phe, Tyr, Trp and Pro in place of any one of the three residues in order to abolish Clq binding. In addition, it is also be possible to use such polar non-ionic residues as Ser, Thr, Cys, and Met in place of residues 320 and 322, but not 318, in order to abolish Clq binding activity.

It is also noted that the side chains on ionic or non-ionic polar residues will be able to form hydrogen bonds in a similar manner to the bonds formed by the Glu residue. Therefore, replacement of the 318 (Glu) residue by a polar residue may modify but not abolish Clq binding activity.

It is also known that replacing residue 297 (Asn) with Ala results in removal of lytic activity while only slightly reducing (about three fold weaker) affinity for Clq. This alteration destroys the glycosylation site and the presence of carbohydrate that is required for complement activation. Any other substitution at this site will also destroy the glycosylation site.

The invention also provides an antibody having an altered effector function wherein the antibody has a modified hinge region. The modified hinge region may comprise a complete hinge region derived from an antibody of different antibody class or subclass from that of the CH1 domain. For example, the constant domain (CH1) of a class IgG1 antibody can be attached to a hinge region of a class IgG4 antibody. Alternatively, the new hinge region may comprise part of a natural hinge or a repeating unit in which each unit in the repeat is derived from a natural hinge region. In one example, the natural hinge region is altered by converting one or more cysteine residues into a neutral residue, such as alanine, or by converting suitably placed residues into cysteine residues. Such alterations are carried out using art recognized protein chemistry and, preferably, genetic engineering techniques, as described herein.

In one embodiment of the invention, the number of cysteine residues in the hinge region of the antibody is reduced, for example, to one cysteine residue. This modification has the advantage of facilitating the assembly of the antibody, for example, bispecific antibody molecules and antibody molecules wherein the Fc portion has been replaced by an effector or reporter molecule, since it is only necessary to form a single disulfide bond. This modification also provides a specific target for attaching the hinge region either to another hinge region or to an effector or reporter molecule, either directly or indirectly, for example, by chemical means.

Conversely, the number of cysteine residues in the hinge region of the antibody is increased, for example, at least one more than the number of normally occurring cysteine residues. Increasing the number of cysteine residues can be used to stabilize the interactions between adjacent hinges. Another advantage of this modification is that it facilitates the use of cysteine thiol groups for attaching effector or reporter molecules to the altered antibody, for example, a radiolabel.

Accordingly, the invention provides for an exchange of hinge regions between antibody classes, in particular, IgG classes, and/or an increase or decrease in the number of cysteine residues in the hinge region in order to achieve an altered effector function (see for example U.S. Pat. No. 5,677,425 which is expressly incorporated herein). A determination of altered antibody effector function is made using the assays described herein or other art recognized techniques.

In yet another aspect, the isotype of the antibody is IgG4. In another aspect, an antibody of the invention is engineered to have an isotype having reduced effector function (e.g., reduced Fc-mediated phagocytosis, reduced ability to opsonize plaques etc.). In a particular embodiment, an antibody of the invention is a humanized 12A11 antibody (e.g., humanized 12A11 v.1) having an IgG4 isotype.

Importantly, the resultant antibody can be subjected to one or more assays to evaluate any change in biological activity compared to the starting antibody. For example, the ability of the antibody with an altered Fc region to bind complement or Fc receptors can be assessed using the assays disclosed herein as well as any art recognized assay.

Production of the antibodies of the invention is carried out by any suitable technique including techniques described herein as well as techniques known to those skilled in the art. For example an appropriate protein sequence, e.g. forming part of or all of a relevant constant domain, e.g., Fc region, i.e., CH2, and/or CH3 domain(s), of an antibody, and include appropriately altered residue(s) can be synthesized and then chemically joined into the appropriate place in an antibody molecule.

Preferably, genetic engineering techniques are used for producing an altered antibody. Preferred techniques include, for example, preparing suitable primers for use in polymerase chain reaction (PCR) such that a DNA sequence which encodes at least part of an IgG heavy chain, e.g., an Fc or constant region (e.g., CH2, and/or CH3) is altered, at one or more residues. The segment can then be operably linked to the remaining portion of the antibody, e.g., the variable region of the antibody and required regulatory elements for expression in a cell.

The present invention also includes vectors used to transform the cell line, vectors used in producing the transforming vectors, cell lines transformed with the transforming vectors, cell lines transformed with preparative vectors, and methods for their production.

Preferably, the cell line which is transformed to produce the antibody with an altered Fc region (i.e., of altered effector function) is an immortalized mammalian cell line (e.g., CHO cell).

Although the cell line used to produce the antibody with an altered Fc region is preferably a mammalian cell line, any other suitable cell line, such as a bacterial cell line or a yeast cell line, may alternatively be used.

13. Affinity Maturation

Antibodies (e.g., humanized antibodies) of the invention can be modified for improved function using any of a number of affinity maturation techniques. Typically, a candidate molecule with a binding affinity to a given target molecule is identified and then further improved or “matured” using mutagenesis techniques resulting in one or more related candidates having a more desired binding interaction with the target molecule. Typically, it is the affinity of the antibody (or avidity, i.e., the combined affinities of the antibody for a target antigen) that is modified, however, other properties of the molecule, such as stability, effector function, clearance, secretion, or transport function, may also be modified, either separately or in parallel with affinity, using affinity maturation techniques.

In exemplary embodiments, the affinity of an antibody (e.g., a humanized antibody of the instant invention) is increased. For example, antibodies having binding affinities of at least 10⁷M⁻¹, 10⁸M⁻¹ or 10⁹M⁻¹ can be matured such that their affinities are at least 10⁹M⁻¹, 10¹⁰M⁻¹ or 10¹²M⁻¹.

One approach for affinity maturing a binding molecule is to synthesize a nucleic acid encoding the binding molecule, or portion thereof, that encodes the desired change or changes. Oligonucleotide synthesis is well known in the art and readily automated to produce one or more nucleic acids having any desired codon change(s). Restriction sites, silent mutations, and favorable codon usage may also be introduced in this way. Alternatively, one or more codons can be altered to represent a subset of particular amino acids, e.g., a subset that excludes cysteines which can form disulfide linkages, and is limited to a defined region, for example, a CDR region or portion thereof. Alternatively, the region may be represented by a partially or entirely random set of amino acids (for additional details, see, e.g., U.S. Pat. Nos. 5,830,650; 5,798,208; 5,824,514; 5,817,483; 5,814,476; 5,723,323; 4,528,266; 4,359,53; 5,840,479; and 5,869,644).

It is understood that the above approaches can be carried out in part or in full using polymerase chain reaction (PCR) which is well known in the art and has the advantage of incorporating oligonucleotides, e.g., primers or single stranded nucleic acids having, e.g., a desired alteration(s), into a double stranded nucleic acid and in amplified amounts suitable for other manipulations, such as genetic engineering into an appropriate expression or cloning vector. Such PCR can also be carried out under conditions that allow for misincorporation of nucleotides to thereby introduce additional variability into the nucleic acids being amplified. Experimental details for carrying out PCR and related kits, reagents, and primer design can be found, e.g., in U.S. Pat. Nos. 4,683,202; 4,683,195; 6,040,166; and 6,096,551. Methods for introducing CDR regions into antibody framework regions using primer-based PCR is described in, e.g., U.S. Pat. No. 5,858,725. Methods for primer-based PCR amplification of antibody libraries (and libraries made according to method) employing a minimal set of primers capable of finding sequence homology with a larger set of antibody molecules, such that a larger and diverse set of antibody molecules can be efficiently amplified, is described, e.g., in U.S. Pat. Nos. 5,780,225; 6,303,313; and 6,479,243. Non PCR-based methods for performing site directed mutagenesis can also be used and include “Kunkel” mutagenesis that employs single-stranded uracil containing templates and primers that hybridize and introduce a mutation when passed through a particular strain of E. coli (see, e.g., U.S. Pat. No. 4,873,192).

Additional methods for varying an antibody sequence, or portion thereof, include nucleic acid synthesis or PCR of nucleic acids under nonoptimal (i.e., error-prone) conditions, denaturation and renaturation (annealing) of such nucleic acids, exonuclease and/or endonuclease digestion followed by reassembly by ligation or PCR (nucleic acid shuffling), or a combination of one or more of the foregoing techniques as described, for example, in U.S. Pat. Nos. 6,440,668; 6,238,884; 6,171,820; 5,965,408; 6,361,974; 6,358,709; 6,352,842; 4,888,286; 6,337,186; 6,165,793; 6,132,970; 6,117,679; 5,830,721; and 5,605,793.

In certain embodiment, antibody libraries (or affinity maturation libraries) comprising a family of candidate antibody molecules having diversity in certain portions of the candidate antibody molecule, e.g., in one or more CDR regions (or a portion thereof), one or more framework regions, and/or one or more constant regions (e.g., a constant region having effector function) can be expressed and screened for desired properties using art recognized techniques (see, e.g., U.S. Pat. Nos. 6,291,161; 6,291,160; 6,291,159; and 6,291,158). For example, expression libraries of antibody variable domains having a diversity of CDR3 sequences and methods for producing human antibody libraries having a diversity of CDR3 sequences by introducing, by mutagenesis, a diversity of CDR3 sequences and recovering the library can be constructed (see, e.g., U.S. Pat. No. 6,248,516).

Other techniques for antibody engineering include antibody affinity engineering by serial epitope guided complementarity replacement, as described in WOO 2004/072266 by Kalobios, Inc.

Finally, for expressing the affinity matured antibodies, nucleic acids encoding the candidate antibody molecules can be introduced into cells in an appropriate expression format, e.g., as full length antibody heavy and light chains (e.g., IgG), antibody Fab fragments (e.g., Fab, F(ab′)₂), or as single chain antibodies (scFv) using standard vector and cell transfection/transformation technologies (see, e.g., U.S. Pat. Nos. 6,331,415; 6,103,889; 5,260,203; 5,258,498; and 4,946,778).

B. Nucleic Acid Encoding Immunologic and Therapeutic Agents

Immune responses against amyloid deposits can also be induced by administration of nucleic acids encoding antibodies and their component chains used for passive immunization. Such nucleic acids can be DNA or RNA. A nucleic acid segment encoding an immunogen is typically linked to regulatory elements, such as a promoter and enhancer, that allow expression of the DNA segment in the intended target cells of a patient. For expression in blood cells, as is desirable for induction of an immune response, exemplary promoter and enhancer elements include those from light or heavy chain immunoglobulin genes and/or the CMV major intermediate early promoter and enhancer (Stinski, U.S. Pat. Nos. 5,168,062 and 5,385,839). The linked regulatory elements and coding sequences are often cloned into a vector. For administration of double-chain antibodies, the two chains can be cloned in the same or separate vectors.

A number of viral vector systems are available including retroviral systems (see, e.g., Lawrie and Tumin, Cur. Opin. Genet. Develop. 3:102-109 (1993)); adenoviral vectors (see, e.g., Bett et al., J. Virol. 67:5911 (1993)); adeno-associated virus vectors (see, e.g., Zhou et al., J. Exp. Med. 179:1867 (1994)), viral vectors from the pox family including vaccinia virus and the avian pox viruses, viral vectors from the alpha virus genus such as those derived from Sindbis and Semliki Forest Viruses (see, e.g., Dubensky et al., J. Virol. 70:508 (1996)), Venezuelan equine encephalitis virus (see Johnston et al., U.S. Pat. No. 5,643,576) and rhabdoviruses, such as vesicular stomatitis virus (see Rose, U.S. Pat. No. 6,168,943) and papillomaviruses (Ohe et al., Human Gene Therapy 6:325 (1995); Woo et al., WO 94/12629 and Xiao & Brandsma, Nucleic Acids. Res. 24, 2630-2622 (1996)).

DNA encoding an immunogen, or a vector containing the same, can be packaged into liposomes. Suitable lipids and related analogs are described by Eppstein et al., U.S. Pat. No. 5,208,036, Felgner et al., U.S. Pat. No. 5,264,618, Rose, U.S. Pat. No. 5,279,833, and Epand et al., U.S. Pat. No. 5,283,185. Vectors and DNA encoding an immunogen can also be adsorbed to or associated with particulate carriers, examples of which include polymethyl methacrylate polymers and polylactides and poly(lactide-co-glycolides), see, e.g., McGee et al., J Micro Encap. (1996).

Gene therapy vectors or naked polypeptides (e.g., DNA) can be delivered in vivo by administration to an individual patient, typically by systemic administration (e.g., intravenous, intraperitoneal, nasal, gastric, intradermal, intramuscular, subdermal, or intracranial infusion) or topical application (see e.g., Anderson et al., U.S. Pat. No. 5,399,346). The term “naked polynucleotide” refers to a polynucleotide not delivered in association with a transfection facilitating agent. Naked polynucleotides are sometimes cloned in a plasmid vector. Such vectors can further include facilitating agents such as bupivacaine (Weiner et al., U.S. Pat. No. 5,593,972). DNA can also be administered using a gene gun. See Xiao & Brandsma, supra. The DNA encoding an immunogen is precipitated onto the surface of microscopic metal beads. The microprojectiles are accelerated with a shock wave or expanding helium gas, and penetrate tissues to a depth of several cell layers. For example, The Accel™ Gene Delivery Device manufactured by Agricetus, Inc. Middleton Wis. is suitable. Alternatively, naked DNA can pass through skin into the blood stream simply by spotting the DNA onto skin with chemical or mechanical irritation (see Howell et al., WO 95/05853).

In a further variation, vectors encoding immunogens can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

II. Prophylactic and Therapeutic Methods

The present invention is directed inter alia to the treatment of Aβ-related diseases or disorders, including amyloidogenic disorders and diseases characterized by soluble Aβ (e.g. Alzheimer's). The invention is also directed to use of the disclosed immunological reagents (e.g., humanized immunoglobulins) in the manufacture of a medicament for the treatment or prevention of an Aβ-related disease or disorder or amyloidogenic disease or disorder. The treatment methods of the invention comprise administration of the disclosed immunological reagents (e.g., humanized immunoglobulins against specific epitopes within Aβ) to a patient under conditions that generate a beneficial therapeutic response in a patient (e.g., rapid improvement in cognition, induction of phagocytosis of Aβ, reduction of plaque burden, inhibition of plaque formation, reduction of neuritic dystrophy, and/or reversing, treating or preventing cognitive decline) in the patient, for example, for the prevention or treatment of the Aβ-related diseases or disorders or amyloidogenic diseases or disorders. Such diseases include Alzheimer's disease, Down's syndrome and mild cognitive impairment. The latter can occur with or without other characteristics of an amyloidogenic disease.

It will be appreciated by those in the art that the immunological reagents of the invention may be used to treat any disorder for which treatment with said immunological reagents is shown to provide a therapeutic benefit to a patient suffering from the disorder. For example, the disorder may be any cognitive disorder, e.g. a dementia disorder. Such cognitive deficits may have a number of origins: a functional mechanism (anxiety, depression), physiological aging (age-associated memory impairment), drugs, or anatomical lesions. Indications for which the immunotherapeutic agents of the invention can be useful include learning disabilities or memory deficits due to toxicant exposure, brain injury leading to amnesia, age, schizophrenia, epilepsy, mental retardation, alcoholic blackouts, Korsakoff's syndrome, medication-induced amnesia (e.g. Halcion), basilar artery migraines, or amnesias associated with Herpes simplex encephalitis.

In certain embodiments, the methods of the invention involve the administration of an immunological reagent comprising an Aβ antibody, wherein the Aβ antibody is specific for an epitope within residues 1-10 of Aβ and preferentially binds to soluble oligomeric Aβ as compared to monomeric Aβ, such that the rapid improvement in cognition is achieved. In exemplary embodiments, the immunological reagent is an Aβ antibody that is specific for an epitope within residues 1-10 of Aβ and effects a rapid improvement in cognition in an animal model of an Aβ-related disorder as determined in a Contextual Fear Conditioning (CFC) assay. In other exemplary embodiments, the immunological reagent is an Aβ antibody that is specific for an epitope within residues 1-10 of Aβ, preferentially binds to soluble oligomeric Aβ as compared to monomeric Aβand effects a rapid improvement in cognition in an animal model of an Aβ-related disorder as determined in a Contextual Fear Conditioning (CFC) assay.

In other exemplary embodiments, the Aβ antibodies bind to an epitope within residues 3-7 of Aβ. Exemplary Aβ antibodies are selected from the group consisting of a 3D6 antibody, a 3A3 antibody, a 6C6 antibody, a 10D5 antibody, and a 12A11 antibody. In one embodiment, the Aβ antibody is not a 3D6 antibody.

In other exemplary embodiments, the methods of the invention involve administration of immunological reagent comprising an Aβ antibody, wherein the Aβantibody is specific for an epitope within residues 13-28 of Aβ and preferentially binds to soluble oligomeric Aβ as compared to monomeric Aβ, such that the rapid improvement in cognition is achieved. In other embodiments, the immunological reagent comprises an Aβ antibody, wherein the Aβ antibody is specific for an epitope within residues 13-28 of Aβ and effects a rapid improvement in cognition in an animal model of an Aβ-related disorder as determined in a Contextual Fear Conditioning (CFC) assay, such that the rapid improvement in cognition is achieved. In still other embodiments, the immunological reagent is an Aβ antibody, wherein the Aβ antibody is specific for an epitope within residues 13-28 of Aβ, preferentially binds to soluble oligomeric Aβ as compared to monomeric Aβ and effects a rapid improvement in cognition in an animal model of an Aβ-related disorder as determined in a Contextual Fear Conditioning (CFC) assay, such that the rapid improvement in cognition is achieved.

In certain exemplary embodiments, the Aβ antibodies bind to an epitope within residues 16-24 of Aβ. Exemplary Aβ antibodies are selected from the group consisting of a 2B1 antibody, a 1C2 antibody, a 15C11 antibody and a 9G8 antibody. In a preferred embodiment, the Aβ antibody is not a 266 antibody.

In certain embodiments, the methods of the invention comprise the administration of an immunological reagent to a subject that has or is at risk for an Aβ-related disease or disorder, in order to rapidly improve cognition in said subject. In other embodiments, the methods of the invention comprise the administration of an immunological reagent to a subject that is substantially free of amyloid deposits. In other embodiments, the methods of the invention comprise the administration of an immunological reagent to a subject prior to substantial plaque deposition in the subject: In one embodiment, the Aβ-related disease or disorder is associated with or characterized by soluble Aβ. In another embodiment, the Aβ-related disease or disorder is associated with or characterized by insoluble Aβ. In another embodiment, the Aβ-related disease or disorder is an amyloidogenic disease. In another embodiment, the Aβ-related disease or disorder is Alzheimer's disease. In another embodiment, the Aβ-related disease or disorder is an Aβ-related cognitive disorder, such as, for example a mild cognitive impairment.

In certain embodiments, the methods of the invention comprise the administration of an immunological reagent (e.g. an Aβ antibody) to a subject as a single dose. In other embodiments, the methods of the invention comprise the administration of an immunological reagent (e.g. an Aβ antibody) to a subject in multiple doses. In one embodiment, the dose of Aβ antibody is from about 100 μg/kg to 100 mg/kg body weight of the patient. In another embodiment, the dose of Aβ antibody is from about 300 μg/kg to 30 mg/kg body weight of the patient. In still another embodiment, the dose of Aβ antibody is from about 1 mg/kg to 10 mg/kg body weight of the patient.

When administering an effective dose of an immunological reagent (e.g., antibody or antigen-binding fragment thereof) to a human patient for effecting a rapid improvement in cognition in said patient, the effective dose may be a dose equivalent to that necessary to achieve a rapid improvement in cognition in an appropriate animal model. For example, a human patient can be administered a dose equivalent to that necessary to achieve a rapid improvement in cognition is an animal model of AD (as determined in a CFC assay, as described herein). The dose administered need not be an identical g/kg body weight dose as administered to a model animal. Rather, doses for human administration are those sufficient to achieve an equivalent treatment effect as that seen in the appropriate animal model.

In certain embodiments, the instant invention provides methods for effecting a rapid improvement in cognition in a subject comprising administration of an immunological reagent to the subject such that a rapid improvement is achieved within one month after administration of the antibody. In other embodiments, the rapid improvement in cognition is achieved within one week after administration of the antibody. In other embodiments, the rapid improvement in cognition is achieved within one day after administration of the antibody. In still other embodiments, the rapid improvement in cognition is achieved within 12 hours after administration of the antibody.

In certain embodiments, the methods for effecting rapid improvement in cognition in a subject comprise administering to the subject an effective dose of an Aβ antibody, wherein the antibody is specific for an epitope within residues 13-28 of Aβ and preferentially binds to soluble oligomeric Aβ as compared to monomeric Aβ, such that the rapid improvement in cognition is achieved. In other embodiments, the methods for effecting rapid improvement in cognition in a subject comprise administering to the subject an effective dose of an Aβ antibody, wherein the antibody is specific for an epitope within residues 13-28 of Aβ and effects a rapid improvement in cognition in an animal model of an Aβ-related disorder as determined in a Contextual Fear Conditioning (CFC) assay, such that the rapid improvement in cognition is achieved. In still other embodiments, the methods for effecting rapid improvement in cognition in a subject comprise administering to the subject an effective dose of an Aβ antibody, wherein the antibody is specific for an epitope within residues 13-28 of Aβ, preferentially binds to soluble oligomeric Aβ as compared to monomeric Aβ and effects a rapid improvement in cognition in an animal model of an Aβ-related disorder as determined in a Contextual Fear Conditioning (CFC) assay, such that the rapid improvement in cognition is achieved. In one embodiment, the Aβ antibody binds to an epitope within residues 16-24 of Aβ. In another embodiment, the Aβ antibody is selected from the group consisting of a 2B1 antibody, a 1C2 antibody, a 15C11 antibody and a 9G8 antibody. In another embodiment, the Aβ antibody is not a 266 antibody.

The methods can be used on both asymptomatic patients and those currently showing symptoms of disease. The antibodies used in for passive immunization or immunotherapy of human subjects with Aβ-related diseases or disorders or amyloidogenic diseases or disorders can be human, humanized, chimeric or nonhuman antibodies, or fragments thereof (e.g., antigen binding fragments) and can be monoclonal or polyclonal, as described herein. In another aspect, the invention features administering an antibody with a pharmaceutical carrier as a pharmaceutical composition. Alternatively, the antibody can be administered to a patient by administering a polynucleotide encoding at least one antibody chain. The polynucleotide is expressed to produce the antibody chain in the patient. Optionally, the polynucleotide encodes heavy and light chains of the antibody. The polynucleotide is expressed to produce the heavy and light chains in the patient. In exemplary embodiments, the patient is monitored for level of administered antibody in the blood of the patient.

In another aspect, the invention features administering an antibody with a pharmaceutical carrier as a pharmaceutical composition. Alternatively, the antibody can be administered to a patient by administering a polynucleotide encoding at least one antibody chain. The polynucleotide is expressed to produce the antibody chain in the patient. Optionally, the polynucleotide encodes heavy and light chains of the antibody. The polynucleotide is expressed to produce the heavy and light chains in the patient. In exemplary embodiments, the patient is monitored for level of administered antibody in the blood of the patient.

The invention thus fulfills a longstanding need for therapeutic regimes for preventing or ameliorating the neuropathology and, in some patients, the cognitive impairment associated with an Aβ-related disease or disorder or amyloidogenic disease or disorder (e.g., AD).

A. Rapid Improvement in Cognition

The present invention provides methods for effecting rapid improvement in cognition in a patient having or at risk for suffering from an Aβ-related disease or disorder or amyloidogenic disease or disorder (e.g., AD). In preferred aspects, the methods feature administering an effective dose of an immunological reagent (e.g., an Aβ antibody) such that rapid improvement in cognition is achieved. In exemplary aspects of the invention, improvement in one or more cognitive deficits in the patient (e.g., procedural learning and/or memory, deficits) is achieved. The cognitive deficit can be an impairment in explicit memory (also known as “declarative” or “working” memory), which is defined as the ability to store and retrieve specific information that is available to consciousness and which can therefore be expressed by language (e.g. the ability to remember a specific fact or event). Alternatively, the cognitive deficit can be an impairment in procedural memory (also known as “implicit” or “contextual” memory), which is defined as the ability to acquire, retain, and retrieve general information or knowledge that is not available to consciousness and which requires the learning of skills, associations, habits, or complex reflexes to be expressed, e.g. the ability to remember how to execute a specific task. Individuals suffering from procedural memory deficits are much more impaired in their ability to function normally. As such, treatments which are effective in improving deficits in procedural memory are highly desirable and advantageous.

B. Patients Amenable to Treatment

Patients amenable to treatment include individuals at risk of an Aβ-related disease or disorder or amyloidogenic disease or disorder but not showing symptoms, as well as patients presently showing symptoms. In the case of Alzheimer's disease, virtually anyone is at risk of suffering from Alzheimer's disease if he or she lives long enough. Therefore, the present methods can be administered prophylactically to the general population without the need for any assessment of the risk of the subject patient.

The present methods are especially useful for individuals who are at risk for AD, e.g., those who exhibit risk factors of AD. The main risk factor for AD is increased age. As the population ages, the frequency of AD continues to increase. Current estimates indicate that up to 10% of the population over the age of 65 and up to 50% of the population over the age of 85 have AD.

Although rare, certain individuals can be identified at an early age as being genetically predisposed to developing AD. Individuals carrying the heritable form of AD, known as “familial AD” or “early-onset AD”, can be identified from a well documented family history of AD, of the analysis of a gene that is known to confer AD when mutated, for example the APP or presenilin gene. Well characterized APP mutations include the “Hardy” mutations at codons 716 and 717 of APP770 (e.g., valine⁷¹⁷ to isoleucine (Goate et al., (1991), Nature 349:704); valine⁷¹⁷ to glycine (Chartier et al. (1991) Nature 353:844; Murrell et al. (1991), Science 254:97); valine⁷¹⁷ to phenylalanine (Mullan et al. (1992), Nature Genet. 1:345-7)), the “Swedish” mutations at codon 670 and 671 of APP770, and the “Flemish” mutation at codon 692 of APP770. Such mutations are thought to cause Alzheimer's disease by increased or altered processing of APP to Aβ, particularly processing of APP to increased amounts of the long form of Aβ (i.e., Aβ1-42 and Aβ1-43). Mutations in other genes, such as the presenilin genes, PS1 and PS2, are thought indirectly to affect processing of APP to generate increased amounts of long form Aβ (see Hardy, TINS 20: 154 (1997); Kowalska et al., (2004), Polish J. Pharmacol., 56: 171-8). In addition to AD, mutations at amino acid 692 or 693 of the 770-amino acid isoform of APP has been implicated in cerebral amyloidogenic disorder called Hereditary Cerebral Hemorrhage with Amyloidosis of the Dutch-type (HCHWA-D).

More commonly, AD is not inherited by a patient but develops due to the complex interplay of a variety of genetic factors. These individuals are said to have “sporadic AD” (also known as “late-onset AD”), a form which is much more difficult to diagnose. Nonetheless, the patient population can be screened for the presence of susceptibility alleles or traits that do not cause AD but are known to segregate with AD at a higher frequency than in the general population, e.g., the ε2, ε3, and ε4 alleles of apolipoprotein E (Corder et. al. (1993), Science, 261: 921-923). In particular, patients lacking the ε4 allele, preferably in addition to some other marker for AD, may be identified as “at risk” for AD. For example, patients lacking the ε4 allele who have relatives who have AD or who suffer from hypercholesterolemia or atherosclerosis may be identified as “at risk” for AD. Another potential biomarker is the combined assessment of cerebral spinal fluid (CSF) Aβ42 and tau levels. Low Aβ42 and high tau levels have a predictive value in identifying patients at risk for AD.

Other indicators of patients at risk for AD include in vivo dynamic neuropathological data, for example, in vivo detection of brain beta amyloid, patterns of brain activation, etc. Such data can be obtained using, for example, three-dimensional magnetic resonance imaging (MRI), positron emission tomography (PET) scan and single-photon emission computed tomography (SPECT). Indicators of patients having probable AD include, but are not limited to, patients (1) having dementia, (2) of an age of 40-90 years old, (3) cognitive deficits, e.g., in two or more cognitive domains, (4) progression of deficits for more than six months, (5) consciousness undisturbed, and/or (6) absence of other reasonable diagnoses.

Individuals suffering either sporadic or familial forms of AD are usually, however, diagnosed following presentation of one or more characteristic symptoms of AD. Common symptoms of AD include cognitive deficits that affect the performance of routine skills or tasks, problems with language, disorientation to time or place, poor or decreased judgment, impairments in abstract thought, loss of motor control, mood or behavior alteration, personality change, or loss of initiative. The number deficits or the degree of the cognitive deficit displayed by the patient usually reflects the extent to which the disease has progressed. For example, the patient may exhibit only a mild cognitive impairment, such that the patient exhibits problems with memory (e.g. contextual memory) but is otherwise able to function well.

The present methods are also useful for individuals who have an Aβ-related cognitive deficit, e.g. Aβ-related dementia. In particular, the present methods are especially useful for individuals who have a cognitive deficit or aberrancy caused by or attributed to the presence of soluble oligomeric Aβ in the central nervous system (CNS), for example, in the brain or CSF. Cognitive deficits caused by or associated with Aβ also include those caused by or associated with: (1) the development of β-amyloid plaques in the brain; (2) abnormal rates of Aβ synthesis, processing, degradation or clearance; (3) the formation or activity of soluble oligomeric Aβ species (e.g., in the brain); and/or (4) the formation of abnormal forms of Aβ. It is not necessary that an actual causative link be established between an Aβ abnormality and cognitive deficit in a particular patient, however, some the link should be indicated, for example, by one of the above-described markers of AD to distinguish patients suffering from non-Aβ related cognitive deficits who would not be expected to benefit from treatment with an Aβ immunotherapeutic agent.

Several tests have been developed to assess cognitive skills or performance in human subjects, for example, subjects at risk for or having symptoms or pathology of dementia disorders (e.g., AD). Cognitive deficits can be identified by impaired performance of these tests, and many treatments have been proposed based on their ability to improve performance in these tests. Although some tasks have evaluated behaviors or motor function of subjects, most tasks have been designed to test learning or memory.

Cognition in humans may be assessed using a wide variety of tests including, but not limited to, the following tests. The ADAS-Cog (Alzheimer Disease Assessment Scale-Cognitive) is 11-part test that takes 30 minutes to complete. The ADAS-Cog is a preferred brief exam for the study of language and memory skills. See Rosen et al. (1984) Am J Psychiatry. 141(11):1356-64; Ihl et al. (2000) Neuropsychobiol. 41(2):102-7; and Weyer et al. (1997) Int Psychogeriatr. 9(2):123-38.

The Blessed Test is another quick (˜10 minute) test of cognition which assesses activities of daily living and memory, concentration and orientation. See Blessed et al. (1968) Br J Psychiatry 114(512):797-811.

The Cambridge Neuropsychological Test Automated Battery (CANTAB) is used for the assessment of cognitive deficits in humans with neurodegenerative diseases or brain damage. It consists of thirteen interrelated computerized tests of memory, attention, and executive function, and is administered via a touch sensitive screen from a personal computer. The tests are language and largely culture free, and have shown to be highly sensitive in the early detection and routine screening of Alzheimer's disease. See Swainson et al. (2001) Dement Geriatr Cogn Disord. 12:265-280; and Fray and Robbins (1996) Neurotoxicol Teratol. 18(4):499-504. Robbins et al. (1994) Dementia 5(5):266-81.

The Consortium to Establish a Registry for Alzheimer's Disease (CERAD) Clinical and Neuropsychological Tests include a verbal fluency test, Boston Naming Test, Mini Mental State Exam (MMSE), ten-item word recall, constructional praxis, and delayed recall of praxis items. The test typically takes 20-30 minutes and is convenient and effective at assessing and tracking cognitive decline. See Morris et al. (1988) Psychopharmacol Bull. 24(4):641-52; Morris et al. (1989) Neurology 39(9):1159-65; and Welsh et al. (1991) Arch Neurol. 48(3):278-81.

The Mini Mental State Exam (MMSE) developed in 1975 by Folestein et al, is a brief test of mental status and cognition function. It does not measure other mental phenomena and is therefore not a substitute for a full mental status examination. It is useful in screening for dementia and its scoring system is helpful in following progress over time. The Mini-Mental State Examination MMSE is widely used, with norms adjusted for age and education. It can be used to screen for cognitive impairment, to estimate the severity of cognitive impairment at a given point in time, to follow the course of cognitive changes in an individual over time, and to document an individual's response to treatment. Cognitive assessment of subjects may require formal neuropsychological testing, with follow-up testing separated by nine months or more (in humans). See Folstein et al. (1975) J Psychiatr Res. 12:196-198; Cockrell and Folstein (1988) Psychopharm Bull. 24(4):689-692; and Crum et al. (1993) J. Am. Med. Association 18:2386-2391.

The Seven-Minute Screen is a screening tool to help identify patients who should be evaluated for Alzheimer's disease. The screening tool is highly sensitive to the early signs of AD, using a series of questions to assess different types of intellectual functionality. The test consists of 4 sets of questions that focus on orientation, memory, visuospatial skills and expressive language. It can distinguish between cognitive changes due to the normal aging process and cognitive deficits due to dementia. See Solomon and Pendlebury (1998) Fam Med. 30(4):265-71, Solomon et al. (1998) Arch Neurol. 55(3):349-55.

Individuals presently suffering from Alzheimer's disease can be recognized from characteristic dementia, as well as the presence of risk factors described above. In addition, a number of diagnostic tests are available for identifying individuals who have AD. These include measurement of CSF tau and Aβ42 levels. Elevated tau and decreased Aβ42 levels signify the presence of AD. Individuals suffering from Alzheimer's disease can also be diagnosed by ADRDA criteria as discussed in the Examples section.

C. Treatment Regimes and Dosages

In prophylactic applications, pharmaceutical compositions or medicaments are administered to a patient susceptible to, or otherwise at risk of, Alzheimer's disease in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions or medicaments are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease.

In some methods, administration of an immunological reagent (e.g., an Aβ antibody) reduces or eliminates myocognitive impairment in patients that have not yet developed characteristic Alzheimer's pathology. An amount adequate to accomplish therapeutic or prophylactic treatment is defined as a therapeutically- or prophylactically-effective dose. In both prophylactic and therapeutic regimes, reagents are usually administered in several dosages until a sufficient immune response has been achieved. The term “immune response” or “immunological response” includes the development of a humoral (antibody mediated) and/or a cellular (mediated by antigen-specific T cells or their secretion products) response directed against an antigen in a recipient subject. Such a response can be an active response, i.e., induced by administration of immunogen, or a passive response, i.e., induced by administration of immunoglobulin or antibody or primed T-cells. Typically, the immune response is monitored and repeated dosages are given if the immune response starts to wane.

Effective doses of the compositions of the present invention, for the treatment of the above described conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages need to be titrated to optimize safety and efficacy.

For passive immunization with an antibody, the dosage ranges from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. In another example, dosages can be 0.5 mg/kg body weight or 15 mg/kg body weight or within the range of 0.5-15 mg/kg, preferably at least 1 mg/kg. In another example, dosages can be 0.5 mg/kg body weight or 20 mg/kg body weight or within the range of 0.5-20 mg/kg, preferably at least 1 mg/kg. In another example, dosages can be 0.5 mg/kg body weight or 30 mg/kg body weight or within the range of 0.5-30 mg/kg, preferably at least 1 mg/kg. In a preferred example, dosages can be about 30 kg/mg. In a particularly preferred example, the Aβ antibody is administered intraperitoneally at a dose range from approximately 0.3 mg/kg to approximately 30 mg/kg.

Doses intermediate in the above ranges are also intended to be within the scope of the invention. Subjects can be administered such doses daily, on alternative days, weekly or according to any other schedule determined by empirical analysis. An exemplary treatment involves administration in multiple dosages over a prolonged period, for example, of at least six months. Additional exemplary treatment regimes involve administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated.

Antibody is usually administered on multiple occasions. Intervals between single dosages can be weekly, monthly or yearly. Intervals can also be irregular as indicated by measuring blood levels of antibody to Aβ in the patient. In some methods, dosage is adjusted to achieve a plasma antibody concentration of 1-1000 μg/ml and in some methods 25-300 μg/ml. Alternatively, antibody can be administered as a sustained release formulation, in which case less frequent administration is required. Dosage and frequency vary depending on the half-life of the antibody in the patient. In general, humanized antibodies show the longest half-life, followed by chimeric antibodies and nonhuman antibodies.

The dosage and frequency of administration can vary depending on whether the treatment is prophylactic or therapeutic. In prophylactic applications, compositions containing the present antibodies or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In asymptomatic patients, treatment can begin at any age (e.g., 10, 20, 30). Usually, however, it is not necessary to begin treatment until a patient reaches 40, 50, 60 or 70. Treatment typically involves multiple dosages over a period of time. Treatment can be monitored by assaying antibody levels over time. If the response falls, a booster dosage is indicated. In the case of potential Down's syndrome patients, treatment can begin antenatally by administering therapeutic reagent to the mother or shortly after birth.

In therapeutic applications, a relatively high dosage (e.g., from about 1 to 200 mg of antibody per dose, with dosages of from 5 to 25 mg being more commonly used) at relatively short intervals is sometimes required until progression of the disease is reduced or terminated, and preferably until the patient shows partial or complete amelioration of symptoms of disease. Thereafter, the patent can be administered a prophylactic regime.

Doses for nucleic acids encoding antibodies range from about 10 ng to 1 g, 100 ng to 100 mg, 1 μg to 10 mg, or 30-300 μg DNA per patient. Doses for infectious viral vectors vary from 10-100, or more, virions per dose.

Therapeutic reagents can be administered by parenteral, topical, intravenous, oral, subcutaneous, intraarterial, intracranial, intraperitoneal, intranasal or intramuscular means for prophylactic and/or therapeutic treatment. The most typical route of administration of an immunogenic agent is subcutaneous although other routes can be equally effective. The next most common route is intramuscular injection. This type of injection is most typically performed in the arm or leg muscles. In some methods, reagents are injected directly into a particular tissue where deposits have accumulated, for example intracranial injection. Intramuscular injection or intravenous infusion are preferred for administration of antibody. In some methods, particular therapeutic antibodies are injected directly into the cranium. In some methods, antibodies are administered as a sustained release composition or device, such as a Medipad™ device.

Immunological reagents of the invention can optionally be administered in combination with other agents that are at least partly effective in treatment of amyloidogenic disease. In certain embodiments, a humanized antibody of the invention (e.g., humanized Aβ antibody) is administered in combination with a second immunogenic or immunologic agent. For example, a humanized Aβ antibody of the invention can be administered in combination with another humanized Aβ antibody. In other embodiments, a humanized Aβ antibody is administered to a patient who has received or is receiving an Aβ vaccine. In the case of Alzheimer's and Down's syndrome, in which amyloid deposits occur in the brain, agents of the invention can also be administered in conjunction with other agents that increase passage of the agents of the invention across the blood-brain barrier. Agents of the invention can also be administered in combination with other agents that enhance access of the therapeutic agent to a target cell or tissue, for example, liposomes and the like. Coadministering such agents can decrease the dosage of a therapeutic agent (e.g., therapeutic antibody or antibody chain) needed to achieve a desired effect.

D. Pharmaceutical Compositions

Immunological reagents of the invention are often administered as pharmaceutical compositions comprising an active therapeutic agent, i.e., and a variety of other pharmaceutically acceptable components. See Remington's Pharmaceutical Science (15th ed., Mack Publishing Company, Easton, Pa. (1980)). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose (TM), agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, agents of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation, which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249: 1527 (1990) and Hanes, Advanced Drug Delivery Reviews 28:97 (1997)). The agents of this invention can be administered in the form of a depot injection or implant preparation, which can be formulated in such a manner as to permit a sustained or pulsatile release of the active ingredient.

Additional formulations suitable for other modes of administration include oral, intranasal, and pulmonary formulations, suppositories, and transdermal applications. For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins (See Glenn et al., Nature 391, 851 (1998)). Co-administration can be achieved by using the components as a mixture or as linked molecules obtained by chemical crosslinking or expression as a fusion protein.

Alternatively, transdermal delivery can be achieved using a skin patch or using transferosomes (Paul et al., Eur. J. Immunol. 25:3521 (1995); Cevc et al., Biochem. Biophys. Acta 1368:201-15 (1998)).

E. Monitoring the Course of Treatment

The invention provides methods of monitoring treatment in a patient suffering from or susceptible to Alzheimer's, i.e., for monitoring a course of treatment being administered to a patient. The methods can be used to monitor both therapeutic treatment on symptomatic patients and prophylactic treatment on asymptomatic patients. In particular, the methods are useful for monitoring passive immunization (e.g., measuring level of administered antibody).

Some methods involve determining a baseline value, for example, of an antibody level or profile in a patient, before administering a dosage of agent, and comparing this with a value for the profile or level after treatment. A significant increase (i.e., greater than the typical margin of experimental error in repeat measurements of the same sample, expressed as one standard deviation from the mean of such measurements) in value of the level or profile signals a positive treatment outcome (i.e., that administration of the agent has achieved a desired response). If the value for immune response does not change significantly, or decreases, a negative treatment outcome is indicated.

In other methods, a control value (i.e., a mean and standard deviation) of level or profile is determined for a control population. Typically the individuals in the control population have not received prior treatment. Measured values of the level or profile in a patient after administering a therapeutic agent are then compared with the control value. A significant increase relative to the control value (e.g., greater than one standard deviation from the mean) signals a positive or sufficient treatment outcome. A lack of significant increase or a decrease signals a negative or insufficient treatment outcome. Administration of agent is generally continued while the level is increasing relative to the control value. As before, attainment of a plateau relative to control values is an indicator that the administration of treatment can be discontinued or reduced in dosage and/or frequency.

In other methods, a control value of the level or profile (e.g., a mean and standard deviation) is determined from a control population of individuals who have undergone treatment with a therapeutic agent and whose levels or profiles have plateaued in response to treatment. Measured values of levels or profiles in a patient are compared with the control value. If the measured level in a patient is not significantly different (e.g., more than one standard deviation) from the control value, treatment can be discontinued. If the level in a patient is significantly below the control value, continued administration of agent is warranted. If the level in the patient persists below the control value, then a change in treatment may be indicated.

In other methods, a patient who is not presently receiving treatment but has undergone a previous course of treatment is monitored for antibody levels or profiles to determine whether a resumption of treatment is required. The measured level or profile in the patient can be compared with a value previously achieved in the patient after a previous course of treatment. A significant decrease relative to the previous measurement (i.e., greater than a typical margin of error in repeat measurements of the same sample) is an indication that treatment can be resumed. Alternatively, the value measured in a patient can be compared with a control value (mean plus standard deviation) determined in a population of patients after undergoing a course of treatment. Alternatively, the measured value in a patient can be compared with a control value in populations of prophylactically treated patients who remain free of symptoms of disease, or populations of therapeutically treated patients who show amelioration of disease characteristics. In all of these cases, a significant decrease relative to the control level (i.e., more than a standard deviation) is an indicator that treatment should be resumed in a patient.

The tissue sample for analysis is typically blood, plasma, serum, mucous fluid or cerebrospinal fluid from the patient. The sample is analyzed, for example, for levels or profiles of antibodies to Aβ peptide, e.g., levels or profiles of humanized antibodies. ELISA methods of detecting antibodies specific to Aβ are described in the Examples section. In some methods, the level or profile of an administered antibody is determined using a clearing assay, for example, in an in vitro phagocytosis assay, as described herein. In such methods, a tissue sample from a patient being tested is contacted with amyloid deposits (e.g., from a PDAPP mouse) and phagocytic cells bearing Fc receptors. Subsequent clearing of the amyloid deposit is then monitored. The existence and extent of clearing response provides an indication of the existence and level of antibodies effective to clear Aβ in the tissue sample of the patient under test.

The antibody profile following passive immunization typically shows an immediate peak in antibody concentration followed by an exponential decay. Without a further dosage, the decay approaches pretreatment levels within a period of days to months depending on the half-life of the antibody administered.

In some methods, a baseline measurement of antibody to Aβ in the patient is made before administration, a second measurement is made soon thereafter to determine the peak antibody level, and one or more further measurements are made at intervals to monitor decay of antibody levels. When the level of antibody has declined to baseline or a predetermined percentage of the peak less baseline (e.g., 50%, 25% or 10%), administration of a further dosage of antibody is administered. In some methods, peak or subsequent measured levels less background are compared with reference levels previously determined to constitute a beneficial prophylactic or therapeutic treatment regime in other patients. If the measured antibody level is significantly less than a reference level (e.g., less than the mean minus one standard deviation of the reference value in population of patients benefiting from treatment) administration of an additional dosage of antibody is indicated.

Additional methods include monitoring, over the course of treatment, any art-recognized physiologic symptom (e.g., physical or mental symptom) routinely relied on by researchers or physicians to diagnose or monitor amyloidogenic diseases (e.g., Alzheimer's disease). For example, one can monitor cognitive impairment. The latter is a symptom of Alzheimer's disease and Down's syndrome but can also occur without other characteristics of either of these diseases. For example, cognitive impairment can be monitored by determining a patient's score on the Mini-Mental State Exam in accordance with convention throughout the course of treatment.

F. Kits

The invention further provides kits for performing the monitoring methods described above. Typically, such kits contain an agent that specifically binds to antibodies to Aβ. The kit can also include a label. For detection of antibodies to Aβ, the label is typically in the form of labeled anti-idiotypic antibodies. For detection of antibodies, the agent can be supplied prebound to a solid phase, such as to the wells of a microtiter dish. Kits also typically contain labeling providing directions for use of the kit. The labeling may also include a chart or other correspondence regime correlating levels of measured label with levels of antibodies to Aβ. The term labeling refers to any written or recorded material that is attached to, or otherwise accompanies a kit at any time during its manufacture, transport, sale or use. For example, the term labeling encompasses advertising leaflets and brochures, packaging materials, instructions, audio or videocassettes, computer discs, as well as writing imprinted directly on kits.

The invention also provides diagnostic kits, for example, research, detection and/or diagnostic kits (e.g., for performing in vivo imaging). Such kits typically contain an antibody for binding to an epitope of Aβ, preferably within residues 1-10. Preferably, the antibody is labeled or a secondary labeling reagent is included in the kit. Preferably, the kit is labeled with instructions for performing the intended application, for example, for performing an in vivo imaging assay. Exemplary antibodies are those described herein.

G. In Vivo Imaging

The invention provides methods of in vivo imaging amyloid deposits in a patient. Such methods are useful to diagnose or confirm diagnosis of Alzheimer's disease, or susceptibility thereto. For example, the methods can be used on a patient presenting with symptoms of dementia. If the patient has abnormal amyloid deposits, then the patient is likely suffering from Alzheimer's disease. The methods can also be used on asymptomatic patients. Presence of abnormal deposits of amyloid indicates susceptibility to future symptomatic disease. The methods are also useful for monitoring disease progression and/or response to treatment in patients who have been previously diagnosed with Alzheimer's disease.

The methods work by administering a reagent, such as antibody that binds to Aβ, to the patient and then detecting the agent after it has bound. Preferred antibodies bind to Aβ deposits in a patient without binding to full length APP polypeptide. Antibodies binding to an epitope of Aβ within amino acids 1-10 are particularly preferred. In some methods, the antibody binds to an epitope within amino acids 7-10 of Aβ. Such antibodies typically bind without inducing a substantial clearing response. In other methods, the antibody binds to an epitope within amino acids 1-7 of Aβ. Such antibodies typically bind and induce a clearing response to Aβ. However, the clearing response can be avoided by using antibody fragments lacking a full-length constant region, such as Fabs. In some methods, the same antibody can serve as both a treatment and diagnostic reagent. In general, antibodies binding to epitopes C-terminal to residue 10 of Aβ do not show as strong a signal as antibodies binding to epitopes within residues 1-10, presumably because the C-terminal epitopes are inaccessible in amyloid deposits. Accordingly, such antibodies are less preferred.

Diagnostic reagents can be administered by intravenous injection into the body of the patient, or directly into the brain by intracranial injection or by drilling a hole through the skull. The dosage of reagent should be within the same ranges as for treatment methods. Typically, the reagent is labeled, although in some methods, the primary reagent with affinity for Aβ is unlabelled and a secondary labeling agent is used to bind to the primary reagent. The choice of label depends on the means of detection. For example, a fluorescent label is suitable for optical detection. Use of paramagnetic labels is suitable for tomographic detection without surgical intervention. Radioactive labels can also be detected using PET or SPECT.

Diagnosis is performed by comparing the number, size, and/or intensity of labeled loci, to corresponding baseline values. The base line values can represent the mean levels in a population of undiseased individuals. Baseline values can also represent previous levels determined in the same patient. For example, baseline values can be determined in a patient before beginning treatment, and measured values thereafter compared with the baseline values. A decrease in values relative to baseline signals a positive response to treatment.

H. Clinical Trials

A single-dose phase I trial can be performed to determine safety in humans. A therapeutic agent (e.g., an antibody of the invention) is administered in increasing dosages to different patients starting from about 0.01 the level of presumed efficacy, and increasing by a factor of three until a level of about 10 times the effective mouse dosage is reached.

A phase II trial can further performed to determine therapeutic efficacy. Patients with early to mid Alzheimer's Disease defined using Alzheimer's disease and Related Disorders Association (ADRDA) criteria for probable AD are selected. Suitable patients score in the 12-26 range on the Mini-Mental State Exam (MMSE). Other selection criteria are that patients are likely to survive the duration of the study and lack complicating issues such as use of concomitant medications that may interfere. Baseline evaluations of patient function are made using classic psychometric measures, such as the MMSE, and the ADAS, which is a comprehensive scale for evaluating patients with Alzheimer's Disease status and function. These psychometric scales provide a measure of progression of the Alzheimer's condition. Suitable qualitative life scales can also be used to monitor treatment. Disease progression can also be monitored by MRI. Blood profiles of patients can also be monitored including assays of immunogen-specific antibodies and T-cells responses.

Following baseline measurements, patients begin receiving treatment. They are randomized and treated with either therapeutic agent or placebo in a blinded fashion. Patients are monitored at least every six months. Efficacy is determined by a significant reduction in progression of a treatment group relative to a placebo group.

A second phase II trial can be performed to evaluate conversion of patients from non-Alzheimer's Disease early memory loss, sometimes referred to as age-associated memory impairment (AAMI) or mild cognitive impairment (MCI), to probable Alzheimer's disease as defined as by ADRDA criteria. Patients with high risk for conversion to Alzheimer's Disease are selected from a non-clinical population by screening reference populations for early signs of memory loss or other difficulties associated with pre-Alzheimer's symptomatology, a family history of Alzheimer's Disease, genetic risk factors, age, sex, and other features found to predict high-risk for Alzheimer's Disease. Baseline scores on suitable metrics including the MMSE and the ADAS together with other metrics designed to evaluate a more normal population are collected. These patient populations are divided into suitable groups with placebo comparison against dosing alternatives with the agent. These patient populations are followed at intervals of about six months, and the endpoint for each patient is whether or not he or she converts to probable Alzheimer's Disease as defined by ADRDA criteria at the end of the observation.

The present invention will be more fully described by the following non-limiting examples.

### EXAMPLES

The following Sequence identifiers are used throughout the Examples section to refer to immunoglobulin chain variable region nucleotide and amino acid sequences.

TABLE 4
Sequence Identifier Key

| | VL nucleotide | VL amino acid | VH nucleotide | VH amino acid |
|---|---|---|---|---|
| | sequence | sequence | sequence | sequence |
| Murine 3D6 | SEQ ID NO: 1 | SEQ ID NO: 2 | SEQ ID NO: 3 | SEQ ID NO: 4 |
| Humanized | SEQ ID NO: 5 | SEQ ID NO: 6 | SEQ ID NO: 7 | SEQ ID NO: 8 |
| 3D6v1 | | | | |
| Humanized | SEQ ID NO: 9 | SEQ ID NO: 10 | SEQ ID NO: 11 | SEQ ID NO: 12 |
| 3D6v2 | | | | |
| Murine 10D5 | SEQ ID NO: 13 | SEQ ID NO: 14 | SEQ ID NO: 15 | SEQ ID NO: 16 |
| Murine 12B4 | SEQ ID NO: 17 | SEQ ID NO: 18 | SEQ ID NO: 19 | SEQ ID NO: 20 |
| humanized | SEQ ID NO: 21 | SEQ ID NO: 22 | SEQ ID NO: 23 | SEQ ID NO: 24 |
| 12B4v1 | | | | |
| humanized | SEQ ID NO: 17 | SEQ ID NO: 18 | | SEQ ID NO: 25 |
| 12B4v2 | | | | |
| humanized | SEQ ID NO: 17 | SEQ ID NO: 18 | | SEQ ID NO: 26 |
| 12B4v3 | | | | |
| Murine 12A11 | SEQ ID NO: 27 | SEQ ID NO: 28 | SEQ ID NO: 29 | SEQ ID NO: 30 |
| Humanized | SEQ ID NO: 31 | SEQ ID NO: 32 | SEQ ID NO: 33 | SEQ ID NO: 34 |
| 12A11v1 | | | | |
| Humanized | SEQ ID NO: 31 | SEQ ID NO: 32 | | SEQ ID NO: 35 |
| 12A11v2 | | | | |
| Humanized | SEQ ID NO: 31 | SEQ ID NO: 32 | | SEQ ID NO: 36 |
| 12A11v2.1 | | | | |
| Humanized | SEQ ID NO: 31 | SEQ ID NO: 32 | | SEQ ID NO: 37 |
| 12A11v3 | | | | |
| Humanized | SEQ ID NO: 31 | SEQ ID NO: 32 | SEQ ID NO: 38 | SEQ ID NO: 39 |
| 12A11v3.1 | | | | |
| Humanized | SEQ ID NO: 31 | SEQ ID NO: 32 | | SEQ ID NOs: 40-55 |
| 12A11v4.1-8 | | | | |
| Murine 15C11 | SEQ ID NO: 56 | SEQ ID NO: 57 | SEQ ID NO: 58 | SEQ ID NO: 59 |

As used herein, an antibody or immunoglobulin sequence comprising a VL and/or VH sequence as set forth in any one of SEQ ID NOs: 1-59 can comprise (or encode) either the full sequence or can comprise the mature sequence (i.e., mature peptide without the signal or leader peptide).

### Example I

### In Vivo and Ex Vivo Efficacy of Anti-Aβ Antibodies mAb 3D6 and 10D5

The 3D6 and 10D5 antibodies were tested for a variety of activities important in amyloidosis. Experimental details can be found in WO 02/46237, the entire content of which is incorporated herein by reference.

In a first experiment, 10D5 was shown to inhibit accumulation of Aβ in the brains of heterozygotic PDAPP mice (8.5 to 10.5 months of age). mAbs 266, 21F12 and 2H3, and a mouse polyclonal antibody (pab) directed to Aβ1-42, were included for comparison purposes. Test antibodies were administered intraperitoneally (weekly at a dose of about ˜10 mg/kg). Control mice received diluent alone (PBS). Antibody titers were monitored and all titers were significant with the exception of 2H3 which was eliminated from the study. Treatment was continued over a six-month period at which point the mice were euthanized in order to perform biochemical and pathological studies.

Aβ and APP levels were assayed by ELISA in guanidine extracts from hippocampal, cortical, and cerebellar regions of the brains of the animals. The pAb and mAb 10D5 significantly reduced Aβ in the cortex (65%, p<0.05 and 55%, p=0.0433, respectively). The pAb and mAbs 10D5 and 266 also reduced Aβ in the hippocampus, 50%, p=0.0055, 33%, p=0.0543 and 21%, p=0.0990, respectively. In the cerebellum, the pAb and mAb 266 showed significant reductions of the levels of total Aβ (43%, p=0.0033 and 46%, p=0.0184, respectively) and 10D5 showed a near significant reduction (29%, p=0.0675). In summary, Aβ levels were significantly reduced in the cortex, hippocampus and cerebellum in animals treated with the polyclonal antibody raised against Aβ1-42. To a lesser extent monoclonal antibodies raised to the amino terminal and midregion of Aβ1-42, specifically amino acids 1-16 and 13-28, also showed significant treatment effects. The effect was specific for Aβ as APP levels were virtually unchanged in all of the treated compared to the control animals (also determined by ELISA).

Immunohistochemical analysis of brain sections (hippocampal, cortical, and cerebellar regions) were also performed to determine the effect of antibody administration on the extent of Aβ-immunoreactive plaques in the brains of the animals. Relative to control-treated animals, the pAb and mAb 10D5 reduced plaque burden by 93% and 81% (p<0.005), respectively. 21F12 also appeared to have a relatively modest effect on plaque burden.

In a second study, treatment with 10D5, 3D6 and 16C11 were compared. Control groups received either PBS or an irrelevant isotype-matched antibody (TM2a). The mice were older (11.5-12 month old heterozygotes) than in the previous study, otherwise the experimental design was the same. The mAb 10D5 again reduced plaque burden by greater than 80% (p=0.003) relative to the controls. Moreover, mAb 3D6 produced an 86% (p=0.003) reduction in plaque burden whereas mAb 16C11 failed to have any effect on plaque burden. Similar findings were obtained with Aβ42 ELISA measurements. These results demonstrate that passive administration of the N-terminal Aβ antibodies, 3D6 and 10D5, reduces the extent of plaque deposition in a mouse model of Alzheimer's disease.

It was further shown that the peripherally administered antibodies entered the CNS with the concentration of antibody in the brain parenchyma representing 0.1% of the antibody concentration in serum, indicating that the antibodies can enter the brain where they can directly trigger amyloid clearance. Plaque clearance was demonstrated to be via a mechanism of Fc-receptor mediated phagocytosis. The antibodies were assayed in an ex vivo phagocytosis assay described in detail in WO 02/46237 (see also Bard et al. (2000) Nat. Med. 6:916-919). Briefly, primary microglial cells obtained from the cerebral cortices of neonate DBA/2N mice (1-3 days) were cultured in 24-well plates with unfixed cryostat sections of either PDAPP mouse or human AD brains (post-mortem interval<3 hr) mounted onto poly-lysine coated round glass coverslips. Antibodies were added at a concentration of 5 μg/ml final for 1 hour prior to addition of cells. After 24 hours of incubation, cultures were fixed, permeabilized and immunostained for Aβ peptide. Exogenous microglial cells were visualized by a nuclear stain (DAPI). For Aβ quantitation, the cultures were urea extracted and proteins immunoblotted using the Aβ1-42 pAb.

Data from the ex vivo assays showed that antibodies binding to epitopes within residues 1-7 of Aβ (e.g., mAbs 3D6 (1-5), 10D5 (3-7) and 22C8 (3-7)) both bind and clear amyloid deposits, whereas antibodies binding to epitopes within amino acids 4-10 (e.g., mAbs 6E10 (5-10) and 14A8 (4-10)) bind without clearing amyloid deposits. Antibodies binding to epitopes C-terminal to residue 10 (e.g., mAbs 18G11 (10-18), 266 (16-24), 22D12 (18-21), 2G3 (−40), 16C11 (−40/−42) and 21F12 (−42)) neither bind nor clear amyloid deposits.

The ability of several of the Aβ antibodies to induce phagocytosis in the ex vivo assay was further compared to their ability to reduce in vivo plaque burden in passive transfer studies. Although 16C11 and 21F12 bound to aggregated synthetic Aβ peptide with high avidity, these antibodies did not react with β-amyloid plaques in unfixed brain sections, did not trigger phagocytosis in the ex vivo assay, and were not efficacious at clearing plaques in vivo. mAbs 10D5 and 3D6, as well as the pAb, were active by all three measures. These results show that efficacy in vivo is due, at least in part, to direct antibody mediated clearance of the plaques within the CNS, and that the ex vivo assay is predictive of this in vivo efficacy.

### Example II

### Efficacy of mAb 3D6, 10D5 and 12B4 on Various Neuropathological Endpoints in PDAPP Mice

PDAPP mice were passively immunized with either mAb 12B4 or mAb 3D6, both of the IgG1 isotype. 12B4 was tested at 10 mg/kg. mAb 3D6 was tested at three different doses, 10 mg/kg, 1 mg/kg and 10 mg/kg once a month (1×4). An unrelated IgG1 antibody (TY 11/15) and PBS injections served as controls. Active immunization with Aβ peptide served as a comparison. Between 20 and 35 animals were analyzed in each group. The neuropathological endpoints assayed include amyloid burden and neuritic burden.

The extent of the frontal cortex occupied by amyloid deposits was determined by immunostaining with 3D6 followed by quantitative image analysis. Each of the immunotherapies (i.e., administration with 12B4, 3D6 (all doses tested) and Aβ peptide) led to a significant reduction of frontal cortex amyloid burden (i.e., compared to control Ab exhibiting a 12% reduction).

Previously, it had been observed that 10D5 was unable to significantly reduce neuritic burden, suggesting that antibodies of the IgG1 isotype, but not other isotypes, are able to reduce neuritic burden in animal models of Alzheimer's disease (data not shown). Neuritic burden following passive immunization with 12B4 versus 3D6 (both of the IgG1 isotype) was thus determined in PDAPP mice by immunostaining of brain sections with anti-APP antibody 8E5 followed by quantitative image analysis. Neuritic dystrophy is indicated by the appearance of dystrophic neurites (e.g., neurites with a globular appearance) located in the immediate vicinity of amyloid plaques. The results of this analysis indicated that treatment with 12B4 most significantly reduced neuritic burden. By contrast, 3D6 did not significantly reduce neuritic burden.

When the 12B4 and 3D6 antibodies were tested in the ex vivo phagocytosis assay described supra, both antibodies cleared amyloid deposits in PDAPP brain sections, and the microglial cells showed numerous phagocytic vesicles containing Aβ. Similar results were obtained with AD brain sections; 3D6 (a humanized version) and chimeric 12B4 induced phagocytosis of AD plaques, while control IgG 1 was ineffective.

### Example III

### Mouse 3D6 Variable Region Sequences

The VL and VH regions of 3D6 from hybridoma cells were cloned by RT-PCR and 5′ RACE using mRNA from hybridoma cells and standard cloning methodology. The nucleotide sequences encoding the VL and VH regions of 3D6 are set forth as SEQ ID NOs: 1 and 3, respectively (and in Tables 5 and 7, respectively). The amino acid sequences of the VL and VH regions of 3D6 are set forth as SEQ ID NOs: 2 and 4, respectively (and in Tables 6 and 8, respectively, and in FIGS. 1 and 2, respectively). From N-terminal to C-terminal, both light and heavy chains comprise the domains FR1, CDR1, FR2, CDR2, FR3, CDR3 and FR4. The assignment of amino acids to each domain is in accordance with the numbering convention of Kabat et al., supra.

TABLE 5
Mouse 3D6 VL Nucleotide Sequence

| | |
|---|---|
| ATGATGAGTCCTGCCCAGTTCCTGTTTCTGTTAGTGC | (SEQ ID NO:1) |
| TCTGGATTCGGGAAACCAACGGTTATGTTGTGATGAC | |
| CCAGACTCCACTCACTTTGTCGGTTACCATTGGACAA | |
| CCAGCCTCCATCTCTTGCAAGTCAAGTCAGAGCCTCT | |
| TAGATAGTGATGGAAAGACATATTTGAATTGGTTGTT | |
| ACAGAGGCCAGGCCAGTCTCCAAAGCGCCTAATCTAT | |
| CTGGTGTCTAAACTGGACTCTGGAGTCCCTGACAGGT | |
| TCACTGGCAGTGGATCAGGGACAGATTTTACACTGAA | |
| AATCAGCAGAATAGAGGCTGAGGATTTGGGACTTTAT | |
| TATTGCTGGCAAGGTACACATTTTCCTCGGACGTTCG | |
| GTGGAGGCACCAAGCTGGAAATCAAA | |

*Leader peptide is underlined

TABLE 6
Mouse 3D6 VL Amino Acid Sequence

| | |
|---|---|
| mmspaqflfllvlwiretngYVVMTQTPLTLSVTIGQ | (SEQ ID NO:2) |
| PASISCkssqslldsdgktylnWLLQRPGQSPKRLIY | |
| lvskldsGVPDRFTGSGSGTDFTLKISRIEAEDLGLY | |
| YCwqgthfprtFGGGTKLEIK | |

*Leader peptide and CDRs are in lower case.

TABLE 7
Mouse 3D6 VH Nucleotide Sequence

| | |
|---|---|
| ATGAACTTCGGGCTCAGCTTGATTTTCCTTGTCCTTG | (SEQ ID NO:3) |
| TTTTAAAAGGTGTCCAGTGTGAAGTGAAGCTGGTGGA | |
| GTCTGGGGGAGGCTTAGTGAAGCCTGGAGCGTCTCTG | |
| AAACTCTCCTGTGCAGCCTCTGGATTCACTTTCAGTA | |
| ACTATGGCATGTCTTGGGTTCGCCAGAATTCAGACAA | |
| GAGGCTGGAGTGGGTTGCATCCATTAGGAGTGGTGGT | |
| GGTAGAACCTACTATTCAGACAATGTAAAGGGCCGAT | |
| TCACCATCTCCAGAGAGAATGCCAAGAACACCCTGTA | |
| CCTGCAAATGAGTAGTCTGAAGTCTGAGGACACGGCC | |
| TTGTATTATTGTGTCAGATATGATCACTATAGTGGTA | |
| GCTCCGACTACTGGGGCCAGGGCACCACT | |

*Leader peptide is underlined.

TABLE 8
Mouse 3D6 VH amino acid sequence

| | |
|---|---|
| mnfglsliflvlvlkgvqcEVKLVESGGGLVKPGASL | (SEQ ID NO:4) |
| KLSCAASGFTFSnygmsWVRQNSDKRLEWVAsirsgg | |
| grtyysdnvkgRFTISRENAKNTLYLQMSSLKSEDTA | |
| LYYCVRydhysgssdyWGQGTTVTVSS | |

*Leader peptide and CDRs in lower case.

For expression of a chimeric 3D6 antibody, the variable heavy and light chain regions were re-engineered to encode splice donor sequences downstream of the respective VDJ or VJ junctions, and cloned into the mammalian expression vector pCMV-hγ1 (SEQ ID NOs:91 and 92) for the heavy chain, and pCMV-hκ1 (SEQ ID NOs:93 and 94) for the light chain (see e.g., Maeda et al. (1991) Hum. Antibod. Hybridomas. 2:124-134). These vectors encode human γ1 and Ck constant regions as exonic fragments downstream of the inserted variable region cassette. Following sequence verification, the heavy chain and light chain expression vectors were co-transfected into COS cells. Conditioned media was collected 48 hrs post transfection and assayed by western blot analysis for antibody production or ELISA for Aβ binding. Chimeric 3D6 was found to bind to Aβ with high avidity, similar to that demonstrated by murine 3D6. Furthermore, an ELISA based competitive inhibition assay revealed that the chimeric 3D6 and the murine 3D6 antibody competed equally with biotinylated-3D6 binding to Aβ. Moreover, both murine and chimeric 3D6 were effective at clearing Aβ plaques in the ex vivo assay, described supra.

### Example IV

### Mouse 10D5 Variable Region Sequences

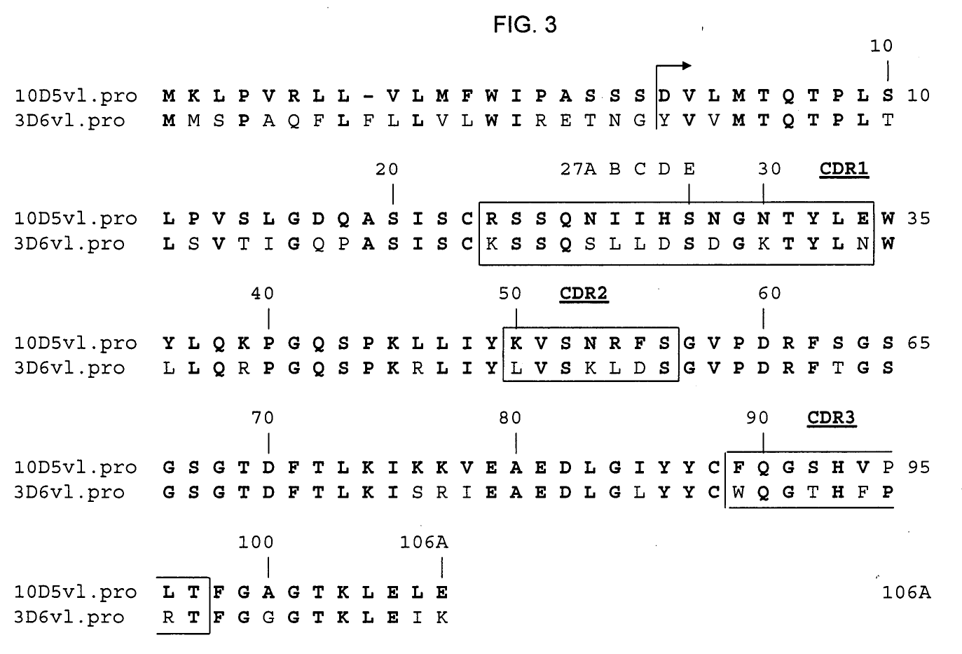

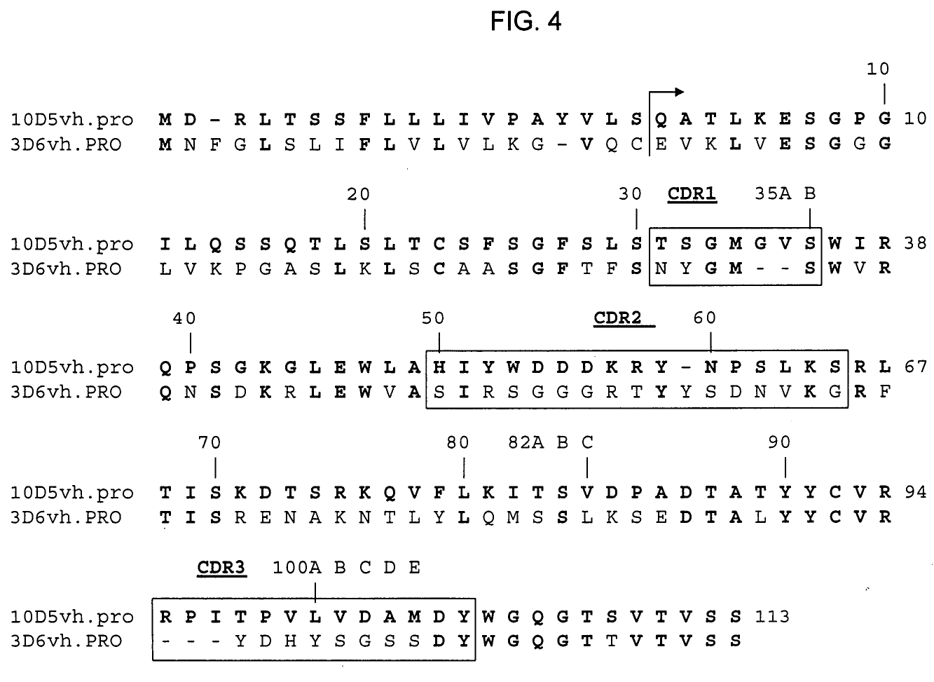

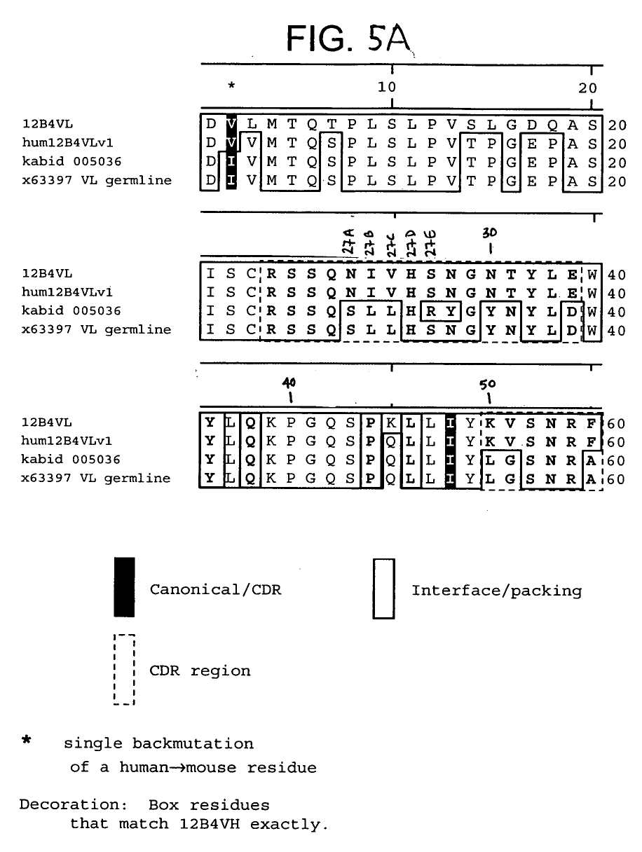

The VL and VH regions of 10D5 from hybridoma cells were cloned by RT-PCR using 5′ RACE procedures. The nucleotide sequences encoding the VL and VH regions of 10D5 are set forth as SEQ ID NOs: 13 and 15, respectively (and in Tables 9 and 11, respectively). The amino acid sequences of the VL and VH regions of 10D5 are set forth as SEQ ID NOs: 14 and 16, respectively (and in Tables 10 and 12, respectively, and in FIGS. 3 and 4, respectively). From N-terminal to C-terminal, both light and heavy chains comprise the domains FR1, CDR1, FR2, CDR2, FR3, CDR3 and FR4. The assignment of amino acids to each domain is in accordance with the numbering convention of Kabat et al., supra.

TABLE 9
Mouse 10D5 VL DNA sequence

| | |
|---|---|
| ATGAAGTTGCCTGTTAGGCTGTTGGTACTGATGTTC | (SEQ ID NO:13) |
| TGGATTCCTGCTTCCAGCAGTGATGTTTTGATGACC | |
| CAAACTCCACTCTCCCTGCCTGTCAGTCTTGGAGAT | |
| CAAGCCTCCATCTCTTGCAGATCTAGTCAGAACATT | |
| ATACATAGTAATGGAAACACCTATTTAGAATGGTAC | |
| CTGCAGAAACCAGGCCAGTCTCCAAAGCTCCTGATC | |
| TACAAAGTTTCCAACCGATTTTCTGGGGTCCCAGAC | |
| AGGTTCAGTGGCAGTGGATCAGGGACAGATTTCACA | |
| CTCAAGATCAAGAAAGTGGAGGCTGAGGATCTGGGA | |
| ATTTATTACTGCTTTCAAGGTTCACATGTTCCGCTC | |
| ACGTTCGGTGCTGGGACCAAGCTGGAGCTGGAA | |

*Leader peptide underlined

TABLE 10
Mouse 10D5 VL Amino Acid Sequence

| | |
|---|---|
| mklpvrllvlmfwipasssdvlmtqtplslpvslgd | (SEQ ID NO:14) |
| qasiscRSSQNIIHSNGNTYLEwylqkpgqspklli | |
| yKVSNRFSgvpdrfsgsgsgtdftlkikkveaedlg | |
| iyycFQGSHVPLTfgagtkleleradaaptvsifpp | |
| strd | |

*Leader peptide underlined and CDRs are in upper case.

TABLE 11
Mouse 10D5 VH DNA sequence.

| | |
|---|---|
| ATGGACAGGCTTACTTCCTCATTCCTGCTGCTGATT | (SEQ ID NO:15) |
| GTCCCTGCATATGTCCTGTCCCAGGCTACTCTGAAA | |
| GAGTCTGGCCCTGGAATATTGCAGTCCTCCCAGACC | |
| CTCAGTCTGACTTGTTCTTTCTCTGGGTTTTCACTG | |
| AGCACTTCTGGTATGGGAGTGAGCTGGATTCGTCAG | |
| CCTTCAGGAAAGGGTCTGGAGTGGCTGGCACACATT | |
| TACTGGGATGATGACAAGCGCTATAACCCATCCCTG | |
| AAGAGCCGGCTCACAATCTCCAAGGATACCTCCAGA | |
| AAGCAGGTATTCCTCAAGATCACCAGTGTGGACCCT | |
| GCAGATACTGCCACATACTACTGTGTTCGAAGGCCC | |
| ATTACTCCGGTACTAGTCGATGCTATGGACTACTGG | |
| GGTCAAGGAACCTCAGTCACCGTCTCCTCA | |

*Leader peptide underlined.

TABLE 12
Mouse 10D5 VH Amino Acid Sequence

| | |
|---|---|
| mdrltssflllivpayvlsqatlkesgpgilqssqt | (SEQ ID NO:16) |
| lsltcsfsgfslsTSGMGVSwirqpsgkglewlaHI | |
| YWDDDKRYNPSLKSrltiskdtsrkqvflkitsvdp | |
| adtatyycvRRPITPVLVDAMDYwgqgtsvtvssak | |
| ttppsvyplardpggs | |

*Leader peptide underlined and CDRs are in upper case.

Chimeric 10D5 antibody expression vectors can be engineered as described for 3D6 supra and co-transfected into COS cells. Conditioned media is assayed by western blot analysis for antibody production or ELISA for Aβ binding.

### Example V

### Mouse 12B4 Variable Region Sequences

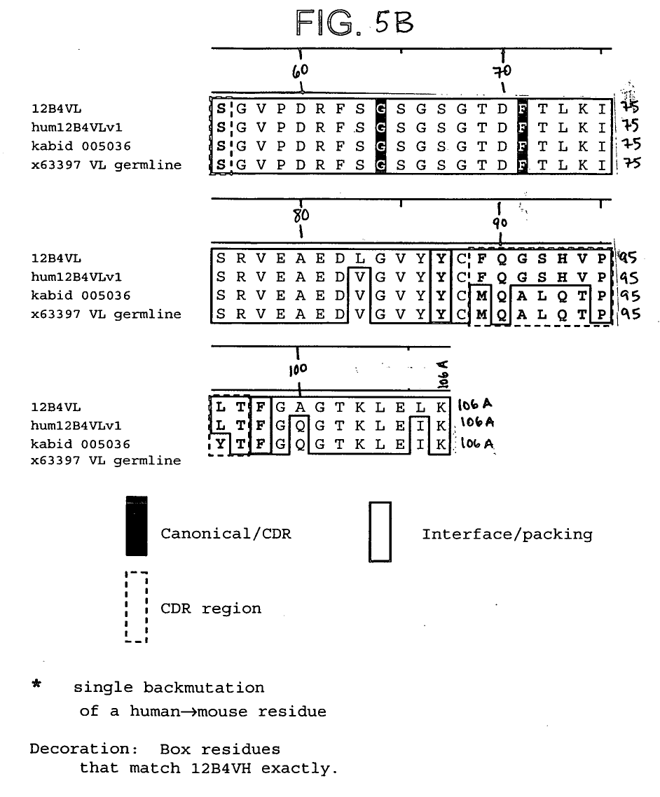

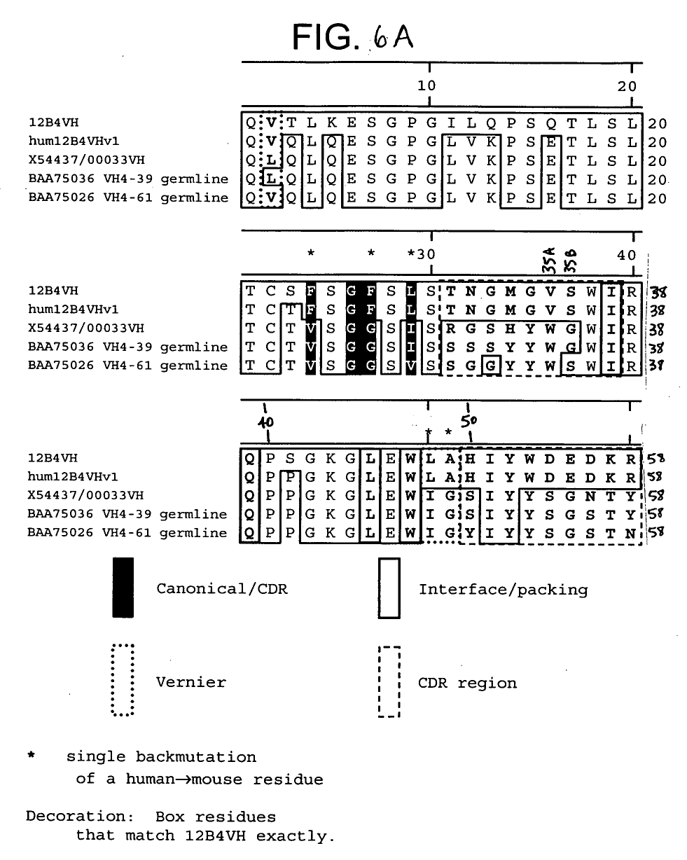

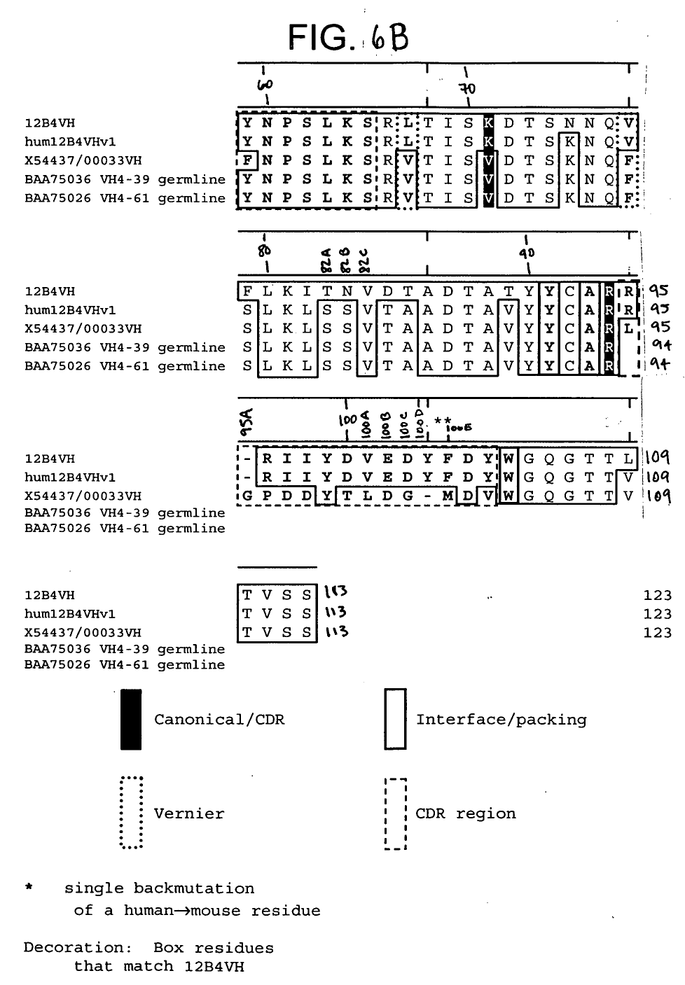

The VL and VH regions of 12B4 from hybridoma cells were cloned by RT-PCR and 5′ RACE using mRNA from hybridoma cells and standard cloning methodology. The nucleotide sequences encoding the VL and VH regions of 12B4 are set forth as SEQ ID NOs: 17 and 19, respectively (and in Tables 13 and 15, respectively). The amino acid sequences of the VL and VH regions of 12B4 are set forth as SEQ ID NOs: 18 and 20, respectively (and in Tables 14 and 16, respectively, and in FIGS. 5 and 6, respectively). From N-terminal to C-terminal, both light and heavy chains comprise the domains FR1, CDR1, FR2, CDR2, FR3, CDR3 and FR4. The assignment of amino acids to each domain is in accordance with the numbering convention of Kabat et al., supra.

TABLE 13
Mouse 12B4 VL DNA sequence

| | |
|---|---|
| ATGAAGTTGCCTGTTAGGCTGTTGGTGCTGATGTTC | (SEQ ID NO:17) |
| TGGATTCCTGCTTCCAGCAGTGATGTTTTGATGACC | |
| CAAACTCCACTCTCCCTGCCTGTCAGTCTTGGAGAT | |
| CAAGCCTCCATCTCTTGCAGATCTAGTCAGAACATT | |
| GTTCATAGTAATGGAAACACCTATTTAGAATGGTAC | |
| CTGCAGAAACCAGGCCAGTCTCCAAAGCTCCTGATC | |
| TACAAAGTTTCCAACCGATTTTCTGGGGTCCCAGAC | |
| AGGTTCAGTGGCAGTGGATCAGGGACAGATTTCACA | |
| CTCAAGATCAGCAGAGTGGAGGCTGAGGATCTGGGA | |
| GTTTATTACTGCTTTCAAGGTTCACATGTTCCGCTC | |
| ACGTTCGGTGCTGGGACCAAGCTGGAGCTGAAAC | |

*Leader peptide underlined

TABLE 14
Mouse 12B4 VL amino acid sequence

| | |
|---|---|
| mklpvrllvlmfwipasssDVLMTQTPLSLPVSLGD | (SEQ ID NO:18) |
| QASISCrssqnivhsngntyleWYLQKPGQSPKLLI | |
| YkvsnrfSGVPDRFSGSGSGTDFTLKISRVEAEDLG | |
| VYYCfqgshvpltFGAGTKLELK | |

*Leader peptide and CDRs in lower case.

TABLE 15
Mouse 12B4 VH DNA sequence.

| | |
|---|---|
| ATGGACAGGCTTACTTCCTCATTCCTGCTGCTGATT | (SEQ ID NO:19) |
| GTCCCTGCATATGTCCTGTCCCAGGTTACTCTGAAA | |
| GAGTCTGGCCCTGGGATATTGCAGCCCTCCCAGACC | |
| CTCAGTCTGACTTGTTCTTTCTCTGGGTTTTCACTG | |
| AGCACTAATGGTATGGGTGTGAGCTGGATTCGTCAG | |
| CCTTCAGGAAAGGGTCTGGAGTGGCTGGCACACATT | |
| TACTGGGATGAGGACAAGCGCTATAACCCATCCCTG | |
| AAGAGCCGGCTCACAATCTCCAAGGATACCTCTAAC | |
| AATCAGGTATTCCTCAAGATCACCAATGTGGACACT | |
| GCTGATACTGCCACATACTACTGTGCTCGAAGGAGG | |
| ATCATCTATGATGTTGAGGACTACTTTGACTACTGG | |
| GGCCAAGGCACCACTCTCACAGTCTCCTCAG | |

*Leader peptide underlined.

TABLE 16
Mouse 12B4 VH amino acid sequence

| | |
|---|---|
| mdrltssflllivpayvlsqVTLKESGPGILQPSQT | (SEQ ID NO:20) |
| LSLTCSFSGFSLStngmgvsWIRQPSGKGLEWLAhi | |
| ywdedkrynpslksRLTISKDTSNNQVFLKITNVDT | |
| ADTATYYCARrriiydvedyfdyWGQGTTLTVSS | |

*Leader peptide and CDRs in lower case.

Chimeric 12B4 antibody expression vectors were engineered as described for 3D6 supra and co-transfected into COS cells. Conditioned media was assayed by western blot analysis for antibody production or ELISA for Aβ binding. Chimeric 12B4 was found to bind to Aβ with high avidity, similar to that demonstrated by chimeric 3D6. Furthermore, an ELISA based competitive inhibition assay revealed that the chimeric 12B4 and the murine 12B4 antibody competed equally with biotinylated murine and chimeric 3D6, as well as 10D5, in binding to Aβ.

### Example VI

### 3D6 Humanization

In order to identify key structural framework residues in the murine 3D6 antibody, a three-dimensional model was generated based on the closest murine antibodies for the heavy and light chains. For this purpose, an antibody designated 1CR9 was chosen as a template for modeling the 3D6 light chain (PDB ID: 1CR9, Kanyo et al., supra), and an antibody designated 1OPG was chosen as the template for modeling the heavy chain. (PDB ID: 1OPG Kodandapani et al., supra). Amino acid sequence alignment of 3D6 with the light chain and heavy chain of these antibodies revealed that, with the exception of CDR3 of the heavy chain, the 1CR9 and 1OPG antibodies share significant sequence homology with 3D6. In addition, the CDR loops of the selected antibodies fall into the same canonical Chothia structural classes as do the CDR loops of 3D6, again excepting CDR3 of the heavy chain. Therefore, 1CR9 and 1OPG were initially selected as antibodies of solved structure for homology modeling of 3D6.

A first pass homology model of 3D6 variable region based on the antibodies noted above was constructed using the Look & SegMod Modules GeneMine (v3.5) software package. This software was purchased under a perpetual license from Molecular Applications Group (Palo Alto, Calif.). This software package, authored by Drs. Michael Levitt and Chris Lee, facilitates the process of molecular modeling by automating the steps involved in structural modeling a primary sequence on a template of known structure based on sequence homology. Working on a Silicon Graphics IRIS workstation under a UNIX environment, the modeled structure is automatically refined by a series of energy minimization steps to relieve unfavorable atomic contacts and optimize electrostatic and van der Walls interactions.

A further refined model was built using the modeling capability of QUANTA®. A query of the PDB database with CDR3 of the heavy chain of 3D6 identified 1qkz as most homologous and having the identical number of residues as 3D6. Hence, CDR3 of the heavy chain of 3D6 was modeled using the crystal structure of 1qkz as template.

Suitable human acceptor antibody sequences were identified by computer comparisons of the amino acid sequences of the mouse variable regions with the sequences of known human antibodies. The comparison was performed separately for the 3D6 heavy and light chains. In particular, variable domains from human antibodies whose framework sequences exhibited a high degree of sequence identity with the murine VL and VH framework regions were identified by query of the Kabat Database using NCBI BLAST (publicly accessible through the National Institutes of Health NCBI internet server) with the respective murine framework sequences.

Two candidate sequences were chosen as acceptor sequences based on the following criteria: (1) homology with the subject sequence; (2) sharing canonical CDR structures with the donor sequence; and (3) not containing any rare amino acid residues in the framework regions. The selected acceptor sequence for VL is Kabat ID Number (KABID) 019230 (Genbank Accession No. S40342), and for VH is KABID 045919 (Genbank Accession No. AF115110). First versions of humanized 3D6 antibody utilize these selected acceptor antibody sequences.

As noted supra, the humanized antibodies of the invention comprise variable framework regions substantially from a human immunoglobulin (acceptor immunoglobulin) and complementarity determining regions substantially from a mouse immunoglobulin (donor immunoglobulin) termed 3D6. Having identified the complementarity determining regions of 3D6 and appropriate human acceptor immunoglobulins, the next step was to determine which, if any, residues from these components to substitute to optimize the properties of the resulting humanized antibody. The criteria described supra were used to select residues for substitution.

FIGS. 1 and 2 depict alignments of the original murine 3D6 VL and VH, respectively, with the respective version 1 of the humanized sequence, the corresponding human framework acceptor sequence and, lastly, the human germline V region sequence showing highest homology to the human framework acceptor sequence. The shaded residues indicate the canonical (solid fill), vernier (dotted outline), packing (bold), and rare amino acids (bold italics), and are indicated on the figure. The asterisks indicate residues backmutated to murine residues in the human acceptor framework sequence, and CDR regions are shown overlined. In the 3D6 VL region, the following residues were selected as candidates for backmutation: residue 1—rare mouse, possibly contacting CDR; residue 2—canonical/CDR contacting residue; and residues 36 and 46-packing residues. In the 3D6 VH region, the following residues were selected as candidates for backmutation or substitution: residues 40 and 42—rare for mouse, replace with human; residue 49, CDR contacting, veneer; residue 93—packing residue; and residue 94—canonical residue.

A first version of humanized 3D6 (h3D6 v1) was generated having each of the substitutions indicated in FIGS. 1 and 2. PCR-mediated assembly was used to generate the humanized VL and VH chains. The nucleotide sequences encoding the h3D6 v1 VL and VH regions are set forth as SEQ ID NOs:5 and 7, respectively. The amino acid sequences of the h3D6 v1 VL and VH regions are set forth as SEQ ID NOs:6 and 8, respectively. A summary of the changes incorporated into version 1 of humanized 3D6 VH and VL is presented in Table 17.

TABLE 17
Summary of changes in humanized 3D6.v1

| Changes | VL (112 residues) | | VH (119 residues) | |
|---|---|---|---|---|
| Hu->Mu: Framework | 4/112 | | 3/119 (1 canonical, 1 packing) | |
| CDR1 | 6/16 | | 3/5 | |
| CDR2 | 4/7 | | 7/14 | |
| CDR3 | 5/8 | | 4/10 | |
| Hu->Mu | 19/112 (17%) | | 17/119 (14%) | |
| Mu->Hu: Framework | 13/112 | | 14/119 | |
| Backmutation notes | 1. | I2V which is a canonical | 4. | S49A Vernier/beneath the |
| | | position. | | CDRs. |
| | 2. | Y36L which is a packing | 5. | A93V which is a packing |
| | | residue and also lies under | | and vernier zone residue |
| | | the CDRs | 6. | K94R which is a canonical |
| | 3. | L46R which is a packing | | residue |
| | | residue and lies beneath the | | |
| | | CDRs | | |
| Acceptor notes | 7. | KABID 019230/Genbank | 11. | KABID045919/Genbank |
| | | Acc#S40342 | | Acc#AF115110 |
| | 8. | Hu κ LC subgroup II | 12. | Hu HC subgroup III |
| | 9. | CDRs from same canonical | 13. | CDRs from same canonical |
| | | structural group as donor | | structural group as donor |
| | | (m3D6) | | (m3D6) |
| | | L1 = class 4 | | H1 = class 1 |
| | | L2 = class 1 | | H2 = class 3 |
| | | L3 = class1 | 14. | Recognizes capsular |
| | 10. | Unknown specificity | | polysaccharide of Neisseria |
| | | | | meningitidis |
| Acceptor Germline | 15. | VH3-23 | 16. | A3 & A19 |

A second version of humanized 3D6 was created having each of the substitutions indicated for version 1, except for the D→Y substitution at residue 1. Substitution at this residue was performed in version 1 because the residue was identified as a CDR interacting residue. However, substitution deleted a residue which was rare for human immunoglobulins at that position. Hence, a version was created without the substitution. Moreover, non-germline residues in the heavy chain framework regions were substituted with germline residues, namely, H74=S, H77=T and H89=V. The nucleotide sequences encoding the h3D6 v2 VL and VH regions are set forth as SEQ ID NOs:9 and 11, respectively. The amino acid sequences of the h3D6 v2 VL and VH regions are set forth as SEQ ID NOs:10 and 12, respectively.

### Example VII

### 12B4 Humanization

In order to identify key structural framework residues in the murine 12B4 antibody, a three-dimensional model was generated based on the closest murine antibodies for the heavy and light chains. For this purpose, an antibody designated 2PCP was chosen as a template for modeling the 12B4 light chain (PDB ID: 2PCP, Lim et al. (1998) J. Biol. Chem. 273:28576), and an antibody designated 1ETZ was chosen as the template for modeling the heavy chain. (PDB ID: 1ETZ, Guddat et al. (2000) J. Mol. Biol. 302:853). Amino acid sequence alignment of 12B4 with the light chain and heavy chain of these antibodies revealed that the 2PCP and 1ETZ antibodies share significant sequence homology with 12B4. In addition, the CDR loops of the selected antibodies fall into the same canonical Chothia structural classes as do the CDR loops of 12B4. Therefore, 2PCP and 1ETZ were initially selected as antibodies of solved structure for homology modeling of 12B4.

Homology modeling and selection of acceptor sequences were performed as described for 3D6, supra. The selected acceptor sequence for VL is Kabat ID Number (KABID) 005036 (Genbank Accession No. X67904), and for VH is KABID 000333 (Genbank Accession No. X54437). First versions of humanized 12B4 antibody utilize these selected acceptor antibody sequences.

As noted supra, the humanized antibodies of the invention comprise variable framework regions substantially from a human immunoglobulin (acceptor immunoglobulin) and complementarity determining regions substantially from a mouse immunoglobulin (donor immunoglobulin) termed 12B4. Having identified the complementarity determining regions of 12B4 and appropriate human acceptor immunoglobulins, the next step was to determine which, if any, residues from these components to substitute to optimize the properties of the resulting humanized antibody.

The amino acid alignment of the reshaped light chain V region is shown in FIG. 5A-B. The choice of the acceptor framework (KABID 005036) is from the same human subgroup as that which corresponds to the murine V region, has no rarel framework residues, and the CDRs belong to the same Chothia canonical structure groups. A single back mutation (I2V) is dictated as this residue falls into the canonical classification. Version 1 of the reshaped VL is fully germline.

The amino acid alignment of the reshaped heavy chain V region is shown in FIG. 6A-B. The choice for the acceptor framework (KABID 000333) is from the same human subgroup as that which corresponds to the murine V region, has no rare framework residues, and the CDRs belong to the same Chothia canonical groups. Structural modeling of the murine VH chain, in conjunction with the amino acid alignment of KABID 000333 to the murine sequence dictates 9 back-mutations in version 1 (v1) of the reshaped heavy chain: L2V, V24F, G27F, 129L, 148L, G49A, V67L, V71K, & F78V (Kabat numbering). The back mutations are highlighted by asterisks in the amino-acid alignment shown in FIG. 6A-B.

Of the 9 back mutations, 4 are dictated by the model because the residues are canonical residues (V24F, G27F, 129L, & V71K, indicated by solid filled boxes), i.e. framework residues which may contribute to antigen binding by virtue of proximity to CDR residues. There are no back mutations necessary in the next most important class of residues, the interface residues involved in VH-VL packing interactions (indicated by open boxes). The remaining 5 residues targeted for back mutation (L2V, 148L, G49A, V67L, F78V, Kabat numbering) all fall into the vernier class (indirect contribution to CDR conformation, dense stippled boxes in FIG. 6A-B).

Version 2 was designed to retain the lowest number of non-CDR murine residues. The L2V backmutation introduces a non-germline change (when using VH4-61 as the germline reference), and this backmutation is eliminated in version 2 of the heavy chain to restore it to germ line. The remaining 4 vernier class backmutations are also restored in version 2 of the heavy chain (I48L, G49A, V67L, F78V). Version 2 thus contain a total of 5 non-CDR murine residues (1 in VL, and 4 in VH). Version 3 was designed to restore 2 of the 5 vernier residues (148L, & F78V), which the model indicates may be the more important vernier residues. Hence version 3 contains a total of 7 non CDR murine residues.

A summary of the changes incorporated into versions 1, 2 and 3 of humanized 12B4 are presented in Table 18.

TABLE 18
Summary of changes in humanized 12B4.v1

| Changes | VL (111 residues) | | VH (123 residues) | |
|---|---|---|---|---|
| Hu->Mu: Framework | 1/111 | | 9/123 | |
| CDR1 | 8/16 | | 7/7 | |
| CDR2 | 3/7 | | 8/16 | |
| CDR3 | 6/8 | | 10/13 | |
| Total Hu->Mu | 18/111 (16%) | | 34/123 (28%, v2 = 23%) | |
| Mu->Hu: Framework | 10/111 | | 16/123 | |
| Backmutation notes | 17. | I2V: Canonical: I2V†. | 18. | Canonical: V24F†, G27F†, |
| | | | | I29L†, & V71K† |
| | | | 19. | Packing: none |
| | | | 20. | Vernier: L2V*, I48L#, |
| | | | | G49A*, V67L*, F78V# |
| Acceptor notes | 21. | KABID 005036/Genbank | 24. | KABID 000333/Genbank |
| | | Acc#-x67904 | | Acc#x54437 |
| | 22. | CDRs from same canonical | 25. | CDRs from same canonical |
| | | structural group as donor | | structural group as donor |
| | | mouse; | | mouse; |
| | 23. | anti-cardiolipin/ss DNA | 26. | rheumatoid factor mAb |
| | | autoantibody from SLE | | from RA patient |
| | | patient | | |

†backmutate in v1, v2 and v3.
*backmutate in v1 only, eliminate in v2 and v3.
#backmutate in v1, eliminate in v2, restore in v3.

Kabat numbering for the 12B4 VL and VH chains is indicated in FIGS. 5 and 6, respectively. The nucleotide sequences encoding humanized 12B4VL (version 1) and 12B4VH (version 1) are set forth as SEQ ID NOs: 21 and 23, respectively. The amino acid sequences of humanized 12B4VL (version 1) and 12B4VH (version 1) are set forth as SEQ ID NOs: 22 and 24, respectively. The amino acid sequences for h12B4 v2 VH and h12B4 v3 VH are set forth as SEQ ID NOs: 25 and 26, respectively.

### Example VIII

### Functional Activities of Humanized 3D6 Antibodies

Functional testing of humanized 3D6v1 was conducted using conditioned media from COS cells transiently transfected with fully chimeric antibody, a mixture of either chimeric heavy chain+humanized light chain, or chimeric light chain+humanized heavy chain, and lastly, fully humanized antibody. The conditioned media was tested for binding to aggregated Aβ1-42 by ELISA assay. The humanized antibody showed good activity within experimental error, and displayed binding properties indistinguishable from the chimeric 3D6 reference sample. Similar testing of humanized 3D6v2 showed that the antibody had Aβ binding properties nearly identical to those of 3D6v1. 3D6v2 was also shown to have a virtually identical epitope map as compared to the murine 3D6 antibody. Humanized 3D6v1 antibody recognized Aβ in cryostat brain sections prepared from PDAPP mice (immunohistochemistry performed as described supra). In identical experiments, h3D6v2 stained PDAPP and AD brain sections in a manner similar to 3D6v1 (e.g., highly decorated plaques).

The ability of h3D6 antibodies v1 and v2 to compete with murine 3D6 was measured by ELISA using a biotinylated 3D6 antibody. Competitive binding analysis revealed that h3D6v1, h3D6v2, and a chimeric 3D6 all competed with m3D6 to bind Aβ. h3D6v1 and h3D6v2 were identical in their ability to compete with 3D6 to Aβ. The 10D5 antibody was used as a negative control, as it has a different binding epitope than 3D6. BIAcore analysis also revealed a high affinity of h3D6v1 and h3D6v2 for Aβ. In comparison to 3D6, which has a Kd of 0.88 nM, both h3D6v1 and h3D6v2 had about a 2 to 3 fold less binding affinity, measured at 2.06 nM and 2.24 nM for h3D6v1 and h3D6v2, respectively. The ELISA competitive binding assay revealed an approximate 6-fold less binding affinity for h3D6v1 and h3D6v2. Typically humanized antibodies lose about 3-4 fold in binding affinity in comparison to their murine counterparts. Therefore, a loss of about 3 fold (average of ELISA and BIAcore results) for h3D6v1 and h3D6v2 is within the accepted range.

The ability of h3D6v2 to stimulate microglial cells was tested in the ex vivo phagocytosis assay (described supra). h3D6v2 was as effective as chimeric 3D6 at inducing phagocytosis of Aβ aggregates from PDAPP mouse brain tissue. IgG was used as a negative control because it is incapable of binding Aβ and therefore cannot induce phagocytosis.

¹²⁵I labeled h3D6v2, m3D6, and antibody DAE13 were each IV-injected into 14 individual PDAPP mice in separate experiments. Mice were sacrificed after day 7 and perfused for further analysis. Their brain regions were dissected and measured for ¹²⁵I activity in specific brain regions. Radiolabel activity in the brain was compared with activity in serum samples. The data showed that h3D6v2 localized to the brain, and was particularly concentrated in the hippocampal region where Aβ is known to aggregate. Brain counts for m3D6 and DAE13 were comparable to h3D6v2. All three antibodies were able to cross the blood barrier as demonstrated by Aβ plaque binding in vivo.

### Example IX

### Efficacy of Various N-Terminal Aβ Antibodies

Binding of the monoclonal antibodies 6C6, 10D5, 2C1, 12B4, 3A3 and 12A11 to aggregated synthetic Aβ 1-42 was performed by ELISA, as described in Schenk, et al. (Nature 400:173 (1999)). Soluble Aβ 1-42 (in this example) refers to the synthetic Aβ 1-42 peptide sonicated in dimethyl sulfoxide (DMSO). Serial dilutions of the antibodies at 20 μg/ml were incubated with 50,000 cpm [¹²⁵I]Aβ1′-42 (190 μCi/μmol; labeling with Iodogen™ reagent, Pierce) overnight at room temperature. Fifty microliters of a slurry containing 75 mg/ml protein A Sepharose (Amersham Pharmacia) and 200 μg of rabbit anti-mouse IgG (H+L) (Jackson ImmunoResearch) were incubated with the diluted antibodies for 1 hour at room temperature, washed twice, and counted on a Wallac gamma counter (Perkin-Elmer). All steps were performed in RIA buffer consisting of 10 mM Tris, 0.5 M NaCl, 1 mg/ml gelatin, and 0.5% Nonidet P-40, pH 8.0. Results from the avidity study are shown below in Table 19.

TABLE 19

| | | | ED₅₀ on aggregated | % Capture of |
|---|---|---|---|---|
| Antibody | Epitope | Isotype | Aβ1-42, pM | soluble Aβ1-42 |
| 6C6 | Aβ3-7 | IgG1 | 40 | 1 |
| 10D5^{†} | Aβ3-7 | IgG1 | 53 | 1 |
| 2C1 | Aβ3-7 | IgG2a | 333 | 1 |
| 12B4^{†} | Aβ3-7 | IgG2a | 667 | 8 |
| 3A3 | Aβ3-7 | IgG2b | 287 | 1 |
| 12A11^{†} | Aβ3-7 | IgG1 | 233 | 30 |

^{†}Antibodies 10D5 and 12B4 are described in detail in WO 02/46237 and WO 03/077858, respectively. The 12A11 antibody is described in detail in International Patent Application Serial No. PCT/US2004/017514.
*As a comparison, the antibody 266 at 10 μg/ml would capture 70% of Aβ1-42.

All of the antibodies tested exhibited a high avidity for aggregated Aβ1-42 (<1 nM). Moreover, antibodies 12B4 and 12A11 appreciably captured soluble Aβ1-42 at antibody concentrations of 20 μg/ml. As shown in Table 19, the IgG1 antibody 12A11 captured Aβ1-42 more efficiently than the IgG2a antibody 12B4, while the IgG1 antibodies 6C6 and 10D5, the IgG2a antibody 2C1 and the IgG2b antibody 3A3 did not appreciably capture soluble Aβ.

As a measure of their ability to trigger Fc-mediated plaque clearance, the antibodies were also compared in the ex vivo phagocytosis assay using sections of brain tissue from PDAPP mice, as described supra. Irrelevant IgG1 and IgG2a antibodies, having no reactivity toward Aβ or other components of the assay, were used as isotype-matched negative controls. The two IgG2a antibodies, 12B4 and 2C1 reduced Aβ levels efficiently (73% for 12B4 and 69% for 2C1; P<0.001) with 12A11 and 3A3 showing somewhat less, albeit statistically significant, efficiency (48% for 12A11, P<0.05 and 59% for 3A3, P<0.001). The 10D5 and 6C6 antibodies did not significantly reduce Aβlevels. The performance of 12A11 in the ex vivo phagocytosis assay may be improved upon conversion to the IgG2a isotype which is a preferred isotype for microglial phagocytosis.

### Example X

### In Vivo Efficacy of Various N-Terminal Antibodies: Reduction of AD Neuropathology

To determine the in vivo efficacy of various N-terminal Aβ antibodies, 12A11, 12B4 and 10D5 were administered to separate groups of mice at 10 mg/kg by weekly intraperitoneal injection for 6 months as described in Bard et al. (2000) Nat. Med. 6:916. At the end of the study, total levels of cortical Aβ were determined by ELISA. Each of the antibodies significantly reduced total Aβ levels compared with the PBS control (P<0.001), i.e., 12B4 showed a 69% reduction, 10D5 showed a 52% reduction, and 12A11 showed a 31% reduction.

The level of neuritic dystrophy was then examined in sections of brain tissue from the above-mentioned mice to determine the association between plaque clearance and neuronal protection. Brain image analyses examining the percentage of frontal cortex occupied by neuritic dystrophy was determined for individual animals and expressed as the percentage of neuritic dystrophy relative to the mean of the control (set at 100%). The data showed that antibodies 10D5 and 12A11 were not effective at reducing neuritic dystrophy whereas 12B4 significantly reduced neuritic dystrophy (12B4, P<0.05; ANOVA followed by post hoc Dunnett's test). Experiments demonstrating the binding properties and in vivo efficacy of antibody 12A11 are also described in Bard, et al. PNAS 100:2023 (2003), incorporated by reference herein.

In summary, all antibodies had significant avidity for aggregated Aβ and triggered plaque clearance in an ex vivo assay. The IgG2a isotype (affinity for Fc receptors, in particular, FcγRI) appears to be an important attribute for both clearance of Aβ and protection against neuritic dystrophy. The antibody 12A11 (IgG1) captured soluble monomeric Aβ1-42 more efficiently than 12B4 (IgG2a) or 10D5 (IgG1) but was not as effective at reducing neuritic dystrophy. Enhanced efficacy in reducing plaque burden and reducing neuritic dystrophy may be achieved by engineering antibodies to have an isotype which maximally supports phagocytosis. Particularly efficacious antibodies bind to epitopes within the N-terminus of Aβ.

In a further study, a 12A11 IgG2a isotype antibody was tested for the ability to reduce AD-like neuropathology in PDAPP mice. 12-13 month old PDAPP mice were injected weekly for 6 months with 3 mg/kg 12A11 antibody. At the end of six months, animals were sacrificed and brain samples were analyzed for various end points including, Aβ burden, neuritic burden and synaptophysin levels. Administration of 12A11 antibody significantly reduced the level of amyloid burden in PDAPP brain samples. Administration of 12A11 antibodies also significantly reduced the degree of neuritic dystrophy (abnormal neuronal processes surrounding plaques). Likewise 12A11 administration significantly protected against the loss of synaptophysin (measure of synaptic integrity).

In further experiments 12A11 IgG2a isotype antibody was tested in ex vivo phagocytosis assays for the ability to clear amyloid. 12A11 antibody administration resulted in complete clearance of parenchymal plaque deposition and also resulted in partial clearing of vascular amyloid. The results were dose dependent.

### Example XI

### Capture Ability of Various Aβ Antibodies

Previous studies have shown that it is possible to predict in vivo efficacy of various Aβ antibodies in reducing AD-associated neuropathology (e.g., plaque burden) by the ability of antibodies to bind plaques ex vivo (e.g., in PDAPP or AD brain sections) and/or trigger plaque clearance in an ex vivo phagocytosis assay (Bard et al. (2000) Nat. Med. 6:916-919). The correlation supports the notion that Fc-dependent phagocytosis by microglial cells and/or macrophages is important to the process of plaque clearance in vivo. However, it has also been reported that antibody efficacy can also be obtained in vivo by mechanisms that are independent of Fc interactions (Bacskai et al. (2002) J. Neurosci. 22:7873-7878). Studies have indicated that an antibody directed against the midportion of Aβ, which cannot recognize amyloid plaques, appears to bind to soluble Aβ and reduce plaque deposition (DeMattos et al. (2001) Proc. Natl. Acad. Sci. USA 98:8850-8855).

The ability of various antibodies to capture soluble Aβ was further assayed as follows. Various concentrations of antibody (up to 10 μg/ml) were incubated with 50,000 CPM of ¹²⁵I-Aβ 1-42 (or ¹²⁵I-Aβ 1-40). The concentration of antibody sufficient to bind 25% of the radioactive counts was determined in a capture radioimmunoassay. Certain antibodies did not bind 25% of the counts at the highest concentration tested (i.e., 10 μg/ml). For such antibodies, the percentage of counts bound at 10 μg/ml was determined. The 12A11 bound 20% of the radioactive counts (i.e., ¹²⁵I-Aβ) at 10 μg/ml. This was greater than the amount bound by two other Aβ 3-7 antibodies tested, namely 12B4 and 10D5 (binding 7% and 2% at 10 μg/ml, respectively). Thus, of the N-terminal (epitope Aβ 3-7) antibodies tested, 12A11 exhibited the most appreciable ability to capture Aβ. At 3 μg/ml, 15C11 bound 25% of the radioactive counts (i.e., ¹²⁵I-Aβ). This capture was significant as compared to other monoclonal antibodies raised against central Aβ fragments (e.g., Aβ 13-28 or Aβ 17-28). The range of concentrations necessary to capture 25% of the labeled Aβ for such antibodies is from about 0.1 μg/ml to 10 μg/ml with some antibodies capturing less than 25% labeled Aβ (e.g., 10-20%) when assayed at 10 μg/ml.

### Example XII

### In Vitro Efficacy of Various Aβ Antibodies: Binding Soluble, Oligomeric Aβ

In this Example, the Aβ preparation was derived from synthetic Aβ oligomers substantially as follows:
(1) lyophilized Aβ₁₋₄₂ peptide was dissolved to 1 mM in 100% hexafluoroisopropanol (HFIP) (mixed then incubated at room temperature for 1 hour) and separated into aliquots in microcentrifuge tubes (each tube containing 0.5 mg of Aβ₁₋₄₂ peptide);
(2) the HFIP was removed by evaporation followed by lyophilization to remove residual HFIP;
(3) the resultant Aβ peptide film/residue was stored, desiccated, at −20° C.;
(4) the Aβ peptide residue was resuspended in DMSO to a final concentration of 5 mM of peptide then added to ice cold Ham's F-12 (phenol red free) culture media to bring the peptide to a final concentration of 100 μM;
(5) the peptide was incubated at 4° C. for 24 h to produce synthetic Aβ oligomers at an approximately 100 μM concentration; and
(6) the synthetic Aβ oligomers were treated with peroxynitrite.

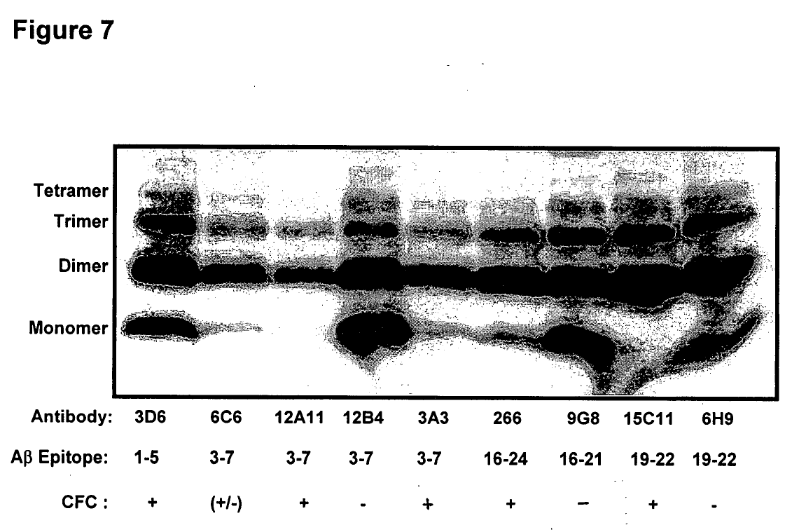

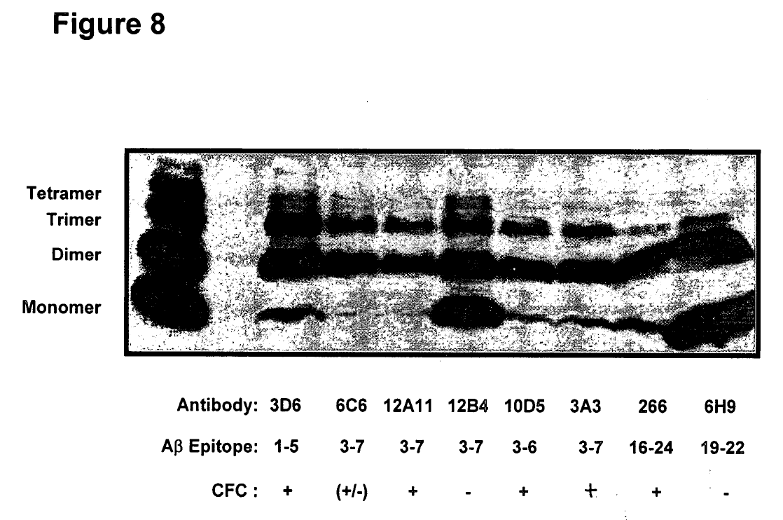

Portions of the Aβ preparation were then each contacted with a test immunological reagent, in this case antibodies, and the Aβ monomers and one or more Aβ oligomers which bound to the test immunological reagent were extracted from the Aβ preparation by immunoprecipitation. The various immunoprecipitates were separated by gel electrophoresis and immunoblotted with the 3D6 antibody substantially as follows. Immunoprecipitate samples of FIGS. 7-8 were diluted in sample buffer and separated by SDS-PAGE on a 16% Tricine gel. The protein was transferred to nitrocellulose membranes, the membranes boiled in PBS, and then blocked overnight at 4° C. in a solution of TBS/Tween/5% Carnation dry milk. The membranes were then incubated with 3D6, a mouse monoclonal Aβ antibody to residues 1-5. For detection, the membranes were incubated with anti-mouse Ig-HRP, developed using ECL Plus, and visualized using film. Molecular mass was estimated by SeeBlue™ Plus2 molecular weight markers.

FIGS. 7-8 depict the results of contacting samples of the above Aβ₁₋₄₂ preparation with various Aβ antibodies to determine the binding to Aβ monomers, dimers, trimers, tetramers, pentamers, etc. in the Aβ preparation. FIGS. 7-8 depict Western blots (imaged with 3D6) of immunoprecipitates of a peroxynitrite treated oligomeric Aβ₁₋₄₂ preparation contacted with various Aβ antibodies. The approximate positions of Aβ₁₋₄₂ monomer, dimer, trimer and tetramer bands are indicated on the left-hand side of each figure. Indicated below each Aβ antibody is the Aβ epitope recognized by the antibody and CFC assay results for the antibody (see Example XIII). A “+” notation indicates an observation of increased cognition upon treatment with the antibody, a “−” notation indicates an observation of no change in cognition upon treatment with the antibody, and a “+/−” notation indicates an observation of a trend of increased cognition upon treatment with the antibody but which is not statistically significant enough to be indicated as an observation of increased cognition.

In FIGS. 7-8, an increased binding of an Aβ antibody for Aβ dimers or higher ordered oligomers in the Aβ preparation, relative to the binding of the Aβ antibody for Aβ monomers in the Aβ preparation, predicts that the Aβ antibody has therapeutic efficacy for the treatment of Alzheimer's disease. Notably, Aβ antibodies 3D6, 3A3, 15C11, 10D5, 12A11 and 266 exhibited preferential binding for oligomeric Aβ species as compared to monomeric Aβ with 12A11 exhibiting the most significant preferential binding to oligomeric Aβ. Accordingly, these antibodies are predicted to have therapeutic efficacy in the treatment cognitive deficits, e.g., those associated with AD.

### Example XIII

### In Vivo Efficacy of Various Aβ Antibodies: Rapid Improvement in Cognition in a Tg2576 Mouse Model of AD

Wild-type and Tg2576 mice were administered a single dose of phosphate buffered saline (PBS) or treatment antibody by intraperitoneal injection. Treatment antibodies included antibodies raised to N-terminal, central, and C-terminal portions of Aβ peptide. To assess rapid improvement in cognition (i.e. contextual and cue-dependent memory), each mouse was administered a CFC training session immediately following treatment and a CFC testing session within 24 hours of treatment (i.e. Day 1 post-treatment). Therapeutic efficacy was expressed both in terms of memory deficit reversal and memory impairment status. “Memory deficit reversal” was determined by comparing the freezing behavior of mAb- vs. PBS control-treated Tg2576 animals. “Memory impairment status” was determined by comparing the freezing behavior of Wild-type vs. Tg2576 mAb-treated animals.

The therapeutic efficacy of several mAbs raised against the N-terminus of Aβ are tabulated in Table 20. The results of the CFC testing session conducted on Day 1 post-treatment indicate that the mAbs 3D6, 10D5, and 12A11 caused a rapid and significant (**) improvement in contextual memory of Tg2576 mice relative to a control treatment (p value<0.05). Moreover, Tg2576 mice treated with the mAbs 3D6, 10D5, and 12A11 exhibited no significant memory impairment (##) with respect to wild-type mice (p value>0.1). An additional N-terminal mAb designated 3A3 (not listed in Table 20) was also found to be efficacious at reversing cognitive memory deficits in Tg2576 mice in the CFC assay. Additionally, the antibodies 6C6, 10D5, and 12B4 displayed a trend towards either memory deficit reversal (*) or no memory impairment (#) (0.1>p value>0.05).

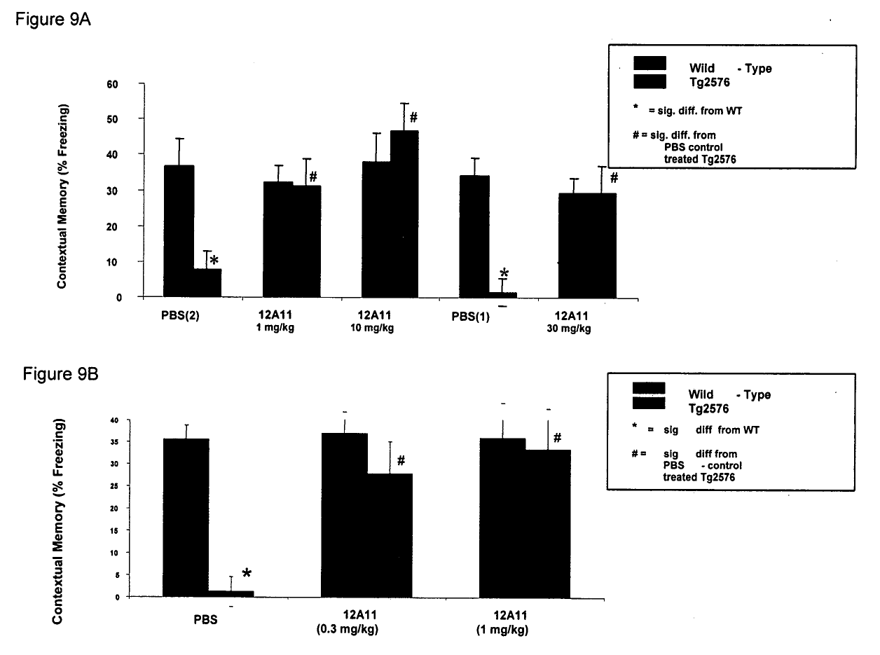

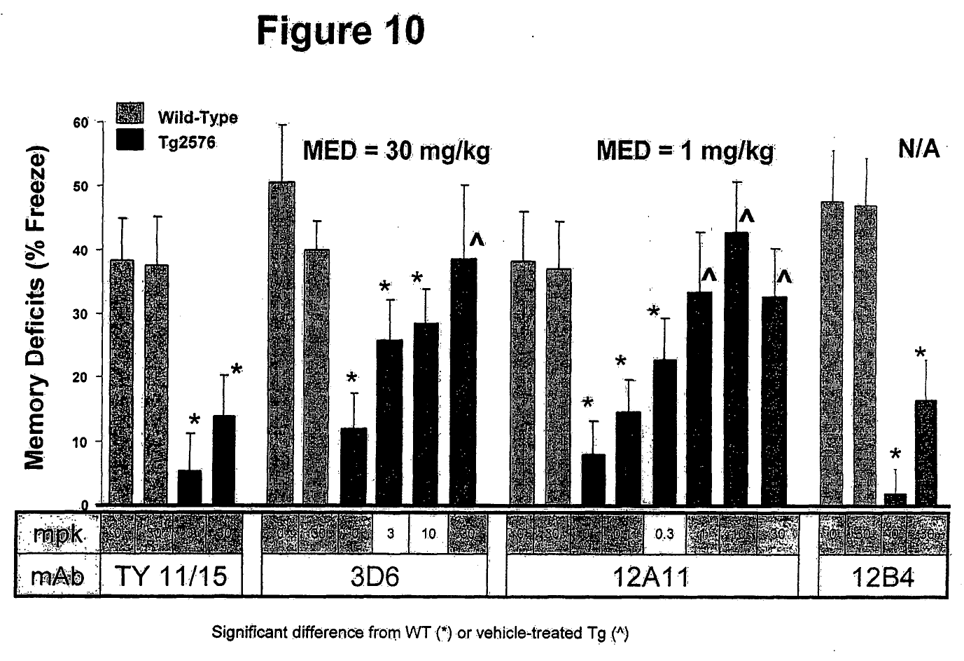

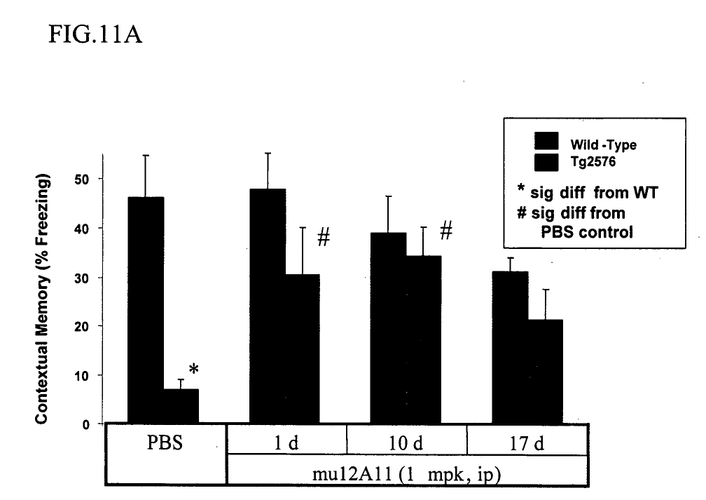

Tg2576 mice displayed a particularly prominent, significant, and rapid improvement in contextual memory when administered the N-terminal, murine IgG2a mAb designated 12A11. For example, the 12A11 resulted in a memory deficit reversal at every dose tested (0.3, 1, 10, or 30 mg/kg) (see FIGS. 9A and 9B). In contrast, untreated (PBS) Tg2576 mice displayed a significant deficit in contextual-dependent memory (*) in comparison with wild-type mice (FIG. 9A). However, Tg2576 mice exhibited a full and significant memory deficit reversal (#) when administered 1, 10, or 30 mg/kg (i.p.) of 12A11. The improvement in cognitive performance persisted when mice were administered lower doses (0.1 and 1 mg/kg i.p.) of 12A11 (FIG. 9B).

To confirm that the observed response was due to amyloid binding, Tg2576 mice were administered 30 mg/kg of IgG2a isotype control mAb raised against an unrelated antigen from E. tennela. As expected, Tg2576 mice treated with the control antibody exhibited profound defects in contextual memory in relation to wild-type mice.

In another experiment, the effect of the N-terminal antibodies 3D6, 12A11, and 12B4 were compared directly in a CFC assay with Tg2576 mice (see FIG. 2). Consistent with previous results, 12A11 induced a prominent and significant memory deficit reversal at 1, 10, or 30 mg/kg (ˆ). Morever, 3D6 induced significant memory deficit reversal at 30 mg/kg. In contrast, both 12B4 antibodies and an unrelated IgG1 antibody (TY 11/15) failed to induce significant memory deficit reversal.

TABLE 20
Effect of N-terminal Aβ mAbs on Contextual Memory of Tg2576 mice

| | | | | | | |
|---|---|---|---|---|---|---|
| mAb tested | Epitope | 0.3 | 1 | 3 | 10 | 30 |

Memory Deficit Reversal per Ab dosage (mg/kg)
(p value WRT PBS Control)

| | | | | | | |
|---|---|---|---|---|---|---|
| 3D6 | 1-5 | ND | ND | 0.3680 | 0.1586 | 0.0004** |
| 6C6-1 | 3-7 | ND | ND | ND | ND | 0.0588* |
| 6C6-2 | 3-7 | ND | ND | ND | ND | 0.6567 |
| 10D5 | 3-6 | ND | ND | 0.7045 | 0.9661 | 0.0189** |
| 2H3 | 2-7 | ND | ND | ND | ND | 0.3007 |
| 12B4-1 | 3-7 | ND | ND | ND | ND | 0.1122 |
| 12B4-2 | 3-7 | ND | ND | ND | ND | 0.1015 |
| 12A11-1 | 3-7 | ND | 0.02** | ND | 0.0002** | 0.0007** |
| 12A11-2 | 3-7 | 0.0055** | 0.001** | ND | ND | ND |

Impairment Status per Ab dosage (mg/kg)
(p value WRT WT mice)

| | | | | | | |
|---|---|---|---|---|---|---|
| 3D6 | 1-5 | ND | ND | 0.0529# | 0.2585## | 0.8972## |
| 6C6-1 | 3-7 | ND | ND | ND | ND | 0.0056 |
| 6C6-2 | 3-7 | ND | ND | ND | ND | 0.0088 |
| 10D5 | 3-6 | ND | ND | 0.0009 | 0.002 | 0.0752# |
| 2H3 | 2-7 | ND | ND | ND | ND | 0.1333## |
| 12B4-1 | 3-7 | ND | ND | ND | ND | 0.0013 |
| 12B4-2 | 3-7 | ND | ND | ND | ND | 0.756# |
| 12A11-1 | 3-7 | ND | 0.9092## | ND | 0.3838## | 0.9901## |
| 12A11-2 | 3-7 | 0.3341## | 0.7773## | ND | ND | ND |

*Dash 1 (−1) and dash 2 (−2) indicate two different animals tested.

The therapeutic efficacy of several mAbs raised against the central amino acid sequence of Aβ are tabulated in Table 21. The results of the CFC testing session conducted on Day 1 post-treatment indicate that the mAbs 266 and 15C11 caused a significant improvement in contextual memory (**) of Tg2576 mice relative to control treatment (p value<0.05), and no significant memory impairment (##) with respect to wild-type mice (p value>0.1). Furthermore, the antibodies 1C2 and 2B1 displayed a trend towards memory deficit reversal (*). (0.1>p value>0.05).

TABLE 21
Effect of Central anti-Aβ mAbs on Contextual Memory in Tg2576 mice

| | | | | | |
|---|---|---|---|---|---|
| mAb | Epitope | 1 (mg/kg) | 3 (mg/kg) | 10 (mg/kg) | 30 (mg/kg) |

Memory Deficit Reversal
(p value WRT PBS control)

| | | | | | |
|---|---|---|---|---|---|
| 266 | 16-24 | 0.1269 | 0.0082** | 0.0002** | ND |
| 6H9 | 19-22 | ND | ND | ND | 0.1122 |
| 1C2 | 16-23 | ND | ND | ND | 0.0695* |
| 15C11 | 19-22 | ND | 0.1246 | 0.1156 | 0.0274** |
| 2B1 | 19-23 | ND | ND | ND | 0.0578* |

Impairment Status
(p value WRT WT mice)

| | | | | | |
|---|---|---|---|---|---|
| 266 | 16-24 | 0.1635## | 0.1084## | 0.8348## | ND |
| 6H9 | 19-22 | ND | ND | ND | 0.0044 |
| 1C2 | 16-23 | ND | ND | ND | 0.0626# |
| 15C11 | 19-22 | ND | 0.6228## | 0.3399## | 0.8907## |
| 2B1 | 19-23 | ND | ND | ND | 0.4020## |

The therapeutic efficacy of several mAbs raised against the carboxy terminal amino acid sequence of Aβ are tabulated in Table 22. The results of the CFC testing session conducted on Day 1 post-treatment indicate that most antibodies raised against the C-terminus of Aβ were relatively ineffective in treating cognitive impairment at the single dose tested (30 mg/kg). Of the four monoclonals tested, none produced any improvement in the contextual memory of Tg2576 mice relative to control treatment (p value>0.1), although three (2G3, 14C2, and 16C11) displayed a trend toward no impairment (#) with respect to wild-type mice (0.1>p value>0.05).

TABLE 22
Effect of C-terminal anti-Aβ mAbs on Contextual Memory in
Tg2576 mice

| mAb | Epitope | 1 (mg/kg) | 3 (mg/kg) | 10 (mg/kg) | 30 (mg/kg) |
|---|---|---|---|---|---|
| | | Memory Deficit Reversal | | | |
| | | (p value WRT PBS control) | | | |
| 2G3 | 33-40 | ND | ND | ND | 0.7521 |
| 14C2 | 33-40 | ND | ND | ND | 0.79654 |
| 21F12 | 33-42 | ND | ND | ND | 0.2026 |
| 16C11 | 33-42 | ND | ND | ND | 0.1523 |
| | | Impairment Status | | | |
| | | (p value WRT WT mice) | | | |
| 2G3 | 33-40 | ND | ND | ND | 0.0589# |
| 14C2 | 33-40 | ND | ND | ND | 0.0719# |
| 21F12 | 33-42 | ND | ND | ND | 0.0151 |
| 16C11 | 33-42 | ND | ND | ND | 0.0985# |

In the above studies, mice displaying memory deficit reversal did so within a short time period. This rapid improvement in cognition in mice administered various efficacious Aβ antibodies suggests a mechanism of action that involves the capture of soluble Aβ in the blood and the subsequent removal of Aβ from the CNS into the plasma.

### Example XIV

### In Vivo Efficacy of a Mouse 12A11 Antibody: Prolonged Improvement in Cognition of a Tg2576 Mouse

The duration of the cognitive improvements that were observed within 24 hours following treatment with the N-terminal, murine 12A11 antibody (“mu12A11”) was assessed in a second extended CFC study. Tg2575 and wild-type mice were again administered a PBS control or a low dose of 12A11 antibody (1 mg/kg ip) and their cognitive status was assessed by CFC assay on Day 0-1, 9-10, and 16-17 post-treatment (i.e. with the CFC training sessions performed on Days 0, 9, 16 and CFC testing sessions performed on Days 1, 10, and 17).

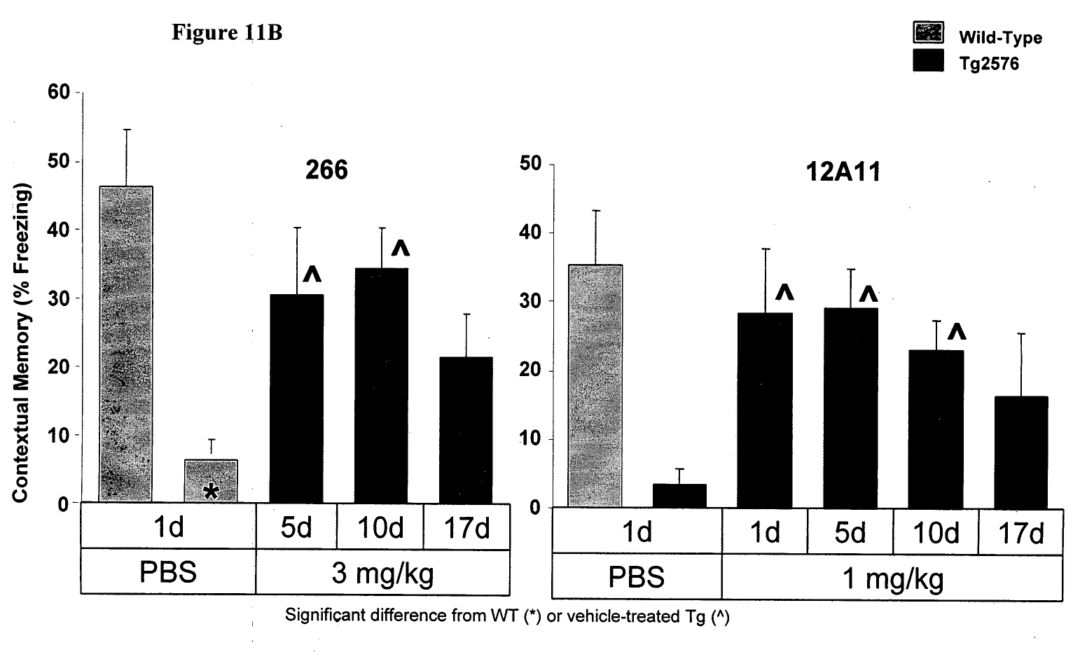

As observed in Example XIII, Tg2576 mice again displayed prominent, significant, and rapid improvement in contextual memory on Day 1 following treatment with mu12A11 (see FIG. 11A). For example, Tg2576 treated with mu12A11 exhibited a significant memory deficit reversal (when compared with PBS-treated Tg2576 mice) and memory impairment status that approached parity with that of wild-type mice. These improvements in contextual memory persisted and were even more pronounced when assessed at Day 10 post-treatment. Furthermore, when assessed at Day 17 post-treatment, mu12A11 continued to display a trend towards no memory impairment. These results indicated that a single low dose of the mu12A11 N-terminal antibody can result in a durable and prolonged improvement in cognitive performance in the mouse model of AD.

### Example XV

### In Vivo Efficacy Comparison of Mouse 266 Antibody and Mouse 12A11 Antibody: Prolonged Improvement in Cognition of a Tg2576 Mouse

The duration of the cognitive improvements that were observed within 24 hours following treatment with the N-terminal, murine 12A11 antibody (“mu12A11”) was assessed in a comparison extended CFC study with mid-region, murine 266 antibody (“mu266”). Tg2575 and wild-type mice were again administered a PBS control or a low dose of 12A11 antibody (1 mg/kg ip) and their cognitive status was assessed by CFC assay on Day 0-1, 4-5, 9-10, and 16-17 post-treatment (i.e. with the CFC training sessions performed on Days 0, 4, 9, 16 and CFC testing sessions performed on Days 1, 5, 10, and 17).

A second group of Tg2575 and wild-type mice were administered a PBS control or a low dose of 266 antibody (3 mg/kg ip) and their cognitive status was assessed by CFC assay on Day 0-1, 4-5, 9-10, and 16-17 post-treatment (i.e. with the CFC training sessions performed on Days 0, 4, 9, 16 and CFC testing sessions performed on Days 1, 5, 10, and 17).

As described in Example IV, Tg2576 mice treated with both mu12A11 and mu266 displayed prominent, significant, and rapid improvement in contextual memory on Day 5 following treatment with the respected antibodies [see FIG. 11B]. For example, both groups of Tg2576 treated with mu12A11 and mu266 exhibited a significant memory deficit reversal (when compared with PBS-treated Tg2576 mice) and memory impairment status that approached parity with that of wild-type mice. Both groups exhibited improvements in contextual memory which persisted and were even more pronounced when assessed at Day 10 post-treatment. Furthermore, when assessed at Day 17 post-treatment, both the mu12A11 and mu266 groups continued to display a trend towards no memory impairment. These results indicated that a single low dose of both the mu12A11 N-terminal antibody and the mu266 mid-terminal antibody separately can result in a durable and prolonged improvement in cognitive performance of mouse model of AD.

In a final experiment, wild-type mice and doubly transgenic AD mice were administered a single dose of phosphate buffered saline (PBS) or treatment antibody (C-terminal 266 antibody or N-terminal 12A11 antibody) by intraperitoneal injection at 24 hours prior to the training phase of the CFC. The doubly transgenic AD mice used in the experiment were approximately 18-20 months of age and displayed prominent cognitive defects, as well a dense accumulation of plaque.

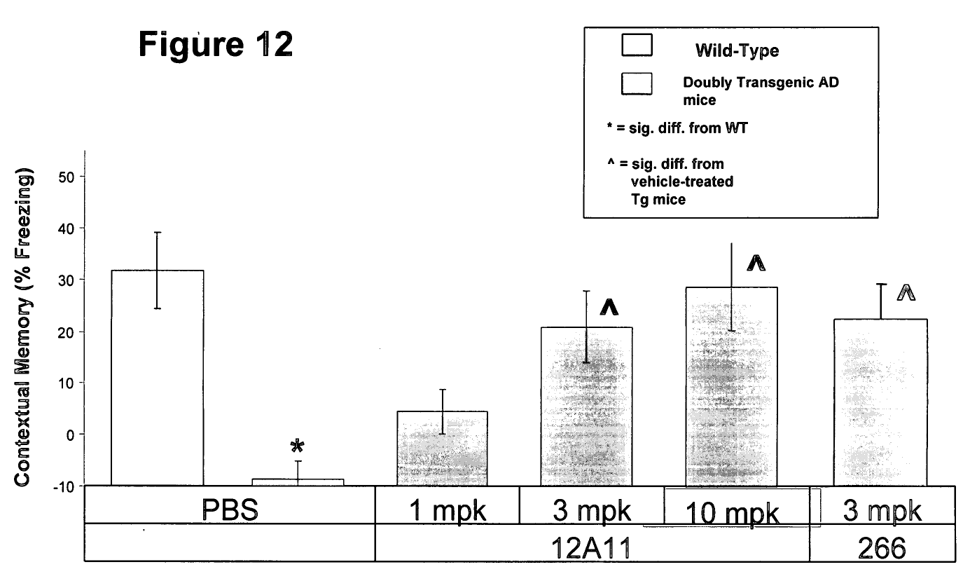

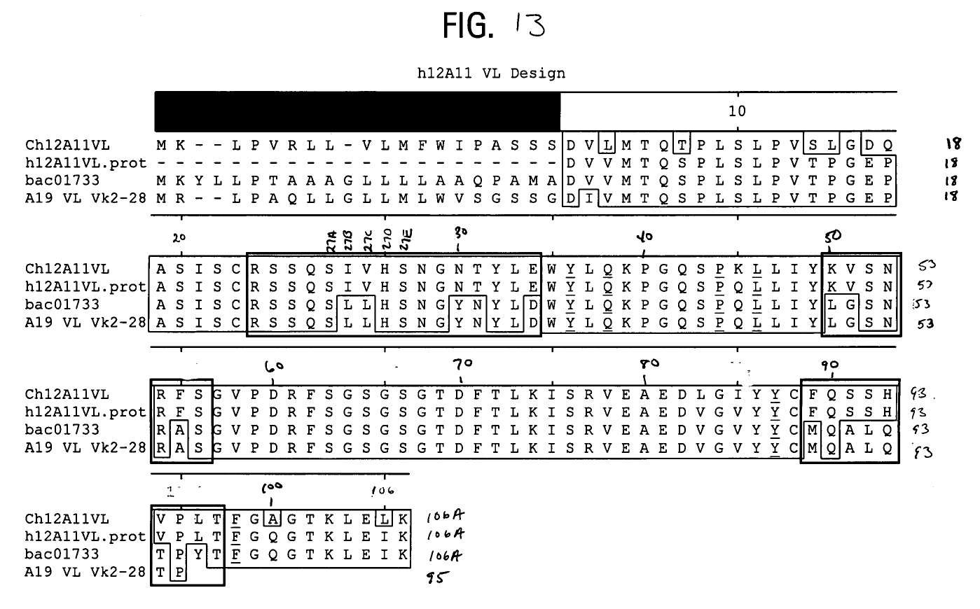

The doubly transgenic AD mice displayed prominent and significant reversal of contextual memory deficit when administered the N-terminal, murine IgG2a mAb designated 12A11, and this mAb was effective treatment at several low doses (3 and 10 mg/kg). (see FIG. 12). Low-dose treatment with the central terminal antibody designated 266 also resulted in a significant reversal of the contextual-memory deficit in the doubly transgenic mice. In contrast, untreated (PBS) doubly transgenic mice displayed a significant deficit in contextual-dependent memory (ie. a significant memory impairment status) in comparison with wild-type mice (*). These results demonstrate that acute reversal of contextual memory deficits is maintained in aged mice exhibiting prominent Alzheimer's disease pathology (AD).

### Example XVI

### Mouse 12A11 Variable Region Sequences

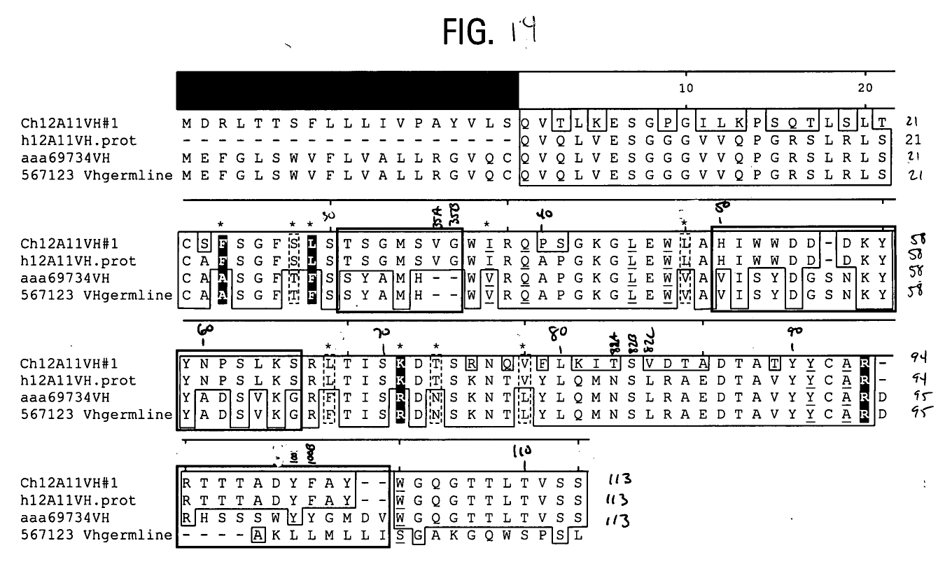

The VL and VH regions of mu 12A11 from hybridoma cells were cloned by RT-PCR and 5′ RACE using mRNA from hybridoma cells and standard cloning methodology. The nucleotide sequences encoding the VL and VH regions of 12A11 are set forth as SEQ ID NOs: 27 and 29, respectively (and in Tables 23 and 25, respectively). The amino acid sequences of the VL and VH regions of 12A11 are set forth as SEQ ID NOs: 28 and 30, respectively (and in Tables 24 and 26, respectively, and in FIGS. 13 and 14, respectively).

TABLE 23
Mouse 12A11 VL DNA sequence

| | |
|---|---|
| ATGAAGTTGCCTGTTAGGCTGTTGGTGCTGATGTTC | (SEQ ID NO:27) |
| TGGATTCCTGCTTCCAGCAGTGATGTTTTGATGACC | |
| CAAACTCCACTCTCCCTGCCTGTCAGTCTTGGAGAT | |
| CAAGCCTCCATCTCTTGCAGATCTAGTCAGAGCATT | |
| GTACATAGTAATGGAAACACCTACTTAGAATGGTAC | |
| CTGCAGAAACCAGGCCAGTCTCCAAAGCTCCTGATC | |
| TACAAAGTTTCCAACCGATTTTCTGGGGTCCCAGAC | |
| AGGTTCAGTGGCAGTGGATCAGGGACAGATTTCACA | |
| CTCAAGATCAGCAGAGTGGAGGCTGAGGATCTGGGA | |
| ATTTATTACTGCTTTCAAAGTTCACATGTTCCTCTC | |
| ACGTTCGGTGCTGGGACCAAGCTGGAGCTGAAA | |

*Leader peptide is underlined.

TABLE 24
Mouse 12A11 VL amino acid sequence

| | |
|---|---|
| mklpvrllvlmfwipasssDVLMTQTPLSLPVSLGD | (SEQ ID NO:28) |
| QASISCrssqsivhsngntyleWYLQKPGQSPKLLI | |
| YkvsnrfsGVPDRFSGSGSGTDFTLKISRVEAEDLG | |
| IYYCfqsshvpltFGAGTKLELK | |

*Leader peptide and CDRs in lower case.

TABLE 25
Mouse 12A11 VH DNA sequence.

| | |
|---|---|
| ATGGACAGGCTTACTACTTCATTCCTGCTGCTGATT | (SEQ ID NO:29) |
| GTCCCTGCATATGTCTTGTCCCAAGTTACTCTAAAA | |
| GAGTCTGGCCCTGGGATATTGAAGCCCTCACAGACC | |
| CTCAGTCTGACTTGTTCTTTCTCTGGGTTTTCACTG | |
| AGCACTTCTGGTATGAGTGTAGGCTGGATTCGTCAG | |
| CCTTCAGGGAAGGGTCTGGAGTGGCTGGCACACATT | |
| TGGTGGGATGATGATAAGTACTATAACCCATCCCTG | |
| AAGAGCCGGCTCACAATCTCCAAGGATACCTCCAGA | |
| AACCAGGTATTCCTCAAGATCACCAGTGTGGACACT | |
| GCAGATACTGCCACTTACTACTGTGCTCGAAGAACT | |
| ACTACGGCTGACTACTTTGCCTACTGGGGCCAAGGC | |
| ACCACTCTCACAGTCTCCTCA | |

*Leader peptide underlined.

TABLE 26
Mouse 12A11 VH amino acid sequence

| | |
|---|---|
| mdrlttsflllivpayvlsQVTLKESGPGILKPSQT | (SEQ ID NO:30) |
| LSLTCSFSGFSLStsgmsvgWIRQPSGKGLEWLAhi | |
| wwdddkyynpslksRLTISKDTSRNQVFLKITSVDT | |
| ADTATYYCARrtttadyfayWGQGTTLTVSS | |

*Leader peptide and CDRs in lower case.

The 12A11 VL and VH sequences meet the criteria for functional V regions in so far as they contain a contiguous ORF from the initiator methionine to the C-region, and share conserved residues characteristic of immunoglobulin V region genes. From N-terminal to C-terminal, both light and heavy chains comprise the domains FR1, CDR1, FR2, CDR2, FR3, CDR3 and FR4.

Variable heavy and light chain expression vectors were engineered as described supra for 3D6 and 12B4 and were co-transfected into COS cells. Conditioned media was assayed by western blot analysis for antibody production or ELISA for Aβbinding. Chimeric 12A11 was found to bind to Aβ with high avidity, similar to that demonstrated by chimeric and humanized 3D6. Binding avidity was also similar to that demonstrated by chimeric and humanized 12B4.

### Example XVII

### Mouse 15C11 Variable Region Sequences

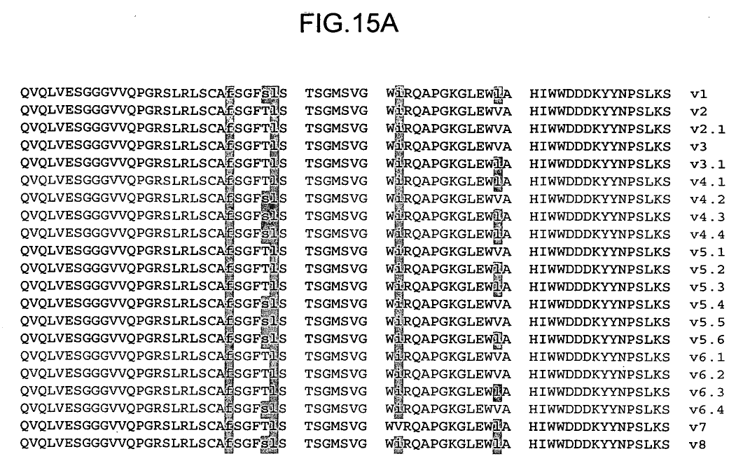

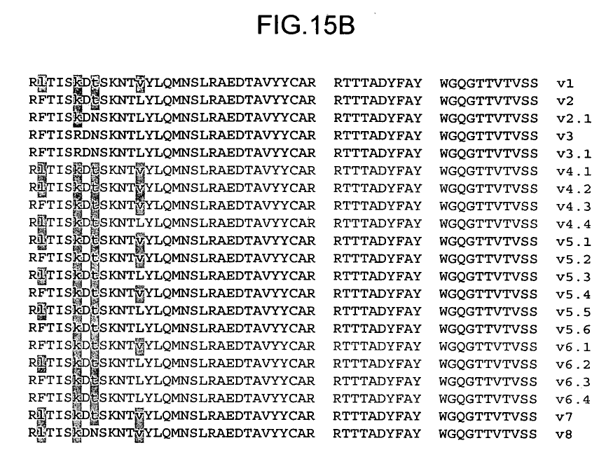

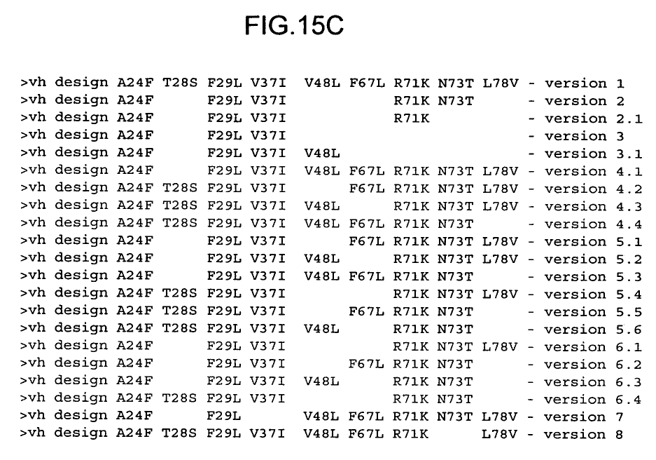

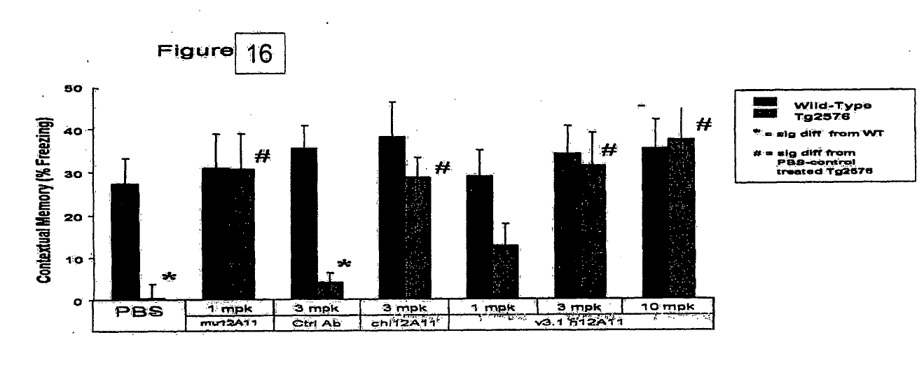

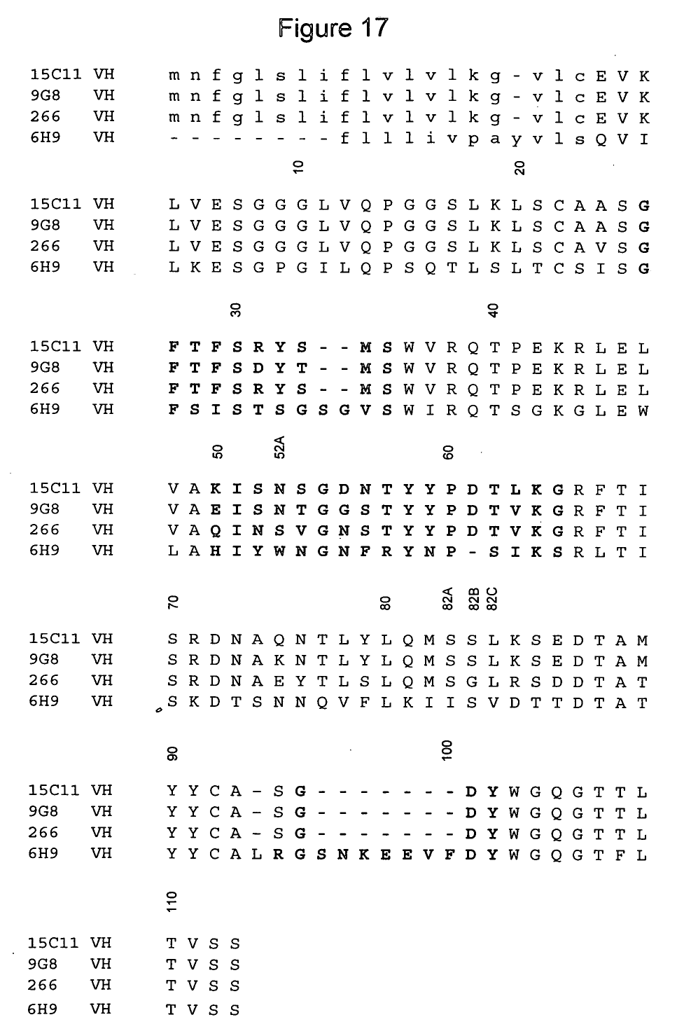

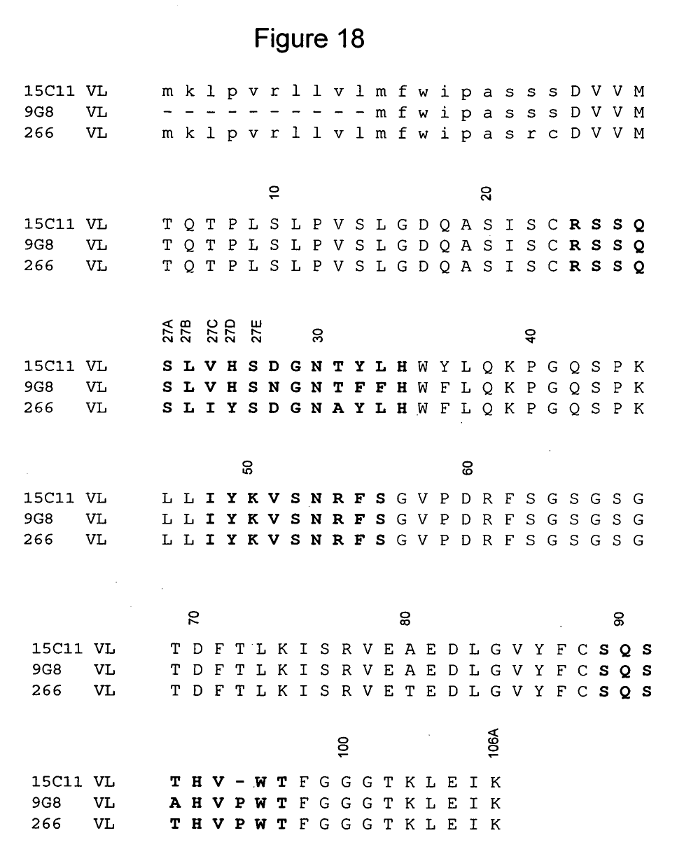

The VL and VH regions of 15C11 from hybridoma cells were cloned by RT-PCR and 5′ RACE using mRNA from hybridoma cells and standard cloning methodology. The nucleotide sequences encoding the VL and VH regions of 15C11 are set forth as SEQ ID NOs: 39 and 41, respectively (and in Tables 27 and 29, respectively). The amino acid sequences of the VL and VH regions of 15C11 are set forth as SEQ ID NOs: 40 and 42, respectively (and in Tables 28 and 30, respectively, and in FIGS. 15 and 16, respectively). From N-terminal to C-terminal, both light and heavy chains comprise the domains FR1, CDR1, FR2, CDR2, FR3, CDR3 and FR4. From N-terminal to C-terminal, both light and heavy chains comprise the domains FR1, CDR1, FR2, CDR2, FR3, CDR3 and FR4. An epitope map assay was performed which identified residues 19-22 of Aβ as the epitope for 15C11.

TABLE 27
Mouse 15C11 VL DNA sequence

| | |
|---|---|
| ATGAAGTTGCCTGTTAGGCTGTTGGTGCTGATGTTC | (SEQ ID NO:39) |
| TGGATTCCTGCTTCCAGCAGTGATGTTGTGATGACC | |
| CAAACTCCACTCTCCCTGCCTGTCAGTCTTGGAGAT | |
| CAAGCCTCCATCTCTTGCAGATCTAGTCAGAGCCTT | |
| GTACACAGTGATGGAAACACCTATTTACATTGGTAC | |
| CTGCAGAAGCCAGGCCAGTCTCCAAAACTCCTGATC | |
| TACAAAGTTTCCAACCGATTTTCTGGGGTCCCAGAC | |
| AGGTTCAGTGGCAGTGGATCAGGGACAGATTTCACA | |
| CTCAAGATCAGCAGAGTGGAGGCTGAGGATCTGGGA | |
| GTTTATTTCTGCTCTCAAAGTACACATGTGTGGACG | |
| TTCGGTGGAGGCACCAAGCTGGAAATCAAA | |

*Leader peptide is underlined.

TABLE 28
Mouse 15C11 VL amino acid sequence

| | |
|---|---|
| mklpvrllvlmfwipasssDVVMTQTPLSLPVSLGD | (SEQ ID NO:40) |
| QASISCrssqslvhsdgntylhWYLQKPGQSPKLLi | |
| ykvsnrfsGVPDRFSGSGSGTDFTLKISRVEAEDLG | |
| VYFCsqsthvwtFGGGTKLEIK | |

*Leader peptide and CDRs in lower case.

TABLE 29
Mouse 15C11 VH DNA sequence.

| | |
|---|---|
| ATGAATTTCGGGCTCAGCTTGATTTTCCTTGTCCTT | (SEQ ID NO:41) |
| GTTTTAAAAGGTGTCCTGTGTGAAGTGAAGCTGGTG | |
| GAGTCTGGGGGAGGTTTAGTGCAGCCTGGAGGGTCC | |
| CTGAAACTCTCCTGTGCAGCCTCTGGATTTACTTTC | |
| AGTAGATATAGTATGTCTTGGGTTCGCCAGACTCCA | |
| GAGAAGAGGCTGGAGTTGGTCGCAAAAATTAGTAAT | |
| AGTGGTGATAACACCTACTATCCAGACACTTTAAAG | |
| GGCCGATTCACCATCTCCAGAGACAATGCCCAGAAC | |
| ACCCTGTACCTGCAAATGAGCAGTCTGAAGTCTGAG | |
| GACACGGCCATGTATTACTGTGCAAGCGGGGACTAC | |
| TGGGGCCAAGGCACCACTCTCACAGTCTCCTCA | |

*Leader peptide is underlined.

TABLE 30
Mouse 15C11 VH amino acid sequence

| | |
|---|---|
| mnfglsliflvlvlkgvlcEVKLVESGGGLVQPGGS | (SEQ ID NO:42) |
| LKLSCAASgftfsrysmsWVRQTPEKRLELVAkisn | |
| sgdntyypdtlkgRFTISRDNAQNTLYLQMSSLKSE | |
| DTAMYYCASgdyWGQGTTLTVSS | |

*Leader peptide and CDRs in lower case.

### Example XVIII

### 12A11 Humanization

In order to identify key structural framework residues in the murine 12A11 antibody, three-dimensional models were studied for solved murine antibodies having homology to the 12A11 heavy and light chains. An antibody designated 1KTR was chosen having close homology to the 12A11 light chain and two antibodies designated 1ETZ and 1JRH were chosen having close homology to the 12A11 heavy chain. These mouse antibodies show strong sequence conservation with 12A11 (94% identity in 112 amino acids for Vk and 83% identity in 126 amino acids and 86% identity in 121 amino acids respectively for Vh). The heavy chain structure of 1ETZ was superimposed onto that of 1KTR. In addition, for Vk the CDR loops of the selected antibody fall into the same canonical Chothia structural classes as do the CDR loops of 12A11 VL. The crystal structures of these antibodies were examined for residues (e.g., FR residues important for CDR conformation, etc.) predicted be important for function of the antibody, and by comparison, function of the similar 12A11 antibody

Suitable human acceptor antibody sequences were identified as described supra. The selected acceptor sequence for VL is BAC01733 in the NCBI Ig non-redundant database. The selected acceptor sequence for VH is AAA69734 in the NCBI Ig non-redundant database. AAA69734 is a human subgroup III antibody (rather than subgroup II) but was selected as an initial acceptor antibody based at least in part on the reasoning in Saldanha et al. (1999) Mol. Immunol. 36:709. First versions of humanized 12A11 antibody utilize these selected acceptor antibody sequences. The antibody is described in Schroeder and Wang (1990) Proc. Natl. Acad. Sci. USA 872:6146.

As noted supra, the humanized antibodies of the invention comprise variable framework regions substantially from a human immunoglobulin (acceptor immunoglobulin) and complementarity determining regions substantially from a mouse immunoglobulin (donor immunoglobulin) termed 12A11. Having identified the complementarity determining regions of 12A11 and appropriate human acceptor immunoglobulins, the next step was to determine which, if any, residues from these components to substitute to optimize the properties of the resulting humanized antibody.

The amino acid alignment of the reshaped light chain V region is shown in FIG. 13. The choice of the acceptor framework (BAC01733) is from the same human subgroup as that which corresponds to the murine V region, has no rare framework residues, and the CDRs belong to the same Chothia canonical structure groups. No backmutations were made in Version 1 of humanized 12A11

The amino acid alignment of the reshaped heavy chain V region is shown in FIG. 14. The choice for the acceptor framework (AAA69734) is from human subgroup III (as described previously) and has no rare framework residues. Structural analysis of the murine VH chain (1ETZ and 1JRH), in conjunction with the amino acid alignment of AAA69734 to the murine sequence dictates 9 backmutations in version 1 (v1) of the reshaped heavy chain: A24F, T28S, F29L, V37I, V48L, F67L, R71K, N73T, L78V (Kabat numbering). The back mutations are highlighted by asterisks in the amino-acid alignment shown in FIG. 14.

Of the 9 back mutations, 3 are dictated by the model because the residues are canonical residues (A24F, F29L, & R71K, solid fill), i.e. framework residues which may contribute to antigen binding by virtue of proximity to CDR residues. There is one back mutation in the next most important class of residues, the interface residues involved in VH-VL packing interactions (underlined), i.e., V37I. The N73T mutation is at a vernier residue (dotted fill) on the edge of the binding site, possibly interacting with S30 adjacent to CDR1. The remaining 4 residues targeted for back mutation (T28S, V48L, F67L, L78V, Kabat numbering) also fall into the vernier class (indirect contribution to CDR conformation, dotted fill in FIG. 14).

A summary of the changes incorporated into version 1 of humanized 12A11 is presented in Table 31.

TABLE 31
Summary of changes in humanized 12A11.v1

| Changes | VL (112 residues) | | VH (120 residues) | |
|---|---|---|---|---|
| Hu->Mu: Framework | 0/112 | | 9/120 | |
| CDR1 | 5/16 | | 6/7 | |
| CDR2 | 3/7 | | 10/16 | |
| CDR3 | 6/8 | | 8/11 | |
| Total Hu->Mu | 14/112 (12.5%) | | 33/120 (27.5%) | |
| Mu->Hu: Framework | 11/112 | | 26/120 | |
| Backmutation notes | none | | 27. | Canonical: A24F, F29L, |
| | | | | R71K |
| | | | 28. | Packing: V37I |
| | | | 29. | Vernier: T28S, V48L, |
| | | | | F67L, N73T, L78V |
| Acceptor notes | 30. | Genbank Acc. no. | 33. | Genbank Acc. no. |
| | | BAC01733 | | AAA69734 (H1 - class 1, |
| | 31. | CDRs from same canonical | | H2 = class 3) |
| | | structural group as donor | 34. | CDRs from same canonical |
| | | mouse; | | structural group as donor |
| | 32. | Immunoglobulin kappa | | mouse |
| | | light chain K64(AIMS4) | 35. | fetal Ig |

Kabat numbering for the 12A11 light and heavy chains is set forth in FIGS. 13 and 14, respectively. Kabat numbering for the heavy chain acceptor sequence AAA69734 and the germline sequence 567123 can be determined using art-recognized methods (see e.g., Kabat, Sequences of Proteins of Immunological Interest, supra.) The nucleotide sequence encoding humanized 12A11 VH (version 1) is set forth as SEQ ID NO:33 and the amino acid sequence of humanized 12A11 (version 1) is set forth as SEQ ID NO:34. The following residues in the light chain were identified as candidates for backmutation: V2, 148, G64 and F71, canonical; M4, P40, L47, Y49, G66, G68 and T69, vernier; Y36, Q38, P44, L46, Y87 and F98, packing. However, as none of these residues differed between donor and acceptor, no backmutations were made in the first versions of humanized 12A11.

PCR-mediated assembly was used to generate h12A11v1 using appropriate oligonucleotide primers. The nucleotide sequences of humanized 12A11VL (version 1) and 12A11VH (version 1) are set forth as SEQ ID NOs: 31 and 32, respectively. For the variable light chain, the leader peptide encoded by A 19 germline sequence (Accession No. X63397) was used. For the variable heavy chain, the leader peptide was derived from the M72 acceptor sequence (Accession No. AAA69734).

The vernier residues (e.g., S28T, V48L, F67L, L78V) contribute indirectly to CDR conformation and were postulated to be of least significance for conformational perturbation. The targeted residues were mutated by site-directed mutagenesis and h12A11 VHv1 in a pCRS plasmid as the mutagenesis template to arise at clones corresponding to version 2. A sequence-verified V-region insert of version 2 was subcloned into the BamHI/HindIII sites of the heavy chain expression vector pCMV-Cgamma1 (SEQ ID NOs: 91 and 92) to produce recombinant h12A11v2 antibody. A version 2.1 antibody was similarly created having each of the above vernier residue mutations (i.e., elimination of backmutations) in addition to mutation at position T73N. A version 3 antibody likewise had each of the above mutations, T28S, L48V, L67F, V78L, in addition to a mutation at position K71R.

Additional humanized 12A11 versions were designed which retained backmutations at canonical and packing residues but eliminated backmutations at one (versions 4.1 to 4.4), two (versions 5.1 to 5.6) or three (versions 6.1 to 6.4) vernier residues. Site-directed mutagenesis and clone construction was performed as described in subpart C, above. Recombinant antibodies were expressed in COS cells and purified from COS cell supernatants. The amino acid sequences of the humanized 12A11 antibodies, versions 4.1 to 6.4, are set forth as SEQ ID NOs: 40-53. Additional versions are contemplated which include combinations of the above, for example, human residues at 1, 2, 3, 4 or 5 vernier residues in combination with at least one packing and/or canonical residue (e.g., human residues at positions 28, 37, 48, 67, 71 and 78 or human residues at positions 28, 37, 48, 67, 71, 73 and 78). For example, a version 3.1 antibody was created having human residues at 1 vernier residue in combination with one packing residue and two canonical residues (i.e., human residues at positions 28, 48, 67, 71, 73 and 78). As compared to version 1 which has 21% mouse variable region residues (VL+VH), version 3.1 has only 17% mouse variable region residues (i.e., has a lower murine content). The nucleotide and amino acid sequences of the humanized 12A11 version 3.1 heavy chain variable region are set forth as SEQ ID NOs: 38 and 39, respectively.

A seventh version of humanized 12A11 is created having each of the backmutations indicated for version 1, except for the T→S backmutation at residue 28 (vernier), and the V→I backmutation at residue 37 (packing). An eighth version of humanized 12A11 is created having each of the backmutations indicated for version 1, except for the N→T backmutation at residue 73 (vernier). The amino acid sequences of humanized 12A11 version 7 and 8 heavy chains are set forth as SEQ ID NOs: 54 and 55 respectively. FIG. 15A-C depicts the amino acid sequences of h12A11 v1 to v8.

As compared to version 1, version 7 contains only 7 backmutations. The T28S backmutation is conservative and is eliminated in version 7 of the heavy chain. The backmutation at packing residue V37I is also eliminated in version 7. As compared to version 1, version 7 contains only 8 backmutations. In version 8, the N73T (vernier) backmutation is eliminated.

Additional versions may include combinations of the above, for example, human residues (e.g., elimination of backmutations) at 1, 2, 3, 4 (or 5) residues selected from positions 28, 48, 78 and 73, optionally in combination with elimination of backmutation at at least one packing residue (e.g., position 37) and/or at least one canonical residue.

### Example XIX

### Functional Testing of Humanized 12A11 Antibodies

All humanized 12A11 versions were cloned into appropriate expression vectors. The coding sequence for each antibody was operably linked to a germline leader sequence to facilitate extracellular secretion. Antibodies were transiently expressed in COS cells for production of analytical quantities of antibody used in the functional testing described infra. CHO and HEK293 cell lines were stably transfected and cultured in suspension to provide production levels of antibody for use in vivo. Antibodies were purified according to art-recognized methodologies.

In some experiments, expression of h12A11v3.1 in transiently-transfected COS cells was increased by manipulation of heavy chain introns. In other experiments, expression of h12A11v3.1 in a stably transfected pool was increased by manipulation of heavy chain intron content (i.e., deletion of introns between CH1 and hinge region, intron between the hinge region and CH2, and intron between CH2 and CH3) and signal sequence (i.e., use of the generic signal sequence MGWSCIILFLVATGAHS (SEQ ID NO:87).

Humanized 12A11 version 1 was further compared to its murine and chimeric counterparts for two properties: antigen binding (quantitative Aβ ELISA) and relative affinity. The binding activity of h12A11v1 was demonstrated in the quantitative Aβ ELISA and found to be undistinguishable from murine and chimeric forms of 12A11.

The affinity of h12A11v1 antibody was also compared with murine and chimeric 12A11 antibodies by a competitive Aβ ELISA. For the competitive binding assay, a biotin conjugated recombinant mouse 12A11Cγ2a (isotype switched 12A11) was used. The binding activity of the biotinylated m 12A11 Cγ2a for aggregate Aβ 1-42 was comparable to that of the original Cγ1 mouse antibody. The humanized 12A11v1 competed within 2× IC50 value with its murine and chimeric counterparts. This data is consistent with affinity determination using Biacore technology which indicated KD values of 38 nM and 23 nM for the murine Cγ2a and h12A11v1, respectively. In summary, the findings suggest h12A11v1 retains the antigen binding properties and affinity of its original murine counterpart. When tested in the quantitative Aβ ELISA assay, h12A11v2, v2.1 and v3 are comparable to h12A11v1 and to chimeric 12A11 for antigen binding. Moreover, versions 5.1-5.6 and 6.1-6.3 exhibit similar binding activities when tested in this binding assay. Version 6.4 showed some loss of activity in the assay but activity was notably restored in v2.

Binding properties for murine 12A11 and the various humanized 12A11 antibodies were also compared using BIAcore technology. Table 32 includes a summary of kinetic analysis of Aβ binding of the various humanized 12A11 antibodies.

TABLE 32
Binding Properties of 12A11 Antibodies

| | Langmuir Model (global analysis) | | | | |
|---|---|---|---|---|---|
| Antibody | ka (1/Ms) | kd (1/s) | KA (1/M) | KD (nM) | Chi2 |
| mu12A11 | 5.28E+05 | 1.65E−03 | 3.21E+08 | 3.12 | 1.71 |
| chi12A11 | 4.89E+05 | 1.54E−03 | 3.17E+08 | 3.15 | 2.26 |
| h12A11v1 | 4.08E+05 | 1.17E−03 | 3.48E+08 | 2.87 | 8.01 |
| h12A11v2 | 2.83E+05 | 4.12E−03 | 6.86E+07 | 14.6 | 0.96 |
| h12A11v2.1 | 3.06E+05 | 2.61E−03 | 1.17E+08 | 8.54 | 0.88 |
| h12A11v3 | 3.59E+05 | 5.50E−03 | 6.53E+07 | 15.3 | 0.90 |
| h12A11v3.1 | 5.23E+05 | 3.13E−03 | 1.67E+08 | 5.99 | 1.10 |

The data indicate that humanized 12A11 v1 and humanized 12A11 v3.1 have similar affinities for Aβ peptide when compare with parental murine 12A11 (h12A11v1>m12A11>chi12A11>h12A11v3.1>h12A11v2.1>h12A11v2>h12A11v3.1). Notably, the affinity of humanized 12A11 v1 and humanized 12A11 v3.1 are within 2-3× that of the chimeric 12A11 antibody as determined by competitive binding and/or BIAcore analysis.

Similar results were seen in competition binding studies performed as described above. Notably, restoring the backmutation at vernier residue 48 in VH (V48L) increases the affinity of h12A11 v3.1 to near that of h12A11v1.

Humanized 12A11 v1 and humanized 12A11v3 were further tested for the ability to bind plaque and to clear plaques in an ex vivo assay. Immunohistochemistry was performed on cryostat sections from PDAPP and human AD brains. Humanized 12A11 v1 and humanized 12A11 v3 were compared to chimeric 12A11 and found to stain plaque in both PDAPP and human cryostat sections at all concentrations tested (i.e., 0.3 μg/ml, 1 μg/ml and 3 μg/ml). As a measure of their ability to trigger Fc-mediated plaque clearance, humanized 12A11 v1 and humanized 12A11v3 were also tested in an ex vivo phagocytosis assay with primary mouse microglial cells and sections of brain tissue from PDAPP mice for their ability to clear plaque. Irrelevant IgG1 antibody, having no reactivity toward Aβ or other components of the assay, was used as a negative control. Each of the humanized 12A11 v1, humanized 12A11v3 and chimeric 12A11 antibodies efficiently reduced Aβ levels when tested at a concentration of 0.3 μg/ml.

The binding specificity of chimeric 12A11, humanized 12A11 v1 and humanized 12A11v3 were compared by replacement NET (rNET) analysis as described supra. Notably, the specificity of humanized 12A11 v1 and humanized 12A11 v3.1 were the same or similar to the parent murine 12A11 antibody.

Further description of the 12A11 antibody can be found in U.S. patent application Ser. No. 10/858,855, International Patent Application No. PCT/US04/17514, U.S. Patent Application 60/636,776, filed Dec. 15, 2004 (bearing Attorney Docket No. ELN-060-1), entitled “HUMANIZED Aβ ANTIBODIES FOR USE IN IMPROVING COGNITION”.

### Example XX

### In vivo Efficacy of a Humanized 12A11 Antibody: Rapid Improvement in Cognition of a Tg2576 Mouse

The therapeutic efficacy of murine 12A11 (mu12A11), chimeric 12A11 (chi12A11), and a humanized form of 12A11 (v3.1 hu 12A11) antibody were compared in a CFC assay. As in Example XVIII, both wild-type and Tg2576 mice were administered a single dose of phosphate buffered saline (PBS) or treatment antibody by intraperitoneal injection.

To assess any rapid improvement in cognition (i.e. contextual and cue-dependent memory), each mouse was administered a CFC training session immediately following treatment and a CFC testing session within 24 hours of treatment (i.e. Day 1 post-treatment). The results of CFC assay are depicted in FIG. 16. The data clearly indicate that mu12A11, chi12A11 and v3.1 hu12A11 have a similar potency in rapidly improving cognition. For example, a 3 mg/kg dose or greater of either chi12A11 or v3.1 hu12A11 resulted in a memory deficit reversal that was similar in magnitude to the results obtained with a 1 mg/kg dose of mu12A11. Humanized 12A11 antibodies v1.0 through v3.1, also proved efficacious in the CFC assays, in particular, v1.0, v3.0 and v3.1, with v1.0 and v3.1 having efficacy similar to that of murine 12A11, and v3.0 further exhibiting significant efficacy. Moreover, h12A11 v1.0 was efficacious in the CFC assay when doubly transgenic AD mice were tested (MED=3 mg/kg), indicating that the efficacy of passively administered antibody was not titrated out due to plaque binding in these mice. The mouse antibody 266 (non-plaque binding) was included as a positive control (MED=3 mg/kg). The ability of an IgG isotype h12A11 v1.0 antibody was also tested in CFC and proved to be efficacious at MED 0.1 mg/kg. The isotype switch demonstrated to have greatly reduced (but not eliminated) Fc-mediated activity, indicating that efficacy is not solely dependent on Fc function.

Cell lines producing the antibodies 3D6 and 10D5, having the ATCC accession numbers PTA-5129 and PTA-5130, respectively, were deposited on Apr. 8, 2003, under the terms of the Budapest Treaty and cell lines producing the antibodies 1C2, 2B1, 6C6 and 9G8, having the the ATCC accession numbers ______, ______, ______ and ______, respectively, were deposited on Oct. 31, 2005, under the terms of the Budapest Treaty. Also, cell lines producing the antibodies 2H3, 12A11, 15C11 and 3A3, having the A TCC accession numbers ______, ______, ______, and ______, respectively, were deposited on Dec. 12, 2005, under the terms of the Budapest Treaty.

From the foregoing it will be apparent that the invention provides for a number of uses. For example, the invention provides for the use of any of the antibodies to Aβ described above in the treatment, prophylaxis or diagnosis of amyloidogenic disease, or in the manufacture of a medicament or diagnostic composition for use in the same. Although the foregoing invention has been described in detail for purposes of clarity of understanding, it will be obvious that certain modifications may be practiced within the scope of the appended claims. All publications and patent documents cited herein, as well as text appearing in the figures and sequence listing, are hereby incorporated by reference in their entirety for all purposes to the same extent as if each were so individually denoted.

What is claimed is:

1. A method for effecting rapid improvement in cognition in a subject, comprising administering to the subject an effective dose of an Aβ antibody, wherein the antibody is specific for an epitope within residues 1-10 of Aβ and preferentially binds to soluble oligomeric Aβ as compared to monomeric Aβ, such that the rapid improvement in cognition is achieved.

2. A method for effecting rapid improvement in cognition in a subject, comprising administering to the subject an effective dose of an Aβ antibody, wherein the antibody is specific for an epitope within residues 1-10 of Aβ and effects a rapid improvement in cognition in an animal model of an Aβ-related disorder as determined in a Contextual Fear Conditioning (CFC) assay, such that the rapid improvement in cognition is achieved.

3. A method for effecting rapid improvement in cognition in a subject, comprising administering to the subject an effective dose of an Aβ antibody, wherein the antibody is specific for an epitope within residues 1-10 of Aβ, preferentially binds to soluble oligomeric Aβ as compared to monomeric Aβ and effects a rapid improvement in cognition in an animal model of an Aβ-related disorder as determined in a Contextual Fear Conditioning (CFC) assay, such that the rapid improvement in cognition is achieved.

4. The method of any one of claims 1-3, wherein the Aβ antibody binds to an epitope within residues 3-7 of Aβ.

5. The method of any one of claims 1-3, wherein the Aβ antibody is selected from the group consisting of a 3D6 antibody, a 6C6 antibody, a 10D5 antibody, and a 12A11 antibody.

6. The method of any one of claims 1-3, provided that the Aβ antibody is not a 3D6 antibody.

7. A method for effecting rapid improvement in cognition in a subject, comprising administering to the subject an effective dose of an Aβ antibody, wherein the antibody is specific for an epitope within residues 13-28 of Aβ and preferentially binds to soluble oligomeric Aβ as compared to monomeric Aβ, such that the rapid improvement in cognition is achieved.

8. A method for effecting rapid improvement in cognition in a subject, comprising administering to the subject an effective dose of an Aβ antibody, wherein the antibody is specific for an epitope within residues 13-28 of Aβ and effects a rapid improvement in cognition in an animal model of an Aβ-related disorder as determined in a Contextual Fear Conditioning (CFC) assay, such that the rapid improvement in cognition is achieved.

9. A method for effecting rapid improvement in cognition in a subject, comprising administering to the subject an effective dose of an Aβ antibody, wherein the antibody is specific for an epitope within residues 13-28 of Aβ, preferentially binds to soluble oligomeric Aβ as compared to monomeric Aβ and effects a rapid improvement in cognition in an animal model of an Aβ-related disorder as determined in a Contextual Fear Conditioning (CFC) assay, such that the rapid improvement in cognition is achieved.

10. The method of any one of claims 7-9, wherein the Aβ antibody binds to an epitope within residues 16-24 of Aβ.

11. The method of any one of claims 7-9, wherein the Aβ antibody is selected from the group consisting of a 2B1 antibody, a 1C2 antibody, and a 15C11 antibody.

12. The method of any one of claims 7-9, provided that the Aβ antibody is not a 266 antibody.

13. The method of any one of the preceding claims, wherein the improvement in cognition in the animal model is an improvement in memory impairment status or a reversal of memory deficit.

14. The method of any one of the preceding claims, wherein the subject has or is at risk for an Aβ-related disease or disorder.

15. The method of claim 14, wherein the Aβ-related disease or disorder is associated with or characterized by soluble Aβ.

16. The method of claim 15, wherein the Aβ-related disease or disorder is associated with or characterized by insoluble Aβ.

17. The method of claim 15, wherein the Aβ-related disease or disorder is an amyloidogenic disease.

18. The method of claim 17, wherein the Aβ-related disease or disorder is Alzheimer's disease.

19. The method of claim 15, wherein the Aβ-related disease or disorder is an Aβ-related cognitive disorder.

20. The method of claim 19, wherein the Aβ-related cognitive disorder is mild cognitive impairment.

21. The method of any one of the preceding claims, wherein the subject is substantially free of amyloid deposits.

22. The method of any one of the preceding claims, wherein the Aβ antibody is administered to the subject prior to substantial plaque deposition in the subject.

23. The method of any one of the preceding claims, wherein the subject has been diagnosed with Alzheimer's Disease.

24. The method of any one of claims 1-21, wherein the Aβ antibody is administered to the subject subsequent to substantial plaque deposition in the subject.

25. The method of any one of the preceding claims, wherein the Aβ antibody is administered to the subject as a single dose.

26. The method of any one of claims 1-24, wherein the Aβ antibody is administered to the subject in multiple doses.

27. The method of any one of the preceding claims, wherein the dose of Aβ antibody is from about 100 μg/kg to 100 mg/kg body weight of the patient.

28. The method of any one of claims 1-26, wherein the dose of Aβ antibody is from about 300 μg/kg to 30 mg/kg body weight of the patient.

29. The method of any one of claims 1-26, wherein the dose of Aβ antibody is from about 1 mg/kg to 10 mg/kg body weight of the patient.

30. The method of any one of the preceding claims, wherein the rapid improvement in cognition is achieved within one month after administration of the antibody.

31. The method of any one of the preceding claims, wherein the rapid improvement in cognition is achieved within one week after administration of the antibody.

32. The method of any one of claims 1-30, wherein the rapid improvement in cognition is achieved within one day after administration of the antibody.

33. The method of any one of claims 1-30, wherein the rapid improvement in cognition is achieved within 12 hours after administration of the antibody.

34. The method of any one of the preceding claims, wherein the subject is a human.

35. A composition comprising an Aβ antibody in an amount effective to rapidly improve cognition in a subject, wherein the antibody is specific for an epitope within residues 1-10 of Aβ and preferentially binds to soluble oligomeric Aβ as compared to monomeric Aβ.

36. A composition comprising an Aβ antibody in an amount effective to rapidly improve cognition in a subject, wherein the antibody is specific for an epitope within residues 1-10 of Aβ and effects a rapid improvement in cognition in an animal model of an Aβ-related disorder as determined in a Contextual Fear Conditioning (CFC) assay.

37. A composition comprising an Aβ antibody in an amount effective to rapidly improve cognition in a subject, wherein the antibody is specific for an epitope within residues 1-10 of Aβ, preferentially binds to soluble oligomeric Aβ as compared to monomeric Aβ and effects a rapid improvement in cognition in an animal model of an Aβ-related disorder as determined in a Contextual Fear Conditioning (CFC) assay.

38. The composition of any one of claims 35-37, wherein the Aβ antibody binds to an epitope within residues 3-7 of Aβ.

39. The composition of any one of claims 35-37, wherein the Aβ antibody is selected from the group consisting of a 3D6 antibody, a 6C6 antibody, a 10D5 antibody, and a 12A11 antibody.

40. The composition of any one of claims 35-37, provided that the Aβ antibody is not a 3D6 antibody.

41. A composition for effecting rapid improvement in cognition in a subject, comprising an effective dose of an Aβ antibody, wherein the antibody is specific for an epitope within residues 13-28 of Aβ and preferentially binds to soluble oligomeric Aβ as compared to monomeric Aβ, such that the rapid improvement in cognition is achieved.

42. A composition for effecting rapid improvement in cognition in a subject, comprising an effective dose of an Aβ antibody, wherein the antibody is specific for an epitope within residues 13-28 of Aβ and effects a rapid improvement in cognition in an animal model of an Aβ-related disorder as determined in a Contextual Fear Conditioning (CFC) assay, such that the rapid improvement in cognition is achieved.

43. A composition for effecting rapid improvement in cognition in a subject, comprising an effective dose of an Aβ antibody, wherein the antibody is specific for an epitope within residues 13-28 of Aβ, preferentially binds to soluble oligomeric Aβ as compared to monomeric Aβ and effects a rapid improvement in cognition in an animal model of an Aβ-related disorder as determined in a Contextual Fear Conditioning (CFC) assay, such that the rapid improvement in cognition is achieved.

44. The composition of any one of claims 41-43, wherein the Aβ antibody binds to an epitope within residues 16-24 of Aβ.

45. The composition of any one of claims 41-43, wherein the Aβ antibody is selected from the group consisting of a 2B1 antibody, a 1C2 antibody, and a 15C11 antibody.

46. The composition of any one of claims 41-43, provided that the Aβ antibody is not a 266 antibody.

47. The composition of any one of claims 41-46, wherein the improvement in cognition in the animal model is an improvement in memory impairment status or a reversal of memory deficit.

48. The composition of any one of claims 41-47, wherein the antibody neutralizes one or more neuroactive Aβ species.

49. The composition of any one of claims 41-48, wherein the Aβ antibody clears plaques.

50. The composition of any one of claims 41-49, formulated for single dose administration.

51. The composition of any one of claims 41-49, formulated for multiple dose administration.

52. A humanized immunoglobulin comprising complementarity determining regions (CDRs) the 6C6, antibody produced by the cell line having ATCC Accession Number ______.

53. A humanized version of the monoclonal antibody 6C6 produced by the cell line having ATCC Accession Number ______.

54. A humanized immunoglobulin comprising complementarity determining regions (CDRs) the 2B1, antibody produced by the cell line having ATCC Accession Number ______.

55. A humanized version of the monoclonal antibody 2B1 produced by the cell line having ATCC Accession Number ______.

56. A humanized immunoglobulin comprising complementarity determining regions (CDRs) the 1C2, antibody produced by the cell line having ATCC Accession Number ______.

57. A humanized version of the monoclonal antibody 1C2 produced by the cell line having ATCC Accession Number ______.

58. A humanized immunoglobulin comprising complementarity determining regions (CDRs) the 9G8, antibody produced by the cell line having ATCC Accession Number ______.

59. A humanized version of the monoclonal antibody 9G8 produced by the cell line having ATCC Accession Number ______.

60. A method for effecting rapid improvement in cognition in a subject, comprising administering to the subject an effective dose of the antibody of any one of claims 52-59, such that the rapid improvement in cognition is achieved.


SEQUENCE LISTING (95 sequences)

SEQ ID NO: 1 - DNA, 396 bp, Mus musculus
  CDS (1)...(396)
  sig_peptide (1)...(60)
atgatgagtcctgcccagttcctgtttctgttagtgctctggattcgggaaaccaacggttatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagattttacactgaaaatcagcagaatagaggctgaggatttgggactttattattgctggcaaggtacacattttcctcggacgttcggtggaggcaccaagctggaaatcaaa

SEQ ID NO: 2 - protein, 132 aa, Mus musculus
  SIGNAL (1)...(20)
MMSPAQFLFLLVLWIRETNGYVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIK

SEQ ID NO: 3 - DNA, 414 bp, Mus musculus
  CDS (1)...(414)
  sig_peptide (1)...(57)
atgaacttcgggctcagcttgattttccttgtccttgttttaaaaggtgtccagtgtgaagtgaagctggtggagtctgggggaggcttagtgaagcctggagcgtctctgaaactctcctgtgcagcctctggattcactttcagtaactatggcatgtcttgggttcgccagaattcagacaagaggctggagtgggttgcatccattaggagtggtggtggtagaacctactattcagacaatgtaaagggccgattcaccatctccagagagaatgccaagaacaccctgtacctgcaaatgagtagtctgaagtctgaggacacggccttgtattattgtgtcagatatgatcactatagtggtagctccgactactggggccagggcaccactgtcacagtctcctca

SEQ ID NO: 4 - protein, 138 aa, Mus musculus
  SIGNAL (1)...(19)
MNFGLSLIFLVLVLKGVQCEVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVSS

SEQ ID NO: 5 - DNA, 402 bp, Artificial Sequence
  Synthetic construct,  h3D6 version 1 VL
atggacatgcgcgtgcccgcccagctgctgggcctgctgatgctgtgggtgtccggctcctccggctacgtggtgatgacccagtcccccctgtccctgcccgtgacccccggcgagcccgcctccatctcctgcaagtcctcccagtccctgctggactccgacggcaagacctacctgaactggctgctgcagaagcccggccagtccccccagcgcctgatctacctggtgtccaagctggactccggcgtgcccgaccgcttctccggctccggctccggcaccgacttcaccctgaagatctcccgcgtggaggccgaggacgtgggcgtgtactactgctggcagggcacccacttcccccgcaccttcggccagggcaccaaggtggagatcaag

SEQ ID NO: 6 - protein, 132 aa, Artificial Sequence
  SIGNAL (1)...(20): Synthetic construct, humanized 3D6 light chain variable region
MMSPAQFLFLLVLWIRETNGYVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIK

SEQ ID NO: 7 - DNA, 414 bp, Artificial Sequence
  Synthetic construct,  h3D6 version 1 VH
atggagtttgggctgagctggctttttcttgtggctattttaaaaggtgtccagtgtgaggtgcagctgctggagtccggcggcggcctggtgcagcccggcggctccctgcgcctgtcctgcgccgcctccggcttcaccttctccaactacggcatgtcctgggtgcgccaggcccccggcaagggcctggagtgggtggcctccatccgctccggcggcggccgcacctactactccgacaacgtgaagggccgcttcaccatctcccgcgacaacgccaagaactccctgtacctgcagatgaactccctgcgcgccgaggacaccgccctgtactactgcgtgcgctacgaccactactccggctcctccgactactggggccagggcaccctggtgaccgtgtcctcc

SEQ ID NO: 8 - protein, 138 aa, Artificial Sequence
  Synthetic construct,  Humanized 3D6 heavy chain variable region
MNFGLSLIFLVLVLKGVQCEVQLLESGGGLVQPGGSLRLSCAASGFTFSNYGMSWVRQAPGKGLEWVASIRSGGGRTYYSDNVKGRFTISRDNAKNSLYLQMNSLRAEDTALYYCVRYDHYSGSSDYWGQGTLVTVSS

SEQ ID NO: 9 - DNA, 402 bp, Artificial Sequence
  Synthetic construct,  h3D6 version 2 VL
atggacatgcgcgtgcccgcccagctgctgggcctgctgatgctgtgggtgtccggctcctccggcgacgtggtgatgacccagtcccccctgtccctgcccgtgacccccggcgagcccgcctccatctcctgcaagtcctcccagtccctgctggactccgacggcaagacctacctgaactggctgctgcagaagcccggccagtccccccagcgcctgatctacctggtgtccaagctggactccggcgtgcccgaccgcttctccggctccggctccggcaccgacttcaccctgaagatctcccgcgtggaggccgaggacgtgggcgtgtactactgctggcagggcacccacttcccccgcaccttcggccagggcaccaaggtggagatcaag

SEQ ID NO: 10 - protein, 132 aa, Artificial Sequence
  SIGNAL (1)...(20)
  Synthetic construct, humanized 3D6 light chain variable region
MMSPAQFLFLLVLWIRETNGDVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIK

SEQ ID NO: 11 - DNA, 414 bp, Artificial Sequence
  Synthetic construct,  h3D6 version 2 VH
atggagtttgggctgagctggctttttcttgtggctattttaaaaggtgtccagtgtgaggtgcagctgctggagtccggcggcggcctggtgcagcccggcggctccctgcgcctgtcctgcgccgcctccggcttcaccttctccaactacggcatgtcctgggtgcgccaggcccccggcaagggcctggagtgggtggcctccatccgctccggcggcggccgcacctactactccgacaacgtgaagggccgcttcaccatctcccgcgacaactccaagaacaccctgtacctgcagatgaactccctgcgcgccgaggacaccgccgtgtactactgcgtgcgctacgaccactactccggctcctccgactactggggccagggcaccctggtgaccgtgtcctcc

SEQ ID NO: 12 - protein, 138 aa, Artificial Sequence
  Synthetic construct,  Humanized 3D6 light chain variable region
MNFGLSLIFLVLVLKGVQCEVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSS

SEQ ID NO: 13 - DNA, 393 bp, Mus musculus
  CDS (1)...(393)
  sig_peptide (1)...(57)
atgaagttgcctgttaggctgttggtactgatgttctggattcctgcttccagcagtgatgttttgatgacccaaactccactctccctgcctgtcagtcttggagatcaagcctccatctcttgcagatctagtcagaacattatacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcaagaaagtggaggctgaggatctgggaatttattactgctttcaaggttcacatgttccgctcacgttcggtgctgggaccaagctggagctggaa

SEQ ID NO: 14 - protein, 131 aa, Mus musculus
  SIGNAL (1)...(19)
MKLPVRLLVLMFWIPASSSDVLMTQTPLSLPVSLGDQASISCRSSQNIIHSNGNTYLEWYLQKPGQSPKLLIYKVSNRFSGVPDRFSGSGSGTDFTLKIKKVEAEDLGIYYCFQGSHVPLTFGAGTKLELE

SEQ ID NO: 15 - DNA, 426 bp, Mus musculus
  CDS (1)...(426)
  sig_peptide (1)...(57)
atggacaggcttacttcctcattcctgctgctgattgtccctgcatatgtcctgtcccaggctactctgaaagagtctggccctggaatattgcagtcctcccagaccctcagtctgacttgttctttctctgggttttcactgagcacttctggtatgggagtgagctggattcgtcagccttcaggaaagggtctggagtggctggcacacatttactgggatgatgacaagcgctataacccatccctgaagagccggctcacaatctccaaggatacctccagaaagcaggtattcctcaagatcaccagtgtggaccctgcagatactgccacatactactgtgttcgaaggcccattactccggtactagtcgatgctatggactactggggtcaaggaacctcagtcaccgtctcctca

SEQ ID NO: 16 - protein, 142 aa, Mus musculus
  SIGNAL (1)...(19)
MDRLTSSFLLLIVPAYVLSQATLKESGPGILQSSQTLSLTCSFSGFSLSTSGMGVSWIRQPSGKGLEWLAHIYWDDDKRYNPSLKSRLTISKDTSRKQVFLKITSVDPADTATYYCVRRPITPVLVDAMDYWGQGTSVTVSS

SEQ ID NO: 17 - DNA, 393 bp, Mus musculus
  sig_peptide (1)...(57)
  CDS (1)...(393)
atgaagttgcctgttaggctgttggtgctgatgttctggattcctgcttccagcagtgatgttttgatgacccaaactccactctccctgcctgtcagtcttggagatcaagcctccatctcttgcagatctagtcagaacattgttcatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgctcacgttcggtgctgggaccaagctggagctgaaa

SEQ ID NO: 18 - protein, 131 aa, Mus musculus
  SIGNAL (1)...(19)
MKLPVRLLVLMFWIPASSSDVLMTQTPLSLPVSLGDQASISCRSSQNIVHSNGNTYLEWYLQKPGQSPKLLIYKVSNRFSGVPDRFSGSGSGTDFTLKISRVEAEDLGVYYCFQGSHVPLTFGAGTKLELK

SEQ ID NO: 19 - DNA, 426 bp, Mus musculus
  CDS (1)...(426)
  sig_peptide (1)...(57)
atggacaggcttacttcctcattcctgctgctgattgtccctgcatatgtcctgtcccaggttactctgaaagagtctggccctgggatattgcagccctcccagaccctcagtctgacttgttctttctctgggttttcactgagcactaatggtatgggtgtgagctggattcgtcagccttcaggaaagggtctggagtggctggcacacatttactgggatgaggacaagcgctataacccatccctgaagagccggctcacaatctccaaggatacctctaacaatcaggtattcctcaagatcaccaatgtggacactgctgatactgccacatactactgtgctcgaaggaggatcatctatgatgttgaggactactttgactactggggccaaggcaccactctcacagtctcctca

SEQ ID NO: 20 - protein, 142 aa, Mus musculus
  SIGNAL (1)...(19)
MDRLTSSFLLLIVPAYVLSQVTLKESGPGILQPSQTLSLTCSFSGFSLSTNGMGVSWIRQPSGKGLEWLAHIYWDEDKRYNPSLKSRLTISKDTSNNQVFLKITNVDTADTATYYCARRRIIYDVEDYFDYWGQGTTLTVSS

SEQ ID NO: 21 - DNA, 396 bp, Artificial Sequence
  Synthetic construct - humanized 12B4VLv1
atgaggctccctgctcagctcctggggctgctaatgctctgggtctctggatccagtggggatgttgtgatgactcagtctccactctccctgcccgtcacccctggagagccggcctccatctcctgcaggtctagtcagaacattgttcatagtaatggaaacacctatttggaatggtacctgcagaagccagggcagtctccacagctcctgatctacaaagtttccaaccgattttctggggtccctgacaggttcagtggcagtggatcaggcacagattttacactgaaaatcagcagagtggaggctgaggatgttggggtttattactgctttcaaggttcacatgttccgctcacgttcggtcaggggaccaagctggagatcaaa

SEQ ID NO: 22 - protein, 132 aa, Artificial Sequence
  Synthetic construct,  humanized 12B4VLv1
MRLPAQLLGLLMLWVSGSSGDVVMTQSPLSLPVTPGEPASISCRSSQNIVHSNGNTYLEWYLQKPGQSPQLLIYKVSNRFSGVPDRFSGSGSGTDFTLKISRVEAEDVGVYYCFQGSHVPLTFGQGTKLEIK

SEQ ID NO: 23 - DNA, 426 bp, Artificial Sequence
  Synthetic construct,  humanized 12B4VHv1
atgaagcacctgtggttcttcctcctgctggtggcagctcccagatgggtcctgtcccaggtgcagctgcaggagtcgggcccaggactggtgaagccttcggagaccctgtccctcacctgcactttctctggtttttccctgagcactaatggtatgggtgtgagctggatccggcagcccccagggaagggactggagtggctggcacacatctattgggatgaggacaagcgctataacccatccctcaagagtcgactcaccatatcaaaggacacgtccaagaaccaggtatccctgaagctgagctctgtgaccgctgcagacacggccgtgtattactgtgcgagaaggaggatcatctatgatgttgaggactactttgactactggggccaagggaccacggtcaccgtctcctca

SEQ ID NO: 24 - protein, 142 aa, Artificial Sequence
  Synthetic construct,  humanized 12B4VHv1
MKHLWFFLLLVAAPRWVLSQVQLQESGPGLVKPSETLSLTCTFSGFSLSTNGMGVSWIRQPPGKGLEWLAHIYWDEDKRYNPSLKSRLTISKDTSKNQVSLKLSSVTAADTAVYYCARRRIIYDVEDYFDYWGQGTTVTVSS

SEQ ID NO: 25 - protein, 142 aa, Artificial Sequence
  synthetic construct,  humanized 12B4VLv2
MKHLWFFLLLVAAPRWVLSQLQLQESGPGLVKPSETLSLTCTFSGFSLSTNGMGVSWIRQPPGKGLEWIGHIYWDEDKRYNPSLKSRVTISKDTSKNQFSLKLSSVTAADTAVYYCARRRIIYDVEDYFDYWGQGTTVTVSS

SEQ ID NO: 26 - protein, 142 aa, Artificial Sequence
  synthetic construct,  humanized 12B4VLv3
MKHLWFFLLLVAAPRWVLSQLQLQESGPGLVKPSETLSLTCTFSGFSLSTNGMGVSWIRQPPGKGLEWLGHIYWDEDKRYNPSLKSRVTISKDTSKNQVSLKLSSVTAADTAVYYCARRRIIYDVEDYFDYWGQGTTVTVSS

SEQ ID NO: 27 - DNA, 393 bp, Murine
  CDS (1)...(393)
  sig_peptide (1)...(57)
atgaagttgcctgttaggctgttggtgctgatgttctggattcctgcttccagcagtgatgttttgatgacccaaactccactctccctgcctgtcagtcttggagatcaagcctccatctcttgcagatctagtcagagcattgtacatagtaatggaaacacctacttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggaatttattactgctttcaaagttcacatgttcctctcacgttcggtgctgggaccaagctggagctgaaa

SEQ ID NO: 28 - protein, 131 aa, Murine
  SIGNAL (1)...(19)
MKLPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAEDLGIYYCFQSSHVPLTFGAGTKLELK

SEQ ID NO: 29 - DNA, 417 bp, Murine
  CDS (1)...(417)
  sig_peptide (1)...(57)
atggacaggcttactacttcattcctgctgctgattgtccctgcatatgtcttgtcccaagttactctaaaagagtctggccctgggatattgaagccctcacagaccctcagtctgacttgttctttctctgggttttcactgagcacttctggtatgagtgtaggctggattcgtcagccttcagggaagggtctggagtggctggcacacatttggtgggatgatgataagtactataacccatccctgaagagccggctcacaatctccaaggatacctccagaaaccaggtattcctcaagatcaccagtgtggacactgcagatactgccacttactactgtgctcgaagaactactacggctgactactttgcctactggggccaaggcaccactctcacagtctcctca

SEQ ID NO: 30 - protein, 139 aa, Murine
  SIGNAL (1)...(19)
MDRLTTSFLLLIVPAYVLSQVTLKESGPGILKPSQTLSLTCSFSGFSLSTSGMSVGWIRQPSGKGLEWLAHIWWDDDKYYNPSLKSRLTISKDTSRNQVFLKITSVDTADTATYYCARRTTTADYFAYWGQGTTLTVSS

SEQ ID NO: 31 - DNA, 396 bp, Artificial Sequence
  synthetic construct,  humanized 12A11 v1 VL sequence
atgaggctccctgctcagctcctggggctgctgatgctctgggtctctggctccagtggggatgttgtgatgacccaatctccactctccctgcctgtcactcctggagagccagcctccatctcttgcagatctagtcagagcattgtgcatagtaatggaaacacctacctggaatggtacctgcagaaaccaggccagtctccacagctcctgatctacaaagtttccaaccgattttctggggtcccagacaggttcagtggcagtggatcagggacagatttcacactcaagatcagcagagtggaggctgaggatgtgggagtttattactgctttcaaagttcacatgttcctctcaccttcggtcaggggaccaagctggagatcaaa

SEQ ID NO: 32 - protein, 112 aa, Artificial Sequence
  synthetic construct,  synthetic h12A11v1 - VL region
DVVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQSSHVPLTFGQGTKLEIK

SEQ ID NO: 33 - DNA, 417 bp, Artificial Sequence
  synthetic construct,  humanized 12A11 v1 NH sequence
atggagtttgggctgagctgggttttcctcgttgctcttctgagaggtgtccagtgtcaagttcagctggtggagtctggcggcggggtggtgcagcccggacggtccctcaggctgtcttgtgctttctctgggttttcactgagcacttctggtatgagtgtgggctggattcgtcaggctccagggaagggtctggagtggctggcacacatttggtgggatgatgataagtactataacccatccctgaagagccggctcacaatctccaaggatacctccaaaaacaccgtgtacctccagatgaacagtctgcgggctgaagatactgccgtgtactactgtgctcgaagaactactaccgctgactactttgcctactggggccaaggcaccactgtcacagtctcctca

SEQ ID NO: 34 - protein, 120 aa, Artificial Sequence
  synthetic construct,  h12A11v1 - VH region
QVQLVESGGGVVQPGRSLRLSCAFSGFSLSTSGMSVGWIRQAPGKGLEWLAHIWWDDDKYYNPSLKSRLTISKDTSKNTVYLQMNSLRAEDTAVYYCARRTTTADYFAYWGQGTTVTVSS

SEQ ID NO: 35 - protein, 120 aa, Artificial Sequence
  synthetic construct, synthetic h12A11v2
QVQLVESGGGVVQPGRSLRLSCAFSGFTLSTSGMSVGWIRQAPGKGLEWVAHIWWDDDKYYNPSLKSRFTISKDTSKNTLYLQMNSLRAEDTAVYYCARRTTTADYFAYWGQGTTVTVSS

SEQ ID NO: 36 - protein, 120 aa, Artificial Sequence
  synthetic construct,  synthetic h12A11v2.1
QVQLVESGGGVVQPGRSLRLSCAFSGFTLSTSGMSVGWIRQAPGKGLEWVAHIWWDDDKYYNPSLKSRFTISKDNSKNTLYLQMNSLRAEDTAVYYCARRTTTADYFAYWGQGTTVTVSS

SEQ ID NO: 37 - protein, 120 aa, Artificial Sequence
  synthetic construct,  synthetic h12A11v3
QVQLVESGGGVVQPGRSLRLSCAFSGFTLSTSGMSVGWIRQAPGKGLEWVAHIWWDDDKYYNPSLKSRFTISRDNSKNTLYLQMNSLRAEDTAVYYCARRTTTADYFAYWGQGTTVTVSS

SEQ ID NO: 38 - DNA, 417 bp, Artificial Sequence
  synthetic construct,  humanized 12A11 v3.1 VH sequence
atggagtttgggctgagctgggttttcctcgttgctcttctgagaggtgtccagtgtcaagttcagctggtggagtctggcggcggggtggtgcagcccggacggtccctcaggctgtcttgtgctttctctgggttcacactgagcacttctggtatgagtgtgggctggattcgtcaggctccagggaagggtctggagtggctggcacacatttggtgggatgatgataagtactataacccatccctgaagagccgattcacaatctccagggacaactccaaaaacacgctgtacctccagatgaacagtctgcgggctgaagatactgccgtgtactactgtgctcgaagaactactaccgctgactactttgcctactggggccaaggcaccactgtcacagtctcctca

SEQ ID NO: 39 - protein, 120 aa, Artificial Sequence
  synthetic construct,  humanized 12A11 VH (version 3.1)
QVQLVESGGGVVQPGRSLRLSCAFSGFTLSTSGMSVGWIRQAPGKGLEWLAHIWWDDDKYYNPSLKSRFTISKDNSKNTLYLQMNSLRAEDTAVYYCARRTTTADYFAYWGQGTTVTVSS

SEQ ID NO: 40 - protein, 120 aa, Artificial Sequence
  synthetic construct,  synthetic h12A11v4.1
QVQLVESGGGVVQPGRSLRLSCAFSGFTLSTSGMSVGWIRQAPGKGLEWLAHIWWDDDKYYNPSLKSRLTISKDTSKNTVYLQMNSLRAEDTAVYYCARRTTTADYFAYWGQGTTVTVSS

SEQ ID NO: 41 - protein, 120 aa, Artificial Sequence
  synthetic construct,  synthetic h12A11v4.2
QVQLVESGGGVVQPGRSLRLSCAFSGFSLSTSGMSVGWIRQAPGKGLEWVAHIWWDDDKYYNPSLKSRLTISKDTSKNTVYLQMNSLRAEDTAVYYCARRTTTADYFAYWGQGTTVTVSS

SEQ ID NO: 42 - protein, 120 aa, Artificial Sequence
  synthetic construct,  synthetic h12A11v4.3
QVQLVESGGGVVQPGRSLRLSCAFSGFSLSTSGMSVGWIRQAPGKGLEWLAHIWWDDDKYYNPSLKSRFTISKDTSKNTVYLQMNSLRAEDTAVYYCARRTTTADYFAYWGQGTTVTVSS

SEQ ID NO: 43 - protein, 120 aa, Artificial Sequence
  synthetic construct,  synthetic h12A11v4.4
QVQLVESGGGVVQPGRSLRLSCAFSGFSLSTSGMSVGWIRQAPGKGLEWLAHIWWDDDKYYNPSLKSRLTISKDTSKNTLYLQMNSLRAEDTAVYYCARRTTTADYFAYWGQGTTVTVSS

SEQ ID NO: 44 - protein, 120 aa, Artificial Sequence
  synthetic construct, synthetic h12A11v5.1
QVQLVESGGGVVQPGRSLRLSCAFSGFTLSTSGMSVGWIRQAPGKGLEWVAHIWWDDDKYYNPSLKSRLTISKDTSKNTVYLQMNSLRAEDTAVYYCARRTTTADYFAYWGQGTTVTVSS

SEQ ID NO: 45 - protein, 120 aa, Artificial Sequence
  synthetic construct,  synthetic h12A11v5.2
QVQLVESGGGVVQPGRSLRLSCAFSGFTLSTSGMSVGWIRQAPGKGLEWLAHIWWDDDKYYNPSLKSRFTISKDTSKNTVYLQMNSLRAEDTAVYYCARRTTTADYFAYWGQGTTVTVSS

SEQ ID NO: 46 - protein, 121 aa, Artificial Sequence
  synthetic construct,  synthetic h12A11v5.3
QVQLVESGGGVVQPGRSLRLSCAFSGFTLSTSGMSVGWIRQAPGKGLEWLAHIWWDDDKYYNPSLKSRLTISKDTSKNTLYLQMNSLRAEDTAVYYCARRTTTADYFAYWGQGTTVTVSSV

SEQ ID NO: 47 - protein, 121 aa, Artificial Sequence
  synthetic construct, synthetic h12A11v5.4
QVQLVESGGGVVQPGRSLRLSCAFSGFSLSTSGMSVGWIRQAPGKGLEWVAHIWWDDDKYYNPSLKSRFTISKDTSKNTVYLQMNSLRAEDTAVYYCARRTTTADYFAYWGQGTTVTVSSV

SEQ ID NO: 48 - protein, 120 aa, Artificial Sequence
  synthetic construct, synthetic h12A11v5.5
QVQLVESGGGVVQPGRSLRLSCAFSGFSLSTSGMSVGWIRQAPGKGLEWVAHIWWDDDKYYNPSLKSRLTISKDTSKNTLYLQMNSLRAEDTAVYYCARRTTTADYFAYWGQGTTVTVSS

SEQ ID NO: 49 - protein, 120 aa, Artificial Sequence
  synthetic construct, synthetic h12A11v5.6
QVQLVESGGGVVQPGRSLRLSCAFSGFSLSTSGMSVGWIRQAPGKGLEWLAHIWWDDDKYYNPSLKSRFTISKDTSKNTLYLQMNSLRAEDTAVYYCARRTTTADYFAYWGQGTTVTVSS

SEQ ID NO: 50 - protein, 120 aa, Artificial Sequence
  synthetic construct, synthetic h12A116.1
QVQLVESGGGVVQPGRSLRLSCAFSGFTLSTSGMSVGWIRQAPGKGLEWVAHIWWDDDKYYNPSLKSRFTISKDTSKNTVYLQMNSLRAEDTAVYYCARRTTTADYFAYWGQGTTVTVSS

SEQ ID NO: 51 - protein, 120 aa, Artificial Sequence
  synthetic construct, synthetic h12A11v6.2
QVQLVESGGGVVQPGRSLRLSCAFSGFTLSTSGMSVGWIRQAPGKGLEWVAHIWWDDDKYYNPSLKSRLTISKDTSKNTLYLQMNSLRAEDTAVYYCARRTTTADYFAYWGQGTTVTVSS

SEQ ID NO: 52 - protein, 120 aa, Artificial Sequence
  synthetic construct, synthetic h12A11v6.3
QVQLVESGGGVVQPGRSLRLSCAFSGFTLSTSGMSVGWIRQAPGKGLEWLAHIWWDDDKYYNPSLKSRFTISKDTSKNTLYLQMNSLRAEDTAVYYCARRTTTADYFAYWGQGTTVTVSS

SEQ ID NO: 53 - protein, 120 aa, Artificial Sequence
  synthetic construct, synthetic h12A11v6.4
QVQLVESGGGVVQPGRSLRLSCAFSGFSLSTSGMSVGWIRQAPGKGLEWVAHIWWDDDKYYNPSLKSRFTISKDTSKNTLYLQMNSLRAEDTAVYYCARRTTTADYFAYWGQGTTVTVSS

SEQ ID NO: 54 - protein, 120 aa, Artificial Sequence
  synthetic construct, synthetic h12A11v7
QVQLVESGGGVVQPGRSLRLSCAFSGFTLSTSGMSVGWVRQAPGKGLEWLAHIWWDDDKYYNPSLKSRLTISKDTSKNTVYLQMNSLRAEDTAVYYCARRTTTADYFAYWGQGTTVTVSS

SEQ ID NO: 55 - protein, 120 aa, Artificial Sequence
  synthetic construct, synthetic h12A11v8
QVQLVESGGGVVQPGRSLRLSCAFSGFSLSTSGMSVGWIRQAPGKGLEWLAHIWWDDDKYYNPSLKSRLTISKDNSKNTVYLQMNSLRAEDTAVYYCARRTTTADYFAYWGQGTTVTVSS

SEQ ID NO: 56 - DNA, 390 bp, Murine
  sig_peptide (1)...(57)
  CDS (1)...(390)
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtacacagtgatggaaacacctatttacattggtacctgcagaagccaggccagtctccaaaactcctgatctacaaagtttccaaccgattttctggggtcccagacaggttcagtggcagtggatcagggacagatttcacactcaagatcagcagagtggaggctgaggatctgggagtttatttctgctctcaaagtacacatgtgtggacgttcggtggaggcaccaagctggaaatcaaa

SEQ ID NO: 57 - protein, 130 aa, Murine
  SIGNAL (1)...(19)
MKLPVRLLVLMFWIPASSSDVVMTQTPLSLPVSLGDQASISCRSSQSLVHSDGNTYLHWYLQKPGQSPKLLIYKVSNRFSGVPDRFSGSGSGTDFTLKISRVEAEDLGVYFCSQSTHVWTFGGGTKLEIK

SEQ ID NO: 58 - DNA, 393 bp, Murine
  sig_peptide (1)...(57)
  CDS (1)...(393)
atgaatttcgggctcagcttgattttccttgtccttgttttaaaaggtgtcctgtgtgaagtgaagctggtggagtctgggggaggtttagtgcagcctggagggtccctgaaactctcctgtgcagcctctggatttactttcagtagatatagtatgtcttgggttcgccagactccagagaagaggctggagttggtcgcaaaaattagtaatagtggtgataacacctactatccagacactttaaagggccgattcaccatctccagagacaatgcccagaacaccctgtacctgcaaatgagcagtctgaagtctgaggacacggccatgtattactgtgcaagcggggactactggggccaaggcaccactctcacagtctcctca

SEQ ID NO: 59 - protein, 131 aa, Murine
  SIGNAL (1)...(19)
MNFGLSLIFLVLVLKGVLCEVKLVESGGGLVQPGGSLKLSCAASGFTFSRYSMSWVRQTPEKRLELVAKISNSGDNTYYPDTLKGRFTISRDNAQNTLYLQMSSLKSEDTAMYYCASGDYWGQGTTLTVSS

SEQ ID NO: 60 - protein, 112 aa, Homo sapiens
  SIGNAL (1)...(20)
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGQGTKVEIK

SEQ ID NO: 61 - protein, 120 aa, Homo sapiens
  SIGNAL (1)...(20)
MRLPAQLLGLLMLWVSGSSGDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTP

SEQ ID NO: 62 - protein, 121 aa, Artificial Sequence
  synthetic construct, Kabat ID 045919 Heavy Chain amino acid sequence
EVQLLESGGGLVQPGGSLRLSCAASGFTFSSYAVSWVRQAPGKGLEWVSAISGSGGSTYYADSVKGRFTISRDNAKNSLYLQMNSLRAEDTALYYCAKDNYDFWSGTFDYWGQGTLVTVSS

SEQ ID NO: 63 - protein, 98 aa, Artificial Sequence
  synthetic construct, Germline VH3-23 Heavy Chain amino acid sequence
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 64 - protein, 100 aa, Homo sapiens
DIVMTQSPLSLPVTPGEPASISCRSSQSLLHRYGYNYLDWYLQKPGQSPQLLIYLGSNRASGVPDRFSGSGSGTDFTLKISRVEAEDVGVYYCMQALQTP

SEQ ID NO: 65 - protein, 135 aa, Homo sapiens
  SIGNAL (1)...(12)
LLLVAAPRWVLSQLQLQESGPGLVKPSETLSLTCTVSGGSISRGSHYWGWIRQPPGKGLEWIGSIYYSGNTYFNPSLKSRVTISVDTSKNQFSLKLSSVTAADTAVYYCARLGPDDYTLDGMDVWGQGTTVTVSS

SEQ ID NO: 66 - protein, 118 aa, Homo sapiens
  SIGNAL (1)...(19)
MKHLWFFLLLVAAPRWVLSQLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAR

SEQ ID NO: 67 - protein, 118 aa, Homo sapiens
  SIGNAL (1)...(19)
MKHLWFFLLLVAAPRWVLSQVQLQESGPGLVKPSETLSLTCTVSGGSVSSGGYYWSWIRQPPGKGLEWIGYIYYSGSTNYNPSLKSRVTISVDTSKNQFSLKLSSVTAADTAVYYCAR

SEQ ID NO: 68 - protein, 134 aa, Homo sapiens
  SIGNAL (1)...(22)
MKYLLPTAAAGLLLLAAQPAMADVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPYTFGQGTKLEIK

SEQ ID NO: 69 - protein, 141 aa, Homo sapiens
  SIGNAL (1)...(19)
MEFGLSWVFLVALLRGVQCQVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDRHSSSWYYGMDVWGQGTTVTVSS

SEQ ID NO: 70 - protein, 137 aa, Homo sapiens
  SIGNAL (1)...(19)
MEFGLSWVFLVALLRGVQCQVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDAKLLMLLISGAKGQWSPSL

SEQ ID NO: 71 - protein, 5 aa, Artificial sequence
  synthetic construct, Hinge-link region
LLGGP

SEQ ID NO: 72 - protein, 5 aa, Artificial sequence
  synthetic construct, Hinge-link region
LEGGP

SEQ ID NO: 73 - protein, 109 aa, Artificial Sequence
  synthetic construct, consensus sequence for kappa chain
DIQMTQSPSSLSASVGDRVTITCRASQGISSYLAWYQQKPGKAPKLLIYAASSLQSGVPSRFSGSGSGTDFTLTISSLQPEDFATYYCQQHYTTPPTFGQGTKVEIKRT

SEQ ID NO: 74 - protein, 114 aa, Artificial Sequence
  synthetic construct, consensus sequence for kappa chain
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCQQHYTTPPTFGQGTKVEIKRT

SEQ ID NO: 75 - protein, 110 aa, Artificial Sequence
  synthetic construct, consensus sequence for kappa chain
DIVLTQSPATLSLSPGERATLSCRASQSVSSSYLAWYQQKPGQAPRLLIYGASSRATGVPARFSGSGSGTDFTLTISSLEPEDFAVYYCQQHYTTPPTFGQGTKVEIKRT

SEQ ID NO: 76 - protein, 115 aa, Artificial Sequence
  synthetic construct, consensus sequence for kappa chain
DIVMTQSPDSLAVSLGERATINCRSSQSVLYSSNNKNYLAWYQQKPGQPPKLLIYWASTRESGVPDRFSGSGSGTDFTLTISSLQAEDVAVYYCQQHYTTPPTFGQGTKVEIKRT

SEQ ID NO: 77 - protein, 109 aa, Artificial Sequence
  synthetic construct, consensus sequence for lambda chain
QSVLTQPPSVSGAPGQRVTISCSGSSSNIGSNYVSWYQQLPGTAPKLLIYDNNQRPSGVPDRFSGSKSGTSASLAITGLQSEDEADYYCQQHYTTPPVFGGGTKLTVLG

SEQ ID NO: 78 - protein, 110 aa, Artificial Sequence
  synthetic construct, consensus sequence for lambda chain
QSALTQPASVSGSPGQSITISCTGTSSDVGGYNYVSWYQQHPGKAPKLMIYDVSNRPSGVSNRFSGSKSGNTASLTISGLQAEDEADYYCQQHYTTPPVFGGGTKLTVLG

SEQ ID NO: 79 - protein, 107 aa, Artificial Sequence
  synthetic construct, consensus sequence for lambda chain
SYELTQPPSVSVAPGQTARISCSGDALGDKYASWYQQKPGQAPVLVIYDDSDRPSGIPERFSGSNSGNTATLTISGTQAEDEADYYCQQHYTTPPVFGGGTKLTVLG

SEQ ID NO: 80 - protein, 120 aa, Artificial Sequence
  synthetic construct, consensus sequence for heavy chain framework region
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARWGGDGFYAMDYWGQGTLVTVSS

SEQ ID NO: 81 - protein, 120 aa, Artificial Sequence
  synthetic construct, consensus sequence for heavy chain framework region
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCARWGGDGFYAMDYWGQGTLVTVSS

SEQ ID NO: 82 - protein, 121 aa, Artificial Sequence
  synthetic construct, consensus sequence for heavy chain framework region
QVQLKESGPALVKPTQTLTLTCTFSGFSLSTSGVGVGWIRQPPGKALEWLALIDWDDDKYYSTSLKTRLTISKDTSKNQVVLTMTNMDPVDTATYYCARWGGDGFYAMDYWGQGTLVTVSS

SEQ ID NO: 83 - protein, 120 aa, Artificial Sequence
  synthetic construct, consensus sequence for heavy chain framework region
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARWGGDGFYAMDYWGQGTLVTVSS

SEQ ID NO: 84 - protein, 119 aa, Artificial Sequence
  synthetic construct, consensus sequence for heavy chain framework region
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARWGGDGFYAMDYWGQGTLVTVSS

SEQ ID NO: 85 - protein, 120 aa, Artificial Sequence
  synthetic construct, consensus sequence for heavy chain framework region
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARWGGDGFYAMDYWGQGTLVTVSS

SEQ ID NO: 86 - protein, 123 aa, Artificial Sequence
  synthetic construct, consensus sequence for heavy chain framework region
QVQLQQSGPGLVKPSQTLSLTCAISGDSVSSNSAAWNWIRQSPGRGLEWLGRTYYRSKWYNDYAVSVKSRITINPDTSKNQFSLQLNSVTPEDTAVYYCARWGGDGFYAMDYWGQGTLVTVSS

SEQ ID NO: 87 - protein, 17 aa, Artificial Sequence
  synthetic construct, generic signal sequence
MGWSCIILFLVATGAHS

SEQ ID NO: 88 - protein, 450 aa, Artificial Sequence
  synthetic construct, variable heavy chain of 12A11v.1 linked to Fc portion of an IgG1 isotype
QVQLVESGGGVVQPGRSLRLSCAFSGFSLSTSGMSVGWIRQAPGKGLEWLAHIWWDDDKYYNPSLKSRLTISKDTSKNTVYLQMNSLRAEDTAVYYCARRTTTADYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 447 aa, Artificial Sequence
  synthetic construct, variable heavy chain of 12A11v.1 linked to the Fc portion of an IgG4 isotype
QVQLVESGGGVVQPGRSLRLSCAFSGFSLSTSGMSVGWIRQAPGKGLEWLAHIWWDDDKYYNPSLKSRLTISKDTSKNTVYLQMNSLRAEDTAVYYCARRTTTADYFAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 90 - protein, 450 aa, Artificial Sequence
  synthetic construct, variable heavy chain of 12A11v3.1 linked to IgG1 constant region
QVQLVESGGGVVQPGRSLRLSCAFSGFTLSTSGMSVGWIRQAPGKGLEWLAHIWWDDDKYYNPSLKSRFTISKDNSKNTLYLQMNSLRAEDTAVYYCARRTTTADYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 567 aa, Artificial Sequence [sequence omitted]

SEQ ID NO: 92 - DNA, 993 bp, Artificial Sequence
  Synthetic construct, Heavy chain constant region DNA (codons only)
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 93 - protein, 330 aa, Artificial Sequence [sequence omitted]

SEQ ID NO: 94 - DNA, 324 bp, Artificial Sequence
  Synthetic construct, light chain constant region DNA (codons only)
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 95 - protein, 106 aa, Artificial Sequence
  Synthetic construct, light chain constant region protein
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC